SEQUENCE LISTING (198 sequences)

SEQ ID NO: 1 - protein, 93 aa, synthetic construct
  REGION 1..93: note = Synthetic: FRB*
ILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 2 - protein, 93 aa, synthetic construct [sequence omitted]

SEQ ID NO: 3 - protein, 107 aa, synthetic construct
  REGION 1..107: note = Synthetic: FKBP12
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 4 - protein, 107 aa, synthetic construct
  REGION 1..107: note = Synthetic: FKBP12 F36V
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 5 - protein, 107 aa, synthetic construct
  REGION 1..107: note = Synthetic: CH3-Ab (S354C T366W)
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 107 aa, synthetic construct
  REGION 1..107: note = Synthetic: CH3-IA (Y349C T366S L368A Y407V)
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 7 - protein, 107 aa, synthetic construct
  REGION 1..107: note = Synthetic: CH3-DE (L351D L368E)
GQPREPQVYTDPPSRDELTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 107 aa, synthetic construct
  REGION 1..107: note = Synthetic: CH3-KK (L351K T366K)
GQPREPQVYTKPPSRDELTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 106 aa, synthetic construct
  REGION 1..106: note = Synthetic: CH3-GA (SEED 1)
GQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDR

SEQ ID NO: 10 - protein, 106 aa, synthetic construct
  REGION 1..106: note = Synthetic: CH3-AG (SEED 2)
GQPFRPEVHLLPPSREEMTKNQWSLTCLARGFYPKDIAVEWESNGQPENNYKTTPSRQEPSQGTTTFAVTSKLTVDKSRWQQGNWFSCSVMHEALHNHYTQKTISL

SEQ ID NO: 11 - protein, 5 aa, synthetic construct
  REGION 1..5: note = Synthetic: 1xG4S linker
GGGGS

SEQ ID NO: 12 - protein, 10 aa, synthetic construct
  REGION 1..10: note = Synthetic: 2xG4S linker
GGGGSGGGGS

SEQ ID NO: 13 - protein, 15 aa, synthetic construct
  REGION 1..15: note = Synthetic: 3xG4S linker
GGGGSGGGGSGGGGS

SEQ ID NO: 14 - protein, 20 aa, synthetic construct
  REGION 1..20: note = Synthetic: 4xG4S linker
GGGGSGGGGSGGGGSGGGGS

SEQ ID NO: 15 - protein, 25 aa, synthetic construct
  REGION 1..25: note = Synthetic: 5xG4S linker
GGGGSGGGGSGGGGSGGGGSGGGGS

SEQ ID NO: 16 - protein, 5 aa, synthetic construct
  REGION 1..5: note = Synthetic: Linker
DGGGS

SEQ ID NO: 17 - protein, 5 aa, synthetic construct
  REGION 1..5: note = Synthetic: Linker
TGEKP

SEQ ID NO: 18 - protein, 4 aa, synthetic construct
  REGION 1..4: note = Synthetic: Linker
GGRR

SEQ ID NO: 19 - protein, 14 aa, synthetic construct
  REGION 1..14: note = Synthetic: Linker
EGKSSGSGSESKVD

SEQ ID NO: 20 - protein, 18 aa, synthetic construct
  REGION 1..18: note = Synthetic: Linker
KESGSVSSEQLAQFRSLD

SEQ ID NO: 21 - protein, 8 aa, synthetic construct
  REGION 1..8: note = Synthetic: Linker
GGRRGGGS

SEQ ID NO: 22 - protein, 9 aa, synthetic construct
  REGION 1..9: note = Synthetic: Linker
LRQRDGERP

SEQ ID NO: 23 - protein, 12 aa, synthetic construct
  REGION 1..12: note = Synthetic: Linker
LRQKDGGGSERP

SEQ ID NO: 24 - protein, 16 aa, synthetic construct
  REGION 1..16: note = Synthetic: Linker
LRQKDGGGSGGGSERP

SEQ ID NO: 25 - protein, 18 aa, synthetic construct
  REGION 1..18: note = Synthetic: Linker
GSTSGSGKPGSGEGSTKG

SEQ ID NO: 26 - protein, 18 aa, synthetic construct
  REGION 1..18: note = Synthetic: linker
GSTSGSGKSSEGSGSTKG

SEQ ID NO: 27 - protein, 14 aa, synthetic construct
  REGION 1..14: note = Synthetic: linker
GSTSGSGKSSEGKG

SEQ ID NO: 28 - protein, 15 aa, synthetic construct
  REGION 1..15: note = Synthetic: linker
GSTSGSGKPGSGEGS

SEQ ID NO: 29 - protein, 4 aa, synthetic construct
  REGION 1..4: note = Synthetic: linker
GGGS

SEQ ID NO: 30 - protein, 4 aa, synthetic construct
  REGION 1..4: note = Synthetic: yuTCR linker (uLNK)
LEKT

SEQ ID NO: 31 - protein, 9 aa, synthetic construct
  REGION 1..9: note = Synthetic: uLNK + G4S linker
LEKTGGGGS

SEQ ID NO: 32 - protein, 185 aa, synthetic construct
  REGION 1..185: note = Synthetic: CD3e
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 33 - protein, 160 aa, synthetic construct
  REGION 1..160: note = Synthetic: CD3y
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 34 - protein, 170 aa, synthetic construct
  REGION 1..170: note = Synthetic: CD3d
EHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 35 - protein, 66 aa, synthetic construct
  REGION 1..66: note = Synthetic: FceR1y - v1
GEPLCYILDAILFLYGIVLTLLYCRLKIQVRKAAITSYEKSDGVYTGLSTRNQETYETLKHEKPPQ

SEQ ID NO: 36 - protein, 68 aa, synthetic construct
  REGION 1..68: note = Synthetic: FceR1y - v2
LGEPQLCYILDAILFLYGIVLTLLYCRLKIQVRKAAITSYEKSDGVYTGLSTRNQETYETLKHEKPPQ

SEQ ID NO: 37 - protein, 194 aa, synthetic construct
  REGION 1..194: note = Synthetic: Iga/CD79a (BCR)
LWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 38 - protein, 79 aa, synthetic construct
  REGION 1..79: note = Synthetic: Igb/CD79b (BCR)
MARLALSPVPSHWMVALLLLLSGTEPTTRPVGFALISSCLGPAALPPACSTLLLCLSLFTACLPLMALGPGSVGVSCTW

SEQ ID NO: 39 - protein, 75 aa, synthetic construct
  REGION 1..75: note = Synthetic: DAP10
QTTPGERSSLPAFYPGTSGSCSGCGSLSLPLLAGLVAADAVASLLIVGAVFLCARPRRSPAQEDGKVYINMPGRG

SEQ ID NO: 40 - protein, 92 aa, synthetic construct
  REGION 1..92: note = Synthetic: DAP12
LRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 41 - protein, 15 aa, synthetic construct
  REGION 1..15: note = Synthetic: Minimal CD4 hinge
SNIKVLPTWSTPVQP

SEQ ID NO: 42 - protein, 17 aa, synthetic construct
  REGION 1..17: note = Synthetic: Minimal CD28 hinge
KGKHLCPSPLFPGPSKP

SEQ ID NO: 43 - protein, 12 aa, synthetic construct
  REGION 1..12: note = Synthetic: IgG4 hinge
ESKYGPPCPSCP

SEQ ID NO: 44 - protein, 45 aa, synthetic construct
  REGION 1..45: note = Synthetic: CD8 hinge
TTTPAPRPPTPAPTIASQPLSLRPEACRPAAGGAVHTRGLDFACD

SEQ ID NO: 45 - protein, 22 aa, synthetic construct
  REGION 1..22: note = Synthetic: CD4 transmembrane
MALIVLGGVAGLLLFIGLGIFF

SEQ ID NO: 46 - protein, 27 aa, synthetic construct
  REGION 1..27: note = Synthetic: CD28 transmembrane
FWVLVVVGGVLACYSLLVTVAFIIFWV

SEQ ID NO: 47 - protein, 27 aa, synthetic construct
  REGION 1..27: note = Synthetic: CD8 transmembrane
AGTGSDIYIWAPLAGTCGVLLLSLVIT

SEQ ID NO: 48 - protein, 4 aa, synthetic construct
  REGION 1..4: note = Synthetic: Truncated intracellular CD4 v1
CVRC

SEQ ID NO: 49 - protein, 10 aa, synthetic construct
  REGION 1..10: note = Synthetic: Truncated intracellular CD4 v2
CVRCRHRRRQ

SEQ ID NO: 50 - protein, 246 aa, synthetic construct
  REGION 1..246: note = Synthetic: anti-BCMA_scFv_1
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 51 - protein, 246 aa, synthetic construct
  REGION 1..246: note = Synthetic: anti-BCMA_scFv_2
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 52 - protein, 247 aa, synthetic construct
  REGION 1..247: note = Synthetic: anti-BCMA_scFv_3
DIQMTQSPSSLSASVGDRITITCRASQDIRNYLGWYQQKPGKAPKVLIFAASSLQSGVPSRFSGSGSGTDFTLTISSLQPEDFATYYCLQDYIYPWTFAQGTKVEIKGGGGSGGGGSGGGGSQVQLVESGGGVVQPGRSLRLSCAASGFTFSSYGMHWVRQAPGKGLEWVAVISYDGRNKNYADSVKGRFTISRDNSKNTLYLQMNSLRAEDTAVYYCAREGEATYYDILTGPFDYWGQGTLVTVSS

SEQ ID NO: 53 - protein, 242 aa, synthetic construct
  REGION 1..242: note = Synthetic: anti-CD19_scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 54 - protein, 243 aa, synthetic construct
  REGION 1..243: note = Synthetic: anti-CD20_scFv_1
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 55 - protein, 243 aa, synthetic construct
  REGION 1..243: note = Synthetic: anti-CD20_scFv_2
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKR

SEQ ID NO: 56 - protein, 249 aa, synthetic construct
  REGION 1..249: note = Synthetic: anti-CD20_scFv_3
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSGGGGSGGGGSGGGGSDIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 57 - protein, 236 aa, synthetic construct
  REGION 1..236: note = Synthetic: anti-CD22_scFv
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 58 - protein, 245 aa, synthetic construct
  REGION 1..245: note = Synthetic: anti-CD33_scFv
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKGGGGSGGGGSGGGGSQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 59 - protein, 248 aa, synthetic construct
  REGION 1..248: note = Synthetic: anti-CD79A_scFv
DVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKRGGGGSGGGGSGGGGSQVQLQQSGPELVKPGASVKISCKASGYTFSTSWMNWVKQRPGQGLEWIGRIYPGDGDTNYNGKFKGKATLTADKSSNTAYMQLSSLTSVDSAVYFCERFYYGNTFAMDYWGQGTSVTVSS

SEQ ID NO: 60 - protein, 238 aa, synthetic construct
  REGION 1..238: note = Synthetic: anti-CD79B_scFv_1
QIQLVQSGPELKKPGETVKISCKASGYTFTDYSMHWVKQAPGEGLKWMGWINTETGEPTYADDFKGRFAFSLETSASTAYLQINNLKNEDTATYFCYYGSYWGQGTLVTVSAGGGGSGGGGSGGGGSDIVLTQSPASLAVSLGQRATISCKASQSVDYDGDGYMDWYQQKPGQPPKLLIFAASNLKSGIPARFSGSGSGTDFTLNIHPVEEEDAATYYCQQTNEYPWTFGGGTKLEIK

SEQ ID NO: 61 - protein, 244 aa, synthetic construct
  REGION 1..244: note = Synthetic: Anti-CD79B_scFv_2
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRGGGGSGGGGSGGGGSEVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSS

SEQ ID NO: 62 - protein, 242 aa, synthetic construct
  REGION 1..242: note = Synthetic: anti-B7H3_scFv
DIVMTQSHKFMSTSIGARVSITCKASQDVRTAVAWYQQKPGQSPKLLIYSASYRYTGVPDRFTGSGSGTDFTFTISSVQAEDLAVYYCQQHYGTPPWTFGGGTKLEIKGGGGSGGGGSGGGGSEVQLVESGGGLVKPGGSLKLSCEASRFTFSSYAMSWVRQTPEKRLEWVAAISGGGRYTYYPDSMKGRFTISRDNAKNFLYLQMSSLRSEDTAMYYCARHYDGYLDYWGQGTTLTVSSTR

SEQ ID NO: 63 - protein, 250 aa, synthetic construct
  REGION 1..250: note = Synthetic: anti-Muc16_scFv
VKLQESGGGFVKPGGSLKVSCAASGFTFSSYAMSWVRLSPEMRLEWVATISSAGGYIFYSDSVQGRFTISRDNAKNTLHLQMGSLRSGDTAMYYCARQGFGNYGDYYAMDYWGQGTTVTVSSGGGGSGGGGSGGGGSDIELTQSPSSLAVSAGEKVTMSCKSSQSLLNSRTRKNQLAWYQQKPGQSPELLIYWASTRQSGVPDRFTGSGSGTDFTLTISSVQAEDLAVYYCQQSYNLLTFGPGTKLEVKR

SEQ ID NO: 64 - protein, 243 aa, synthetic construct
  REGION 1..243: note = Synthetic: anti-HER2_scFv
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGSGKPGSGEGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 65 - protein, 241 aa, synthetic construct
  REGION 1..241: note = Synthetic: anti-EGFR_scFv
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSS

SEQ ID NO: 66 - protein, 236 aa, synthetic construct
  REGION 1..236: note = Synthetic: anti-FN-EDB_scFv
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 67 - protein, 246 aa, synthetic construct
  REGION 1..246: note = Synthetic: anti-CLDN18.2_scFv
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 68 - protein, 241 aa, synthetic construct
  REGION 1..241: note = Synthetic: anti-DLL3_scFv
QVQLQESGPGLVKPSETLSLTCTVSGDSISSYYWTWIRQPPGKGLEWIGYIYYSGTTNYNPSLKSRVTISVDTSKSQFSLKLSSVTAADTAVYYCASIAVRGFFFDYWGQGTLVTVSSGGGGSGGGGSGGGGSEIVLTQSPGTLSLSPGERATLSCRASQSVSSSYLAWYQQKPGQAPRLLIYGASTRATGIPDRFSGSGSGTDFTLTISRLEPEDFAVYYCQQYGTSPLTFGGGTKVEIK

SEQ ID NO: 69 - protein, 249 aa, synthetic construct
  REGION 1..249: note = Synthetic: anti-FLT3_scFv_1
QVTLKESGPVLVKPTETLTLTCTVSGFSLINARMGVSWIRQPPGKALEWLAHIFSNAEKSYRTSLKSRLTISKDTSKSQVVLTMTNMDPVDTATYYCARIPGYGGNGDYHYYGMDVWGQGTTVTVSSGGGGSGGGGSGGGGSDIQMTQSPSSLSASLGDRVTITCRASQGIRNDLGWYQQKPGKAPKRLIYASSTLQSGVPSRFSGSGSGTEFTLTISSLQPEDFATYYCLQHNNFPWTFGQGTKVEIK

SEQ ID NO: 70 - protein, 243 aa, synthetic construct
  REGION 1..243: note = Synthetic: anti-FLT3_scFv_2
EVQLVESGGGLVQPGGSLRLSCAASGFTFSSYGMHWVRQAPGKGLEWVAVISYDGSNKYYADSVKGRFTISRDNSKNTLYLQMNSLRAEDTAVYYCANLAPWAAYWGQGTLVTVSSGGGGSGGGGSGGGGSEIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPHTFGQGTKLEIK

SEQ ID NO: 71 - protein, 239 aa, synthetic construct
  REGION 1..239: note = Synthetic: anti-ROR1 scFv
DIQMTQSPSFLSASVGDRVTINCKASQNIDRYLNWYQQKLGEAPKRLLYNTNKLQTGIPSRFSGSGSATDFTLTISSLQPEDFATYFCLQYNSLPLTFGSGTKLEIKGGGGSGGGGSGGGGSEVQLVESGGGLVQPGRSLKLSCAASGFIFSEHNMAWVRQAPKKGLEWVATISDDGRNTYYRDSMRGRFTISRENARSTLYLQLDSLRSEDTATYYCASHRYNLFDSWGQGVMVTVSS

SEQ ID NO: 72 - protein, 116 aa, synthetic construct
  REGION 1..116: note = Synthetic: anti-CD33_VHH_1
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKP

SEQ ID NO: 73 - protein, 120 aa, synthetic construct
  REGION 1..120: note = Synthetic: anti-CD33_VHH_2
EVQLVESGGGEVQPGGSLRLSCAASGRTFSGYIMGWFRQAPGKERELVARISGNNLSTEYAESVKGRFTISRDNAKNTLYLQMSSLRAEDTAVYYCAAEYDYSSGDFVYWGQGTLVTVKP

SEQ ID NO: 74 - protein, 120 aa, synthetic construct
  REGION 1..120: note = Synthetic: anti-CD33_VHH_3
EVQLVESGGGEVQPGGSLRLSCAASGSTLNIDHIGWYRQAPGKERELVGVISSGAGPNYAESVKGRFTISRDNAKNTVYLQMSSLRAEDTAVYYCNAWIDYGSGLPQNYWGQGTLVTVKP

SEQ ID NO: 75 - protein, 120 aa, synthetic construct
  REGION 1..120: note = Synthetic: anti-CLL1_VHH_1
EVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKP

SEQ ID NO: 76 - protein, 116 aa, synthetic construct
  REGION 1..116: note = Synthetic: anti-CLL1_VHH_2
EVQLVESGGGEVQPGGSLRLSCAASGLLFSIYDMNWYRQAPGKEREWVAGITNNGYSTAYAESVKGRFTISRDNAKNTVYLQMSSLRAEDTAVYYCHTDEWGREYWGQGTLVTVKP

SEQ ID NO: 77 - protein, 125 aa, synthetic construct
  REGION 1..125: note = Synthetic: anti-CD123_VHH_1
EVQLVESGGGEVQPGGSLRLSCTASGRAINMYAMGWFRQAPGKEREFVAAINWNGAYTQYAESVKGRFTISRDNAKNTLYLQMSSLRAEDTAVYYCSADADYNTYVSPNKRVSYWGQGTLVTVKP

SEQ ID NO: 78 - protein, 126 aa, synthetic construct
  REGION 1..126: note = Synthetic: anti-CD123_VHH_2
EVQLVESGGGLVQPGGSLRLSCAASGRAINAYNMGWFRQAPGKGREFVSAINWNAARTYYAESVKGRFTISRDNAKNTLYLQMSSLRAEDTAVYYCAASGRWSAAVPSGEDQYNFWGQGTLVTVKP

SEQ ID NO: 79 - protein, 121 aa, synthetic construct
  REGION 1..121: note = Synthetic: anti-CD20_VHH
QVQLQESGGGLVQAGGSLRLSCAASGRTFSNYNMGWFRQAPGKEREFVAAIDWSGGSPYYAASVRGRFTISRDNAENTVYLQMNSLKPEDTAVYYCAAPLSYGSTWLADYWGQGTQVTVSS

SEQ ID NO: 80 - protein, 124 aa, synthetic construct
  REGION 1..124: note = Synthetic: anti-EGFR_VHH
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 81 - protein, 119 aa, synthetic construct
  REGION 1..119: note = Synthetic: anti-BCMA_VHH_1
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSS

SEQ ID NO: 82 - protein, 118 aa, synthetic construct
  REGION 1..118: note = Synthetic: anti-BCMA_VHH_2
EVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSS

SEQ ID NO: 83 - protein, 117 aa, synthetic construct
  REGION 1..117: note = Synthetic: anti-CD19_VHH
QVKLEESGGELVQPGGPLRLSCAASGNIFSINRMGWYRQAPGKQRAFVASITVRGITNYADSVKGRFTISVDKSKNTIYLQMNALKPEDTAVYYCNAVSSNRDPDYWGQGTQVTVSS

SEQ ID NO: 84 - protein, 114 aa, synthetic construct
  REGION 1..114: note = Synthetic: PD1 ectodomain
NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTE

SEQ ID NO: 85 - protein, 114 aa, synthetic construct
  REGION 1..114: note = Synthetic: PD1 high affinity ectodomain
NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKVQIKESLRAELRVTE

SEQ ID NO: 86 - protein, 135 aa, synthetic construct
  REGION 1..135: note = Synthetic: Human A Proliferation-Inducing Ligand (APRIL)
SVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 87 - protein, 435 aa, synthetic construct
  REGION 1..435: note = Synthetic: Trimerized human APRIL
SVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGGGGSGGGGSGGGGSSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGGGGSGGGGSGGGGSSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 88 - protein, 134 aa, synthetic construct
  REGION 1..134: note = Synthetic: NKG2D membrane protein
FNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 89 - protein, 9 aa, synthetic construct
  REGION 1..9: note = Synthetic: anti-CD33_VHH_1 CDR1
RSSGIDVMG

SEQ ID NO: 90 - protein, 9 aa, synthetic construct
  REGION 1..9: note = Synthetic: anti-CD33_VHH_1 CDR2
EISGVGDTN

SEQ ID NO: 91 - protein, 9 aa, synthetic construct
  REGION 1..9: note = Synthetic: anti-CD33_VHH_1 CDR3
HSFLDLVGA

SEQ ID NO: 92 - protein, 10 aa, synthetic construct
  REGION 1..10: note = Synthetic: anti-CLL1_VHH_1 CDR1
GFLFSIYDMN

SEQ ID NO: 93 - protein, 10 aa, synthetic construct
  REGION 1..10: note = Synthetic: anti-CLL1_VHH_1 CDR2
GITNNGYSTA

SEQ ID NO: 94 - protein, 11 aa, synthetic construct
  REGION 1..11: note = Synthetic: anti-CLL1_VHH_1 CDR3
DLTKAYDVEYA

SEQ ID NO: 95 - protein, 19 aa, synthetic construct
  REGION 1..19: note = Synthetic: IgK signal sequence
ETPAQLLFLLLLWLPDTTG

SEQ ID NO: 96 - protein, 20 aa, synthetic construct
  REGION 1..20: note = Synthetic: CD8 signal sequence
ALPVTALLLPLALLLHAARP

SEQ ID NO: 97 - protein, 22 aa, synthetic construct
  REGION 1..22: note = Synthetic: PD1 signal sequence
QIPQAPWPVVWAVLQLGWRPGW

SEQ ID NO: 98 - protein, 42 aa, synthetic construct
  REGION 1..42: note = Synthetic: 41BB intracellular signaling domain
KRGRKKLLYIFKQPFMRPVQTTQEEDGCSCRFPEEEEGGCEL

SEQ ID NO: 99 - protein, 41 aa, synthetic construct
  REGION 1..41: note = Synthetic: CD28 intracellular signaling domain
RSKRSRLLHSDYMNMTPRRPGPTRKHYQPYAPPRDFAAYRS

SEQ ID NO: 100 - protein, 40 aa, synthetic construct
  REGION 1..40: note = Synthetic: CD4 coreceptor domain
CVRCRHRRRQAERMSQIKRLLSEKKTCQCPHRFQKTCSPI

SEQ ID NO: 101 - protein, 32 aa, synthetic construct
  REGION 1..32: note = Synthetic: CD8 coreceptor domain
LYCNHRNRRRVCKCPRPVVKSGDKPSLSARYV

SEQ ID NO: 102 - protein, 235 aa, synthetic construct
  REGION 1..235: note = Synthetic: LAT domain
HCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 103 - protein, 195 aa, synthetic construct
  REGION 1..195: note = Synthetic: IL7 receptor alpha signaling domain
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 104 - protein, 72 aa, synthetic construct
  REGION 1..72: note = Synthetic: IL2 receptor beta signaling domain, truncated
NCRNTGPWLKKVLKCNTPDPSKFFSQLSSEHGGDVQKWLSSPFPSSSFSPGGLAPEISPLEVLERDKVTQLL

SEQ ID NO: 105 - protein, 286 aa, synthetic construct
  REGION 1..286: note = Synthetic: IL2 receptor beta signaling domain
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 106 - protein, 86 aa, synthetic construct
  REGION 1..86: note = Synthetic: Common gamma chain signaling domain
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 107 - protein, 291 aa, synthetic construct
  REGION 1..291: note = Synthetic: FRB.3xG4S.CD3e
WHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 108 - protein, 266 aa, synthetic construct
  REGION 1..266: note = Synthetic: FRB.3xG4S.CD3g
WHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSGGGGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 109 - protein, 295 aa, synthetic construct
  REGION 1..295: note = Synthetic: FKBP12.G4S.CD3e
QVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 110 - protein, 281 aa, synthetic construct
  REGION 1..281: note = Synthetic: FRB.G4S.CD3e
WHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 111 - protein, 291 aa, synthetic construct
  REGION 1..291: note = Synthetic: FRBstar.3xG4S.CD3e
WHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 112 - protein, 307 aa, synthetic construct
  REGION 1..307: note = Synthetic: IACH3.3xG4S.CD3e
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 113 - protein, 306 aa, synthetic construct
  REGION 1..306: note = Synthetic: CH3AG.3xG4S.CD3e
GQPFRPEVHLLPPSREEMTKNQWSLTCLARGFYPKDIAVEWESNGQPENNYKTTPSRQEPSQGTTTFAVTSKLTVDKSRWQQGNWFSCSVMHEALHNHYTQKTISLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 114 - protein, 307 aa, synthetic construct
  REGION 1..307: note = Synthetic: CH3DE.3xG4S.CD3e
GQPREPQVYTDPPSRDELTKNQVSLTCEVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 115 - protein, 167 aa, synthetic construct
  REGION 1..167: note = Synthetic: FRBstar.2xG4S.FCER1G
WHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGEPLCYILDAILFLYGIVLTLLYCRLKIQVRKAAITSYEKSDGVYTGLSTRNQETYETLKHEKPPQ

SEQ ID NO: 116 - protein, 282 aa, synthetic construct
  REGION 1..282: note = Synthetic: antiCD33.G4S.FKBP.minCD28hinge.CD28tm.CD4ic
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEKGKHLCPSPLFPGPSKPFWVLVVVGGVLACYSLLVTVAFIIFWVCVRCRHRRRQ

SEQ ID NO: 117 - protein, 275 aa, synthetic construct
  REGION 1..275: note = Synthetic: antiCD33.G4S.FKBP.minCD4hinge.CD4tm.CD4ic
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 118 - protein, 263 aa, synthetic construct
  REGION 1..263: note = Synthetic: antiCD33.G4S.FKBP.GGR.CD4tm.CD4ic
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 119 - protein, 264 aa, synthetic construct
  REGION 1..264: note = Synthetic: FRB.2xG4S.antiCD33.minCD4hinge.CD4tm.CD4ic
WHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPSNIKVLPTWSTPVQPMALIVLGGVAGLLLFIGLGIFFCVRCRHRRRQ

SEQ ID NO: 120 - protein, 280 aa, synthetic construct
  REGION 1..280: note = Synthetic: FKBP.2xG4S.antiCD33.minCD4hinge.CD4tm.CD4ic
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGGGSGGGGSEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPSNIKVLPTWSTPVQPMALIVLGGVAGLLLFIGLGIFFCVRCRHRRRQ

SEQ ID NO: 121 - protein, 279 aa, synthetic construct
  REGION 1..279: note = Synthetic: antiCLL1.G4S.FKBP.minCD4hinge.CD4tm.CD4ic
EVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 122 - protein, 400 aa, synthetic construct
  REGION 1..400: note = Synthetic: antiCLL1.G4S.antiCD33.G4S.FKBP.minCD4hinge.CD4tm.CD4ic
EVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKPGGGGSEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 123 - protein, 268 aa, synthetic construct
  REGION 1..268: note = Synthetic: FRB.2xG4S.antiCLL1.minCD4hinge.CD4tm.CD4ic
WHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSEVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKPSNIKVLPTWSTPVQPMALIVLGGVAGLLLFIGLGIFFCVRCRHRRRQ

SEQ ID NO: 124 - protein, 390 aa, synthetic construct
  REGION 1..390: note = Synthetic: antiCLL1.G4S.FRB.2xG4S.antiCD33.minCD4hinge.CD4tm.CD4ic
EVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKPGGGGSLWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPSNIKVLPTWSTPVQPMALIVLGGVAGLLLFIGLGIFFCVRCRHRRRQ

SEQ ID NO: 125 - protein, 310 aa, synthetic construct
  REGION 1..310: note = Synthetic: antiCD33.G4S.FKBP.minCD4hinge.CD4tm.CD28
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRSKRSRLLHSDYMNMTPRRPGPTRKHYQPYAPPRDFAAYRS

SEQ ID NO: 126 - protein, 311 aa, synthetic construct
  REGION 1..311: note = Synthetic: antiCD33.G4S.FKBP.minCD4hinge.CD4tm.41BB
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCKRGRKKLLYIFKQPFMRPVQTTQEEDGCSCRFPEEEEGGCEL

SEQ ID NO: 127 - protein, 275 aa, synthetic construct
  REGION 1..275: note = Synthetic: antiCD33.G4S.AbCH3.minCD4hinge.CD4tm.CD4ic
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSNIKVLPTWSTPVQPMALIVLGGVAGLLLFIGLGIFFCVRCRHRRRQ

SEQ ID NO: 128 - protein, 274 aa, synthetic construct
  REGION 1..274: note = Synthetic: antiCD33.G4S.CH3GA.minCD4hinge.CD4tm.CD4ic
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSNIKVLPTWSTPVQPMALIVLGGVAGLLLFIGLGIFFCVRCRHRRRQ

SEQ ID NO: 129 - protein, 275 aa, synthetic construct
  REGION 1..275: note = Synthetic: antiCD33.G4S.CH3KK.minCD4hinge.CD4tm.CD4ic
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGQPREPQVYTKPPSRDELTKNQVSLKCLVKGFYPSDIAVEWESNGQPENNYKTTPPVLDSDGSFFLYSKLTVDKSRWQQGNVFSCSVMHEALHNHYTQKSLSLSPGKSNIKVLPTWSTPVQPMALIVLGGVAGLLLFIGLGIFFCVRCRHRRRQ

SEQ ID NO: 130 - protein, 305 aa, synthetic construct
  REGION 1..305: note = Synthetic: antiCD33.G4S.FKBP.minCD4hinge.CD4tm.CD4ic-full
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQAERMSQIKRLLSEKKTCQCPHRFQKTCSPI

SEQ ID NO: 131 - protein, 297 aa, synthetic construct
  REGION 1..297: note = Synthetic: antiCD33.G4S.FKBP.minCD4hinge.CD4tm.CD8ic-full
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFLYCNHRNRRRVCKCPRPVVKSGDKPSLSARYV

SEQ ID NO: 132 - protein, 401 aa, synthetic construct
  REGION 1..401: note = Synthetic: CD19scFv.G4S.FKBP.minCD4hinge.CD4tm.CD4ic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 133 - protein, 306 aa, synthetic construct
  REGION 1..306: note = Synthetic: PD1(A132V)ex.G4S.FKBP.minCD4hinge.CD4tm.CD4ic
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKVQIKESLRAELRVTERRAEVPTAHPSPSPRPAGQFQTLVGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 134 - protein, 306 aa, synthetic construct
  REGION 1..306: note = Synthetic: PD1ex.G4S.FKBP.minCD4hinge.CD4tm.CD4ic
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 135 - protein, 359 aa, synthetic construct
  REGION 1..359: note = Synthetic: antiCD33.G4S.FKBP.CD4htm.trIL2Rb
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 136 - protein, 398 aa, synthetic construct
  REGION 1..398: note = Synthetic: antiROR1.G4S.FKBP.minCD4hinge.CD4tm.CD4ic
DIQMTQSPSFLSASVGDRVTINCKASQNIDRYLNWYQQKLGEAPKRLLYNTNKLQTGIPSRFSGSGSATDFTLTISSLQPEDFATYFCLQYNSLPLTFGSGTKLEIKGGGGSGGGGSGGGGSEVQLVESGGGLVQPGRSLKLSCAASGFIFSEHNMAWVRQAPKKGLEWVATISDDGRNTYYRDSMRGRFTISRENARSTLYLQLDSLRSEDTATYYCASHRYNLFDSWGQGVMVTVSSGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 137 - protein, 325 aa, synthetic construct
  REGION 1..325: note = Synthetic: FRBstar.CD4htm.IL7Ra
ILWHEMWHEGLEEASRLYFGERNVKGMFEVLEPLHAMMERGPQTLKETSFNQAYGRDLMEAQEWCRKYMKSGNVKDLLQAWDLYYHVFRRISKSNIKVLPTWSTPVQPMALIVLGGVAGLLLFIGLGIFFKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 138 - protein, 216 aa, synthetic construct
  REGION 1..216: note = Synthetic: FRBstar.CD4htm.IL2Rg
ILWHEMWHEGLEEASRLYFGERNVKGMFEVLEPLHAMMERGPQTLKETSFNQAYGRDLMEAQEWCRKYMKSGNVKDLLQAWDLYYHVFRRISKSNIKVLPTWSTPVQPMALIVLGGVAGLLLFIGLGIFFERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 139 - protein, 641 aa, synthetic construct
  REGION 1..641: note = Synthetic: SR020 - CD8ss.FRB.CD3e.P2A.IgKss.antiCD3 3.FKBP.minCD28hinge.CD28tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEKGKHLCPSPLFPGPSKPFWVLVVVGGVLACYSLLVTVAFIIFWVCVRCRHRRRQ

SEQ ID NO: 140 - protein, 616 aa, synthetic construct
  REGION 1..616: note = Synthetic: SR021 - CD8ss.FRB.CD3g.P2A.IgKss.antiCD3 3.FKBP.minCD28hinge.CD28tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSGGGGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNSGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEKGKHLCPSPLFPGPSKPFWVLVVVGGVLACYSLLVTVAFIIFWVCVRCRHRRRQ

SEQ ID NO: 141 - protein, 634 aa, synthetic construct
  REGION 1..634: note = Synthetic: SR022 - CD8ss.FRB.CD3e.P2A.IgKss.antiCD3 3.FKBP.minCD4hinge.CD4tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 142 - protein, 609 aa, synthetic construct
  REGION 1..609: note = Synthetic: SR023 - CD8ss.FRB.CD3g.P2A.IgKss.antiCD3 3.FKBP.minCD4hinge.CD4tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSGGGGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNSGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 143 - protein, 622 aa, synthetic construct
  REGION 1..622: note = Synthetic: SR024 - CD8ss.FRB.CD3e.P2A.IgKss.antiCD33.FKBP.CD4tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 144 - protein, 628 aa, synthetic construct
  REGION 1..628: note = Synthetic: SR026 - CD8ss.FKBP.CD3e.P2A.IgKss.FRB.an tiCD33.minCD4hinge.CD4tm.CD4ic
ALPVTALLLPLALLLHAARPGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGLWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPSNIKVLPTWSTPVQPMALIVLGGVAGLLLFIGLGIFFCVRCRHRRRQ

SEQ ID NO: 145 - protein, 628 aa, synthetic construct
  REGION 1..628: note = Synthetic: SR028 - CD8ss.FRB.CD3e.P2A.IgKss.FKBP.an tiCD33.minCD4hinge.CD4tm.CD4ic
ALPVTALLLPLALLLHAARPGSLWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGGGSGGGGSEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPSNIKVLPTWSTPVQPMALIVLGGVAGLLLFIGLGIFFCVRCRHRRRQ

SEQ ID NO: 146 - protein, 634 aa, synthetic construct
  REGION 1..634: note = Synthetic: SR001 - CD8ss.FRBstar.CD3e.P2A.IgKss.ant iCD33.FKBP.minCD4hinge.CD4tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 147 - protein, 638 aa, synthetic construct
  REGION 1..638: note = Synthetic: SR003 - CD8ss.FRB.CD3e.P2A.IgKss.antiCLL 1.FKBP.minCD4hinge.CD4tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 148 - protein, 759 aa, synthetic construct
  REGION 1..759: note = Synthetic: SR004 - CD8ss.FRB.CD3e.P2A.IgKss.antiCLL 1.antiCD33.FKBP.minCD4hinge.CD4tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKPGGGGSEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 149 - protein, 632 aa, synthetic construct
  REGION 1..632: note = Synthetic: SR005 - CD8ss.FKBP.CD3e.P2A.IgKss.FRB.an tiCLL1.minCD4hinge.CD4tm.CD4ic
ALPVTALLLPLALLLHAARPGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGLWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSEVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKPSNIKVLPTWSTPVQPMALIVLGGVAGLLLFIGLGIFFCVRCRHRRRQ

SEQ ID NO: 150 - protein, 753 aa, synthetic construct
  REGION 1..753: note = Synthetic: SR006 - CD8ss.FKBP.CD3e.P2A.IgKss.antiCLL1.FRB. antiCD33.minCD4hinge.CD4tm.CD4ic
ALPVTALLLPLALLLHAARPGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKPGGGGSLWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPSNIKVLPTWSTPVQPMALIVLGGVAGLLLFIGLGIFFCVRCRHRRRQ

SEQ ID NO: 151 - protein, 759 aa, synthetic construct
  REGION 1..759: note = Synthetic: SR008 - CD8ss.FRBstar.CD3e.P2A.IgKss.antiCLL1. antiCD33.FKBP.minCD4hinge.CD4tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKPGGGGSEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 152 - protein, 795 aa, synthetic construct
  REGION 1..795: note = Synthetic: SR030 - CD8ss.FRBstar.CD3e.P2A.IgKss.antiCLL1. antiCD33.FKBP.minCD4hinge.CD4tm.41BB
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKPGGGGSEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCKRGRKKLLYIFKQPFMRPVQTTQEEDGCSCRFPEEEEGGCEL

SEQ ID NO: 153 - protein, 669 aa, synthetic construct
  REGION 1..669: note = Synthetic: SR001-28 CD8ss.FRBstar.CD3e.P2A.IgKss.an tiCD33.FKBP.minCD4hinge.CD4tm.CD28
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRSKRSRLLHSDYMNMTPRRPGPTRKHYQPYAPPRDFAAYRS

SEQ ID NO: 154 - protein, 680 aa, synthetic construct
  REGION 1..680: note = Synthetic: SR001-41BB CD8ss.FRBstar.CD3e.P2A.IgKss. antiCD33.FKBP.minCD4hinge.CD4tm.41BB
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCKRGRKKLLYIFKQPFMRPVQTTQEEDGCSCRFPEEEEGGCEL

SEQ ID NO: 155 - protein, 648 aa, synthetic construct
  REGION 1..648: note = Synthetic: SR292 - CD8ss.IACH3.CD3e.P2A.IgKss.antiC D33.AbCH3.minCD4hinge.CD4tm.41BB
ALPVTALLLPLALLLHAARPGSGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSNIKVLPTWSTPVQPMALIVLGGVAGLLLFIGLGIFFCVRCRHRRRQ

SEQ ID NO: 156 - protein, 646 aa, synthetic construct
  REGION 1..646: note = Synthetic: SR293 - CD8ss.CH3AG.CD3e.P2A.IgKss.antiC D33.CH3GA.minCD4hinge.CD4tm.CD4ic
ALPVTALLLPLALLLHAARPGSGQPFRPEVHLLPPSREEMTKNQWSLTCLARGFYPKDIAVEWESNGQPENNYKTTPSRQEPSQGTTTFAVTSKLTVDKSRWQQGNWFSCSVMHEALHNHYTQKTISLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSNIKVLPTWSTPVQPMALIVLGGVAGLLLFIGLGIFFCVRCRHRRRQ

SEQ ID NO: 157 - protein, 648 aa, synthetic construct
  REGION 1..648: note = Synthetic: SR296 - CD8ss.CH3DE.CD3e.P2A.IgKss.antiC D33.CH3KK.minCD4hinge.CD4tm.CD4ic
ALPVTALLLPLALLLHAARPGSGQPREPQVYTDPPSRDELTKNQVSLTCEVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGQPREPQVYTKPPSRDELTKNQVSLKCLVKGFYPSDIAVEWESNGQPENNYKTTPPVLDSDGSFFLYSKLTVDKSRWQQGNVFSCSVMHEALHNHYTQKSLSLSPGKSNIKVLPTWSTPVQPMALIVLGGVAGLLLFIGLGIFFCVRCRHRRRQ

SEQ ID NO: 158 - protein, 664 aa, synthetic construct
  REGION 1..664: note = Synthetic: SR001-CD4 - CD8ss.FRB.CD3e.P2A.huIgKss.C D33.FKBP.minCD4hinge.CD4tm.CD4ic-full
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQAERMSQIKRLLSEKKTCQCPHRFQKTCSPI

SEQ ID NO: 159 - protein, 656 aa, synthetic construct
  REGION 1..656: note = Synthetic: SR001-CD8 - CD8ss.FRB.CD3e.P2A.huIgKss.a ntiCD33.FKBP.minCD4hinge.CD4tm.CD8ic-full
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFLYCNHRNRRRVCKCPRPVVKSGDKPSLSARYV

SEQ ID NO: 160 - protein, 1268 aa, synthetic construct
  REGION 1..1268: note = Synthetic: TCR-SR001 - TCR.T2A.SR001
GFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRGSGATNFSLLKQAGDVEENPGPMSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSSGSGEGRGSLLTCGDVEENPGPMALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 161 - protein, 638 aa, synthetic construct
  REGION 1..638: note = Synthetic: SR007 - CD8ss.FRBstar.CD3e.P2A.huIgKss.a ntiCLL1.FKBP.minCD4hinge.CD4tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 162 - protein, 760 aa, synthetic construct
  REGION 1..760: note = Synthetic: SR10168 - CD8ss.FRBstar-CD3e.P2A.IgKss.C D19scFv.FKBP.minCD4hinge.CD4tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 163 - protein, 1003 aa, synthetic construct
  REGION 1..1003: note = Synthetic: SR001+IL7a - CD8ss.FRBstar.CD3eP2A.huIgK ss.FRBstar.CD4htm.IL7Ra.P2A.huIgKss.antiCD33.FKBP.minCD4hi nge.CD4tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPMETPAQLLFLLLLWLPDTTGSILWHEMWHEGLEEASRLYFGERNVKGMFEVLEPLHAMMERGPQTLKETSFNQAYGRDLMEAQEWCRKYMKSGNVKDLLQAWDLYYHVFRRISKSNIKVLPTWSTPVQPMALIVLGGVAGLLLFIGLGIFFKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQSGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 164 - protein, 894 aa, synthetic construct
  REGION 1..894: note = Synthetic: SR001+gC - CD8ss.FRBstar.CD3e.P2A.huIgKs s.FRBstar.CD4htm.IL2Rg.P2A.huIgKss.antiCD33.FKBP.CD4htm.CD 4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPMETPAQLLFLLLLWLPDTTGSILWHEMWHEGLEEASRLYFGERNVKGMFEVLEPLHAMMERGPQTLKETSFNQAYGRDLMEAQEWCRKYMKSGNVKDLLQAWDLYYHVFRRISKSNIKVLPTWSTPVQPMALIVLGGVAGLLLFIGLGIFFERTMPRIPTLKNLEDLVTEYHGNFSAWSGVSKGLAESLQPDYSERLCLVSEIPPKGGALGEGPGASPCNQHSPYWAPPCYTLKPETSGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 165 - protein, 718 aa, synthetic construct
  REGION 1..718: note = Synthetic: SR001+IL2Rb - CD8ss.FRBstar.CD3e.P2A.huI gKss.antiCD33.FKBP.CD4htm.trIL2Rb
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 166 - protein, 978 aa, synthetic construct
  REGION 1..978: note = Synthetic: SR001+IL2Rb+gC - CD8ss.FRBstar.CD3e.P2A. huIgKss.FRBstar.CD4htm.IL2Rg.P2A.huIgKss.antiCD33.FKBP.CD4 htm.trIL2Rb
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPMETPAQLLFLLLLWLPDTTGSILWHEMWHEGLEEASRLYFGERNVKGMFEVLEPLHAMMERGPQTLKETSFNQAYGRDLMEAQEWCRKYMKSGNVKDLLQAWDLYYHVFRRISKSNIKVLPTWSTPVQPMALIVLGGVAGLLLFIGLGIFFERTMPRIPTLKNLEDLVTEYHGNFSAWSGVSKGLAESLQPDYSERLCLVSEIPPKGGALGEGPGASPCNQHSPYWAPPCYTLKPETSGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 167 - protein, 668 aa, synthetic construct
  REGION 1..668: note = Synthetic: SR300 - CD8ss.FRB.CD3e.P2A.PD1ss.PD1(A13 2V)ex.FKBP.minCD4hinge.CD4tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKVQIKESLRAELRVTERRAEVPTAHPSPSPRPAGQFQTLVGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 168 - protein, 668 aa, synthetic construct
  REGION 1..668: note = Synthetic: SR301 - CD8ss.FRB.CD3e.P2A.PD1ss.PD1ex.F KBP.minCD4hinge.CD4tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 169 - protein, 510 aa, synthetic construct
  REGION 1..510: note = Synthetic: SR354 - CD8ss.FRBstar.FCER1G.P2A.huIgKss.antiCD33.FKBP.CD4tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGEPLCYILDAILFLYGIVLTLLYCRLKIQVRKAAITSYEKSDGVYTGLSTRNQETYETLKHEKPPQSGSGATNFSLLKQAGDVEENPGPSMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 170 - protein, 757 aa, synthetic construct
  REGION 1..757: note = Synthetic: SR303 - CD8ss.FRBstar.CD3e.P2A.huIgKss.a ntiROR1.FKBP.minCD4hinge.CD4tm.CD4ic
ALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRISGSGATNFSLLKQAGDVEENPGPSMETPAQLLFLLLLWLPDTTGDIQMTQSPSFLSASVGDRVTINCKASQNIDRYLNWYQQKLGEAPKRLLYNTNKLQTGIPSRFSGSGSATDFTLTISSLQPEDFATYFCLQYNSLPLTFGSGTKLEIKGGGGSGGGGSGGGGSEVQLVESGGGLVQPGRSLKLSCAASGFIFSEHNMAWVRQAPKKGLEWVATISDDGRNTYYRDSMRGRFTISRENARSTLYLQLDSLRSEDTATYYCASHRYNLFDSWGQGVMVTVSSGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESNIKVLPTWSTPVQPMALIVLGGVAGLLLFIGLGIFFCVRCRHRRRQ

SEQ ID NO: 171 - protein, 22 aa, synthetic construct
  REGION 1..22: note = Synthetic
GSGATNFSLLKQAGDVEENPGP

SEQ ID NO: 172 - protein, 19 aa, synthetic construct
  REGION 1..19: note = Synthetic
ATNFSLLKQAGDVEENPGP

SEQ ID NO: 173 - protein, 14 aa, synthetic construct
  REGION 1..14: note = Synthetic
LLKQAGDVEENPGP

SEQ ID NO: 174 - protein, 21 aa, synthetic construct
  REGION 1..21: note = Synthetic
GSGEGRGSLLTCGDVEENPGP

SEQ ID NO: 175 - protein, 18 aa, synthetic construct
  REGION 1..18: note = Synthetic
EGRGSLLTCGDVEENPGP

SEQ ID NO: 176 - protein, 13 aa, synthetic construct
  REGION 1..13: note = Synthetic
LLTCGDVEENPGP

SEQ ID NO: 177 - protein, 23 aa, synthetic construct
  REGION 1..23: note = Synthetic
GSGQCTNYALLKLAGDVESNPGP

SEQ ID NO: 178 - protein, 20 aa, synthetic construct
  REGION 1..20: note = Synthetic
QCTNYALLKLAGDVESNPGP

SEQ ID NO: 179 - protein, 14 aa, synthetic construct
  REGION 1..14: note = Synthetic
LLKLAGDVESNPGP

SEQ ID NO: 180 - protein, 25 aa, synthetic construct
  REGION 1..25: note = Synthetic
GSGVKQTLNFDLLKLAGDVESNPGP

SEQ ID NO: 181 - protein, 22 aa, synthetic construct
  REGION 1..22: note = Synthetic
VKQTLNFDLLKLAGDVESNPGP

SEQ ID NO: 182 - protein, 14 aa, synthetic construct
  REGION 1..14: note = Synthetic
LLKLAGDVESNPGP

SEQ ID NO: 183 - protein, 19 aa, synthetic construct
  REGION 1..19: note = Synthetic
LLNFDLLKLAGDVESNPGP

SEQ ID NO: 184 - protein, 19 aa, synthetic construct
  REGION 1..19: note = Synthetic
TLNFDLLKLAGDVESNPGP

SEQ ID NO: 185 - protein, 14 aa, synthetic construct
  REGION 1..14: note = Synthetic
LLKLAGDVESNPGP

SEQ ID NO: 186 - protein, 17 aa, synthetic construct
  REGION 1..17: note = Synthetic
NFDLLKLAGDVESNPGP

SEQ ID NO: 187 - protein, 20 aa, synthetic construct
  REGION 1..20: note = Synthetic
QLLNFDLLKLAGDVESNPGP

SEQ ID NO: 188 - protein, 24 aa, synthetic construct
  REGION 1..24: note = Synthetic
APVKQTLNFDLLKLAGDVESNPGP

SEQ ID NO: 189 - protein, 40 aa, synthetic construct
  REGION 1..40: note = Synthetic
VTELLYRMKRAETYCPRPLLAIHPTEARHKQKIVAPVKQT

SEQ ID NO: 190 - protein, 18 aa, synthetic construct
  REGION 1..18: note = Synthetic
LNFDLLKLAGDVESNPGP

SEQ ID NO: 191 - protein, 40 aa, synthetic construct
  REGION 1..40: note = Synthetic
LLAIHPTEARHKQKIVAPVKQTLNFDLLKLAGDVESNPGP

SEQ ID NO: 192 - protein, 33 aa, synthetic construct
  REGION 1..33: note = Synthetic
EARHKQKIVAPVKQTLNFDLLKLAGDVESNPGP

SEQ ID NO: 193 - protein, 4 aa, synthetic construct
  REGION 1..4: note = Synthetic
SGSG

SEQ ID NO: 194 - protein, 4 aa, synthetic construct
  REGION 1..4: note = Synthetic
RAKR

SEQ ID NO: 195 - skipped

SEQ ID NO: 196 - protein, 7 aa, synthetic construct
  REGION 1..7: note = Synthetic
ENLYFQG

SEQ ID NO: 197 - protein, 7 aa, synthetic construct
  REGION 1..7: note = Synthetic
ENLYFQS

SEQ ID NO: 198 - DNA, 10 bp, synthetic construct
  misc_feature 1..10: note = Synthetic
  source 1..10: mol_type = other DNA
gccrccatgg

(19) United States
(12) Patent Application Publication
(10) Pub. No.: US 2025/0312451 A1
(43) Pub. Date: Oct. 9, 2025

(54) MULTIPARTITE RECEPTOR AND SIGNALING COMPLEXES

(71) Applicant: Regeneron Pharmaceuticals, Inc., Tarrytown, NY (US)

(72) Inventors: Jordan Jarjour, Seattle, WA (US); Jardin Alexandra Leleux, Seattle, WA (US); Mark Pogson, North Bend, WA (US)

(21) Appl. No.: 18/854,624

(22) PCT Filed: Apr. 7, 2023
(86) PCT No.: PCT/US2023/065552

§ 371 Date: Oct. 7, 2024

Related U.S. Application Data
(60) Provisional application No. 63/329,003, filed on Apr. 8, 2022.

(51) Int. Cl. A61K 40/32; A61K 38/45; A61K 40/11; A61K 40/15; A61K 40/41; A61P 35/00; C07K 14/725; C07K 14/73; C07K 16/28; C12N 5/0783; C12N 9/12; C12N 9/90

(52) U.S. Cl. CPC A61K 40/32; A61K 38/45; A61K 40/11; A61K 40/15; A61K 40/41; A61P 35/00; C07K 14/7051; C07K 14/70514; C07K 16/2803; C07K 16/2851; C12N 5/0636; C12N 5/0646; C12N 9/12; C12N 9/90; C12Y 207/11001; C12Y 502/01008; C07K 2317/53; C07K 2317/622; C07K 2319/03; C12N 2510/00

(57) ABSTRACT

The present disclosure provides adoptive T cell therapies that have improved architectures for targeting antigens and recruiting multimeric immune signaling complexes for treating, preventing, or ameliorating at least one symptom of a cancer, infectious disease, autoimmune disease, inflammatory disease, and immunodeficiency, or condition associated therewith.

### CROSS REFERENCE TO RELATED APPLICATIONS

This application is a 35 U.S.C. § 371 national stage filing of International Application No. PCT/US2023/065552, filed on Apr. 7, 2023, which claims the benefit of priority to U.S. Provisional Application No. 63/329,003, filed on Apr. 8, 2022. The contents of each of the aforementioned applications are hereby incorporated by reference herein in their entireties.

### STATEMENT REGARDING SEQUENCE LISTING

The instant application contains a Sequence Listing which has been submitted electronically in .XML format and is hereby incorporated by reference in its entirety. Said .XML copy, created on Oct. 10, 2023, is named 137080_13902_Sequence_Listing.xml, and is 292,867 bytes in size. The sequence listing contained in this .XML file is part of the specification and is hereby incorporated by reference herein in its entirety.

### BACKGROUND

### Technical Field

The present disclosure relates to improved adoptive cell therapies. More particularly, the disclosure relates to improved immune receptor signaling molecules, cells, and methods of using the same for modulating spatial and/or temporal control of immune receptor signal initiation and downstream responses during adoptive immunotherapy.

### Description of the Related Art

The global burden of cancer doubled between 1975 and 2000. Cancer is the second leading cause of morbidity and mortality worldwide, with approximately 14.1 million new cases and 8.2 million cancer related deaths in 2012. The number of new cancer cases is projected to rise to 22 million within the next two decades.

Although chemotherapies and subsequently biologics have been the standard of care for decades now, adoptive cellular therapy is emerging as a powerful paradigm for delivering complex biological signals to treat cancer. In contrast to small molecule and biologic drug compositions, adoptive cell therapies have the potential to execute unique therapeutic tasks owing to their myriad sensory and response programs and increasingly defined mechanisms of genetic control. To achieve such therapeutic value, cells need to be outfitted with machinery for sensing and integrating chemical and/or biological information associated with local physiological environments.

In recent years, outfitting cells with chimeric antigen receptors (CARs) or transgenic T cell receptors (TCRs) have proven to be a potent way to target immune cells to a particular antigen (e.g., a tumor antigen), stimulate T cell activating signal transduction, and ultimately attack and kill the antigen-associated cell (e.g., cancer cell). Despite these successes there remains fundamental differences and limitations between the two architectures including i) sensitivity ii) antigen recognition, iii) antigen independent signaling activity, and iv) lack of regulatability. Accordingly, there remains a need for improved targeting and signaling machinery that more potently sense and respond to target antigens and associated cells.

### BRIEF SUMMARY

The present disclosure generally relates, in part, to engineered immunoreceptor complexes that can both recognize a target antigen and recruit and activate a natural or a transgenic immunoreceptor signaling complex, e.g., a T cell receptor (TCR) signaling complex, polynucleotides and polypeptides encoding the same, compositions thereof, and methods of making and using the same to treat a disorder or disease (e.g., cancer or autoimmune).

In one aspect, disclosed herein is a non-natural cell, comprising a signaling component comprising (i) a first multimerization domain comprising an FRB polypeptide or variant thereof or a FKBP polypeptide or variant thereof, (ii) a first polypeptide linker, and (iii) a CD3ε polypeptide or variant thereof; and a targeting component comprising (i) an anti-CLL1 scFv or single domain antibody (sdAb), (ii) an anti-CD33 scFv or single domain antibody (sdAb), (iii) a second polypeptide linker, (iv) a second multimerization domain comprising an FRB polypeptide or variant thereof or a FKBP polypeptide or variant thereof, (v) a CD4 hinge polypeptide, (vi) a CD4 transmembrane polypeptide, and (vii) a truncated CD4 intracellular polypeptide.

In some embodiments, the targeting component does not comprise a functional intracellular domain or costimulatory domain having signaling capabilities. In some embodiments, the CD4 hinge polypeptide comprises an amino acid sequence having at least 90%, 95%, 96%, 97%, 98%, or 99% identity to SEQ ID NO: 41. In one embodiment, the CD4 hinge polypeptide comprises an amino acid sequence as set forth in SEQ ID NO: 41.

In some embodiments, the CD3ε polypeptide comprises an amino acid sequence having at least 90%, 95%, 96%, 97%, 98%, or 99% identity to SEQ ID NO: 32. In one embodiment, the CD3ε polypeptide comprises an amino acid sequence as set forth in SEQ ID NO: 32. In some embodiments, the CD3ε polypeptide comprises both extracellular and intracellular portions.

In some embodiments, the FRB and FKBP polypeptides localize extracellularly when the signaling and targeting components are expressed. In some embodiments, the first and second multimerization domains are different. In some embodiments, the first multimerization domain comprises an FRB polypeptide or variant thereof, and the second multimerization domain comprises an FKBP12 polypeptide or variant thereof. In some embodiments, the first multimerization domain comprises an FKBP12 polypeptide or variant thereof, and the second multimerization domain comprises an FRB polypeptide or variant thereof. In one embodiment, the FRB polypeptide is an FRB T2098L variant. In some embodiments, the FRB polypeptide comprises an amino acid sequence having at least 90%, 95%, 96%, 97%, 98%, 99%, identity to, or comprising a sequence set forth as SEQ ID NO: 1. In some embodiments, the FRB polypeptide comprises an amino acid sequence having at least 90%, 95%, 96%, 97%, 98%, 99%, identity to, or comprising a sequence set forth as SEQ ID NO: 2. In some embodiments, the FKBP12 polypeptide comprises an amino acid sequence having at least 90%, 95%, 96%, 97%, 98%, 99%, identity to, or comprising a sequence set forth as SEQ ID NO: 3. In some embodiments, the FKBP12 polypeptide comprises an amino acid sequence having at least 90%, 95%, 96%, 97%, 98%, 99%, identity to, or comprising a sequence set forth as SEQ ID NO: 4.

In some embodiments, the multimerization domains of the signaling component and the targeting component associate with a bridging factor. In some embodiments, the bridging factor is selected from the group consisting of: rapamycin or a rapalog thereof, gibberellin or a derivative thereof, abscisic acid (ABA) or a derivative thereof, methotrexate or a derivative thereof, cyclosporin A or a derivative thereof, FK506/cyclosporin A or a derivative thereof, trimethoprim (Tmp)-synthetic ligand for FK506 binding protein (FKBP) (SLF) or a derivative thereof, wherein the bridging factor promotes the formation of a polypeptide complex, with the bridging factor associated with and disposed between the multimerization domains of the signaling and targeting components. In some embodiments, the bridging factor is AP1903, AP20187, AP21967 (also known as C16-(S)-7-methylindolerapamycin), everolimus, novolimus, pimecrolimus, ridaforolimus, sirolimus, tacrolimus, temsirolimus, umirolimus, zotarolimus, or BPC015.

In some embodiments, the first polypeptide linker is a linker of 2 to 40 amino acids in length. In some embodiments, the first polypeptide linker is selected from the group consisting of: GG, GS, SG, SS, GSS, SSG, GSG, SGS, SGG, G4S, 2×G4S, 3×G4S, 4×G4S, 5×G4S, and any combination thereof. In one embodiment, the first polypeptide linker is a 3×G4S linker. In some embodiments, the second polypeptide linker is a linker of 2 to 40 amino acids in length. In some embodiments, the second polypeptide linker is selected from the group consisting of: GG, GS, SG, SS, GSS, SSG, GSG, SGS, SGG, G4S, 2×G4S, 3×G4s, 4×G4S, and any combination thereof. In one embodiment, the second polypeptide linker is a G4S linker. In some embodiments, the anti-CLL1 scFv or sdAb and the anti-CD33 scFv or sdAb are separated by a third polypeptide linker. In some embodiments, the third polypeptide linker is a linker of 2 to 40 amino acids in length. In some embodiments, the third polypeptide linker is selected from the group consisting of: GG, GS, SG, SS, GSS, SSG, GSG, SGS, SGG, G4S, 2×G4S, 3×G4s, 4×G4S, and any combination thereof. In some embodiments, the third polypeptide linker is a G4S linker.

In some embodiments, the CD4 transmembrane domain comprises an amino acid sequence having at least 90%, 95%, 96%, 97%, 98%, or 99% identity to SEQ ID NO: 45. In some embodiments, the CD4 transmembrane polypeptide comprises an amino acid sequence as set forth in SEQ ID NO: 45. In some embodiments, the truncated intracellular CD4 polypeptide comprises an amino acid sequence as set forth in SEQ ID NO: 48. In one embodiment, the truncated intracellular CD4 polypeptide comprises an amino acid sequence as set forth in SEQ ID NO: 49.

In some embodiments, the anti-CLL1 and anti-CD33 antibodies are each a sdAb. In some embodiments, the sdAb is a camelid VHH, nanobody, or heavy chain-only antibody (HcAb). In one embodiment, the sdAb is a camelid VHH. In some embodiments, the scFv or sdAb is human or humanized. In some embodiments, the anti-CLL1 scFv or sdAb comprises CDR1, CDR2, and CDR3 amino acid sequences as set forth in SEQ ID NOs: 92, 93, and 94, respectively. In some embodiments, the anti-CLL1 scFv or sdAb comprises a sequence having at least 90%, 95%, 96%, 97%, 98%, or 99% identity to SEQ ID NO: 75. In some embodiments, the anti-CLL1 scFv or sdAb comprises a sequence as set forth SEQ ID NO: 75. In some embodiments, the anti-CD33 scFv or sdAb comprises CDR1, CDR2, and CDR3 amino acid sequences as set forth in SEQ ID NOs: 89, 90, and 91, respectively. In some embodiments, the anti-CD33 scFv or sdAb comprises a sequence having at least 90%, 95%, 96%, 97%, 98%, or 99% identity to SEQ ID NO: 72. In some embodiments, the anti-CD33 scFv or sdAb comprises a sequence as set forth SEQ ID NO:72.

In some embodiments, the signaling component further comprises a signal sequence. In some embodiments, the signal sequence is a CD8 signal sequence. In some embodiments, the CD8 signal sequence comprises an amino acid sequence having at least 90%, 95%, 96%, 97%, 98%, or 99% identity to SEQ ID NO: 96. In one embodiment, the CD8 signal sequence comprises an amino acid sequence set forth as SEQ ID NO: 96. In some embodiments, the targeting component further comprises a signal sequence. In some embodiments, the signal sequence is an IgK signal sequence. In some embodiments, the IgK signal sequence comprises an amino acid sequence having at least 90%, 95%, 96%, 97%, 98%, or 99% identity to SEQ ID NO: 95. In one embodiment, the IgK signal sequence comprises an amino acid sequence set forth as SEQ ID NO: 95. In some embodiments, the signaling component comprises a sequence having at least 90%, 95%, 96%, 97%, 98%, or 99% identity to SEQ ID NO: 111. In one embodiment, the signaling component comprises a sequence set forth as SEQ ID NO: 111. In some embodiments, the targeting component comprises a sequence having at least 90%, 95%, 96%, 97%, 98%, or 99% identity to a sequence set forth as SEQ ID NO: 122. In one embodiment, the targeting component comprises a sequence set forth as SEQ ID NO: 122.

In some embodiments, the non-natural cell comprises a fusion polypeptide which comprises the targeting component and the signaling component. In some embodiments, the fusion polypeptide comprises a sequence having at least 90%, 95%, 96%, 97%, 98%, or 99% identity to SEQ ID NO: 151. In one embodiment, the fusion polypeptide comprises a sequence set forth as SEQ ID NO: 151.

In some embodiments, the cell comprises a first nucleic acid molecule encoding the signaling component. In some embodiments, the cell comprises a second nucleic acid molecule encoding the targeting component. In some embodiments, the cell comprises a nucleic acid molecule that encodes both the signaling component and the targeting component.

In some embodiments, the cell further expresses an exogenous costimulatory factor, immunomodulatory factor, agonist for a costimulatory factor, antagonist for an immunosuppressive factor, immune cell engager, flip receptor, or any combination thereof. In some embodiments, the cell further expresses an exogenous lymphocyte receptor or co-receptor. In some embodiments, the exogenous lymphocyte receptor or co-receptor is selected from the group consisting of: TCR alpha (TCRα), TCR beta (TCRβ), TCR gamma (TCRγ), TCR delta (TCRδ), CD4, CD8, pre T cell receptor α (pTα), Fc receptor alpha (FcRα), Fc receptor beta (FcRβ), Fc receptor gamma (FcRγ), natural killer group 2 member D (NKG2D), CD79A, CD79B, and any combination thereof. In some embodiments, the cell further expresses an exogenous TCR. In some embodiments, the exogenous TCR binds a target antigen selected from the group consisting of: α-fetoprotein (AFP), B Melanoma Antigen (BAGE) family members, Brother of the regulator of imprinted sites (BORIS), Cancer-testis antigens, Cancer-testis antigen 83 (CT-83), Carbonic anhydrase IX (CA1X), Carcinoembryonic antigen (CEA), Cytomegalovirus (CMV) antigens, Cytotoxic T cell (CTL)-recognized antigen on melanoma (CAMEL), Epstein-Barr virus (EBV) antigens, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human Epidermal Growth Factor Receptor 2 (HER-2), Human papillomavirus (HPV)-E6, HPV-E7, Human telomerase reverse transcriptase (hTERT), IGF2BP3/A3, K-Ras, K-Ras G12C, K-Ras G12D, K-Ras G12V, Latent membrane protein 2 (LMP2), Melanoma antigen family A, 1 (MAGE-A1), MAGE-A2, MAGE-A3, MAGE-A4, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53,P antigen (PAGE) family members, Placenta-specific 1 (PLAC1), Preferentially expressed antigen in melanoma (PRAME), Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, Tyrosinase, Tyrosinase related protein (TRP)1, TRP2, Wilms tumor protein (WT-1), X Antigen Family Member 1 (XAGE1), and X Antigen Family Member 2 (XAGE2). In some embodiments, the exogenous TCR is an αβ-TCR or γδ-TCR.

In some embodiments, the cell further expresses a CAR, CCR, or flip receptor. In some embodiments, the cell further expresses a zetakine, immune cell engager, or BiTE. In one embodiment, the cell is a hematopoietic cell. In some embodiments, the cell is a T cell, an αβ-T cell, or a γδ-T cell. In some embodiments, the cell is a CD3⁺, CD4⁺, and/or CD8⁺ cell. In some embodiments, the cell is an immune effector cell. In some embodiments, the cell is a cytotoxic T lymphocyte (CTL), a tumor infiltrating lymphocyte (TIL), or a helper T cell. In some embodiments, the cell is a natural killer (NK) cell or natural killer T (NKT) cell. In some embodiments, the source of the cell is peripheral blood mononuclear cells, bone marrow, lymph nodes tissue, cord blood, thymus issue, tissue from a site of infection, ascites, pleural effusion, spleen tissue, or tumors. In one embodiment, the non-natural cell is an isolated non-natural cell. In one embodiment, the non-natural cell is obtained from a subject. In one embodiment, the non-natural cell is a human cell. In some embodiments, the cell further comprises a polypeptide having at least 90%, 95%, 96%, 97%, 98%, or 99% identity to SEQ ID NO: 137. In one embodiment, the cell further comprises an amino acid sequence as set forth in SEQ ID NO: 137. In some embodiments, the cell further comprises a polypeptide having at least 90%, 95%, 96%, 97%, 98%, or 99% identity to SEQ ID NO: 138. In some embodiments, the cell further comprises an amino acid sequence as set forth in SEQ ID NO: 138.

In one aspect, disclosed herein is a fusion polypeptide comprising a signaling component comprising (i) a first multimerization domain comprising an FRB polypeptide or variant thereof or a FKBP polypeptide or variant thereof, (ii) a first polypeptide linker, and (iii) a CD3ε polypeptide or variant thereof; and a polypeptide cleavage signal; and a targeting component comprising (i) an anti-CLL1 scFv or single domain antibody (sdAb), (ii) an anti-CD33 scFv or single domain antibody (sdAb), (iii) a second polypeptide linker, (iv) a second multimerization domain comprising an FRB polypeptide or variant thereof or a FKBP polypeptide or variant thereof, (v) a CD4 hinge polypeptide, (vi) a CD4 transmembrane polypeptide, and (vii) a truncated CD4 intracellular polypeptide.

In some embodiments, the targeting component does not comprise a functional intracellular domain or costimulatory domain having signaling capabilities. In some embodiments, the CD4 hinge polypeptide comprises an amino acid sequence having at least 90%, 95%, 96%, 97%, 98%, or 99% identity to SEQ ID NO: 41. In one embodiment, the CD4 hinge polypeptide comprises an amino acid sequence as set forth in SEQ ID NO: 41.

In some embodiments, the CD3ε polypeptide comprises an amino acid sequence having at least 90%, 95%, 96%, 97%, 98%, or 99% identity to SEQ ID NO: 32. In one embodiment, the CD3ε polypeptide comprises an amino acid sequence as set forth in SEQ ID NO: 32. In some embodiments, the CD3ε polypeptide comprises both extracellular and intracellular portions.

In some embodiments, the FRB and FKBP polypeptides localize extracellularly when the signaling and targeting components are expressed. In some embodiments, the first and second multimerization domains are different. In some embodiments, the first multimerization domain comprises an FRB polypeptide or variant thereof, and the second multimerization domain comprises an FKBP12 polypeptide or variant thereof. In some embodiments, the first multimerization domain comprises an FKBP12 polypeptide or variant thereof, and the second multimerization domain comprises an FRB polypeptide or variant thereof. In one embodiment, the FRB polypeptide is an FRB T2098L variant. In some embodiments, the FRB polypeptide comprises an amino acid sequence having at least 90%, 95%, 96%, 97%, 98%, 99%, identity to, or comprising a sequence set forth as SEQ ID NO: 1. In some embodiments, the FRB polypeptide comprises an amino acid sequence having at least 90%, 95%, 96%, 97%, 98%, 99%, identity to, or comprising a sequence set forth as SEQ ID NO: 2. In some embodiments, the FKBP12 polypeptide comprises an amino acid sequence having at least 90%, 95%, 96%, 97%, 98%, 99%, identity to, or comprising a sequence set forth as SEQ ID NO: 3. In some embodiments, the FKBP12 polypeptide comprises an amino acid sequence having at least 90%, 95%, 96%, 97%, 98%, 99%, identity to, or comprising a sequence set forth as SEQ ID NO: 4.

In some embodiments, the multimerization domains of the signaling component and the targeting component associate with a bridging factor. In some embodiments, the bridging factor is selected from the group consisting of: rapamycin or a rapalog thereof, gibberellin or a derivative thereof, abscisic acid (ABA) or a derivative thereof, methotrexate or a derivative thereof, cyclosporin A or a derivative thereof, FK506/cyclosporin A or a derivative thereof, trimethoprim (Tmp)-synthetic ligand for FK506 binding protein (FKBP) (SLF) or a derivative thereof, wherein the bridging factor promotes the formation of a polypeptide complex, with the bridging factor associated with and disposed between the multimerization domains of the signaling and targeting components. In some embodiments, the bridging factor is AP1903, AP20187, AP21967 (also known as C16-(S)-7-methylindolerapamycin), everolimus, novolimus, pimecrolimus, ridaforolimus, sirolimus, tacrolimus, temsirolimus, umirolimus, zotarolimus, or BPC015.

In some embodiments, the first polypeptide linker is a linker of 2 to 40 amino acids in length. In some embodiments, the first polypeptide linker is selected from the group consisting of: GG, GS, SG, SS, GSS, SSG, GSG, SGS, SGG, G4S, 2×G4S, 3×G4S, 4×G4S, 5×G4S, and any combination thereof. In one embodiment, the first polypeptide linker is a 3×G4S linker. In some embodiments, the second polypeptide linker is a linker of 2 to 40 amino acids in length. In some embodiments, the second polypeptide linker is selected from the group consisting of: GG, GS, SG, SS, GSS, SSG, GSG, SGS, SGG, G4S, 2×G4S, 3×G4s, 4×G4S, and any combination thereof. In some embodiments, the second polypeptide linker is a G4S linker. In some embodiments, the anti-CLL1 scFv or sdAb and the anti-CD33 scFv or sdAb are separated by a third polypeptide linker. In some embodiments, the third polypeptide linker is a linker of 2 to 40 amino acids in length. In some embodiments, the third polypeptide linker is selected from the group consisting of: GG, GS, SG, SS, GSS, SSG, GSG, SGS, SGG, G4S, 2×G4S, 3×G4s, 4×G4S, and any combination thereof. In one embodiment, the third polypeptide linker is a G4S linker.

In some embodiments, the CD4 transmembrane domain comprises an amino acid sequence having at least 90%, 95%, 96%, 97%, 98%, or 99% identity to SEQ ID NO: 45. In one embodiment, the CD4 transmembrane polypeptide comprises an amino acid sequence as set forth in SEQ ID NO: 45. In one embodiment, the truncated intracellular CD4 polypeptide comprises an amino acid sequence as set forth in SEQ ID NO: 48. In one embodiment, the truncated intracellular CD4 polypeptide comprises an amino acid sequence as set forth in SEQ ID NO: 49. In some embodiments, the anti-CLL1 and anti-CD33 antibodies are each a sdAb. In some embodiments, the sdAb is a camelid VHH, nanobody, or heavy chain-only antibody (HcAb). In one embodiment, the sdAb is a camelid VHH. In some embodiments, the scFv or sdAb is human or humanized. In some embodiments, the anti-CLL1 scFv or sdAb comprises CDR1, CDR2, and CDR3 amino acid sequences as set forth in SEQ ID NOs: 92, 93, and 94, respectively. In some embodiments, the anti-CLL1 scFv or sdAb comprises a sequence having at least 90%, 95%, 96%, 97%, 98%, or 99% identity to SEQ ID NO: 75. In some embodiments, the anti-CLL1 scFv or sdAb comprises a sequence as set forth SEQ ID NO: 75. In some embodiments, the anti-CD33 scFv or sdAb comprises CDR1, CDR2, and CDR3 amino acid sequences as set forth in SEQ ID NOs: 89, 90, and 91, respectively. In some embodiments, the anti-CD33 scFv or sdAb comprises a sequence having at least 90%, 95%, 96%, 97%, 98%, or 99% identity to SEQ ID NO: 72. In some embodiments, the anti-CD33 scFv or sdAb comprises a sequence as set forth SEQ ID NO: 72.

In some embodiments, the signaling component further comprises a signal sequence. In some embodiments, the signal sequence is a CD8 signal sequence. In some embodiments, the CD8 signal sequence comprises an amino acid sequence having at least 90%, 95%, 96%, 97%, 98%, or 99% identity to SEQ ID NO: 96. In one embodiment, the CD8 signal sequence comprises an amino acid sequence set forth as SEQ ID NO: 96. In some embodiments, the targeting component further comprises a signal sequence. In some embodiments, the signal sequence is an IgK signal sequence. In some embodiments, the IgK signal sequence comprises an amino acid sequence having at least 90%, 95%, 96%, 97%, 98%, or 99% identity to SEQ ID NO: 95. In one embodiment, the IgK signal sequence comprises an amino acid sequence set forth as SEQ ID NO: 95. In some embodiments, the signaling component comprises a sequence having at least 90%, 95%, 96%, 97%, 98%, or 99% identity to SEQ ID NO: 111. In one embodiment, the signaling component comprises a sequence set forth as SEQ ID NO: 111. In some embodiments, the targeting component comprises a sequence having at least 90%, 95%, 96%, 97%, 98%, or 99% identity to a sequence set forth as SEQ ID NO: 122. In one embodiment, the targeting component comprises a sequence set forth as SEQ ID NO: 122.

In some embodiments, the fusion polypeptide comprises a fusion polypeptide which comprises the targeting component and the signaling component. In some embodiments, fusion polypeptide comprises a sequence having at least 90%, 95%, 96%, 97%, 98%, or 99% identity to SEQ ID NO: 151. In one embodiment, the fusion polypeptide comprises a sequence set forth as SEQ ID NO: 151.

In some embodiments, the polypeptide cleavage signal is a viral self-cleaving polypeptide. In some embodiments, the polypeptide cleavage signal is a viral self-cleaving 2A polypeptide.

In one aspect, the disclosure provides a nucleic acid molecule that encodes any one of the fusion polypeptides disclosed herein.

In another aspect, the disclosure provides a cell comprising any one of the fusion polypeptides disclosed herein.

In another aspect, the disclosure provides a cell comprising any one of the nucleic acid molecules disclosed herein.

In some embodiments, the cell further expresses an exogenous costimulatory factor, immunomodulatory factor, agonist for a costimulatory factor, antagonist for an immunosuppressive factor, immune cell engager, flip receptor, or any combination thereof. In some embodiments, the cell further expresses an exogenous lymphocyte receptor or co-receptor.

In some embodiments, the exogenous lymphocyte receptor or co-receptor is selected from the group consisting of: TCR alpha (TCRα), TCR beta (TCRβ), TCR gamma (TCRγ), TCR delta (TCRδ), CD4, CD8, pre T cell receptor α (pTα), Fc receptor alpha (FcRα), Fc receptor beta (FcRβ), Fc receptor gamma (FcRγ), natural killer group 2 member D (NKG2D), CD79A, CD79B, and any combination thereof. In some embodiments, the cell further expresses an exogenous TCR. In some embodiments, the exogenous TCR binds a target antigen selected from the group consisting of: α-fetoprotein (AFP), B Melanoma Antigen (BAGE) family members, Brother of the regulator of imprinted sites (BORIS), Cancer-testis antigens, Cancer-testis antigen 83 (CT-83), Carbonic anhydrase IX (CA1X), Carcinoembryonic antigen (CEA), Cytomegalovirus (CMV) antigens, Cytotoxic T cell (CTL)-recognized antigen on melanoma (CAMEL), Epstein-Barr virus (EBV) antigens, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human Epidermal Growth Factor Receptor 2 (HER-2), Human papillomavirus (HPV)-E6, HPV-E7, Human telomerase reverse transcriptase (hTERT), IGF2BP3/A3, K-Ras, K-Ras G12C, K-Ras G12D, K-Ras G12V, Latent membrane protein 2 (LMP2), Melanoma antigen family A, 1 (MAGE-A1), MAGE-A2, MAGE-A3, MAGE-A4, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53,P antigen (PAGE) family members, Placenta-specific 1 (PLAC1), Preferentially expressed antigen in melanoma (PRAME), Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, Tyrosinase, Tyrosinase related protein (TRP)1, TRP2, Wilms tumor protein (WT-1), X Antigen Family Member 1 (XAGE1), and X Antigen Family Member 2 (XAGE2). In some embodiments, the exogenous TCR is an αβ-TCR or γδ-TCR.

In some embodiments, the cell further expresses a CAR, CCR, or flip receptor. In some embodiments, the cell further expresses a zetakine, immune cell engager, or BiTE. In some embodiments, the cell is a hematopoietic cell. In some embodiments, the cell is a T cell, an αβ-T cell, or a γδ-T cell. In some embodiments, the cell is a CD3⁺, CD4⁺, and/or CD8⁺ cell. In some embodiments, the cell is an immune effector cell. In some embodiments, the cell is a cytotoxic T lymphocyte (CTL), a tumor infiltrating lymphocyte (TIL), or a helper T cell. In some embodiments, the cell is a natural killer (NK) cell or natural killer T (NKT) cell. In some embodiments, the source of the cell is peripheral blood mononuclear cells, bone marrow, lymph nodes tissue, cord blood, thymus issue, tissue from a site of infection, ascites, pleural effusion, spleen tissue, or tumors. In one embodiment, the cell is an isolated cell. In one embodiment, the cell is obtained from a subject. In one embodiment the cell is a human cell.

In one aspect, disclosed herein is a polypeptide complex, comprising a signaling component comprising (i) a first multimerization domain comprising an FRB polypeptide or variant thereof or a FKBP polypeptide or variant thereof, (ii) a first polypeptide linker, and (iii) a CD3ε polypeptide or variant thereof; and a targeting component comprising (i) an anti-CLL1 scFv or single domain antibody (sdAb), (ii) an anti-CD33 scFv or single domain antibody (sdAb), (iii) a second polypeptide linker, (iv) a second multimerization domain comprising an FRB polypeptide or variant thereof or a FKBP polypeptide or variant thereof, (v) a CD4 hinge polypeptide, (vi) a CD4 transmembrane polypeptide, and (vii) a truncated CD4 intracellular polypeptide.

In some embodiments, the targeting component does not comprise a functional intracellular domain or costimulatory domain having signaling capabilities. In some embodiments, the CD4 hinge polypeptide comprises an amino acid sequence having at least 90%, 95%, 96%, 97%, 98%, or 99% identity to SEQ ID NO: 41. In some embodiments, the CD4 hinge polypeptide comprises an amino acid sequence as set forth in SEQ ID NO: 41. In some embodiments, the CD3ε polypeptide comprises an amino acid sequence having at least 90%, 95%, 96%, 97%, 98%, or 99% identity to SEQ ID NO: 32. In one embodiment, the CD3ε polypeptide comprises an amino acid sequence as set forth in SEQ ID NO: 32. In some embodiments, the CD3ε polypeptide comprises both extracellular and intracellular portions.

In some embodiments, the FRB and FKBP polypeptides localize extracellularly when the signaling and targeting components are expressed. In some embodiments, the first and second multimerization domains are different. In some embodiments, the first multimerization domain comprises an FRB polypeptide or variant thereof, and the second multimerization domain comprises an FKBP12 polypeptide or variant thereof. In some embodiments, the first multimerization domain comprises an FKBP12 polypeptide or variant thereof, and the second multimerization domain comprises an FRB polypeptide or variant thereof. In one embodiment, the FRB polypeptide is an FRB T2098L variant. In some embodiments, the FRB polypeptide comprises an amino acid sequence having at least 90%, 95%, 96%, 97%, 98%, 99%, identity to, or comprising a sequence set forth as SEQ ID NO: 1. In some embodiments, the FRB polypeptide comprises an amino acid sequence having at least 90%, 95%, 96%, 97%, 98%, 99%, identity to, or comprising a sequence set forth as SEQ ID NO: 2. In some embodiments, the FKBP12 polypeptide comprises an amino acid sequence having at least 90%, 95%, 96%, 97%, 98%, 99%, identity to, or comprising a sequence set forth as SEQ ID NO: 3. In some embodiments, the FKBP12 polypeptide comprises an amino acid sequence having at least 90%, 95%, 96%, 97%, 98%, 99%, identity to, or comprising a sequence set forth as SEQ ID NO: 4.

In some embodiments, the multimerization domains of the signaling component and the targeting component associate with a bridging factor. In some embodiments, the bridging factor is selected from the group consisting of: rapamycin or a rapalog thereof, gibberellin or a derivative thereof, abscisic acid (ABA) or a derivative thereof, methotrexate or a derivative thereof, cyclosporin A or a derivative thereof, FK506/cyclosporin A or a derivative thereof, trimethoprim (Tmp)-synthetic ligand for FK506 binding protein (FKBP) (SLF) or a derivative thereof, wherein the bridging factor promotes the formation of a polypeptide complex, with the bridging factor associated with and disposed between the multimerization domains of the signaling and targeting components. In some embodiments, the bridging factor is AP1903, AP20187, AP21967 (also known as C16-(S)-7-methylindolerapamycin), everolimus, novolimus, pimecrolimus, ridaforolimus, sirolimus, tacrolimus, temsirolimus, umirolimus, zotarolimus, or BPC015. In some embodiments, the first polypeptide linker is a linker of 2 to 40 amino acids in length. In some embodiments, the first polypeptide linker is selected from the group consisting of: GG, GS, SG, SS, GSS, SSG, GSG, SGS, SGG, G4S, 2×G4S, 3×G4S, 4×G4S, 5×G4S, and any combination thereof. In one embodiment, the first polypeptide linker is a 3×G4S linker. In some embodiments, the second polypeptide linker is a linker of 2 to 40 amino acids in length. In some embodiments, the second polypeptide linker is selected from the group consisting of: GG, GS, SG, SS, GSS, SSG, GSG, SGS, SGG, G4S, 2×G4S, 3×G4s, 4×G4S, and any combination thereof. In one embodiment, the second polypeptide linker is a G4S linker. In some embodiments, the anti-CLL1 scFv or sdAb and the anti-CD33 scFv or sdAb are separated by a third polypeptide linker. In some embodiments, the third polypeptide linker is a linker of 2 to 40 amino acids in length. In some embodiments, the third polypeptide linker is selected from the group consisting of: GG, GS, SG, SS, GSS, SSG, GSG, SGS, SGG, G4S, 2×G4S, 3×G4s, 4×G4S, and any combination thereof. In one embodiment, the third polypeptide linker is a G4S linker.

In some embodiments, the CD4 transmembrane domain comprises an amino acid sequence having at least 90%, 95%, 96%, 97%, 98%, or 99% identity to SEQ ID NO: 45. In one embodiment, the CD4 transmembrane polypeptide comprises an amino acid sequence as set forth in SEQ ID NO: 45. In one embodiment, the truncated intracellular CD4 polypeptide comprises an amino acid sequence as set forth in SEQ ID NO: 48. In one embodiment, the truncated intracellular CD4 polypeptide comprises an amino acid sequence as set forth in SEQ ID NO: 49.

In some embodiments, the anti-CLL1 and anti-CD33 antibodies are each a sdAb. In some embodiments, the sdAb is a camelid VHH, nanobody, or heavy chain-only antibody (HcAb). In one embodiment, the sdAb is a camelid VHH. In some embodiments, the scFv or sdAb is human or humanized. In some embodiments, the anti-CLL1 scFv or sdAb comprises CDR1, CDR2, and CDR3 amino acid sequences as set forth in SEQ ID NOs: 92, 93, and 94, respectively. In some embodiments, the anti-CLL1 scFv or sdAb comprises a sequence having at least 90%, 95%, 96%, 97%, 98%, or 99% identity to SEQ ID NO: 75. In some embodiments, the anti-CLL1 scFv or sdAb comprises a sequence as set forth SEQ ID NO: 75. In some embodiments, the anti-CD33 scFv or sdAb comprises CDR1, CDR2, and CDR3 amino acid sequences as set forth in SEQ ID NOs: 89, 90, and 91, respectively. In some embodiments, the anti-CD33 scFv or sdAb comprises a sequence having at least 90%, 95%, 96%, 97%, 98%, or 99% identity to SEQ ID NO: 72. In some embodiments, the anti-CD33 scFv or sdAb comprises a sequence as set forth SEQ ID NO:72.

In some embodiments, the signaling component further comprises a signal sequence. In some embodiments, the signal sequence is a CD8 signal sequence. In some embodiments, the CD8 signal sequence comprises an amino acid sequence having at least 90%, 95%, 96%, 97%, 98%, or 99% identity to SEQ ID NO: 96. In one embodiment, the CD8 signal sequence comprises an amino acid sequence set forth as SEQ ID NO: 96. In some embodiments, the targeting component further comprises a signal sequence. In some embodiments, the signal sequence is an IgK signal sequence. In some embodiments, the IgK signal sequence comprises an amino acid sequence having at least 90%, 95%, 96%, 97%, 98%, or 99% identity to SEQ ID NO: 95. In one embodiment, the IgK signal sequence comprises an amino acid sequence set forth as SEQ ID NO: 95. In some embodiments, the signaling component comprises a sequence having at least 90%, 95%, 96%, 97%, 98%, or 99% identity to SEQ ID NO: 111. In one embodiment, the signaling component comprises a sequence set forth as SEQ ID NO: 111. In some embodiments, the targeting component comprises a sequence having at least 90%, 95%, 96%, 97%, 98%, or 99% identity to a sequence set forth as SEQ ID NO: 122. In one embodiment, the targeting component comprises a sequence set forth as SEQ ID NO: 122. In some embodiments, the polypeptide complex comprises a fusion polypeptide which comprises the targeting component and the signaling component. In some embodiments, the fusion polypeptide comprises a sequence having at least 90%, 95%, 96%, 97%, 98%, or 99% identity to SEQ ID NO: 151. In one embodiment, the fusion polypeptide comprises a sequence set forth as SEQ ID NO: 151.

In one aspect, the disclosure provides a nucleic acid molecule that encodes both the signaling component and the targeting component of any one of the polypeptide complexes disclosed herein.

In another aspect, the disclosure provides a cell comprising any one of the polypeptide complexes disclosed herein.

In another aspect, the disclosure provides a cell comprising any one of the nucleic acid molecules disclosed herein.

In some embodiments, the cell further expresses an exogenous costimulatory factor, immunomodulatory factor, agonist for a costimulatory factor, antagonist for an immunosuppressive factor, immune cell engager, flip receptor, or any combination thereof. In some embodiments, the cell further expresses an exogenous lymphocyte receptor or co-receptor.

In some embodiments, the exogenous lymphocyte receptor or co-receptor is selected from the group consisting of: TCR alpha (TCRα), TCR beta (TCRβ), TCR gamma (TCRγ), TCR delta (TCRδ), CD4, CD8, pre T cell receptor α (pTα), Fc receptor alpha (FcRα), Fc receptor beta (FcRβ), Fc receptor gamma (FcRγ), natural killer group 2 member D (NKG2D), CD79A, CD79B, and any combination thereof. In some embodiments, the cell further expresses an exogenous TCR. In some embodiments, the exogenous TCR binds a target antigen selected from the group consisting of: α-fetoprotein (AFP), B Melanoma Antigen (BAGE) family members, Brother of the regulator of imprinted sites (BORIS), Cancer-testis antigens, Cancer-testis antigen 83 (CT-83), Carbonic anhydrase IX (CA1X), Carcinoembryonic antigen (CEA), Cytomegalovirus (CMV) antigens, Cytotoxic T cell (CTL)-recognized antigen on melanoma (CAMEL), Epstein-Barr virus (EBV) antigens, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human Epidermal Growth Factor Receptor 2 (HER-2), Human papillomavirus (HPV)-E6, HPV-E7, Human telomerase reverse transcriptase (hTERT), IGF2BP3/A3, K-Ras, K-Ras G12C, K-Ras G12D, K-Ras G12V, Latent membrane protein 2 (LMP2), Melanoma antigen family A, 1 (MAGE-A1), MAGE-A2, MAGE-A3, MAGE-A4, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53,P antigen (PAGE) family members, Placenta-specific 1 (PLAC1), Preferentially expressed antigen in melanoma (PRAME), Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, Tyrosinase, Tyrosinase related protein (TRP)1, TRP2, Wilms tumor protein (WT-1), X Antigen Family Member 1 (XAGE1), and X Antigen Family Member 2 (XAGE2). In some embodiments, the exogenous TCR is an αβ-TCR or γδ-TCR.

In some embodiments, the cell further expresses a CAR, CCR, or flip receptor. In some embodiments, the cell further expresses a zetakine, immune cell engager, or BiTE. In one embodiment, the cell is a hematopoietic cell. In some embodiments, the cell is a T cell, an αβ-T cell, or a γδ-T cell. In some embodiments, the cell is a CD3⁺, CD4⁺, and/or CD8⁺ cell. In some embodiments, the cell is an immune effector cell. In some embodiments, the cell is a cytotoxic T lymphocyte (CTL), a tumor infiltrating lymphocyte (TIL), or a helper T cell. In some embodiments, the cell is a natural killer (NK) cell or natural killer T (NKT) cell. In some embodiments, the source of the cell is peripheral blood mononuclear cells, bone marrow, lymph nodes tissue, cord blood, thymus issue, tissue from a site of infection, ascites, pleural effusion, spleen tissue, or tumors. In one embodiment, the cell is an isolated cell. In one embodiment, cell is obtained from a subject. In one embodiment, the cell is a human cell.

In one aspect, the disclosure provides a polynucleotide encoding the signaling and targeting component of any one of the fusion polypeptides or polypeptide complexes disclosed herein.

In another aspect, the disclosure provides a cDNA encoding the signaling and targeting component of any one of the fusion polypeptides or polypeptide complexes disclosed herein.

In another aspect, the disclosure provides an RNA encoding any one of the signaling and targeting components, or any one of the fusion polypeptides, or any one of the polypeptide complexes disclosed herein.

In another aspect, the disclosure provides a vector comprising any one of the polynucleotides disclosed herein.

In some embodiments, the vector is an expression vector. In one embodiment, the vector is a transposon. In some embodiments, the vector is a piggyBAC transposon or a Sleeping Beauty transposon. In some embodiments, the vector is a viral vector. In some embodiments, the vector is an adenoviral vector, an adeno-associated viral (AAV) vector, a herpes virus vector, a vaccinia virus vector, or a retroviral vector. In some embodiments, the retroviral vector is a lentiviral vector. In some embodiments, the lentiviral vector is selected from the group consisting of: human immunodeficiency virus 1 (HIV-1); human immunodeficiency virus 2 (HIV-2), visna-maedi virus (VMV) virus; caprine arthritis-encephalitis virus (CAEV); equine infectious anemia virus (EIAV); feline immunodeficiency virus (FIV); bovine immune deficiency virus (BIV); and simian immunodeficiency virus (SIV).

In one aspect, the disclosure provides a cell comprising any one of the fusion polypeptides, polynucleotides, or the vectors disclosed herein.

In one embodiment, the cell is a hematopoietic cell. In some embodiments, the cell is an immune effector cell. In some embodiments, the cell is a T cell, an αβ T cell, or a TS T cell. In some embodiments, the cell expresses CD3⁺, CD4⁺, CD8⁺, or a combination thereof. In some embodiments, the cell is a cytotoxic T lymphocyte (CTL), a tumor infiltrating lymphocyte (TIL), or a helper T cell. In some embodiments, the cell is a natural killer (NK) cell or natural killer T (NKT) cell.

In one aspect, the disclosure provides a composition comprising any one of the cells or vectors disclosed herein.

In another aspect, the disclosure provides a composition comprising a physiologically acceptable carrier and any one of the cells or vectors disclosed herein.

In another aspect, the disclosure provides a method of treating a subject in need thereof comprising administering the subject an effective amount of any one of the compositions disclosed herein.

In another aspect, the disclosure provides a method of treating, preventing, or ameliorating at least one symptom of a cancer, infectious disease, autoimmune disease, inflammatory disease, and immunodeficiency, or condition associated therewith, comprising administering to the subject an effective amount of any one of the compositions disclosed herein.

In another aspect, the disclosure provides a method of treating a solid cancer comprising administering to the subject an effective amount of any one of the compositions disclosed herein.

In some embodiments, the solid cancer is selected from the group consisting of lung cancer, squamous cell carcinoma, colorectal cancer, pancreatic cancer, breast cancer, thyroid cancer, bladder cancer, cervical cancer, esophageal cancer, ovarian cancer, gastric cancer endometrial cancer, or brain cancer. In some embodiments, the solid cancer is a non-small cell lung carcinoma, head and neck squamous cell carcinoma, colorectal cancer, pancreatic cancer, breast cancer, thyroid cancer, bladder cancer, cervical cancer, esophageal cancer, ovarian cancer, gastric cancer endometrial cancer, gliomas, glioblastomas, or oligodendroglioma.

In one aspect, the disclosure provides a method of treating a hematological malignancy comprising administering to the subject an effective amount of any one of the compositions disclosed herein. In some embodiments, the hematological malignancy is a leukemia, lymphoma, or multiple myeloma. In one embodiment, the hematological malignancy is acute myelogenous leukemia (AML).

### BRIEF DESCRIPTION OF SEVERAL VIEWS OF THE DRAWINGS

FIGS. 1A-1H show the architecture of representative engineered immune receptors and fusion constructs. In particular, FIGS. 1A-1C depict expression constructs and components of a few exemplary engineered immune receptors and fusion constructs of the invention. FIGS. 1D-1H are cartoons depicting how representative engineered immune receptors comprising a signaling component and a targeting component may form a complex with an immune receptor (e.g., a TCR) to activate receptor signaling when a target cell expressing the target antigen is present, and, in certain embodiments, when a bridging factor is present. As one non-limiting example, in FIG. 1D, construct SR022/SR001 comprises a signaling component comprising an FRB multimerization domain, a linker, and a CD3ε actuator domain; and a targeting component comprising a CD4 transmembrane domain, a CD4 hinge, an FKBP multimerization domain, a linker, and a CD33 VHH targeting domain. When a cell expressing CD33 is present, the CD33 VHH targeting domain binds to the target cell, bringing it in close contact with the immune cell expressing the engineered immune receptor, e.g., a T cell. When a bridging factor is present, the multimerization domains then interact, allowing the signaling component to activate the immune receptor (e.g., TCR).

FIG. 2 shows the percent positive and median fluorescence intensity of SR024 components on T cells as measured by flow cytometry.

FIG. 3 shows IFNγ, IL2, IL4 and TNF secretion from untransduced and SR024-T cells cocultured with a high CD33 expressing cancer cell line, and in the presence of rapamycin.

FIG. 4 shows IFNγ secretion from untransduced and SR024-T cells cultured alone, with and without rapamycin.

FIGS. 5A and 5B show cytotoxicity and proliferation, respectively, of control and SR024 T cells, each cocultured with a high CD33 expressing cancer cell line.

FIG. 6 shows the percent positive and median fluorescence intensity of SR020, SR022, and SR024 components on T cells as measured by flow cytometry.

FIG. 7A shows IFNγ, IL2, IL4 and TNF secretion from untransduced, SR020-T cells, SR022-T cells, and SR024-T cells, each cocultured with a high CD33 expressing cancer cell line, and in the presence of rapamycin.

FIG. 7B shows IFNγ secretion from untransduced, SR020-T cells, SR022-T cells, and SR024-T cells, each co-cultured with high, medium, and low CD33 expressing cancer cell lines, and in the presence of rapamycin.

FIG. 7C shows IL2 secretion from untransduced, SR020-T cells, SR022-T cells, and SR024-T cells, each co-cultured with high, medium, and low CD33 expressing cancer cell lines, and in the presence of rapamycin.

FIG. 8 shows IFNγ secretion from untransduced, SR020-T cells, SR022-T cells, and SR024-T cells alone, e with and without rapamycin.

FIGS. 9A and 9B show cytotoxicity and proliferation, respectively, of control, SR020-T cells, SR022-T cells, and SR024-T cells, each cocultured with a high CD33 expressing cancer cell line.

FIG. 10 shows the percent positive and median fluorescence intensity of SR021 components on T cells as measured by flow cytometry.

FIG. 11 shows IFNγ, IL2, IL4 and TNF secretion from untransduced and SR021-T cells cocultured with a high CD33 expressing cancer cell line, and in the presence of rapamycin.

FIG. 12 shows IFNγ secretion from untransduced and SR021-T cells cocultured with a high CD33 expressing cancer cell line, alone with and without rapamycin.

FIGS. 13A and 13B show cytotoxicity and proliferation, respectively, of control and SR021-T cells cocultured with a high CD33 expressing cancer cell line.

FIG. 14 shows the percent positive and median fluorescence intensity of SR022 and SR001 components on T cells as measured by flow cytometry.

FIG. 15 shows IFNγ, IL2, and TNF secretion from untransduced, SR022- and SR001-T cells, each cocultured with a high CD33 expressing cancer cell line, and in the presence of rapamycin.

FIG. 16 shows IFNγ secretion from untransduced, SR022- and SR001-T cells, each cocultured with a high CD33 expressing cancer cell line, with and without rapamycin.

FIGS. 17A and 17B show cytotoxicity and proliferation, respectively, of control and SR001-T cells, each cocultured with a high CD33 expressing cancer cell line.

FIG. 18 shows the percent positive and median fluorescence intensity of SR028 components on T cells as measured by flow cytometry.

FIG. 19 shows IFNγ, IL2, IL4 and TNF secretion from untransduced and SR028-T cells cocultured with a high CD33 expressing cancer cell line, and in the presence of rapamycin.

FIG. 20 shows IFNγ secretion from untransduced and SR028-T cells cocultured with a high CD33 expressing cancer cell line, with and without rapamycin.

FIGS. 21A and 21B show cytotoxicity and proliferation, respectively, of control and SR028-T cells, each cocultured with a high CD33 expressing cancer cell line.

FIG. 22 shows the percent positive and median fluorescence intensity of SR004 and SR006 components on T cells as measured by flow cytometry.

FIG. 23 shows IFNγ, IL2, IL4 and TNF secretion from untransduced, SR004- and SR006-T cells, each cocultured with a high CD33 expressing cancer cell line, and in the presence of rapamycin.

FIG. 24 shows IFNγ secretion from untransduced, SR004- and SR006-T cells, each cocultured with a high CD33 expressing cancer cell line, with and without rapamycin.

FIGS. 25A and 25B show cytotoxicity and proliferation, respectively, of control, SR004- and SR006-T cells, each cocultured with a high CD33 expressing cancer cell line.

FIGS. 26A and 26B show the percent positive and median fluorescence intensity of SR008 and SR030 components on T cells as measured by flow cytometry.

FIGS. 26C and 26D show the phenotypes of CD4+ and CD8+ T cells transduced with vectors encoding the indicated constructs.

FIGS. 27A and 27B show IFNγ, IL2, IL4 and TNF secretion from untransduced, SR008-T cells, and SR030-T cells, each cocultured with either a high CD33 expressing cancer cell line or a high CLL1 expressing cancer cell line, and in the presence of rapamycin.

FIG. 27C shows IFNγ secretion of untransduced, SR008-T cells, and SR030-T cells alone, with or without rapamycin.

FIGS. 28A and 28B show cytotoxicity of untransduced, SR008-T cells, and SR030-T cells, each cocultured with a high CD33 expressing cancer cell line or a high CLL1 expressing cancer cell line, respectively.

FIG. 29 shows the percent positive and median fluorescence intensity of SR001 and SR001-28 components on T cells as measured by flow cytometry.

FIG. 30 shows IFNγ, IL2, IL4 and TNF secretion from untransduced, SR001- and SR001-28-T cells, each cocultured with a high CD33 expressing cancer cell line and in the presence of rapamycin.

FIGS. 31A and 31B show cytotoxicity and proliferation, respectively, of control, SR001- and SR001-28-T cells, each cocultured with a high CD33 expressing cancer cell line.

FIGS. 32A and 32B show in vivo tumor growth in NSG mouse xenografts and control following administration of 10E6 SR001±41BB or CD28 costimulation domains. All tumor control is regulated by rapamycin.

FIGS. 32C and 32D show in vivo tumor growth in NSG mouse xenografts and control following administration of 3E6 SR001±41BB or CD28 costimulation domains with rapamycin present.

FIGS. 33A and 33B show the percent positive and median fluorescence intensity of SR022±CD4 or CD8 coreceptor signaling domains as measured by flow cytometry.

FIG. 34 show IFNγ and IL2 secretion from untransduced, SR022-T cells±CD4 or CD8 coreceptor signaling domains, each cocultured with a high CD33 expressing cancer cell line, with or without rapamycin.

FIGS. 35A-35C show the vector copy number, percent positive, and geometric mean fluorescence intensity of a tetramer and TCR beta chain staining for an untransduced TCR, a transgenic TCR, SR001, or the combination of a transgenic TCR and SR001 as measured by flow cytometry.

FIGS. 35D and 35E show the percent positive and geometric mean fluorescence intensity of an anti-VHH staining for an untransduced TCR, a transgenic TCR, SR001, or the combination of a transgenic TCR and SR001 as measured by flow cytometry.

FIGS. 36A and 36B show IFNγ, IL2 and TNFα secretion from untransduced, transgenic TCR, SR001, or the combination of a transgenic TCR and SR001, each cocultured with a high HLA-A2+TCR epitope+ or CD33+ expressing cancer cell line and in the absence and presence of rapamycin.

FIG. 36C shows cytotoxicity of untransduced, transgenic TCR cells, SR001-T cells, or the combination of transgenic TCR and SR001, cocultured with cancer cells high HLA-A2+TCR epitope+ or CD33+ expressing cancer cell lines in the absence or presence of rapamycin.

FIGS. 37A and 37B show IL2 secretion from untransduced or SR001, each cocultured with a very low CD33− or CLL1-expressing cancer cell line in the presence of rapamycin.

FIG. 38 shows in vivo tumor growth in NSG mouse xenografts and control following administration of 10E6 SR007 or comparator regulated CAR T cells (targeting either CD33 or CLL1). All tumor control is regulated by rapamycin.

FIGS. 39A-39C show the vector copy number, percent positive FRB and VHH staining for expression in the absence or presence of rapamycin.

FIGS. 40A and 40B show IFNγ secretion of SR10167 or SR10168-T cells alone±rapamycin, compared with regulated CAR controls.

FIGS. 41A and 41B show IFNγ secretion of SR10167 or SR10168-T cells±rapamycin, compared with regulated CAR controls, when cultured with a CD19+ target cell line.

FIGS. 42A-42D show IL2 and TNFα secretion of SR10167 or SR10168-T cells±rapamycin, compared with regulated CAR controls, when cultured with a CD19+ target cell line.

FIGS. 43A and 43B show cytotoxicity of control, SR10167-T cells, and Regulated CAR comparator-T cells, each cocultured with either CD19+ Jeko-1 or Daudi cell lines. All tumor control is regulated by rapamycin.

FIG. 44 shows cartoons of representative constitutively active (non-regulatable) engineered immune receptors and fusion constructs.

FIGS. 45A and 45B show the percent positive and median fluorescence intensity of VHH staining for expression of SR292, SR293, SR296, SR001 and relevant comparator molecules including a non-regulated CAR, regulated CAR, and non-regulated TCR-based architecture comparator.

FIGS. 46A and 46B show IFNγ and IL2 secretion of SR292, SR293, SR296, SR001 and relevant comparator molecules including a non-regulated CAR, regulated CAR, and non-regulated TCR-based architecture comparator, when cultured with a CD33+ target cell line.

FIG. 47 shows IFNγ secretion of T cells alone expressing SR292, SR293, SR296, SR001 and relevant comparator molecules including a non-regulated CAR, regulated CAR, and non-regulated TCR-based architecture comparator.

FIGS. 48A and 48B show cytotoxicity and T cell proliferation of control, SR292, SR293, SR296 and one non-regulated CAR, following coculture with a CD33+ target line.

FIG. 49 shows the percent positive and median fluorescence intensity of FRB staining for expression of SR001±IL7-receptor-α, common γ chain, IL2-receptor-β, or both common γ chain, and IL2-receptor-β.

FIGS. 50A-50D show IFNγ and IL2 secretion of SR001±IL7-receptor-α, common γ chain, IL2-receptor-β, or both common γ chain and IL2-receptor-β, when cultured with a CD33+ target cell line with and without rapamycin.

FIG. 51 shows T cell proliferation of control or SR001±IL7-receptor-α, common γ chain, IL2-receptor-β, or both common γ chain and IL2-receptor-β, following coculture with a CD33+ target line.

FIG. 52 shows the median fluorescence intensity of FRB or PD-1 staining of T cells expressing either SR300 (affinity enhanced PD1) or SR301 (wild-type PD1).

FIGS. 53A-53D show cytotoxicity of T cells expressing SR300 or SR301 when cultured with a PDL1+ target cell line or the same cell line with PDL1 and PDL2 knocked out, with and without rapamycin.

FIG. 54 shows the percent positive and median fluorescence intensity of VHH staining for expression of SR354 construct.

FIGS. 55A and 55B show IFNγ secretion of NK cells expressing SR354 when cultured with a CD33+ target cell line or the same cell line with CD33 knocked out.

FIGS. 55C and 55D show cytotoxicity of NK cells expressing SR354 when cultured with a CD33+ target cell line or the same cell line with CD33 knocked out.

FIG. 56 shows the percent positive of SR303 components on T cells as measured by flow cytometry.

FIGS. 57A and 57B show IFNγ secretion of SR303 cells when cultured with an ROR1+ target cell line with and without rapamycin.

FIG. 57C shows IFNγ secretion of T cells alone expressing SR303 molecules.

### BRIEF DESCRIPTION THE SEQUENCE IDENTIFIERS

SEQ ID NOs: 1-4 set forth the amino acid sequences of exemplary FRB and FKBP12 polypeptides.
SEQ ID NOs: 5-10 set forth the amino acid sequences of exemplary antibody derived heterodimerization domains.
SEQ ID NOs: 11-31 set forth amino acid sequences of exemplary linkers.
SEQ ID NOs: 32-40 set forth the amino acid sequences of exemplary actuator domains.
SEQ ID NOs: 41-44 set forth the amino acid sequences of illustrative hinge domains.
SEQ ID NOs: 45-47 set forth the amino acid sequences of exemplary transmembrane polypeptides.
SEQ ID NOs: 48 and 49 set forth the amino acid sequences of illustrative truncated intracellular CD4 polypeptides. SEQ ID NOs: 50-94 set forth the amino acid sequences of illustrative targeting domains.
SEQ ID NOs: 95-97 sets forth the amino acid sequences of illustrative signal sequences.
SEQ ID NOs: 98-106 set forth the amino acid sequences of exemplary intracellular signaling domains.
SEQ ID NOs: 107-115 set forth the amino acid sequences of exemplary signaling components.
SEQ ID NOs: 116-136 set forth the amino acid sequences of exemplary targeting components.
SEQ ID NOs: 137 and 138 set forth the amino acid sequences of exemplary co-signaling components.
SEQ ID NOs: 139-170 set forth the amino acid sequences of illustrative fusion polypeptides.
SEQ ID NOs: 171-192 set forth the amino acid sequence of protease cleavage sites and self-cleaving polypeptide cleavage sites.
SEQ ID NO: 193 sets forth a spacer amino acid sequence.
SEQ ID NO: 194 sets forth a furin recognition amino acid sequence.
SEQ ID NOs: 195-197 set forth TEV (tobacco etch virus) protease cleavage sites.
SEQ ID NO: 198 sets forth a kozak amino acid sequence.

In the foregoing sequences, X, if present, refers to any amino acid or the absence of an amino acid.

### DETAILED DESCRIPTION

### A. Overview

Cancer is among the leading causes of death worldwide. Although adoptive cell therapy is being used to successfully treat some hematological malignancies, treatment of solid tumors with both chimeric antigen receptor (CAR) T cells and T cells that express T cell receptors (TCR) against tumor antigens still remains largely ineffective.

Additionally, while TCRs are known to be exquisitely sensitive to low levels of target antigen, they lack the ability to see non-MHC presented antigens. On the other hand, while CARs can be engineered to target almost any extracellular antigen through their antibody-like binding domains, they are generally less sensitive to low levels of target antigen and are prone to tonic and/or antigen-independent signaling. Moreover, T cells engineered to express a CAR or TCR generally lack spatial and temporal control of T cell activity and/or demonstrate insufficient activation of T cell signaling pathways.

Lack of control over engineered T cell activity can trigger a range of side effects, many of which begin subtly but can rapidly worsen. A particularly severe complication is cytokine release syndrome (CRS) or “cytokine storm” where CAR T cells induce massive and potentially fatal cytokine release. CRS can produce dangerously high fevers, extreme fatigue, difficulty breathing, and a sharp drop in blood pressure. CRS can also produce a second wave of side effects that involve the nervous system, including neurotoxicity, tremors, headaches, confusion, loss of balance, trouble speaking, seizures, and hallucinations. Insufficient activation of T cell signaling pathways can result in failure to eradicate a cancer and/or lead to a cancer that becomes refractory to treatment. The compositions and methods contemplated herein offer solutions to these and other problems plaguing adoptive cell therapies.

Thus, the disclosure generally relates to improved compositions and methods for regulating the antigen recognition capabilities, sensitivity, and/or spatial and temporal control of adoptive cell therapies by using engineered immunoreceptor complexes that bind a selected target antigen and that can recruit and activate a natural or transgenic immunoreceptor signaling complex.

Without wishing to be bound by any particular theory, the engineered immune receptor compositions and methods contemplated herein provide numerous advantages over CAR T cell and TCR T cell therapies existing in the art, including but not limited to, both spatial and temporal control over immune effector cell signal transduction, binding and signaling activities, and activating signaling pathways without requiring MHC complex recognition. In some embodiments, temporal control primes the engineered immune receptor machinery for signaling through bridging factor mediated association of a targeting component to a signaling component. In other embodiments, the machinery is primed by association of multimerization domains, without the need for a bridging factor. Spatial control engages the signaling machinery through recognition of a target antigen by an extracellular or targeting domain of a targeting component, whereas the signaling component comprises an actuator domain that forms a complex with a lymphocyte immune receptor. In this manner, immune effector cells activate receptor signaling when a target cell expressing the target antigen is present, and, in certain embodiments, when a bridging factor is present.

In various embodiments, the disclosure contemplates signaling and targeting components that generate an immune receptor-based response against cells that express a target antigen without recognition of the natural immune receptor target antigen. In various embodiments, the disclosure contemplates signaling and targeting components that generate an immune receptor-based anti-cancer response against cancers that express a target antigen without requiring MHC complex recognition of the target antigen and/or the lymphocytic immune receptor's natural or engineered target antigen.

In particular embodiments, the engineered immune receptor complexes include a signaling component that comprises a multimerization domain polypeptide or variant thereof and an actuator domain or variant thereof (e.g., a CD3ε, CD3δ, CD3γ, FcεR1γ, Igα/CD79a, Igβ/CD79b, DAP10, or DAP12 polypeptide); and a targeting component that comprises an extracellular domain that comprises a targeting domain that binds a target antigen expressed on a target cell, a multimerization domain polypeptide or variant thereof, a transmembrane domain, and optionally a hinge domain disposed between the multimerization domain and the transmembrane domain. In some embodiments, in the presence of a bridging factor, the signaling and binding/targeting components associate with one another through the bridging factor to form a functionally active immune receptor. In some embodiments, the components associate without the need for a bridging factor.

In particular embodiments, the multimerization domains of the signaling and targeting components are positioned extracellularly. Extracellular position of the multimerization domains provides numerous advantages over intracellular positioning including, but not limited to, more efficient positioning of the targeting domain, higher temporal sensitivity to bridging factor regulation, and less toxicity due to ability to use non-immunosuppressive doses of particular bridging factors.

Polynucleotides encoding the engineered immune receptors, targeting components, signaling components, protein/polypeptide complexes, and fusion proteins; polypeptides comprising the engineered immune receptors, targeting components, signaling components, protein/polypeptide complexes, and fusion proteins; cells comprising polynucleotides encoding the engineered immune receptors, targeting components, and signaling components and/or expressing the same; vectors encoding the engineered immune receptors, targeting components, signaling components, protein/polypeptide complexes, and fusion proteins; and methods of using the same to treat a disease or disorder (e.g., cancer or an immune disorder) are contemplated herein.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and CC Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant to include any elements listed after the phrase and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

As used herein, the term “TCR complex” refers to a complex formed by the association of CD3 with a TCR. For example, a TCR complex can be composed of a CD3γ chain, a CD3δ chain, two CD3ε chains, a homodimer of CD3ζ chains, a TCRα chain, and a TCRβ chain. In some embodiments, a TCR complex can be composed of a CD3γ chain, a CD3δ chain, two CD3ε chains, a homodimer of CD3ζ chains, a TCRγ chain, and a TCRδ chain.

A “component of a TCR complex,” as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

An “actuator polypeptide”, “actuator domain”, or “actuator” as used herein, refer to a polypeptide that, associates, integrates, or complexes, either directly or indirectly, with a multimeric immune receptor complex to promote signaling and does not itself contain direct antigen-binding properties. In certain embodiments, the actuator domain is part of a protein or protein complex that signals when bound to a target molecule. The actuator domain may directly contribute to a cellular response when it contains signaling domains or motifs, such as an immunoreceptor tyrosine-based activation motif (ITAM). In other embodiments, an actuator domain will indirectly promote a cellular response by associating with one or more other proteins that directly signal and thus promote a cellular response. Illustrative actuator domains include, e.g., a CD3 polypeptide, FcεR1γ polypeptide, Igα/CD79a polypeptide, Igβ/CD79b polypeptide, DAP10 polypeptide, or DAP12, polypeptide, or any combination thereof.

A “multimerization domain,” or “multimerization domain polypeptide” as used herein, refers to a polypeptide that preferentially interacts or associates with another different polypeptide directly or via a bridging molecule, e.g., a chemically inducible dimerizer, wherein the interaction of different multimerization domains substantially contributes to or efficiently promotes multimerization (i.e., the formation of a dimer, trimer, or multipartite complex, which may be a homodimer, heterodimer, homotrimer, heterotrimer, homomultimer, heteromultimer). A multimerization domain may be derived either from a natural, synthetic, semi-synthetic, or recombinant source.

Illustrative examples of multimerization domains suitable for use in particular embodiments contemplated herein include an FK506 binding protein (FKBP) polypeptide or variants thereof, an FKBP-rapamycin binding (FRB) polypeptide or variants thereof, a calcineurin polypeptide or variants thereof, a cyclophilin polypeptide or variants thereof, a bacterial dihydrofolate reductase (DHFR) polypeptide or variants thereof, a PYR1-like 1 (PYL1) polypeptide or variants thereof, an abscisic acid insensitive 1 (ABI1) polypeptide or variants thereof, a GIB1 polypeptide or variants thereof, or a GAI polypeptide or variants thereof.

As used herein, the term “FKBP-rapamycin binding polypeptide” refers to an FRB polypeptide. In particular embodiments, the FRB polypeptide is an FKBP12-rapamycin binding polypeptide. FRB polypeptides suitable for use in particular embodiments contemplated herein generally contain at least about 85 to about 100 amino acid residues. In certain embodiments, the FRB polypeptide comprises a 93 amino acid sequence Ile-2021 through Lys-2113 and a mutation of T2098L (T82L is equivalent position in 93 amino acid FRB polypeptide), with reference to GenBank Accession No. L34075.1. The terms “FRB star”, FRBstar”, or “FRB*” as used herein refer to such FRB T2098L (T82L) mutants. An FRB polypeptide contemplated herein binds to an FKBP polypeptide through a bridging factor, thereby forming a ternary complex.

As used herein, the term “FK506 binding protein” refers to an FKBP polypeptide. In particular embodiments, the FKBP polypeptide is an FKBP12 polypeptide or an FKBP12 polypeptide comprising an F36V mutation. In certain embodiments, an FKBP domain may also be referred to as a “rapamycin binding domain”. Information concerning the nucleotide sequences, cloning, and other aspects of various FKBP species is known in the art (see, e.g., Staendart et al., Nature 346:671, 1990 (human FKBP12); Kay, Biochem. J. 314:361, 1996). An FKBP polypeptide contemplated herein binds to an FRB polypeptide through a bridging factor, thereby forming a ternary complex.

A “bridging factor” refers to a molecule that associates with and that is disposed between two or more multimerization domains. In particular embodiments, multimerization domains substantially contribute to or efficiently promote formation of a polypeptide complex only in the presence of a bridging factor. In particular embodiments, multimerization domains do not contribute to or do not efficiently promote formation of a polypeptide complex in the absence of a bridging factor. Illustrative examples of bridging factors suitable for use in particular embodiments contemplated herein include, but are not limited to AP21967, rapamycin (sirolimus) or a rapalog thereof, coumermycin or a derivative thereof, gibberellin or a derivative thereof, abscisic acid (ABA) or a derivative thereof, methotrexate or a derivative thereof, cyclosporin A or a derivative thereof, FKCsA or a derivative thereof, trimethoprim (Tmp)-synthetic ligand for FKBP (SLF) or a derivative thereof, or any combination thereof.

Rapamycin analogs (rapalogs) include, but are not limited to, those disclosed in U.S. Pat. No. 6,649,595, which rapalog structures are incorporated herein by reference in their entirety. In certain embodiments, a bridging factor is a rapalog with substantially reduced immunosuppressive effect as compared to rapamycin. In a preferred embodiment, the rapalog is AP21967 (also known as C-16-(S)-7-methylindolerapamycin, IC₅₀=10 nM, a chemically modified non-immunosuppressive rapamycin analogue). Other illustrative rapalogs suitable for use in particular embodiments contemplated herein include, but are not limited to, AP1903, AP20187, everolimus, novolimus, pimecrolimus, ridaforolimus, sirolimus, tacrolimus, temsirolimus, umirolimus, zotarolimus, and BPC015.

A “substantially reduced immunosuppressive effect” refers to at least less than 0.1 to 0.005 times the immunosuppressive effect observed or expected for the same dose measured either clinically or in an appropriate in vitro (e.g., inhibition of T cell proliferation) or in vivo surrogate of human immunosuppressive activity.

A “transmembrane domain” or “TM domain” is a domain that anchors a polypeptide to the plasma membrane of a cell. The TM domain may be derived either from a natural, synthetic, semi-synthetic, or recombinant source.

As used herein, the term “extracellular domain” refers to a domain or portion of a polypeptide which is situated or present outside of a cell. The extracellular domain of a targeting component, as disclosed herein, comprises at least one targeting domain, which re-directs activation of an immune receptor complex, e.g., a TCR, to one or more selected target antigens, e.g., on a target cell, such as a cancer cell, when the targeting component is associated with the signaling component, e.g., by a bridging factor connecting the multimerization domains of the targeting component and the signaling component, or by association of multimerization domains of the targeting component and the signaling component without the need for a bridging factor.

The term “effector function” or “effector cell function” refers to a specialized function of an immune effector cell. Effector function includes, but is not limited to, activation, cytokine production, proliferation and cytotoxic activity, including the release of cytotoxic factors, or other cellular responses elicited with antigen binding to the receptor expressed on the immune effector cell. An “intracellular signaling domain” or “endodomain” refers to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces an effector function signal. The term intracellular signaling domain is meant to include any truncated portion of an intracellular signaling domain necessary or sufficient to transduce an effector function signal.

It is known that signals generated through the TCR alone are insufficient for full activation of the T cell and that a secondary or costimulatory signal is also required. Thus, T cell activation can be said to be mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal.

As used herein, the term, “costimulatory signaling domain,” or “costimulatory domain” refers to an intracellular signaling domain of a costimulatory molecule. Costimulatory molecules are cell surface molecules other than antigen receptors or Fc receptors that provide a second signal required for efficient activation and function of T lymphocytes upon binding to antigen. Illustrative examples of such costimulatory molecules from which costimulatory domains may be isolated include, but are not limited to: Toll-like receptor 1 (TLR1), TLR2, TLR3, TLR4, TLR5, TLR6, TLR7, TLR8, TLR9, TLR10, caspase recruitment domain family member 11 (CARD11), CD2, CD3ε, CD3γ, CD3δ, CD7, CD27, CD28, CD30, CD40, CD54 (ICAM), CD83, CD94, CD134 (OX40), CD137 (4-1BB), CD278 (ICOS), DNAX-Activation Protein 10 (DAP10), FYN, Linker for activation of T-cells family member 1 (LAT), SH2 Domain-Containing Leukocyte Protein Of 76 kD (SLP76), LCK, T cell receptor associated transmembrane adaptor 1 (TRAT1), TNFR2, TNF receptor superfamily member 14 (TNFRS14; HVEM), TNF receptor superfamily member 18 (TNFRS18; GITR), TNF receptor superfamily member 25 (TNFRS25; DR3), and zeta chain of T cell receptor associated protein kinase 70 (ZAP70).

A “hinge domain,” refers to a polypeptide that plays a role in spatially positioning a domain away from the effector cell surface to enable proper cell/cell contact, antigen binding and activation. In particular embodiments, polypeptides may comprise one or more hinge domains between the extracellular domain and the transmembrane domain (TM), between the multimerization domain and the transmembrane domain, or between the multimerization domain and the actuator domain. The hinge domain may be derived either from a natural, synthetic, semi-synthetic, or recombinant source. The hinge domain can include the amino acid sequence of a naturally occurring immunoglobulin hinge region or an altered immunoglobulin hinge region.

A “linker” or “linker polypeptide” refers to a plurality of amino acid residues between the various polypeptide domains added for appropriate spacing and conformation of the molecule. In particular embodiments, the linker is a variable region linking sequence. A “variable region linking sequence,” is an amino acid sequence that connects the V_{H} and V_{L} domains and provides a spacer function compatible with interaction of the two sub-binding domains so that the resulting polypeptide retains a specific binding affinity to the same target molecule as an antibody that comprises the same light and heavy chain variable regions. In particular embodiments, a linker separates one or more heavy or light chain variable domains, hinge domains, multimerization domains, transmembrane domains, costimulatory domains, and/or primary signaling domains.

Illustrated examples of linkers suitable for use in particular embodiments contemplated herein include, but are not limited to the following amino acid sequences: GGG; DGGGS (SEQ ID NO: 16); TGEKP (SEQ ID NO: 17) (see, e.g., Liu et al., PNAS 5525-5530 (1997)); GGRR (SEQ ID NO: 18) (Pomerantz et al. 1995, supra); (GGGGS)ₙ wherein n=1, 2, 3, 4 or 5 (SEQ ID NOs: 11-15) (Kim et al., PNAS 93, 1156-1160 (1996.); EGKSSGSGSESKVD (SEQ ID NO: 19) (Chaudhary et al., 1990, Proc. Natl. Acad. Sci. U.S.A. 87:1066-1070); KESGSVSSEQLAQFRSLD (SEQ ID NO: 20) (Bird et al., 1988, Science 242:423-426), GGRRGGGS (SEQ ID NO: 21); LRQRDGERP (SEQ ID NO: 22); LRQKDGGGSERP (SEQ ID NO: 23); LRQKD(GGGS)₂ ERP (SEQ ID NO: 24). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods. In one embodiment, the linker comprises the following amino acid sequence: GSTSGSGKPGSGEGSTKG (SEQ ID NO: 25) (Cooper et al., Blood, 101(4): 1637-1644 (2003)).

A “spacer domain,” refers to a polypeptide that separates two domains. In one embodiment, a spacer domain moves a or targeting domain away from the effector cell surface to enable proper cell/cell contact, antigen binding and activation (Patel et al., Gene Therapy, 1999; 6: 412-419). In particular embodiments, a spacer domain separates one or more heavy or light chain variable domains, multimerization domains, transmembrane domains, costimulatory domains, and/or primary signaling domains. The spacer domain may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region.

An “antigen (Ag)” refers to a compound, composition, or substance that can stimulate the production of antibodies or a T cell response in an animal, including compositions (such as one that includes a cancer-specific protein) that are injected or absorbed into an animal. Exemplary antigens include but are not limited to lipids, carbohydrates, polysaccharides, glycoproteins, peptides, or nucleic acids. An antigen reacts with the products of specific humoral or cellular immunity, including those induced by heterologous antigens, such as the disclosed antigens.

A “target antigen” or “target antigen of interest” refers to a molecule expressed on the cell surface of a target cell that a binding or targeting domain contemplated herein, is designed to bind. In particular embodiments, the target antigen is an epitope of a polypeptide expressed on the surface of a cancer cell.

As used herein, the term, “targeting domain” refers to a domain, e.g., a domain of a targeting component, that provides the polypeptide with the ability to specifically bind to a target antigen of interest. The targeting domain may be derived either from a natural, synthetic, semi-synthetic, or recombinant source. A targeting domain may, e.g., comprise an antibody, or an antigen-binding fragment thereof or, e.g., an ectodomain.

The terms “specific binding affinity” or “specifically binds” or “specifically bound” or “specific binding” or “specifically targets” as used herein, describe binding of a targeting domain to a target antigen at greater binding affinity than background binding. A targeting domain “specifically binds” to a target antigen, if it binds to or associates with the antigen with an affinity or Kₐ (i.e., an equilibrium association constant of a particular binding interaction with units of 1/M) of, for example, greater than or equal to about 10⁵ M⁻¹. In certain embodiments, a targeting domain (or a fusion protein comprising the same) binds to a target with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” targeting domains (or single chain fusion proteins thereof) refer to those targeting domains with a Kₐ of at least 10⁷ M¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater.

The terms “selectively binds” or “selectively bound” or “selectively binding” or “selectively targets” and describe preferential binding of one molecule to a target molecule (on-target binding) in the presence of a plurality of off-target molecules.

An “antibody” refers to a binding agent that is a polypeptide comprising at least a light chain or heavy chain immunoglobulin variable region which specifically recognizes and binds an epitope of an antigen, such as a lipid, carbohydrate, polysaccharide, glycoprotein, peptide, or nucleic acid containing an antigenic determinant, such as those recognized by an immune cell.

An “epitope” or “antigenic determinant” refers to the region of an antigen to which a binding agent binds.

Antibodies include antigen binding fragments thereof, such as a Camel Ig, a Llama Ig, an Alpaca Ig, Ig NAR, a Fab′ fragment, a F(ab′)₂ fragment, a bispecific Fab dimer (Fab2), a trispecific Fab trimer (Fab3), an Fv, an single chain Fv protein (“scFv”), a bis-scFv, (scFv)₂, a minibody, a diabody, a triabody, a tetrabody, a disulfide stabilized Fv protein (“dsFv”), and a single-domain antibody (sdAb, a camelid VHH, Nanobody) and portions of full length antibodies responsible for antigen binding. The term also includes genetically engineered forms such as chimeric antibodies (for example, humanized murine antibodies), heteroconjugate antibodies (such as, bispecific antibodies) and antigen binding fragments thereof. See also, Pierce Catalog and Handbook, 1994-1995 (Pierce Chemical Co., Rockford, IL); Kuby, J., Immunology, 3rd Ed., W. H. Freeman & Co., New York, 1997.

As used herein, the term “cancer” relates generally to a class of diseases or conditions in which abnormal cells divide without control and can invade nearby tissues.

As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor.

As used herein, the term “benign” or “non-malignant” refers to tumors that may grow larger but do not spread to other parts of the body. Benign tumors are self-limited and typically do not invade or metastasize.

A “cancer cell” refers to an individual cell of a cancerous growth or tissue. Cancer cells include both solid cancers and liquid cancers. A “tumor” or “tumor cell” refers generally to a swelling or lesion formed by an abnormal growth of cells, which may be benign, pre-malignant, or malignant. Most cancers form tumors, but liquid cancers, e.g., leukemia, do not necessarily form tumors. For those cancers that form tumors, the terms cancer (cell) and tumor (cell) are used interchangeably. The amount of a tumor in an individual is the “tumor burden” which can be measured as the number, volume, or weight of the tumor.

The term “relapse” refers to the diagnosis of return, or signs and symptoms of return, of a cancer after a period of improvement or remission.

“Remission,” is also referred to as “clinical remission,” and includes both partial and complete remission. In partial remission, some, but not all, signs and symptoms of cancer have disappeared. In complete remission, all signs and symptoms of cancer have disappeared, although cancer still may be in the body.

“Refractory” refers to a cancer that is resistant to, or non-responsive to, therapy with a particular therapeutic agent. A cancer can be refractory from the onset of treatment (i.e., non-responsive to initial exposure to the therapeutic agent), or as a result of developing resistance to the therapeutic agent, either over the course of a first treatment period or during a subsequent treatment period.

“Antigen negative” refers to a cell that does not express antigen or expresses a negligible amount of antigen that is undetectable. In one embodiment, antigen negative cells do not bind receptors directed to the antigen. In one embodiment, antigen negative cells do not substantially bind receptors directed to the antigen.

As used herein, the terms “individual” and “subject” are often used interchangeably and refer to any animal that exhibits a symptom of cancer or other immune disorder that can be treated with the compositions and methods contemplated elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include human patients that have, have been diagnosed with, or are at risk of having, cancer or another immune disorder.

As used herein, the term “patient” refers to a subject that has been diagnosed with cancer or another immune disorder that can be treated with the compositions and methods disclosed elsewhere herein.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction of the disease or condition, or the delaying of the progression of the disease or condition, e.g., delaying tumor outgrowth. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, the phrase “ameliorating at least one symptom of” refers to decreasing one or more symptoms of the disease or condition for which the subject is being treated. In particular embodiments, the disease or condition being treated is a cancer, wherein the one or more symptoms ameliorated include, but are not limited to, weakness, fatigue, shortness of breath, easy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function).

By “enhance” or “promote,” or “increase” or “expand” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, persistence, cytokine secretion, and/or an increase in cancer cell killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

By “maintain,” or “preserve,” or “maintenance,” or “no change,” or “no substantial change,” or “no substantial decrease” refers generally to the ability of a composition contemplated herein to produce, elicit, or cause a substantially similar or comparable physiological response (i.e., downstream effects) in a cell, as compared to the response caused by either vehicle, a control molecule/composition, or the response in a particular cell lineage. A comparable response is one that is not significantly different or measurable different from the reference response.

Additional definitions are set forth throughout this disclosure.

### C. Engineered Immune Receptor Architectures

In particular embodiments, one or more engineered immune receptors that redirect cytotoxicity of immune effector cells toward cells expressing a target antigen and that recruit and an immune receptor complex are contemplated. As used herein, the terms “engineered immune receptor”, “engineered immune receptor complex”, or “engineered immune receptor system” refer to one or more non-naturally occurring polypeptides that facilitates transduction of an immunostimulatory signal in an immune effector cell upon exposure to a pre-defined or selected target antigen and association/multimerization with an immune receptor complex. In some embodiments, a multimerizing agent or bridging factor is required to promote multimerization of the non-naturally occurring polypeptides and thereby stimulating immune effector cell activity and function through activation of an immune receptor complex. In preferred embodiments, an engineered immune receptor or system is a multi-chain chimeric receptor comprising a signaling component that associates with a member of an immune receptor complex, and a targeting component that redirects or complements the antigen specificity of the receptor/complex.

In one embodiment, a signaling component and a targeting component are expressed from the same cell. In another embodiment, a signaling component and a targeting component are expressed from different cells. In another embodiment, a signaling component is expressed from a cell and a targeting component is supplied exogenously, as a polypeptide. In a particular embodiment, a targeting component pre-loaded with a bridging factor is supplied exogenously to a cell expressing a signaling component.

### 1. Signaling Component

A “signaling component” refers to a polypeptide comprising one or more multimerization domains (e.g., FRB or FKBP polypeptides) or variants thereof, and a CD3ε domain/polypeptide, or functional fragment or variant thereof, that is capable of recruiting an immune receptor complex (e.g., a TCR complex).

In various embodiments, the CD3ε peptide or a functional fragment or variant thereof comprising an amino acid sequence having at least 90% identity to SEQ ID NO: 32. In some embodiments, the CD3ε peptide or a functional fragment or variant thereof comprising an amino acid sequence having at least 95% identity to SEQ ID NO: 32. In some embodiments, the CD3ε peptide or a functional fragment or variant thereof comprising an amino acid sequence having at least 96% identity to SEQ ID NO: 32. In some embodiments, the CD3ε peptide or a functional fragment or variant thereof comprising an amino acid sequence having at least 97% identity to SEQ ID NO: 32. In some embodiments, the CD3ε peptide or a functional fragment or variant thereof comprising an amino acid sequence having at least 98% identity to SEQ ID NO: 32. In some embodiments, the CD3ε peptide or a functional fragment or variant thereof comprising an amino acid sequence having at least 99% identity to SEQ ID NO: 32. In particular embodiments, the CD3ε peptide comprising an amino acid sequence as set forth in SEQ ID NO: 32.

In various embodiments, the CD3ε polypeptide is a human CD3ε polypeptide.

In particular embodiments, a signaling component comprises an FRB or FRB T2098L multimerization domain/polypeptide, a linker polypeptide, and a CD3ε polypeptide.

In various embodiments, a signaling component comprises an amino acid sequence having at least 90% identity to SEQ ID NO: 111. In various embodiments, a signaling component comprises an amino acid sequence having at least 95% identity to SEQ ID NO: 111. In various embodiments, a signaling component comprises an amino acid sequence having at least 96% identity to SEQ ID NO: 111. In various embodiments, a signaling component comprises an amino acid sequence having at least 97% identity to SEQ ID NO: 111. In various embodiments, a signaling component comprises an amino acid sequence having at least 98% identity to SEQ ID NO: 111. In various embodiments, a signaling component comprises an amino acid sequence having at least 99% identity to SEQ ID NO: 111. In particular embodiments, a signaling component comprises an amino acid sequence as set forth in SEQ ID NO: 111.

In various embodiments, the multimerization domain (e.g., FRB or FKBP) localizes extracellularly when the signaling component is expressed.

In particular embodiments, a signaling component comprises an FRB polypeptide or variant thereof. In various embodiments, a signaling component comprises an FRB polypeptide comprising a T2098L (T82L) mutation, or variant thereof. In particular embodiments, the FRB polypeptide comprises an amino acid sequence having at least 90% identity to SEQ ID NO: 1. In particular embodiments, the FRB polypeptide comprises an amino acid sequence having at least 95% identity to SEQ ID NO: 1. In particular embodiments, the FRB polypeptide comprises an amino acid sequence having at least 96% identity to SEQ ID NO: 1. In particular embodiments, the FRB polypeptide comprises an amino acid sequence having at least 97% identity to SEQ ID NO: 1. In particular embodiments, the FRB polypeptide comprises an amino acid sequence having at least 98% identity to SEQ ID NO: 1. In particular embodiments, the FRB polypeptide comprises an amino acid sequence having at least 99% identity to SEQ ID NO: 1. In particular embodiments, the FRB polypeptide comprises an amino acid sequence as set forth in SEQ ID NO: 1. In particular embodiments, the FRB polypeptide comprises an amino acid sequence as set forth in SEQ ID NO: 2. In particular embodiments, the FRB polypeptide comprises an amino acid sequence having at least 90% identity to SEQ ID NO: 2. In particular embodiments, the FRB polypeptide comprises an amino acid sequence having at least 95% identity to SEQ ID NO: 2. In particular embodiments, the FRB polypeptide comprises an amino acid sequence having at least 96% identity to SEQ ID NO: 2. In particular embodiments, the FRB polypeptide comprises an amino acid sequence having at least 97% identity to SEQ ID NO: 2. In particular embodiments, the FRB polypeptide comprises an amino acid sequence having at least 98% identity to SEQ ID NO: 2. In particular embodiments, the FRB polypeptide comprises an amino acid sequence having at least 99% identity to SEQ ID NO: 2. In particular embodiments, the FRB polypeptide comprises an amino acid sequence as set forth in SEQ ID NO: 2.

In particular embodiments, the signaling component comprises an FKBP12 polypeptide or variant thereof. In various embodiments, a signaling component comprises an FKBP12 polypeptide comprising a F36V mutation. In one embodiment, the FKBP12 polypeptide comprises an amino acid sequence having at least 90% identity to SEQ ID NO: 3. In one embodiment, the FKBP12 polypeptide comprises an amino acid sequence having at least 95% identity to SEQ ID NO: 3. In one embodiment, the FKBP12 polypeptide comprises an amino acid sequence having at least 96% identity to SEQ ID NO: 3. In one embodiment, the FKBP12 polypeptide comprises an amino acid sequence having at least 97% identity to SEQ ID NO: 3. In one embodiment, the FKBP12 polypeptide comprises an amino acid sequence having at least 98% identity to SEQ ID NO: 3. In one embodiment, the FKBP12 polypeptide comprises an amino acid sequence having at least 99% identity to SEQ ID NO: 3. In particular embodiments, the FKBP12 polypeptide comprises the amino acid sequence as set forth in SEQ ID NO: 3. In particular embodiments, the FKBP12 polypeptide comprises the amino acid sequence as set forth in SEQ ID NO: 4. In one embodiment, the FKBP12 polypeptide comprises an amino acid sequence having at least 90% identity to SEQ ID NO: 4. In one embodiment, the FKBP12 polypeptide comprises an amino acid sequence having at least 95% identity to SEQ ID NO: 4. In one embodiment, the FKBP12 polypeptide comprises an amino acid sequence having at least 96% identity to SEQ ID NO: 4. In one embodiment, the FKBP12 polypeptide comprises an amino acid sequence having at least 97% identity to SEQ ID NO: 4. In one embodiment, the FKBP12 polypeptide comprises an amino acid sequence having at least 98% identity to SEQ ID NO: 4. In one embodiment, the FKBP12 polypeptide comprises an amino acid sequence having at least 99% identity to SEQ ID NO: 4. In particular embodiments, the FKBP12 polypeptide comprises the amino acid sequence as set forth in SEQ ID NO: 4.

In various embodiments, the first multimerization domain (e.g., FRB or FKBP) and the CD3ε domain/polypeptide are separated by a first polypeptide linker of 2 to 40 amino acids in length. In various embodiments, a short oligo- or poly-peptide linker, preferably between 1, 2, 3, 4, 5, 6, 7, 8, 9, 10, 11, 12, 13, 14, 15, 16, 17, 18, 19, or 20 amino acids in length links one or more domains in the signaling component. A glycine-serine based linker provides a particularly suitable linker. In some embodiments, the first polypeptide linker is selected from the group consisting of: GG, GS, SG, SS, GSS, SSG, GSG, SGS, SGG, G4S, 2×G4S, 3×G4S, 4×G4S, and any combination thereof. In some embodiments, the first polypeptide linker is a G4S (GGGGS; SEQ ID NO: 11) linker. In some embodiments, the first polypeptide linker is a 2×G4S (GGGGSGGGGS; SEQ ID NO: 12) linker. In some embodiments, the first polypeptide linker is a 3×G4S (GGGGSGGGGSGGGGS; SEQ ID NO: 13) linker. In some embodiments, the first polypeptide linker is a 4×G4S (GGGGSGGGGSGGGGSGGGGS; SEQ ID NO: 14) linker. In some embodiments, the first polypeptide linker is a 5×G4S (GGGGSGGGGSGGGGSGGGGSGGGGS; SEQ ID NO: 15) linker. In one embodiment, the linker has a sequence set forth as SEQ ID NO: 16. In one embodiment, the linker has a sequence set forth as SEQ ID NO: 17. In one embodiment, the linker has a sequence set forth as SEQ ID NO: 18. In one embodiment, the linker has a sequence set forth as SEQ ID NO: 19. In one embodiment, the linker has a sequence set forth as SEQ ID NO: 20. In one embodiment, the linker has a sequence set forth as SEQ ID NO: 21. In one embodiment, the linker has a sequence set forth as SEQ ID NO: 22. In one embodiment, the linker has a sequence set forth as SEQ ID NO: 23. In one embodiment, the linker has a sequence set forth as SEQ ID NO: 24. In one embodiment, the linker has a sequence set forth as SEQ ID NO: 25. In one embodiment, the linker has a sequence set forth as SEQ ID NO: 26. In one embodiment, the linker has a sequence set forth as SEQ ID NO: 27. In one embodiment, the linker has a sequence set forth as SEQ ID NO: 28. In one embodiment, the linker has a sequence set forth as SEQ ID NO: 29. In one embodiment, the linker has a sequence set forth as SEQ ID NO: 30. In one embodiment, the linker has a sequence set forth as SEQ ID NO: 31.

In various embodiments, a signaling component contemplated herein comprises a signal peptide, e.g., secretion signal polypeptide or signal sequence. Illustrative examples of signal peptides/sequences suitable for use in particular signaling components include but are not limited to an IgG1 heavy chain signal polypeptide, an Igκ light chain signal polypeptide, a CD8a signal polypeptide, or a human GM-CSF receptor alpha signal polypeptide. In various embodiments, a signaling component comprises a Igκ light chain signal polypeptide. In some embodiments, the Igκ light chain signal polypeptide comprises an amino acid sequence as set forth in SEQ ID NO: 95. In various embodiments, a signaling component comprises a CD8a signal polypeptide. In some embodiments, the CD8a light chain signal polypeptide comprises an amino acid sequence as set forth in SEQ ID NO: 96. In various embodiments, a signaling component comprises a PD1 signal polypeptide. In one embodiment, the PD1 signal polypeptide comprises an amino acid sequence set forth as SEQ ID NO: 97.

In particular embodiments, the signaling component comprises an FRB polypeptide or variant thereof, a 3×G4S linker, and a CD3ε polypeptide or variant thereof. In particular embodiments, the signaling component comprises a signaling sequence, an FRB polypeptide or variant thereof, a 3×G4S linker, and a CD3ε polypeptide or variant thereof. In particular embodiments, the signaling component comprises a CD8 signaling sequence, an FRB* polypeptide or variant thereof, a 3×G4S linker, and a CD3ε polypeptide or variant thereof. In particular embodiments, the signaling component comprises a CD8 signaling sequence (e.g., as set for in SEQ ID NO: 96, an FRB* polypeptide or variant thereof (e.g., as set forth in SEQ ID NO: 1, a 3×G4S linker (e.g., as set for in SEQ ID NO: 13), and a CD3ε polypeptide (e.g., as set for in SEQ ID NO: 32).

### 2. Targeting Component

A “targeting component” refers to a polypeptide comprising an extracellular domain, one or more multimerization domains, and a transmembrane domain, that in conjunction with a signaling component re-directs activation of an immune receptor complex to one or more selected target antigens. In some embodiments, the extracellular domain comprises one or more targeting domains that associate with and/or binds one or more target antigens. In particular embodiments, a targeting component comprises a first targeting domain, a second multimerization domain, a hinge domain, and a transmembrane domain.

In particular embodiments, a targeting component comprises an extracellular domain comprising at least one targeting domain, wherein the targeting domain is an antibody or antigen binding fragment thereof directed against one or more target antigens. Antigen binding fragments directed against one or more target antigens suitable for use in particular embodiments contemplated herein include those selected from the group consisting of: a Camel Ig, a Llama Ig, an Alpaca Ig, Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (e.g., sdAb, a camelid VHH, or Nanobody).

In various embodiments, the targeting domain comprises a single-chain variable fragment (scFv) or single domain antibody (sdAb). In various embodiments, the targeting domain comprise a single-chain variable fragment (scFv). In some embodiments, the sdAb is a camelid VHH, nanobody, or heavy chain-only antibody (HcAb). In some embodiments, the sdAb is a camelid VHH. In some embodiments, the scFv or sdAb is human or humanized. In particular embodiments, the VHH is humanized.

The disclosure provides a platform technology which is applicable to numerous classes and fragments of targeting domains and should not be limited to, for example, one particular targeting domain, e.g., antibody, antigen-binding fragment, ectodomain, etc., for targeting a specific target antigen.

For example, in some embodiments, the target antigen is an antigen expressed on a target cell, including, for example, cancer cells. In some embodiments, the targeting domain binds a CD33 target antigen and/or a C-type lectin-like molecule-1 (CLL-1) target antigen. In various embodiments, the targeting component comprises an extracellular domain that comprises a second targeting domain.

In various embodiments, the second targeting domain comprises a single-chain variable fragment (scFv) or single domain antibody (sdAb). In various embodiments, the second targeting domain comprises a single-chain variable fragment (scFv). In some embodiments, the sdAb is a camelid VHH, nanobody, or heavy chain-only antibody (HcAb). In some embodiments, the sdAb is a camelid VHH. In some embodiments, the scFv or sdAb is human or humanized. In particular embodiments, the VHH is humanized.

The extracellular domains contemplated in particular embodiments comprise a second targeting domain that binds a CD33 target antigen and/or a C-type lectin-like molecule-1 (CLL-1) target antigen. In one embodiment, the targeting domain comprises a CD33 targeting domain. In one embodiment, the CD33 targeting domain is a VHH. In one embodiment, the CD33 VHH comprises a CDR1 comprising SEQ ID NO: 89, a CDR2 comprising SEQ ID NO: 90, and a CDR3 comprising SEQ ID NO: 91. In one embodiment, the targeting domain comprises a sequence having at least 90% identity to SEQ ID NO: 72. In one embodiment, the targeting domain comprises a sequence having at least 95% identity to SEQ ID NO: 72. In one embodiment, the targeting domain comprises a sequence having at least 96% identity to SEQ ID NO: 72. In one embodiment, the targeting domain comprises a sequence having at least 97% identity to SEQ ID NO: 72. In one embodiment, the targeting domain comprises a sequence having at least 98% identity to SEQ ID NO: 72. In one embodiment, the targeting domain comprises a sequence having at least 99% identity to SEQ ID NO: 72. In one embodiment, the targeting domain comprises a sequence set forth as SEQ ID NO: 72.

In one embodiment, the targeting domain is a CLL1 targeting domain. In one embodiment, the targeting domain comprises a CLL1 targeting domain. In one embodiment, the CLL1 targeting domain is a VHH. In one embodiment, the CD33 VHH comprises a CDR1 comprising SEQ ID NO: 92, a CDR2 comprising SEQ ID NO: 93, and a CDR3 comprising SEQ ID NO: 94. In one embodiment, the targeting domain comprises a sequence having at least 90% identity to SEQ ID NO: 75. In one embodiment, the targeting domain comprises a sequence having at least 95% identity to SEQ ID NO: 75. In one embodiment, the targeting domain comprises a sequence having at least 96% identity to SEQ ID NO: 75. In one embodiment, the targeting domain comprises a sequence having at least 97% identity to SEQ ID NO: 75. In one embodiment, the targeting domain comprises a sequence having at least 98% identity to SEQ ID NO: 75. In one embodiment, the targeting domain comprises a sequence having at least 99% identity to SEQ ID NO: 75. In one embodiment, the targeting domain comprises a sequence set forth as SEQ ID NO: 75.

In one embodiment, the targeting component comprises a first targeting domain and a second targeting domain. In one embodiment, the first targeting domain is a CD33 targeting domain, and the second targeting domain is a CLL1 targeting domain. In another embodiment, the first targeting domain is a CLL1 targeting domain, and the second targeting domain is a CD33 targeting domain.

In one embodiment, the first targeting domain is a CD33 VHH, wherein, the CD33 VHH comprises a CDR1 comprising SEQ ID NO: 89, a CDR2 comprising SEQ ID NO: 90, and a CDR3 comprising SEQ ID NO: 91; and the second targeting domain is a CLL1 VHH, wherein the CLL1 VHH comprises a CDR1 comprising SEQ ID NO: 92, a CDR2 comprising SEQ ID NO: 93, and a CDR3 comprising SEQ ID NO: 94.

In one embodiment, the second targeting domain is a CD33 VHH, wherein, the CD33 VHH comprises a CDR1 comprising SEQ ID NO: 89, a CDR2 comprising SEQ ID NO: 90, and a CDR3 comprising SEQ ID NO: 91; and the first targeting domain is a CLL1 VHH, wherein the CLL1 VHH comprises a CDR1 comprising SEQ ID NO: 92, a CDR2 comprising SEQ ID NO: 93, and a CDR3 comprising SEQ ID NO: 94.

In various embodiments, the first targeting domain and the second targeting domain are separated by a third polypeptide linker 2 to 40 amino acids in length. In some embodiments, the third polypeptide linker is selected from the group consisting of: GG, GS, SG, SS, GSS, SSG, GSG, SGS, SGG, G4S, 2×G4S, 3×G4s, 4×G4S, and any combination thereof.

In particular embodiments, a targeting component comprises one or more multimerization domains.

Illustrative examples of multimerization domains suitable for use in particular targeting components contemplated herein include, but are not limited to, an FK506 binding protein (FKBP) polypeptide or variants thereof, an FKBP-rapamycin binding (FRB) polypeptide or variants thereof, a calcineurin polypeptide or variants thereof, a cyclophilin polypeptide or variants thereof, a bacterial dihydrofolate reductase (DHFR) polypeptide or variants thereof, a PYR1-like 1 (PYL1) polypeptide or variants thereof and an abscisic acid insensitive 1 (ABI1) polypeptide or variants thereof.

In various embodiments, a targeting component comprises an FRB polypeptide. In various embodiments, a targeting component comprises an FRB polypeptide comprising a T2098L (T82L) mutation, or variant thereof. In particular embodiments, the FRB polypeptide comprises the amino acid sequence as set forth in SEQ ID NO: 1. In particular embodiments, the FRB polypeptide comprises the amino acid sequence as set forth in SEQ ID NO: 2.

In various embodiments, a targeting component comprises an FKBP12 polypeptide or variant thereof. In various embodiments, a targeting component comprises an FKBP12 polypeptide comprising a F36V mutation. In particular embodiments, the FKBP12 polypeptide comprises the amino acid sequence as set forth in SEQ ID NO: 3. In particular embodiments, the FKBP12 polypeptide comprises the amino acid sequence as set forth in SEQ ID NO: 4.

Other illustrative examples of multimerization domains suitable for use in particular targeting components contemplated herein include antibody derived heterodimerization domains.

Such antibody heterodimerization domains include, but are not limited to, lock and key, DEKK, SEEDbody, DuoBody, dual variable domain immunoglobulin (DVD-Ig), and Fabs-in-tandem immunoglobulin (FIT-Ig) multimerization domains (see, e.g., Ma et al., Front Immunol. 2021 May 5; 12:626616.)

In various embodiments, the extracellular domain or first targeting domain, and the second multimerization domain, are separated by a second polypeptide linker 2 to 40 amino acids in length. In various embodiments, a short oligo- or poly-peptide linker, preferably between 1, 2, 3, 4, 5, 6, 7, 8, 9, 10, 11, 12, 13, 14, 15, 16, 17, 18, 19, or 20 amino acids in length links one or more domains in the targeting component. A glycine-serine based linker provides a particularly suitable linker. In some embodiments, the second polypeptide linker is selected from the group consisting of: GG, GS, SG, SS, GSS, SSG, GSG, SGS, SGG, G4S, 2×G4S, 3×G4S, 4×G4S, and any combination thereof. In some embodiments, the second polypeptide linker is a G4S (GGGGS; SEQ ID NO: 11) linker. In some embodiments, the second polypeptide linker is a 2×G4S (GGGGSGGGGS; SEQ ID NO: 12) linker. In some embodiments, the second polypeptide linker is a 3×G4S (GGGGSGGGGSGGGGS; SEQ ID NO: 13) linker. In some embodiments, the second polypeptide linker is a 4×G4S (GGGGSGGGGSGGGGSGGGGS; SEQ ID NO: 14) linker. In some embodiments, the second polypeptide linker is a 5×G4S (GGGGSGGGGSGGGGSGGGGSGGGGS; SEQ ID NO: 15) linker. In some embodiments, the second polypeptide linker comprises an amino acid sequence as set forth in any one or more of SEQ ID NOs: 16-31.

In various embodiments, the second multimerization domain and the transmembrane domain are separated by a hinge domain. In some embodiments, the hinge domain is a CD4 hinge. In some embodiments, the CD4 hinge comprises an amino acid sequence having at least 90% identity to SEQ ID NO: 41. In some embodiments, the CD4 hinge comprises an amino acid sequence having at least 95% identity to SEQ ID NO: 41. In some embodiments, the CD4 hinge comprises an amino acid sequence having at least 96% identity to SEQ ID NO: 41. In some embodiments, the CD4 hinge comprises an amino acid sequence having at least 97% identity to SEQ ID NO: 41. In some embodiments, the CD4 hinge comprises an amino acid sequence having at least 98% identity to SEQ ID NO: 41. In some embodiments, the CD4 hinge comprises an amino acid sequence having at least 99% identity to SEQ ID NO: 41. In some embodiments, the CD4 hinge comprises an amino acid sequence as set forth in SEQ ID NO: 41.

In some embodiments, the targeting component comprises a linker comprising an GGR amino acid sequence. In some embodiments, the targeting component comprises a linker comprising an amino acid sequence as set forth in SEQ ID NO: 13 or SEQ ID NO: 14.

In particular embodiments, a targeting component comprises a transmembrane domain.

Illustrative examples of transmembrane domains suitable for use in particular targeting components contemplated herein include, but are not limited to, the transmembrane region(s) of the alpha, beta, gamma, or delta chain of a T-cell receptor, CD3ε, CD3ζ, CD4, CD5, CD8α, CD9, CD 16, CD22, CD27, CD28, CD33, CD3γ, CD45, CD64, CD71, CD80, CD86, CD 134, CD137, CD152, CD 154, amnionless (AMN), and programmed cell death 1 (PDCD1). In a various embodiment, a targeting component comprises a CD4 transmembrane domain, CD28 transmembrane domain, or a CD8a transmembrane domain.

In a particular embodiment, a targeting component comprises a CD4 transmembrane domain. In some embodiments, the CD4 transmembrane domain comprises an amino acid sequence having at least 90% identity to SEQ ID NO: 45. In some embodiments, the CD4 transmembrane domain comprises an amino acid sequence having at least 95% identity to SEQ ID NO: 45. In some embodiments, the CD4 transmembrane domain comprises an amino acid sequence having at least 96% identity to SEQ ID NO: 45. In some embodiments, the CD4 transmembrane domain comprises an amino acid sequence having at least 97% identity to SEQ ID NO: 45. In some embodiments, the CD4 transmembrane domain comprises an amino acid sequence having at least 98% identity to SEQ ID NO: 45. In some embodiments, the CD4 transmembrane domain comprises an amino acid sequence having at least 99% identity to SEQ ID NO: 45. In some embodiments, the CD4 transmembrane domain comprises an amino acid sequence as set forth in SEQ ID NO: 45.

In particular embodiments, a targeting component comprises a truncated CD4 intracellular (CD4ic) domain. In one embodiment, the truncated CD4ic domain comprises an amino acid sequence having at least 90% identity to SEQ ID NO:48. In one embodiment, the truncated CD4ic domain comprises an amino acid sequence having at least 95% identity to SEQ ID NO: 48. In one embodiment, the truncated CD4ic domain comprises an amino acid sequence having at least 96% identity to SEQ ID NO: 48. In one embodiment, the truncated CD4ic domain comprises an amino acid sequence having at least 97% identity to SEQ ID NO: 48. In one embodiment, the truncated CD4ic domain comprises an amino acid sequence having at least 98% identity to SEQ ID NO: 48. In one embodiment, the truncated CD4ic domain comprises an amino acid sequence having at least 99% identity to SEQ ID NO: 48. In various embodiments, the truncated CD4ic domain comprises an amino acid sequence as set forth in SEQ ID NO: 48. In one embodiment, the truncated CD4ic domain comprises an amino acid sequence having at least 90% identity to SEQ ID NO: 49. In one embodiment, the truncated CD4ic domain comprises an amino acid sequence having at least 95% identity to SEQ ID NO: 49. In one embodiment, the truncated CD4ic domain comprises an amino acid sequence having at least 96% identity to SEQ ID NO: 49. In one embodiment, the truncated CD4ic domain comprises an amino acid sequence having at least 97% identity to SEQ ID NO: 49. In one embodiment, the truncated CD4ic domain comprises an amino acid sequence having at least 98% identity to SEQ ID NO: 49. In one embodiment, the truncated CD4ic domain comprises an amino acid sequence having at least 99% identity to SEQ ID NO: 49. In various embodiments, the truncated CD4ic domain comprises an amino acid sequence as set forth in SEQ ID NO: 49.

In particular embodiments, the targeting component does not comprise a primary signaling domain (e.g., CD3z). In various embodiments, the targeting component does not comprise an intracellular signaling or costimulatory domain.

In various embodiments, the targeting component further comprises a truncated intracellular CD4 polypeptide. In some embodiments, the truncated intracellular CD4 polypeptide comprises an amino acid sequence as set forth in SEQ ID NO: 48. In some embodiments, the truncated intracellular CD4 polypeptide comprises an amino acid sequence as set forth in SEQ ID NO: 49.

In a preferred embodiment, a targeting component comprises an extracellular domain that comprises a first targeting domain comprising a VHH or scFv that binds a CLL1, a linker polypeptide, a second targeting domain comprising a VHH or scFv that binds CD33, a linker polypeptide, an FRB or FKBP12 multimerization domain, a CD4 hinge, a CD4 transmembrane domain, and a truncated CD4ic domain. In a preferred embodiment, a targeting component comprises an extracellular domain that comprises a first targeting domain comprising a VHH or scFv that binds a CD33, a linker polypeptide, a second targeting domain comprising a VHH or scFv that binds CLL1, a linker polypeptide, an FRB or FKBP12 multimerization domain, a CD4 hinge, a CD4 transmembrane domain, and a truncated CD4ic domain.

In one embodiment, the targeting component comprises a sequence having at least 90% identity to SEQ ID NO: 122. In one embodiment, the targeting component comprises a sequence having at least 95% identity to SEQ ID NO: 122. In one embodiment, the targeting component comprises a sequence having at least 96% identity to SEQ ID NO: 122. In one embodiment, the targeting component comprises a sequence having at least 97% identity to SEQ ID NO: 122. In one embodiment, the targeting component comprises a sequence having at least 98% identity to SEQ ID NO: 122. In one embodiment, the targeting component comprises a sequence having at least 99% identity to SEQ ID NO: 122. In one embodiment, the targeting component comprises a sequence set forth as SEQ ID NO: 122.

In various embodiments, a targeting component contemplated herein comprises a signal peptide, e.g., secretion signal polypeptide or sequence. Illustrative examples of signal polypeptides/sequences suitable for use in particular targeting components include but are not limited to an IgG1 heavy chain signal polypeptide, an Igκ light chain signal polypeptide, a CD8a signal polypeptide, or a human GM-CSF receptor alpha signal polypeptide.

In various preferred embodiments, a targeting component comprises a Igκ light chain signal polypeptide. In some embodiments, the Igκ light chain signal polypeptide comprises an amino acid sequence having at least 90% identity to SEQ ID NO: 95. In some embodiments, the Igκ light chain signal polypeptide comprises an amino acid sequence having at least 95% identity to SEQ ID NO: 95. In some embodiments, the Igκ light chain signal polypeptide comprises an amino acid sequence having at least 96% identity to SEQ ID NO: 95. In some embodiments, the Igκ light chain signal polypeptide comprises an amino acid sequence having at least 97% identity to SEQ ID NO: 95. In some embodiments, the Igκ light chain signal polypeptide comprises an amino acid sequence having at least 98% identity to SEQ ID NO: 95. In some embodiments, the Igκ light chain signal polypeptide comprises an amino acid sequence having at least 99% identity to SEQ ID NO: 95. In some embodiments, the IgK light chain signal polypeptide comprises an amino acid sequence as set forth in SEQ ID NO: 95.

In particular embodiments, the targeting component comprises a CLL1 targeting domain, a 3×G4S linker, a CD33 targeting domain, a 3×G4S linker, an FKBP polypeptide or variant thereof, a CD4 hinge domain, a CD4 transmembrane domain, and a truncated intracellular CD4 polypeptide. In particular embodiments, the targeting component comprises a signaling sequence, a CLL1 targeting domain, a 3×G4S linker, a CD33 targeting domain, a 3×G4S linker, an FKBP polypeptide or variant thereof, a CD4 hinge domain, a CD4 transmembrane domain, and a truncated intracellular CD4 polypeptide. In particular embodiments, the targeting component comprises an IgK signaling sequence, a CLL1 VHH targeting domain, a 3×G4S linker, a CD33 VHH targeting domain, a 3×G4S linker, an FKBP polypeptide or variant thereof, a CD4 hinge domain, a CD4 transmembrane domain, and a truncated intracellular CD4 polypeptide. In particular embodiments, the targeting component comprises an IgK signaling sequence (e.g., as set forth as SEQ ID NO: 95), a CLL1 VHH targeting domain (e.g., comprising a CDR1 as set forth as SEQ ID NO: 92, a CDR2 as set forth as SEQ ID NO: 93, and a SEQ ID NO: 94; and/or a VHH as set forth as SEQ ID NO: 75), a 3×G4S linker (e.g., as set forth in SEQ ID NO: 13), a CD33 VHH targeting domain (e.g., comprising a CDR1 set forth as SEQ ID NO: 89, a CDR2 set forth as SEQ ID NO: 90, a CDR3 set forth as SEQ ID NO: 91; and/or a VHH comprising SEQ ID NO: 72), a 3×G4S linker (e.g., as set forth in SEQ ID NO: 13), an FKBP polypeptide or variant thereof (e.g., as set forth as SEQ ID NO: 3), a CD4 hinge domain (e.g., as set forth as SEQ ID NO: 41), a CD4 transmembrane domain (e.g., as set forth as SEQ ID NO: 45), and a truncated intracellular CD4 polypeptide (e.g., as set forth as SEQ ID NO: 48 or SEQ ID NO: 49).

### 3. Bridging Factor

Bridging factors contemplated in particular embodiments herein mediate or promote the association of one or more signaling components with one or more targeting components through multimerization domains in the respective components. A bridging factor associates with and is disposed between the multimerization domains to promote association of a signaling component and a targeting component. In the presence of a bridging factor, the targeting component and the signaling component associate with an immune receptor complex and initiate immune effector cell activity against a target cell when the targeting component is bound to a target antigen on the target cell. In the absence of a bridging factor, the targeting component does not associate with the signaling component, does not recruit an immune receptor complex and the engineered receptor is inactive against the target antigen of the targeting component.

In particular embodiments, a signaling component and a targeting component comprise a cognate pair of multimerization domains selected from the group consisting of: FKBP and FKBP12-rapamycin binding (FRB), FKBP and calcineurin, FKBP and cyclophilin, FKBP and bacterial dihydrofolate reductase (DHFR), calcineurin and cyclophilin, and PYR1-like 1 (PYL1) and abscisic acid insensitive 1 (ABI1).

In certain embodiments, the multimerization domains of signaling and targeting components associate with a bridging factor selected from the group consisting of: rapamycin or a rapalog thereof, coumermycin or a derivative thereof, gibberellin or a derivative thereof, abscisic acid (ABA) or a derivative thereof, methotrexate or a derivative thereof, cyclosporin A or a derivative thereof, FK506/cyclosporin A (FKCsA) or a derivative thereof, and trimethoprim (Tmp)-synthetic ligand for FK506 binding protein (FKBP) (SLF) or a derivative thereof.

In particular embodiments, a signaling component and a targeting component comprise one or more FRB and/or FKBP multimerization domains or variants thereof. In certain embodiments, a signaling component comprises an FKBP12 multimerization domain or variant thereof and a targeting component comprises an FRB multimerization domain or variant thereof. In certain embodiments, a signaling component comprises an FRB multimerization domain or variant thereof and a targeting component comprises an FKBP12 multimerization domain or variant thereof. In particular preferred embodiments, a signaling component comprises an FKBP12 or FKBP12 F36V multimerization domain or variant thereof, and a targeting component comprises an FRB T2098L multimerization domains or variant thereof. In particular preferred embodiments, a CD3 signaling component comprises an FRB T2098L (T82L) multimerization domain or variant thereof, and a targeting component comprises an FKBP12 or FKBP12 F36V multimerization domains or variant thereof.

Illustrative examples of bridging factors suitable for use in particular embodiments contemplated herein include, but are not limited to, AP1903, AP20187, AP21967 (also known as C-16-(S)-7-methylindolerapamycin), everolimus, novolimus, pimecrolimus, ridaforolimus, sirolimus, tacrolimus, temsirolimus, umirolimus, zotarolimus, and BPC015. In particular preferred embodiments, the bridging factor is AP21967. In certain preferred embodiments, the bridging factor is a non-immunosuppressive dose of sirolimus (rapamycin).

### D. Antigen Receptors and Binding Molecules

In particular embodiments, immune effector cells contemplated herein further express an exogenous lymphocyte receptor or engineered antigen receptor and one or more components of an engineered immune receptor complex or system as described herein. In some embodiments, the exogenous lymphocyte receptor or engineered antigen receptor is a chimeric antigen receptor (CAR), a chimeric costimulatory receptor (CCR), a T cell receptor (TCR), an αβ T cell receptor (αβ-TCR), a γδ T cell receptor (γδ-TCR), zetakine, or flip receptor. In some embodiments, the immune effector cells contemplated herein further express an exogenous costimulatory factor, immunomodulatory factor, agonist for a costimulatory factor, antagonist for an immunosuppressive factor, immune cell engager, or any combination thereof. In some embodiments, the immune effector cells contemplated herein further express a BiTE.

In various embodiments, the exogenous lymphocyte receptor or engineered antigen receptor is an engineered αβ or γδ T cell receptor (TCR), a chimeric antigen receptor (CAR), or a chimeric costimulatory receptor (CCR). In particular embodiments, the exogenous lymphocyte receptor is an engineered αβ or γδ T cell receptor. In particular embodiments, the engineered antigen receptor is a chimeric antigen receptor (CAR). In particular embodiments, the engineered antigen receptor is a chimeric costimulatory receptor (CCR). In various embodiments, immune effector cells contemplated herein comprise an engineered/exogenous TCR, and an engineered immune receptor component(s) as described herein. Without wishing to be limited to any particular theory, the signaling components of the engineered immune receptors components/systems contemplated herein comprise a multimerization domain fused to an actuator domain (e.g., CD3ε, CD3δ, CD3γ, or Fcγ subunit) that associates with a multimeric immune receptor complex, and thus, in the presence of bridging factor recruits an immune receptor complex by multimerizing with the targeting component comprising a second multimerization domain.

In one embodiment, T cells are engineered by introducing a polynucleotide or vector encoding an engineered antigen receptor (e.g., TCR, CAR, or CCR) and one or more components of an engineered immune receptor system separated by one or more polypeptide cleavage signals. In one embodiment, T cells are engineered by introducing a polynucleotide or vector encoding an engineered antigen receptor and a polynucleotide or vector encoding one or more components of an immune receptor system. In one embodiment, T cells engineered to express an engineered immune receptor are further engineered by introducing a polynucleotide or vector encoding one or more components of an immune receptor system.

Naturally occurring T cell receptors comprise two subunits, an alpha chain and a beta chain subunit (αβ TCR), or a gamma chain and a delta chain subunit (γδ TCR), each of which is a unique protein produced by recombination event in each T cell's genome. Libraries of TCRs may be screened for their selectivity to particular target antigens. In this manner, natural TCRs, which have a high-avidity and reactivity toward target antigens may be selected, cloned, and subsequently introduced into a population of T cells used for adoptive immunotherapy. In one embodiment, the TCR is an αβ TCR. In one embodiment, the TCR is a γδ TCR.

In one embodiment, T cells are modified by introducing a TCR subunit that has the ability to form TCRs that confer specificity to T cells for cells expressing a target antigen. In particular embodiments, the subunits have one or more amino acid substitutions, deletions, insertions, or modifications compared to the naturally occurring subunit, so long as the subunits retain the ability to form TCRs and confer upon transfected T cells the ability to home to target cells, and participate in immunologically-relevant cytokine signaling. The engineered TCRs preferably also bind target cells displaying the relevant peptide (e.g., a tumor-associated peptide) with high avidity, and optionally mediate efficient killing of target cells presenting the relevant peptide in vivo.

The nucleic acids encoding engineered TCRs are preferably isolated from their natural context in a (naturally-occurring) chromosome of a T cell, and can be incorporated into suitable vectors as described elsewhere herein. Both the nucleic acids and the vectors comprising them can be transferred into a cell, preferably a T cell in particular embodiments. The modified T cells are then able to express one or more chains of a TCR encoded by the transduced nucleic acid or nucleic acids. In preferred embodiments, the TCR is an exogenous TCR because it is introduced into T cells that do not normally express the particular TCR. In particular embodiments, the essential aspect of the TCR is that it has high avidity for an antigen presented by a major histocompatibility complex (MHC) or similar immunological component. In contrast to TCRs, CARs are engineered to bind target antigens in an MHC independent manner.

The TCR can be expressed with additional polypeptides attached to the amino-terminal or carboxyl-terminal portion of the alpha chain or beta chain of a TCR, or of the gamma chain or delta chain of a TCR so long as the attached additional polypeptide does not interfere with the ability of the alpha chain or beta chain to form a functional T cell receptor and the MHC dependent antigen recognition.

Antigens that are recognized by the TCRs contemplated in particular embodiments include, but are not limited to cancer antigens, including antigens on both hematological cancers and solid tumors. Illustrative antigens include, but are not limited to α-fetoprotein (AFP), B Melanoma Antigen (BAGE) family members, Brother of the regulator of imprinted sites (BORIS), Cancer-testis antigens, Cancer-testis antigen 83 (CT-83), Carbonic anhydrase IX (CA1X), Carcinoembryonic antigen (CEA), Cytomegalovirus (CMV) antigens, Cytotoxic T cell (CTL)-recognized antigen on melanoma (CAMEL), Epstein-Barr virus (EBV) antigens, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human Epidermal Growth Factor Receptor 2 (HER-2), Human papillomavirus (HPV)-E6, HPV-E7, Human telomerase reverse transcriptase (hTERT), IGF2BP3/A3, K-Ras, K-Ras G12C, K-Ras G12D, K-Ras G12V, Latent membrane protein 2 (LMP2), Melanoma antigen family A, 1 (MAGE-A1), MAGE-A2, MAGE-A3, MAGE-A4, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53,P antigen (PAGE) family members, Placenta-specific 1 (PLAC1), Preferentially expressed antigen in melanoma (PRAME), Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, Tyrosinase, Tyrosinase related protein (TRP)1, TRP2, Wilms tumor protein (WT-1), X Antigen Family Member 1 (XAGE1), and X Antigen Family Member 2 (XAGE2).

In other embodiments, immune effector cells contemplated herein comprise a CAR and an and an engineered immune receptor component(s) as described herein. Chimeric antigen receptors (CARs) are molecules that combine antibody-based specificity for a target antigen (e.g., tumor antigen) with a T cell receptor-activating intracellular domain to generate a chimeric protein that exhibits a specific anti-tumor cellular immune activity. As used herein, the term, “chimeric,” describes being composed of parts of different proteins or DNAs from different origins.

In other embodiments, immune effector cells contemplated herein comprise a CCR and an engineered immune receptor component(s) as described herein. Unlike CARs, chimeric costimulatory receptors (CCRs) are molecules that combine antibody-based specificity for a desired antigen with a T cell receptor-costimulatory domain but that lacks a primary signaling domain (see, e.g., WO2020/252110 and WO2021/211948).

In other embodiments, immune effector cells contemplated herein express a bispecific T cell engager (BiTE) and an engineered immune receptor component(s) as described herein. BiTEs are molecules that comprise two different antibody fragments (e.g., single-chain variable fragments (scFvs)) connected by a small linker peptide, wherein in one antibody fragment binds a component of the T cell receptor (TCR) complex (e.g., a CD3 chain), and the other antibody fragment binds another antigen (e.g., a tumor associated antigen).

In other embodiments, immune effector cells contemplated herein comprise a flip receptor and an engineered immune receptor component(s) as described herein. A flip receptor comprises an extracellular portion of a receptor, a transmembrane domain, and an intracellular signaling portion of a different receptor. Thus, a flip receptor “converts” the binding properties of one receptor type into the intracellular signaling event of another receptor type. See, e.g., WO2018/094244, WO2016/122738, WO2014/172584, and WO2012/138858.

In certain embodiments, the CAR, CCR, immune cell engager, BiTE, or flip receptor binds a target antigen selected from the group consisting of: alpha folate receptor (FRα), αᵥβ₆ integrin, B cell maturation antigen (BCMA), B7-H3 (CD276), B7-H6, carbonic anhydrase IX (CAIX), CD16, CD19, CD20, CD22, CD30, CD33, CD3γ, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IGF2BP3/A3, IL-10Rα, IL-13Rα2, Kappa, cancer/testis antigen 2 (LAGE-IA), K-Ras, K-Ras G12C, K-Ras G12D, K-Ras G12V, Lambda, Lewis-Y (LeY), L1 cell adhesion molecule (L1-CAM), melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), transmembrane activator and CAML interactor (TACI), tumor endothelial marker 1 (TEM1/CD248), tumor endothelial marker 7-related (TEM7R), trophoblast glycoprotein (TPBG), UL16-binding protein (ULBP) 1, ULBP2, ULBP3, ULBP4, ULBP5, ULBP6, vascular endothelial growth factor receptor 2 (VEGFR2), and Wilms tumor 1 (WT-1).

In particular embodiments, the CAR or CCR binds a target antigen selected from the group consisting of: BCMA, CD33, CD20, CD79a, CD79b, CLL-1, IGF2BP3/A3, MUC16, NY-ESO, PRAME, TACI, and TP53.

In various embodiments, immune effector cells contemplated herein comprise one or more chains of a zetakine receptor and an engineered immune receptor component(s) as described herein. Zetakines are chimeric transmembrane immunoreceptors that comprise an extracellular domain comprising a soluble receptor ligand linked to a support region capable of tethering the extracellular domain to a cell surface, a transmembrane region and an intracellular signaling domain. Zetakines, when expressed on the surface of T lymphocytes, direct T cell activity to those cells expressing a receptor for which the soluble receptor ligand is specific.

Zetakine chimeric immunoreceptors redirect the antigen specificity of T cells, with application to treatment of a variety of cancers, particularly via the autocrine/paracrine cytokine systems utilized by human malignancy.

In other embodiments, immune effector cells are modified by introducing an Fc Receptor (FcR) subunit that has the ability to bind to the fragment crystallizable (Fc) portion/region of an antibody, and an engineered immune receptor component(s) as described herein. In some embodiments, the FcR subunit can be from an Fc-gamma receptor, an Fc-alpha receptor, or an Fc-epsilon receptor. See, e.g., Mkaddem et al., Front Immunol. 2019 Apr. 12; 10:811.

In other embodiments, immune effector cells are modified by introducing NKG2D receptor, and an engineered immune receptor component(s) as described herein.

### E. Polypeptides

Various polypeptides are contemplated herein, including, but not limited to, an engineered immune receptor system, targeting components, signaling components, immune receptors and their associated subunits or adaptor molecules, exogenous lymphocyte receptor, engineered antigen receptors, TCRs, FcRs, CARs, CCRs, BiTEs, zetakines, flip receptors, exogenous costimulatory factors, immunomodulatory factors, agonist for a costimulatory factors, antagonist for an immunosuppressive factors, immune cell engagers, or any combination thereof. Also contemplated herein are fusion proteins comprising the foregoing polypeptides, and fragments thereof. In preferred embodiments, a polypeptide comprises an amino acid sequence set forth in any one of SEQ ID NOs: 107-115, 116-138, or 139-170.

“Polypeptide,” “peptide” and “protein” are used interchangeably, unless specified to the contrary, and according to conventional meaning, i.e., as a sequence of amino acids. In one embodiment, a “polypeptide” includes fusion polypeptides and other variants. Polypeptides can be prepared using any of a variety of well-known recombinant and/or synthetic techniques.

Polypeptides are not limited to a specific length, e.g., they may comprise a full-length protein sequence, a fragment of a full-length protein, or a fusion protein, and may include post-translational modifications of the polypeptide, for example, glycosylations, acetylations, phosphorylations and the like, as well as other modifications known in the art, both naturally occurring and non-naturally occurring. In particular preferred embodiments, fusion polypeptides, polypeptides, fragments and other variants thereof are prepared, obtained, or isolated from one or more human polypeptides.

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. In particular embodiments, an isolated polypeptide is a synthetic polypeptide, a semi-synthetic polypeptide, or a polypeptide obtained or derived from a recombinant source.

Polypeptides include “polypeptide variants.” Polypeptide variants may differ from a naturally occurring polypeptide in one or more substitutions, deletions, additions and/or insertions. Such variants may be naturally occurring or may be synthetically generated, for example, by modifying one or more of the above polypeptide sequences. For example, in particular embodiments, it may be desirable to improve the binding affinity and/or other biological properties of a polypeptide by introducing one or more substitutions, deletions, additions and/or insertions in the polypeptide. In particular embodiments, polypeptides include polypeptides having at least about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% amino acid identity to any of the reference sequences contemplated herein, typically where the variant maintains at least one biological activity of the reference sequence. In particular embodiments, the biological activity is binding affinity. In particular embodiments, the biological activity is enzymatic activity.

In certain embodiments, an engineered immune receptor component(s) as described herein that recruits a multimeric immune receptor complex comprising (i) a signaling component, e.g., first fusion polypeptide, having a first multimerization domain and an actuator domain and (ii) a targeting component, e.g., second fusion polypeptide, having a second multimerization domain, an extracellular domain, and a transmembrane domain. In particular embodiments, the multimerization domains are the same; in certain embodiments, the first multimerization domain is different than the second multimerization domain. The first and second multimerization domains substantially contribute to or efficiently promote formation of the polypeptide complex, e.g., in the presence of a bridging factor. The interaction(s) between the first and second multimerization domains substantially contributes to or efficiently promotes the multimerization of the first and second fusion polypeptides if there is a statistically significant reduction in the association between the first and second fusion polypeptides in the absence of the first multimerization domain, the second multimerization domain, or the bridging factor. In certain embodiments, when the first and second fusion polypeptides are co-expressed, at least about 60%, for instance, at least about 60% to about 70%, at least about 70% to about 80%, at least about 80% to about 90%, 91%, 92%, 93%, 94%, 95%, 96%, 97%, 98%, 99%, or 100%, and at least about 90% to about 92%, 93%, 94%, 95%, 96%, 97%, 98%, or 99% of the first and second single chain polypeptides form multimers with each other in the presence of a bridging factor.

Polypeptide variants include biologically active “polypeptide fragments.” Illustrative examples of biologically active polypeptide fragments include binding domains, intracellular signaling domains, and the like. As used herein, the term “biologically active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring polypeptide activity. In certain embodiments, a polypeptide fragment can comprise an amino acid chain at least 5 to about 1700 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42,43, 44,45,46, 47,48,49, 50, 55, 60, 65, 70,75, 80, 85, 90, 95, 100, 110, 150, 200,250, 300, 350, 400,450, 500, 550, 600, 650, 700,750, 800, 850, 900, 950, 1000, 1100, 1200, 1300, 1400, 1500, 1600, 1700 or more amino acids long.

In particular embodiments, the polypeptides set forth herein may comprise one or more amino acids denoted as “X.” “X” if present in an amino acid SEQ ID NO, refers to any one or more amino acids. In particular embodiments, SEQ ID NOs denoting a fusion protein comprise a sequence of continuous X residues that cumulatively represent any amino acid sequence.

As noted above, polypeptides may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, a polypeptide variant comprises one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides contemplated in particular embodiments and still obtain a functional molecule that encodes a variant or derivative polypeptide with desirable characteristics. When it is desired to alter the amino acid sequence of a polypeptide to create an equivalent, or even an improved, variant polypeptide, one skilled in the art, for example, can change one or more of the codons of the encoding DNA sequence, e.g., according to Table 1.

TABLE 1
Amino Acid Codons

| | One | Three | | | | | | |
|---|---|---|---|---|---|---|---|---|
| Amino | letter | letter | | | | | | |
| Acids | code | code | Codons | | | | | |
| Alanine | A | Ala | GCA | GCC | GCG | GCU | | |
| Cysteine | C | Cys | UGC | UGU | | | | |
| Aspartic acid | D | Asp | GAC | GAU | | | | |
| Glutamic | E | Glu | GAA | GAG | | | | |
| acid | | | | | | | | |
| Phenyl- | F | Phe | UUC | UUU | | | | |
| alanine | | | | | | | | |
| Glycine | G | Gly | GGA | GGC | GGG | GGU | | |
| Histidine | H | His | CAC | CAU | | | | |
| Isoleucine | I | Iso | AUA | AUC | AUU | | | |
| Lysine | K | Lys | AAA | AAG | | | | |
| Leucine | L | Leu | UUA | UUG | CUA | CUC | CUG | CUU |
| Methionine | M | Met | AUG | | | | | |
| Asparagine | N | Asn | AAC | AAU | | | | |
| Proline | P | Pro | CCA | CCC | CCG | CCU | | |
| Glutamine | Q | Gln | CAA | CAG | | | | |
| Arginine | R | Arg | AGA | AGG | CGA | CGC | CGG | CGU |
| Serine | S | Ser | AGC | AGU | UCA | UCC | UCG | UCU |
| Threonine | T | Thr | ACA | ACC | ACG | ACU | | |
| Valine | V | Val | GUA | GUC | GUG | GUU | | |
| Tryptophan | W | Trp | UGG | | | | | |
| Tyrosine | Y | Tyr | UAC | UAU | | | | |

Guidance in determining which amino acid residues can be substituted, inserted, or deleted without abolishing biological activity can be found using computer programs well known in the art, such as DNASTAR, DNA Strider, Geneious, Mac Vector, or Vector NTI software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p.224).

In one embodiment, where expression of two or more polypeptides is desired, the polynucleotide sequences encoding them can be separated by an IRES sequence.

Polypeptides contemplated in particular embodiments include fusion polypeptides. In particular embodiments, fusion polypeptides and polynucleotides encoding fusion polypeptides are provided. Fusion polypeptides and fusion proteins refer to a polypeptide having at least two, three, four, five, six, seven, eight, nine, or ten, or more polypeptide segments. In preferred embodiments, a fusion polypeptide comprises one or more of the components described herein.

In other preferred embodiments, the fusion polypeptide comprises a targeting component and a signaling component.

In particular embodiments, two or more engineered immune receptor components and/or other polypeptides can be expressed as a fusion protein that comprises one or more self-cleaving peptide sequences between the polypeptides as disclosed elsewhere herein.

In particular embodiments, a fusion polypeptide comprises a targeting component and one or more signaling components.

Fusion polypeptides can comprise one or more polypeptide domains or segments including, but are not limited to signal peptides, cell permeable peptide domains (CPP), binding domains, signaling domains, etc., epitope tags (e.g., maltose binding protein (“MBP”), glutathione S transferase (GST), HIS6, MYC, FLAG, V5, VSV-G, and HA), polypeptide linkers, and polypeptide cleavage signals. Fusion polypeptides are typically linked C-terminus to N-terminus, although they can also be linked C-terminus to C-terminus, N-terminus to N-terminus, or N-terminus to C-terminus. In particular embodiments, the polypeptides of the fusion protein can be in any order. Fusion polypeptides or fusion proteins can also include conservatively modified variants, polymorphic variants, alleles, mutants, subsequences, and interspecies homologs, so long as the desired activity of the fusion polypeptide is preserved. Fusion polypeptides may be produced by chemical synthetic methods or by chemical linkage between the two moieties or may generally be prepared using other standard techniques. Ligated DNA sequences comprising the fusion polypeptide are operably linked to suitable transcriptional or translational control elements as disclosed elsewhere herein.

Fusion polypeptides may optionally comprise one or more linkers that can be used to link the one or more polypeptides or domains within a polypeptide. A peptide linker sequence may be employed to separate any two or more polypeptide components by a distance sufficient to ensure that each polypeptide folds into its appropriate secondary and tertiary structures so as to allow the polypeptide domains to exert their desired functions. Such a peptide linker sequence is incorporated into the fusion polypeptide using standard techniques in the art. Suitable peptide linker sequences may be chosen based on the following factors: (1) their ability to adopt a flexible extended conformation; (2) their inability to adopt a secondary structure that could interact with functional epitopes on the signaling component and targeting component; and (3) the lack of hydrophobic or charged residues that might react with the polypeptide functional epitopes. In particular embodiments, preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natd. Acad. Sci. USA 83:8258-8262, 1986; U.S. Pat. Nos. 4,935,233 and 4,751,180. Linker sequences are not required when a particular fusion polypeptide segment contains non-essential N-terminal amino acid regions that can be used to separate the functional domains and prevent steric interference. In particular embodiments, preferred linkers are typically flexible amino acid subsequences which are synthesized as part of a recombinant fusion protein. Linker polypeptides can be between 1 and 200 amino acids in length, between 1 and 100 amino acids in length, or between 1 and 50 amino acids in length, including all integer values in between.

Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO: 195), for example, ENLYFQG (SEQ ID NO: 196) and ENLYFQS (SEQ ID NO: 197), wherein X represents any amino acid (cleavage by TEV occurs between Q and G or Q and S).

In particular embodiments, the polypeptide cleavage signal is a viral self-cleaving peptide or ribosomal skipping sequence.

Illustrative examples of ribosomal skipping sequences include but are not limited to: a 2A or 2A-like site, sequence or domain (Donnelly et al., 2001. J. Gen. Virol. 82:1027-1041). In a particular embodiment, the viral 2A peptide is an aphthovirus 2A peptide, a potyvirus 2A peptide, or a cardiovirus 2A peptide.

In one embodiment, the viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

Illustrative examples of 2A sites are provided in Table 2.

TABLE 2

| | |
|---|---|
| SEQ ID NO: 171 | GSGATNFSLLKQAGDVEENPGP |
| SEQ ID NO: 172 | ATNFSLLKQAGDVEENPGP |
| SEQ ID NO: 173 | LLKQAGDVEENPGP |
| SEQ ID NO: 174 | GSGEGRGSLLTCGDVEENPGP |
| SEQ ID NO: 175 | EGRGSLLTCGDVEENPGP |
| SEQ ID NO: 176 | LLTCGDVEENPGP |
| SEQ ID NO: 177 | GSGQCTNYALLKLAGDVESNPGP |
| SEQ ID NO: 178 | QCTNYALLKLAGDVESNPGP |
| SEQ ID NO: 179 | LLKLAGDVESNPGP |
| SEQ ID NO: 180 | GSGVKQTLNFDLLKLAGDVESNPGP |
| SEQ ID NO: 181 | VKQTLNFDLLKLAGDVESNPGP |
| SEQ ID NO: 182 | LLKLAGDVESNPGP |
| SEQ ID NO: 183 | LLNFDLLKLAGDVESNPGP |
| SEQ ID NO: 184 | TLNFDLLKLAGDVESNPGP |
| SEQ ID NO: 185 | LLKLAGDVESNPGP |
| SEQ ID NO: 186 | NFDLLKLAGDVESNPGP |
| SEQ ID NO: 187 | QLLNFDLLKLAGDVESNPGP |
| SEQ ID NO: 188 | APVKQTLNFDLLKLAGDVESNPGP |
| SEQ ID NO: 189 | VTELLYRMKRAETYCPRPLLAIHPT |
| | EARHKQKIVAPVKQT |
| SEQ ID NO: 190 | LNFDLLKLAGDVESNPGP |
| SEQ ID NO: 191 | LLAIHPTEARHKQKIVAPVKQTLNF |
| | DLLKLAGDVESNPGP |
| SEQ ID NO: 192 | EARHKQKIVAPVKQTLNFDLLKLAG |
| | DVESNPGP |

In particular embodiments, the self-cleaving polypeptides (e.g., 2A peptides) comprise a SGSG (SEQ ID NO: 193) spacer sequence. In particular embodiments, the self-cleaving polypeptides (e.g., 2A peptides) comprise a furin recognition site, e.g., RAKR (SEQ ID NO: 194) spacer sequence.

In preferred embodiments, a polypeptide or fusion polypeptide comprises one or more engineered immune receptors or components. In preferred embodiments, a fusion polypeptide comprises one or more engineered immune receptors or components separated by one or more self-cleaving polypeptides.

In particular embodiments, a fusion polypeptide comprises a signaling component comprising a first multimerization domain and an actuator domain that forms a complex with and recruits an immune receptor complex (e.g., a CD3ε, CD3δ or CD3γ, domain; a viral self-cleaving 2A polypeptide; and a targeting component comprising an extracellular domain (comprising at least a first targeting domain (e.g., an antibody or antigen binding fragment thereof, that binds to a target antigen)), a second multimerization domain, and a transmembrane domain.

In particular embodiments, a fusion polypeptide comprises a signaling component comprising a first multimerization domain and an actuator domain that forms a complex with and recruits an immune receptor complex (e.g., a CD3ε, CD3δ or CD3γ, domain; a viral self-cleaving 2A polypeptide; and a targeting component comprising an extracellular domain (comprising at least a first targeting domain (e.g., an antibody or antigen binding fragment thereof, that binds to a target antigen)), a second multimerization domain, a CD4 hinge domain, and a CD4 transmembrane domain.

In particular embodiments, a fusion polypeptide comprises a signaling component comprising a first multimerization domain and an actuator domain that forms a complex with and recruits an immune receptor complex (e.g., a CD3ε, CD3δ or CD3γ, domain; a viral self-cleaving 2A polypeptide; and a targeting component comprising an extracellular domain (comprising at least a first targeting domain (e.g., an antibody or antigen binding fragment thereof, that binds to a target antigen)), a second multimerization domain, a CD4 hinge domain, a CD4 transmembrane domain, and a truncated CD4ic domain.

In particular embodiments, a fusion polypeptide comprises a signaling component comprising an FRB or FKBP12 multimerization domain and an actuator domain that forms a complex with and recruits an immune receptor complex (e.g., a CD3ε, CD3δ or CD3γ, domain; a viral self-cleaving 2A polypeptide; and a targeting component comprising an extracellular domain comprising a VHH or scFv that binds a target antigen, an FRB or FKBP12 multimerization domain, a CD4 hinge, a CD4 transmembrane domain, and a truncated CD4ic domain.

In particular embodiments, a fusion polypeptide comprises a signaling component comprising an antibody derived heterodimerization domain multimerization domain and an actuator domain that forms a complex with and recruits an immune receptor complex (e.g., a CD3ε, CD3δ or CD3γ, domain; a viral self-cleaving 2A polypeptide; and a targeting component comprising VHH or scFv that binds a target antigen, an antibody derived heterodimerization domain, a CD4 hinge, a CD4 transmembrane domain, a truncated CD4ic domain, and optionally, a costimulatory or cytokine receptor intracellular signaling domain.

In particular embodiments, the fusion polypeptides comprise an amino acid sequence as set forth in any one of SEQ ID NOs: 139-170.

In particular embodiments, a fusion polypeptide comprises a signaling component comprising a first multimerization domain and an actuator domain that forms a complex with and recruits an Fc receptor complex (e.g., a Fcγ domain/polypeptide); a viral self-cleaving 2A polypeptide; and a targeting component comprising an extracellular domain (comprising at least a first targeting domain (e.g., an antibody or antigen binding fragment thereof, that binds to a target antigen)), a second multimerization domain, optionally a hinge domain, and a transmembrane domain.

In particular embodiments, a fusion polypeptide comprises a signaling component comprising a first multimerization domain and an actuator domain that forms a complex with and recruits an Fc receptor complex (e.g., a Fcγ domain/polypeptide); a viral self-cleaving 2A polypeptide; and a targeting component comprising an extracellular domain (comprising at least a first targeting domain (e.g., an antibody or antigen binding fragment thereof, that binds to a target antigen)), a second multimerization domain, a CD4 hinge, and a CD4 transmembrane domain.

In particular embodiments, a fusion polypeptide comprises a signaling component comprising a first multimerization domain and an actuator domain that forms a complex with and recruits an Fc receptor complex (e.g., a Fcγ domain/polypeptide); a viral self-cleaving 2A polypeptide; and a targeting component comprising an extracellular domain (comprising at least a first targeting domain (e.g., an antibody or antigen binding fragment thereof, that binds to a target antigen)), a second multimerization domain, a CD4 hinge, a CD4 transmembrane domain, and a truncated CD4ic domain.

In particular embodiments, a fusion polypeptide comprises a signaling component comprising an FRB or FKBP12 multimerization domain and an actuator domain that forms a complex with and recruits an Fc receptor complex (e.g., a Fcγ domain/polypeptide); a viral self-cleaving 2A polypeptide; and a targeting component comprising an extracellular domain comprising a VHH or scFv that binds a target antigen, an FRB or FKBP12 multimerization domain, a CD4 hinge, a CD4 transmembrane domain, and a truncated CD4ic domain.

In particular embodiments, a fusion polypeptide comprises a signaling component comprising an antibody derived heterodimerization domain and an actuator domain that forms a complex with and recruits an Fc receptor complex (e.g., a Fcγ domain/polypeptide); a viral self-cleaving 2A polypeptide; and a targeting component comprising an extracellular domain comprising a VHH or scFv that binds a target antigen, an antibody derived heterodimerization domain, a CD4 hinge, a CD4 transmembrane domain, a truncated CD4ic domain, and optionally, a costimulatory or cytokine receptor intracellular signaling domain.

In particular embodiments, the fusion polypeptides comprise an amino acid sequence as set forth in any one of SEQ ID NOs: 139-170.

In one embodiment, the fusion polypeptide comprises an amino acid sequence having at least 90% identity to SEQ ID NO: 151. In one embodiment, the fusion polypeptide comprises an amino acid sequence having at least 95% identity to SEQ ID NO: 151. In one embodiment, the fusion polypeptide comprises an amino acid sequence having at least 96% identity to SEQ ID NO: 151. In one embodiment, the fusion polypeptide comprises an amino acid sequence having at least 97% identity to SEQ ID NO: 151. In one embodiment, the fusion polypeptide comprises an amino acid sequence having at least 98% identity to SEQ ID NO: 151. In one embodiment, the fusion polypeptide comprises an amino acid sequence having at least 99% identity to SEQ ID NO: 151. In one embodiment, the fusion polypeptide comprises an amino acid sequence as set forth as SEQ ID NO: 151.

In one embodiment, the fusion polypeptide comprises a sequence having at least 90% identity to SEQ ID NO: 137. In one embodiment, the fusion polypeptide comprises a sequence having at least 95% identity to SEQ ID NO: 137. In one embodiment, the fusion polypeptide comprises a sequence having at least 96% identity to SEQ ID NO: 137. In one embodiment, the fusion polypeptide comprises a sequence having at least 97% identity to SEQ ID NO: 137. In one embodiment, the fusion polypeptide comprises a sequence having at least 98% identity to SEQ ID NO: 137. In one embodiment, the fusion polypeptide comprises a sequence having at least 99% identity to SEQ ID NO: 137. In one embodiment, the fusion polypeptide comprises a sequence set forth as SEQ ID NO: 137.

In one embodiment, the fusion polypeptide comprises a sequence having at least 90% identity to SEQ ID NO: 138. In one embodiment, the fusion polypeptide comprises a sequence having at least 95% identity to SEQ ID NO: 138. In one embodiment, the fusion polypeptide comprises a sequence having at least 96% identity to SEQ ID NO: 138. In one embodiment, the fusion polypeptide comprises a sequence having at least 97% identity to SEQ ID NO: 138. In one embodiment, the fusion polypeptide comprises a sequence having at least 98% identity to SEQ ID NO: 138. In one embodiment, the fusion polypeptide comprises a sequence having at least 99% identity to SEQ ID NO: 138. In one embodiment, the fusion polypeptide comprises a sequence set forth as SEQ ID NO: 138.

### F. Polynucleotides

In particular embodiments, polynucleotides encoding an engineered immune receptor system, one or more engineered immune receptor components, signaling components, targeting components, exogenous lymphocyte receptors, engineered TCRs, CARs, CCRs, BiTEs, zetakines, flip receptors, exogenous costimulatory factors, immunomodulatory factors, agonist for a costimulatory factors, antagonist for an immunosuppressive factors, immune cell engagers, fusion proteins comprising the foregoing polypeptides, and fragments thereof.

As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded and either recombinant, synthetic, or isolated. Polynucleotides include, but are not limited to: pre-messenger RNA (pre-mRNA), messenger RNA (mRNA), RNA, short interfering RNA (siRNA), short hairpin RNA (shRNA), microRNA (miRNA), ribozymes, genomic RNA (gRNA), plus strand RNA (RNA(+)), minus strand RNA (RNA(−)), tracrRNA, crRNA, single guide RNA (sgRNA), synthetic RNA, synthetic mRNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%,76%, 77%, 78%, 79%, 80%, 81%, 82%, 83%, 84%,85%, 86%, 87%, 88%, 89%, 90%, 91%, 92%, 93%, 94%, 95%, 96%, 97%, 98%, 99% or 100% sequence identity to a reference sequence.

As used herein, “isolated polynucleotide” refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” also refers to a complementary DNA (cDNA), a recombinant DNA, or other polynucleotide that does not exist in nature and that has been made by the hand of man. In particular embodiments, an isolated polynucleotide is a synthetic polynucleotide, a semi-synthetic polynucleotide, or a polynucleotide obtained or derived from a recombinant source.

In various embodiments, a polynucleotide comprises an mRNA encoding a polypeptide contemplated herein. In certain embodiments, the mRNA comprises a cap, one or more nucleotides, and a poly(A) tail.

In particular embodiments, polynucleotides described herein, including polynucleotides encoding one or more engineered immune receptor components, may be codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, (x) systematic variation of codon sets for each amino acid, and/or (xi) isolated removal of spurious translation initiation sites.

As used herein the term “nucleotide” refers to a heterocyclic nitrogenous base in N-glycosidic linkage with a phosphorylated sugar. Nucleotides are understood to include natural bases, and a wide variety of art-recognized modified bases. Such bases are generally located at the 1′ position of a nucleotide sugar moiety. Nucleotides generally comprise a base, sugar and a phosphate group. In ribonucleic acid (RNA), the sugar is a ribose, and in deoxyribonucleic acid (DNA) the sugar is a deoxyribose, i.e., a sugar lacking a hydroxyl group that is present in ribose.

Illustrative examples of polynucleotides include, but are not limited to, polynucleotides encoding polypeptides set forth in SEQ ID NOs: 107-170.

In various illustrative embodiments, polynucleotides contemplated herein include, but are not limited to polynucleotides encoding one or more engineered immune receptor components, engineered antigen receptors, exogenous lymphocyte receptors, fusion polypeptides, and expression vectors, viral vectors, and transfer plasmids comprising polynucleotides contemplated herein.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% sequence identity to any of the reference sequences described herein.

The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. Vectors may comprise 1, 2, 3, 4, 5, 6, 7, 8, 9 or 10 or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. The cassette can be removed and inserted into a plasmid or viral vector as a single unit.

Polynucleotides include polynucleotide(s)-of-interest. As used herein, the term “polynucleotide-of-interest” refers to a polynucleotide encoding a polypeptide or fusion polypeptide or a polynucleotide that serves as a template for the transcription of an inhibitory polynucleotide, as contemplated herein.

The polynucleotides contemplated herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

Polynucleotides can be prepared, manipulated, expressed and/or delivered using any of a variety of well-established techniques known and available in the art. In order to express a desired polypeptide, a nucleotide sequence encoding the polypeptide, can be inserted into appropriate vector.

Illustrative examples of vectors include, but are not limited to plasmid, autonomously replicating sequences, and transposable elements, e.g., Sleeping Beauty, PiggyBac.

Additional Illustrative examples of vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses.

Illustrative examples of viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40).

Illustrative examples of expression vectors include, but are not limited to, pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

“Expression control sequences,” “control elements,” or “regulatory sequences” present in an expression vector are those non-translated regions of the vector including an origin of replication, selection cassettes, promoters, enhancers, translation initiation signals (Shine Dalgarno sequence or Kozak sequence) introns, a polyadenylation sequence, 5′ and 3′ untranslated regions, all of which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, a polynucleotide comprises a vector, including but not limited to expression vectors and viral vectors. A vector may comprise one or more exogenous, endogenous, or heterologous control sequences such as promoters and/or enhancers. An “endogenous control sequence” is one which is naturally linked with a given gene in the genome. An “exogenous control sequence” is one which is placed in juxtaposition to a gene by means of genetic manipulation (i.e., molecular biological techniques) such that transcription of that gene is directed by the linked enhancer/promoter. A “heterologous control sequence” is an exogenous sequence that is from a different species than the cell being genetically manipulated. A “synthetic” control sequence may comprise elements of one more endogenous and/or exogenous sequences, and/or sequences determined in vitro or in silico that provide optimal promoter and/or enhancer activity for the particular therapy.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) U3 promoter (Haas et al. Journal of Virology. 2003; 77(17): 9439-9450).

In one embodiment, a vector comprises an MNDU3 promoter.

In one embodiment, a vector comprises an EF1a promoter comprising the first intron of the human EF1a gene.

In one embodiment, a vector comprises an EF1a promoter that lacks the first intron of the human EF1a gene.

In a particular embodiment, it may be desirable to use a cell, cell type, cell lineage or tissue specific expression control sequence to achieve cell type specific, lineage specific, or tissue specific expression of a desired polynucleotide sequence (e.g., to express a particular nucleic acid encoding a polypeptide in only a subset of cell types, cell lineages, or tissues or during specific stages of development).

In a particular embodiment, it may be desirable to express a polynucleotide a T cell specific promoter.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc. Inducer agents include, but are not limited to glucocorticoids, estrogens, mifepristone (RU486), metals, interferons, small molecules, cumate, tetracycline, doxycycline, and variants thereof.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. Examples of IRES generally employed by those of skill in the art include those described in U.S. Pat. No. 6,692,736. Further examples of “IRES” known in the art include, but are not limited to IRES obtainable from picomavirus (Jackson et al., 1990) and IRES obtainable from viral or cellular mRNA sources, such as for example, immunoglobulin heavy-chain binding protein (BiP), the vascular endothelial growth factor (VEGF) (Huez et al. 1998. Mol. Cell. Biol. 18(11):6178-6190), the fibroblast growth factor 2 (FGF-2), and insulin-like growth factor (IGFII), the translational initiation factor eIF4G and yeast transcription factors TFIID and HAP4, the encephelomycarditis virus (EMCV) which is commercially available from Novagen (Duke et al., 1992. J. Virol 66(3):1602-9) and the VEGF IRES (Huez et al., 1998. Mol Cell Biol 18(11):6178-90). IRES have also been reported in viral genomes of Picornaviridae, Dicistroviridae and Flaviviridae species and in HCV, Friend murine leukemia virus (FrMLV) and Moloney murine leukemia virus (MoMLV).

In one embodiment, the IRES used in polynucleotides contemplated herein is an EMCV IRES.

In particular embodiments, the polynucleotides a consensus Kozak sequence. As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO: 198), where R is a purine (A or G) (Kozak, 1986. Cell. 44(2):283-92, and Kozak, 1987. Nucleic Acids Res. 15(20):8125-48).

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal polyA sequence (e.g., AATAAA, ATTAAA, AGTAAA). In particular embodiments, the poly(A) sequence is an SV40 polyA sequence, a bovine growth hormone polyA sequence (BGHpA), a rabbit P-globin polyA sequence (rpgpA), variants thereof, or another suitable heterologous or endogenous polyA sequence known in the art. In particular embodiments, the poly(A) sequence is synthetic.

In particular embodiments, polynucleotides encoding one or more polypeptides, or fusion polypeptides may be introduced into immune effector cells, e.g., T cells, by both non-viral and viral methods. In particular embodiments, delivery of one or more polynucleotides may be provided by the same method or by different methods, and/or by the same vector or by different vectors.

The term “vector” is used herein to refer to a nucleic acid molecule capable of transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to a T cell.

Illustrative examples of non-viral vectors include, but are not limited to plasmids (e.g., DNA plasmids or RNA plasmids), transposons, cosmids, and bacterial artificial chromosomes.

Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al. (2003) Gene Therapy. 10:180-187; and Balazs et al. (2011) Journal of Drug Delivery. 2011:1-12. Antibody-targeted, bacterially derived, non-living nanocell-based delivery is also contemplated in particular embodiments.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term “viral vector” or “lentiviral vector” may refer either to a virus or viral particle capable of transferring a nucleic acid into a cell or to the transferred nucleic acid itself. Viral vectors and transfer plasmids contain structural and/or functional genetic elements that are primarily derived from a virus.

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, viral vectors comprising polynucleotides contemplated herein are administered directly to an organism or subject for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Illustrative examples of viral vector systems suitable for use in particular embodiments contemplated in particular embodiments include, but are not limited to, adeno-associated virus (AAV), retrovirus, herpes simplex virus, adenovirus, and vaccinia virus vectors.

In various embodiments, one or more polynucleotides encoding one or more engineered immune receptor components and/or other polypeptides contemplated herein are introduced into an immune effector cell, e.g., T cell, by transducing the cell with a recombinant adeno-associated virus (rAAV), comprising the one or more polynucleotides.

AAV is a small (˜26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5′ and 3′ AAV inverted terminal repeats (ITRs). The ITR sequences are about 145 bp in length. In particular embodiments, the rAAV comprises ITRs and capsid sequences isolated from AAV1, AAV2, AAV3, AAV4, AAV5, AAV6, AAV7, AAV8, AAV9, or AAV10.

In some embodiments, a chimeric rAAV is used the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may comprise ITRs from AAV2, and capsid proteins from any one of AAV1, AAV2, AAV3, AAV4, AAV5, AAV6, AAV7, AAV8, AAV9, or AAV10. In a preferred embodiment, the rAAV comprises ITR sequences derived from AAV2 and capsid sequences derived from AAV6. In a preferred embodiment, the rAAV comprises ITR sequences derived from AAV2 and capsid sequences derived from AAV2.

In some embodiments, engineering and selection methods can be applied to AAV capsids to make them more likely to transduce cells of interest.

Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

In various embodiments, one or more polynucleotides encoding one or more engineered immune receptor components and/or other polypeptides contemplated herein are introduced into an immune effector cell, e.g., T cell, by transducing the cell with a retrovirus, e.g., lentivirus, comprising the one or more polynucleotides.

As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to, HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

In various embodiments, a lentiviral vector contemplated herein comprises one or more LTRs, and one or more, or all, of the following accessory elements: a cPPT/FLAP, a Psi (Ψ) packaging signal, an export element, poly (A) sequences, and may optionally comprise a WPRE or HPRE, an insulator element, a selectable marker, and a cell suicide gene, as discussed elsewhere herein.

In particular embodiments, lentiviral vectors contemplated herein may be integrative or non-integrating or integration defective lentivirus. As used herein, the term “integration defective lentivirus” or “IDLV” refers to a lentivirus having an integrase that lacks the capacity to integrate the viral genome into the genome of the host cells. Integration-incompetent viral vectors have been described in patent application WO 2006/010834, which is herein incorporated by reference in its entirety.

Illustrative mutations in the HIV-1 pol gene suitable to reduce integrase activity include, but are not limited to: H12N, H12C, H16C, H16V, S81 R, D41A, K42A, H51A, Q53C, D55V, D64E, D64V, E69A, K71A, E85A, E87A, D116N, D1161, D116A, N120G, N1201, N120E, E152G, E152A, D35E, K156E, K156A, E157A, K159E, K159A, K160A, R166A, D167A, E170A, H171A, K173A, K186Q, K186T, K188T, E198A, R199c, R199T, R199A, D202A, K211A, Q214L, Q216L, Q221 L, W235F, W235E, K236S, K236A, K246A, G247W, D253A, R262A, R263A and K264H.

The term “long terminal repeat (LTR)” refers to domains of base pairs located at the ends of retroviral DNAs which, in their natural sequence context, are direct repeats and contain U3, R and U5 regions.

As used herein, the term “FLAP element” or “cPPT/FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173.

As used herein, the term “packaging signal” or “packaging sequence” refers to psi [Ψ] sequences located within the retroviral genome which are required for insertion of the viral RNA into the viral capsid or particle, see e.g., Clever et al., 1995. J. of Virology, Vol. 69, No. 4; pp. 2101-2109.

The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE).

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766).

Lentiviral vectors preferably contain several safety enhancements as a result of modifying the LTRs. “Self-inactivating” (SIN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. Self-inactivation is preferably achieved through the introduction of a deletion in the U3 region of the 3′ LTR of the vector DNA, i.e., the DNA used to produce the vector RNA. Thus, during reverse transcription, this deletion is transferred to the 5′ LTR of the proviral DNA. In particular embodiments, it is desirable to eliminate enough of the U3 sequence to greatly diminish or abolish altogether the transcriptional activity of the LTR, thereby greatly diminishing or abolishing the production of full-length vector RNA in transduced cells. In the case of HIV based lentivectors, it has been discovered that such vectors tolerate significant U3 deletions, including the removal of the LTR TATA box (e.g., deletions from −418 to −18), without significant reductions in vector titers.

An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters.

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4⁺ presenting cells.

In certain embodiments, lentiviral vectors are produced according to known methods. See e.g., Kutner et al., BMC Biotechnol. 2009; 9:10. doi: 10.1186/1472-6750-9-10; Kutner et al. Nat. Protoc. 2009; 4(4):495-505. doi: 10.1038/nprot.2009.22.

According to certain specific embodiments contemplated herein, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid contemplated herein.

In various embodiments, one or more polynucleotides encoding one or more engineered immune receptor components and/or other polypeptides contemplated herein are introduced into an immune effector cell, by transducing the cell with an adenovirus comprising the one or more polynucleotides.

Adenoviral based vectors are capable of very high transduction efficiency in many cell types and do not require cell division. With such vectors, high titer and high levels of expression have been obtained. This vector can be produced in large quantities in a relatively simple system. Most adenovirus vectors are engineered such that a transgene replaces the Ad Ela, Elb, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Generation and propagation of the current adenovirus vectors, which are replication deficient, may utilize a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). Adenovirus vectors have been used in eukaryotic gene expression (Levrero et al., 1991; Gomez-Foix et al., 1992) and vaccine development (Grunhaus & Horwitz, 1992; Graham & Prevec, 1992). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). An example of the use of an Ad vector in a clinical trial involved polynucleotide therapy for antitumor immunization with intramuscular injection (Sterman et al., Hum. Gene Ther. 7:1083-9 (1998)).

In various embodiments, one or more polynucleotides encoding one or more engineered immune receptor components and/or other polypeptides contemplated herein are introduced into an immune effector cell by transducing the cell with a herpes simplex virus, e.g., HSV-1, HSV-2, comprising the one or more polynucleotides.

The mature HSV virion consists of an enveloped icosahedral capsid with a viral genome consisting of a linear double-stranded DNA molecule that is 152 kb. In one embodiment, the HSV based viral vector is deficient in one or more essential or non-essential HSV genes. In one embodiment, the HSV based viral vector is replication deficient. Most replication deficient HSV vectors contain a deletion to remove one or more intermediate-early, early, or late HSV genes to prevent replication. For example, the HSV vector may be deficient in an immediate early gene selected from the group consisting of: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, each of which are incorporated by reference herein in its entirety.

### G. Genetically Modified Cells

In various embodiments, cells are modified to express an engineered immune receptor system, one or more engineered immune receptor components, signaling components, targeting components, engineered TCRs, CARs, CCRs, BiTEs, zetakines, flip receptors, exogenous costimulatory factor, immunomodulatory factor, agonist for a costimulatory factor, antagonist for an immunosuppressive factor, immune cell engager, and/or fusion proteins contemplated herein. In particular embodiments, the cells are for use in the treatment of cancer. Cells may be non-genetically modified to express one or more of the polypeptides contemplated herein, or in particular preferred embodiments, cells may be genetically modified to express one or more of the polypeptides contemplated herein. As used herein, the term “genetically engineered” or “genetically modified” refers to the addition of extra genetic material in the form of DNA or RNA into the total genetic material in a cell. The terms, “genetically modified cells,” “modified cells,” and “redirected cells,” are used interchangeably in particular embodiments.

In particular embodiments, one or more engineered immune receptor components that recruit an immune receptor complex, contemplated herein, are introduced and expressed in immune effector cells to improve the efficacy of the immune effector cells. In particular embodiments, one or more engineered immune receptor components that recruit an immune receptor complex are introduced and expressed in immune effector cells that have been redirected to a target cell by virtue of co-expressing an exogenous lymphocyte receptor or engineered antigen receptor, e.g., an engineered TCR or CAR, in the cell.

In particular embodiments, a dual targeting immune effector cell is contemplated where the target cell expresses a target antigen recognized by engineered immune receptor and an MHC-antigen complex recognized by a TCR, e.g., an engineered/exogenous TCR.

In particular embodiments, a dual targeting immune effector cell is contemplated where the target cell expresses CD33, CD123, CLL1, B7-H3, BCMA, CD19, CD20, CD22, CD79A, CD79B, EGFR, EGFRvIII, or an NKG2D ligand recognized by an engineered immune receptor, and a target antigen recognized by an exogenous lymphocyte receptor or engineered antigen receptor, e.g., an engineered/exogenous TCR or CAR.

An “immune effector cell,” is any cell of the immune system that has one or more effector functions (e.g., cytotoxic cell killing activity, secretion of cytokines, induction of ADCC and/or CDC). The illustrative immune effector cells contemplated herein are T lymphocytes, including but not limited to cytotoxic T cells (CTLs; CD8⁺ T cells), TILs, and helper T cells (HTLs; CD4⁺ T cells. In a particular embodiment, the cells comprise αβ T cells. In a particular embodiment, the cells comprise 78 T cells. In one embodiment, immune effector cells include natural killer (NK) cells. In one embodiment, immune effector cells include natural killer T (NKT) cells.

Immune effector cells can be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are human autologous immune effector cells.

Illustrative immune effector cells suitable for introducing one or more engineered immune receptor receptors or components contemplated herein include T lymphocytes. The terms “T cell” or “T lymphocyte” are art-recognized and are intended to include thymocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), CD4⁺CD8⁺ T cell, CD4-CD8- T cell, or any other subset of T cells. Other illustrative populations of T cells suitable for use in particular embodiments include naïve T cells and memory T cells. For example, populations of T cells suitable for use in particular embodiments include naïve T cells (T_{N}), T memory stem cells (T_{SCM}), central memory T cells (T_{CM}), effector memory T cells (T_{EM}), and effector T cells (T_{EFF}).

As would be understood by the skilled person, other cells may also be used as immune effector cells comprising one or more engineered immune receptor components contemplated herein. In particular embodiments, immune effector cells also include NK cells, NKT cells, neutrophils, and macrophages. Immune effector cells also include progenitors of effector cells wherein such progenitor cells can be induced to differentiate into immune effector cells in vivo or in vitro. Thus, in particular embodiments, immune effector cells include progenitors of immune effectors cells such as hematopoietic stem cells (HSCs) contained within the CD34⁺ population of cells derived from cord blood, bone marrow or mobilized peripheral blood which upon administration in a subject differentiate into mature immune effector cells, or which can be induced in vitro to differentiate into mature immune effector cells.

The term, “CD34⁺ cell,” as used herein refers to a cell expressing the CD34 protein on its cell surface. “CD34,” as used herein refers to a cell surface glycoprotein (e.g., sialomucin protein) that often acts as a cell-cell adhesion factor and is involved in T cell entrance into lymph nodes. The CD34⁺ cell population contains hematopoietic stem cells (HSC), which upon administration to a patient differentiate and contribute to all hematopoietic lineages, including T cells, NK cells, NKT cells, neutrophils and cells of the monocyte/macrophage lineage.

Methods for making the immune effector cells which express one or more engineered immune receptor components contemplated herein are provided in particular embodiments. In one embodiment, the method comprises transfecting or transducing immune effector cells isolated from an individual such that the immune effector cells with one or more nucleic acids and/or vectors or combination thereof comprising one or more engineered immune receptor components contemplated herein. In one embodiment, the method comprises transfecting or transducing immune effector cells isolated from an individual such that the immune effector cells express one or more engineered immune receptor components and an exogenous lymphocyte receptor or engineered antigen receptors contemplated herein (e.g., TCRs, CARs, CCRs, zetakines, or flip receptors). In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified. In this regard, the immune effector cells may be cultured before and/or after being genetically modified.

In particular embodiments, the cells are human cells. In particular embodiments, prior to in vitro manipulation or genetic modification of the immune effector cells described herein, the source of cells is obtained from a subject. In particular embodiments, the modified immune effector cells comprise T cells.

T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation.

In other embodiments, an isolated or purified population of T cells is used. In some embodiments, after isolation of PBMC, both cytotoxic and helper T lymphocytes can be sorted into naïve, memory, and effector T cell subpopulations either before or after activation, expansion, and/or genetic modification.

In one embodiment, an isolated or purified population of T cells expresses one or more of the markers including, but not limited to a CD3⁺, CD4⁺, CD8⁺, or a combination thereof.

In certain embodiments, the T cells are isolated from an individual and first activated and stimulated to proliferate in vitro prior to being modified to express one or more engineered immune receptor components.

In order to achieve sufficient therapeutic doses of T cell compositions, T cells are often subjected to one or more rounds of stimulation, activation and/or expansion. In particular embodiments, T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety. In particular embodiments, T cells are activated and expanded for about 6 hours, about 12 hours, about 18 hours or about 24 hours prior to introduction of vectors or polynucleotides encoding one or more engineered immune receptor components, optionally in combination with an exogenous lymphocyte receptor or engineered antigen receptor contemplated herein.

In one embodiment, T cells are activated at the same time that they are modified.

In various embodiments, a method of generating an immune effector cell comprises activating a population of cells comprising T cells and expanding the population of T cells. T cell activation can be accomplished by providing a primary stimulation signal through the T cell TCR/CD3 complex and by providing a secondary costimulation signal through an accessory molecule, e.g., CD28.

The TCR/CD3 complex may be stimulated by contacting the T cell with a suitable CD3 binding agent, e.g., a CD3 ligand or an anti-CD3 monoclonal antibody. Illustrative examples of CD3 antibodies include, but are not limited to, OKT3, G19-4, BC3, and 64.1.

In addition to the primary stimulation signal provided through the TCR/CD3 complex, induction of T cell responses requires a second, costimulatory signal. In particular embodiments, a CD28 binding agent can be used to provide a costimulatory signal. Illustrative examples of CD28 binding agents include but are not limited to: natural CD28 ligands, e.g., a natural ligand for CD28 (e.g., a member of the B7 family of proteins, such as B7-1(CD80) and B7-2 (CD86); and anti-CD28 monoclonal antibody or fragment thereof capable of crosslinking the CD28 molecule, e.g., monoclonal antibodies 9.3, B-T3, XR-CD28, KOLT-2, 15E8, 248.23.2, and EX5.3D10.

In one embodiment, the molecule providing the primary stimulation signal, for example a molecule which provides stimulation through the TCR/CD3 complex and the costimulatory molecule are coupled to the same surface.

In certain embodiments, binding agents that provide stimulatory and costimulatory signals are localized on the surface of a cell. This can be accomplished by transfecting or transducing a cell with a nucleic acid encoding the binding agent in a form suitable for its expression on the cell surface or alternatively by coupling a binding agent to the cell surface.

In another embodiment, the molecule providing the primary stimulation signal, for example a molecule which provides stimulation through the TCR/CD3 complex and the costimulatory molecule are displayed on antigen presenting cells.

In one embodiment, the molecule providing the primary stimulation signal, for example a molecule which provides stimulation through the TCR/CD3 complex and the costimulatory molecule are provided on separate surfaces.

In a certain embodiment, one of the binding agents that provides stimulatory and costimulatory signals is soluble (provided in solution) and the other agent(s) is provided on one or more surfaces. In a particular embodiment, the binding agents that provide stimulatory and costimulatory signals are both provided in a soluble form (provided in solution). In various embodiments, the methods for making T cells contemplated herein comprise activating T cells with soluble anti-CD3 and/or soluble anti-CD28 antibodies, or fragments thereof. In various embodiments, the methods for making T cells contemplated herein comprise activating T cells with surface bound anti-CD3 and/or surface bound anti-CD28 antibodies, or fragments thereof. In various embodiments, the methods for making T cells contemplated herein comprise activating T cells with bead-bound anti-CD3 and/or bead-bound anti-CD28 antibodies, or fragments thereof.

In one embodiment, expanding T cells activated by the methods contemplated herein further comprises culturing a population of cells comprising T cells for several hours (about 3 hours) to about 7 days to about 28 days or any hourly integer value in between. In another embodiment, the T cell composition may be cultured for 14 days. In a particular embodiment, T cells are cultured for about 21 days. In another embodiment, the T cell compositions are cultured for about 2-3 days. Several cycles of stimulation/activation/expansion may also be desired.

In particular embodiments, conditions appropriate for T cell culture include an appropriate media (e.g., Minimal Essential Media or RPMI Media 1640 or, X-vivo 15, (Lonza)) and one or more factors necessary for proliferation and viability including, but not limited to serum (e.g., fetal bovine or human serum), interleukin-2 (IL-2), insulin, IFN-7, IL-4, IL-7, IL-21, GM-CSF, IL-10, IL-12, IL-15, TGFβ, and TNF-α or any other additives suitable for the growth of cells known to the skilled artisan.

Further illustrative examples of cell culture media include, but are not limited to RPMI 1640, Clicks, AIM-V, DMEM, MEM, a-MEM, IMDM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells.

Antibiotics, e.g., penicillin and streptomycin, are included only in experimental cultures, not in cultures of cells that are to be infused into a subject. The target cells are maintained under conditions necessary to support growth, for example, an appropriate temperature (e.g., 370 C) and atmosphere (e.g., air plus 5% C02).

In particular embodiments, PBMCs or isolated T cells are contacted with a stimulatory agent and costimulatory agent, such as anti-CD3 and anti-CD28 antibodies, generally attached to a bead or other surface, in a culture medium with appropriate cytokines, such as IL-2, IL-7, and/or IL-15.

In other embodiments, artificial APC (aAPC) made by engineering K562, U937, 721.221, T2, and C1R cells to direct the stable expression and secretion, of a variety of costimulatory molecules and cytokines. In a particular embodiment K32 or U32 aAPCs are used to direct the display of one or more antibody-based stimulatory molecules on the AAPC cell surface. Populations of T cells can be expanded by aAPCs expressing a variety of costimulatory molecules including, but not limited to, CD137L (4-1BBL), CD134L (OX40L), and/or CD80 or CD86. Finally, the aAPCs provide an efficient platform to expand genetically modified T cells and to maintain CD28 expression on CD8 T cells. aAPCs provided in WO 03/057171 and US2003/0147869 are hereby incorporated by reference in their entirety.

In a particular embodiment, a polynucleotide encoding one or more engineered immune receptor components is introduced into the population of T cells. In a particular embodiment, a polynucleotide encoding one or more engineered immune receptor components is introduced into a population of T cells that express an exogenous lymphocyte receptor or engineered antigen receptor. The polynucleotides may be introduced into the T cells by microinjection, transfection, lipofection, heat-shock, electroporation, transduction, gene gun, microinjection, DEAE-dextran-mediated transfer, and the like.

In a preferred embodiment, polynucleotides are introduced into a T cell by viral transduction.

Illustrative examples of viral vector systems suitable for introducing a polynucleotide into an immune effector cell or CD34⁺ cell include but are not limited to adeno-associated virus (AAV), retrovirus, herpes simplex virus, adenovirus, vaccinia virus vectors for gene transfer.

In one embodiment, polynucleotides are introduced into a T cell by AAV transduction. In one embodiment, polynucleotides are introduced into a T cell by retroviral transduction. In one embodiment, polynucleotides are introduced into a T cell by lentiviral transduction. In one embodiment, polynucleotides are introduced into a T cell by adenovirus transduction. In one embodiment, polynucleotides are introduced into a T cell by herpes simplex virus transduction. In one embodiment, polynucleotides are introduced into a T cell by vaccinia virus transduction.

### H. Compositions and Formulations

The compositions contemplated herein may comprise one or more engineered immune receptor polypeptides, polynucleotides encoding engineered immune receptor polypeptides, vectors comprising same, genetically modified immune effector cells, bridging factors, etc. Compositions include, but are not limited to, pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” refers to a diluent, adjuvant, excipient, or vehicle with which the bridging factors, polypeptides, polynucleotides, vectors comprising same, or genetically modified immune effector cells are administered. Illustrative examples of pharmaceutical carriers can be sterile liquids, such as cell culture media, water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients in particular embodiments, include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Except insofar as any conventional media or agent is incompatible with the active ingredient, its use in the therapeutic compositions is contemplated. Supplementary active ingredients can also be incorporated into the compositions.

In one embodiment, a composition comprising a pharmaceutically acceptable carrier is suitable for administration to a subject. In particular embodiments, a composition comprising a carrier is suitable for parenteral administration, e.g., intravascular (intravenous or intraarterial), intraperitoneal or intramuscular administration. In particular embodiments, a composition comprising a pharmaceutically acceptable carrier is suitable for intraventricular, intraspinal, or intrathecal administration. Pharmaceutically acceptable carriers include sterile aqueous solutions, cell culture media, or dispersions. The use of such media and agents for pharmaceutically active substances is well known in the art. Except insofar as any conventional media or agent is incompatible with the bridging factors, polypeptides, polynucleotides, vectors comprising same, or genetically modified immune effector cells, use thereof in the pharmaceutical compositions is contemplated.

In particular embodiments, compositions contemplated herein comprise genetically modified T cells and a pharmaceutically acceptable carrier. A composition comprising a cell-based composition contemplated herein can be administered separately by enteral or parenteral administration methods or in combination with other suitable compounds to effect the desired treatment goals.

In particular embodiments, compositions contemplated herein comprise a bridging factor and a pharmaceutically acceptable carrier.

The pharmaceutically acceptable carrier must be of sufficiently high purity and of sufficiently low toxicity to render it suitable for administration to the human subject being treated. It further should maintain or increase the stability of the composition. The pharmaceutically acceptable carrier can be liquid or solid and is selected, with the planned manner of administration in mind, to provide for the desired bulk, consistency, etc., when combined with other components of the composition. For example, the pharmaceutically acceptable carrier can be, without limitation, a binding agent (e.g., pregelatinized maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyacrylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulfate, etc.). Other suitable pharmaceutically acceptable carriers for the compositions contemplated herein include, but are not limited to, water, salt solutions, alcohols, polyethylene glycols, gelatins, amyloses, magnesium stearates, talcs, silicic acids, viscous paraffins, hydroxymethylcelluloses, polyvinylpyrrolidones and the like.

Such carrier solutions also can contain buffers, diluents and other suitable additives. The term “buffer” as used herein refers to a solution or liquid whose chemical makeup neutralizes acids or bases without a significant change in pH. Examples of buffers contemplated herein include, but are not limited to, Dulbecco's phosphate buffered saline (PBS), Ringer's solution, 5% dextrose in water (D5W), normal/physiologic saline (0.9% NaCl).

The pharmaceutically acceptable carriers may be present in amounts sufficient to maintain a pH of the composition of about 7. Alternatively, the composition has a pH in a range from about 6.8 to about 7.4, e.g., 6.8, 6.9, 7.0, 7.1, 7.2, 7.3, and 7.4. In still another embodiment, the composition has a pH of about 7.4.

Compositions contemplated herein may comprise a nontoxic pharmaceutically acceptable medium. The compositions may be a suspension. The term “suspension” as used herein refers to non-adherent conditions in which cells are not attached to a solid support. For example, cells maintained as a suspension may be stirred or agitated and are not adhered to a support, such as a culture dish.

In particular embodiments, compositions contemplated herein are formulated in a suspension, where the modified T cells are dispersed within an acceptable liquid medium or solution, e.g., saline or serum-free medium, in an intravenous (IV) bag or the like. Acceptable diluents include, but are not limited to water, PlasmaLyte, Ringer's solution, isotonic sodium chloride (saline) solution, serum-free cell culture medium, and medium suitable for cryogenic storage, e.g., Cryostor® medium.

In certain embodiments, a pharmaceutically acceptable carrier is substantially free of natural proteins of human or animal origin, and suitable for storing a composition comprising a population of modified T cells. The therapeutic composition is intended to be administered into a human patient, and thus is substantially free of cell culture components such as bovine serum albumin, horse serum, and fetal bovine serum.

In some embodiments, compositions are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better-defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions includes, but is not limited to, QBSF-60 (Quality Biological, Inc.), StemPro-34 (Life Technologies), and X-VIVO 10.

In one embodiment, the compositions comprising modified T cells are formulated in PlasmaLyte.

In various embodiments, compositions comprising modified T cells are formulated in a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CS5, and CryoStor CS2.

In one embodiment, the compositions are formulated in a solution comprising 50:50 PlasmaLyte A to CryoStor CS10.

In particular embodiments, the composition is substantially free of mycoplasma, endotoxin, and microbial contamination. By “substantially free” with respect to endotoxin is meant that there is less endotoxin per dose of cells than is allowed by the FDA for a biologic, which is a total endotoxin of 5 EU/kg body weight per day, which for an average 70 kg person is 350 EU per total dose of cells. In particular embodiments, compositions contemplated herein contain about 0.5 EU/mL to about 5.0 EU/mL, or about 0.5 EU/mL, 1.0 EU/mL, 1.5 EU/mL, 2.0 EU/mL, 2.5 EU/mL, 3.0 EU/mL, 3.5 EU/mL, 4.0 EU/mL, 4.5 EU/mL, or 5.0 EU/mL.

In particular embodiments, formulation of pharmaceutically-acceptable carrier solutions is well-known to those of skill in the art, as is the development of suitable dosing and treatment regimens for using the particular compositions described herein in a variety of treatment regimens, including e.g., enteral and parenteral, e.g., intravascular, intravenous, intrarterial, intraosseously, intraventricular, intracerebral, intracranial, intraspinal, intrathecal, and intramedullary administration and formulation. It would be understood by the skilled artisan that particular embodiments contemplated herein may comprise other formulations, such as those that are well known in the pharmaceutical art, and are described, for example, in Remington: The Science and Practice of Pharmacy, volume I and volume II 22^{nd} Edition. Edited by Loyd V. Allen Jr. Philadelphia, PA: Pharmaceutical Press; 2012, which is incorporated by reference herein, in its entirety.

In particular embodiments, compositions comprise an amount of immune effector cells that express one or more engineered immune receptor components contemplated herein. In particular embodiments, compositions comprise an amount of immune effector cells that express an exogenous lymphocyte receptor or engineered antigen receptor and one or more engineered immune receptor components contemplated herein. As used herein, the term “amount” refers to “an amount effective” or “an effective amount” of cells comprising one or more engineered immune receptor components contemplated herein, etc., to achieve a beneficial or desired prophylactic or therapeutic result in the presence of a bridging factor, including clinical results.

A “prophylactically effective amount” refers to an amount of cells comprising one or more engineered immune receptor components contemplated herein, etc., effective to achieve the desired prophylactic result in the presence of a bridging factor. Typically, but not necessarily, since a prophylactic dose is used in subjects prior to or at an earlier stage of disease, the prophylactically effective amount is less than the therapeutically effective amount.

A “therapeutically effective amount” refers to an amount of cells comprising one or more engineered immune receptor components contemplated herein that is effective to “treat” a subject (e.g., a patient) in the presence of a bridging factor. When a therapeutic amount is indicated, the precise amount of the compositions to be administered, cells, bridging factor, etc., can be determined by a physician with consideration of individual differences in age, weight, tumor size, extent of infection or metastasis, and condition of the patient (subject).

It can generally be stated that a pharmaceutical composition comprising the immune effector cells described herein may be administered at a dosage of 10² to 10¹⁰ cells/kg body weight, preferably 10⁵ to 10⁶ cells/kg body weight, including all integer values within those ranges. The number of cells will depend upon the ultimate use for which the composition is intended as will the type of cells included therein. For uses provided herein, the cells are generally in a volume of a liter or less, can be 500 mLs or less, even 250 mLs or 100 mLs or less.

Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some embodiments, particularly since all the infused cells will be redirected to a particular target antigen, lower numbers of cells, in the range of 10⁶/kilogram (10⁶-10¹¹ per patient) may be administered.

If desired, the treatment may also include administration of mitogens (e.g., PHA) or lymphokines, cytokines, and/or chemokines (e.g., IFN-7, IL-2, IL-12, TNF-alpha, IL-18, and TNF-beta, GM-CSF, IL-4, IL-13, Flt3-L, RANTES, MIP1α, etc.) as described herein to enhance induction of the immune response.

Generally, compositions comprising the cells activated and expanded as described herein may be utilized in the treatment and prevention of diseases that arise in individuals who are immunocompromised. In particular, compositions contemplated herein are used in the treatment of cancer. In particular embodiments, the immune effector cells may be administered either alone, or as a pharmaceutical composition in combination with carriers, diluents, excipients, and/or with other components such as IL-2 or other cytokines or cell populations.

In particular embodiments, pharmaceutical compositions comprise an amount of genetically modified T cells, in combination with one or more pharmaceutically or physiologically acceptable carriers, diluents or excipients.

In particular embodiments, pharmaceutical compositions comprise an amount of bridging factor, in combination with one or more pharmaceutically or physiologically acceptable carriers, diluents or excipients.

In a particular embodiment, compositions comprise an effective amount of immune effector cells comprising one or more engineered immune receptor components contemplated herein, alone or in combination with a bridging factor and/or one or more therapeutic agents, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated include cytokines, growth factors, steroids, NSAIDs, DMARDs, anti-inflammatories, chemotherapeutics, radiotherapeutics, therapeutic antibodies, or other active and ancillary agents.

In a particular embodiment, a composition comprising an effective amount of immune effector cells comprising one or more engineered immune receptor components contemplated herein is administered to a subject, and a composition comprising an effective amount of a bridging factor is administered to the subject, before, during, in combination with or subsequently to the cellular composition, and optionally repetitively administered to the subject.

In certain embodiments, compositions comprising immune effector cells comprising one or more engineered immune receptor components contemplated herein may be administered in conjunction with any number of chemotherapeutic agents.

A variety of other therapeutic agents may be used in conjunction with the compositions described herein. In one embodiment, the composition comprising immune effector cells comprising one or more engineered immune receptor components contemplated herein is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

Illustrative examples of therapeutic antibodies suitable for combination treatment with the modified T cells comprising one or more engineered immune receptor components contemplated herein, include but are not limited to, atezolizumab, avelumab, bavituximab, bevacizumab (avastin), bivatuzumab, blinatumomab, conatumumab, daratumumab, duligotumab, dacetuzumab, dalotuzumab, durvalumab, elotuzumab (HuLuc63), gemtuzumab, ibritumomab, indatuximab, inotuzumab, ipilimumab, lorvotuzumab, lucatumumab, milatuzumab, moxetumomab, nivolumab, ocaratuzumab, ofatumumab, pembrolizumab, rituximab, siltuximab, teprotumumab, and ublituximab.

In certain embodiments, the compositions described herein are administered in conjunction with a cytokine. By “cytokine” as used herein is meant a generic term for proteins released by one cell population that act on another cell as intercellular mediators. Examples of such cytokines are lymphokines, monokines, and traditional polypeptide hormones.

### I. Therapeutic Methods

Immune effector cells modified to express one or more engineered immune receptor components and/or an exogenous lymphocyte receptor or engineered antigen receptor contemplated herein provide improved methods of adoptive immunotherapy for use in the prevention, treatment, and amelioration of, or for preventing, treating, or ameliorating at least one symptom associated with an immune-related disease or disorder, e.g., cancer, an autoimmune disease, an immunodeficiency, an inflammatory disease, GVHD, or an infectious disease.

Immune effector cells comprising an engineered immune receptor system, e.g., a targeting component and a signaling component, provide improved methods of adoptive immunotherapy for use in the prevention, treatment, and amelioration of, or for preventing, treating, or ameliorating at least one symptom associated with an immune-related disease or disorder, e.g., cancer, an autoimmune disease, an immunodeficiency, an inflammatory disease, GVHD, or an infectious disease.

In particular embodiments, immune effector cells modified to express one or more engineered immune receptor components provide improved methods of adoptive immunotherapy to fine-tune the safety and efficacy of a cytotoxic response against target cells, e.g., tumor cells, expressing target antigens while decreasing the risk of on-target antigen, off-target cell cytotoxicity (recognizing the target antigen on a normal, non-target cell).

In particular embodiments, a method of preventing, treating, or ameliorating at least one symptom of an immune-related disease or disorder (e.g., cancer, an autoimmune disease, an immunodeficiency, an inflammatory disease, GVHD, or an infectious disease) comprises administering the subject an effective amount of modified immune effector cells or T cells comprising one or more components of a engineered immune receptor system and an engineered/exogenous TCR, CAR, or other therapeutic transgene to redirect the cells to a target cell. The genetically modified cells are a more efficacious and safe cellular immunotherapy by virtue of transducing a chemically regulatable immunostimulatory signal.

In particular embodiments, one or more immune effector cells, e.g., T cells, are modified to express both a targeting component and a signaling component. In this case, the modified cells are administered to a subject in need thereof and home to the target cells via the interaction of the signaling component expressed on the immune effector cell and the target antigen expressed on the target cell. A bridging factor is administered to the subject before the modified cells, about the same time as the modified cells, or after the modified cells have been administered to the subject. In the presence of the bridging factor, a complex forms between the targeting component bound to the target antigen, the bridging factor, and the signaling component bound to an immune receptor complex. Upon formation of the complex, the signaling and targeting components transduce an immunostimulatory signal to the immune effector cell that synergizes with the immune receptor signal and in turn, elicits a cytotoxic response from the immune effector cell against the target cell.

In various embodiments, immune effector cells comprising one or more engineered immune receptor components and/or an exogenous lymphocyte receptor or engineered antigen receptor fine-tune the safety and efficacy of a cytotoxic response against target cells using a dual targeting strategy wherein one or more target cells express one or more target antigens recognized by the exogenous lymphocyte receptor or engineered antigen receptor and the engineered immune receptor.

In particular embodiments, one or more immune effector cells, e.g., T cells, are modified to express both the targeting component and the signaling component and an exogenous lymphocyte receptor or engineered antigen receptor, e.g., a TCR, CAR, CCR, or flip receptor. In this case, the modified cells are administered to a subject in need thereof and home to the target cells via the interaction of the targeting component that binds a first target antigen and the antigen receptor, which binds a second target antigen, wherein one or both target antigens are expressed on target cells or population of target cells. Interaction of the antigen receptor (e.g., TCR) with a target antigen on the target cell may elicit a cytotoxic response from the immune effector cell against the target cell. In some embodiments, a bridging factor is administered to the subject before the modified cells, about the same time as the modified cells, or after the modified cells have been administered to the subject. In the presence of the bridging factor, a complex forms between the targeting component that binds a first target antigen, the bridging factor, and the signaling component, which binds an immune receptor complex. Upon formation of the complex, the signaling and targeting components transduce an immunostimulatory signal to the immune effector cell that synergizes with the immune receptor signal and in turn, elicits or augments a cytotoxic response from the immune effector cell against the target cell.

In particular embodiments, one or more immune effector cells, e.g., T cells, are modified to express both a targeting component and a signaling component. In this case, the modified cells are administered to a subject in need thereof and home to the target cells via the interaction of the signaling component expressed on the immune effector cell and the target antigen expressed on the target cell. In some embodiments, a bridging factor is administered to the subject before the modified cells, about the same time as the modified cells, or after the modified cells have been administered to the subject. In the presence of the bridging factor, a complex forms between the targeting component bound to the target antigen, the bridging factor, and a signaling component that comprises a multimerization domain, optionally a linker polypeptide, and an actuator domain (e.g., Fcγ, CD3ε, CD3δ, or CD3γ or fragment thereof). Upon formation of the complex, the signaling and targeting components transduce an immunostimulatory signal to the immune effector cell that synergizes with the immune receptor signal and in turn, elicits a cytotoxic response from the immune effector cell against the target cell.

In particular embodiments, one or more immune effector cells, e.g., T cells, are modified to express both the targeting component and the signaling component and an exogenous lymphocyte receptor or engineered antigen receptor, e.g., an engineered/exogenous TCR. In this case, the modified cells are administered to a subject in need thereof and home to the target cells via the interaction of the targeting component that binds a first target antigen and the immune receptor, which binds a second target antigen, wherein one or both target antigens are expressed on target cells or population of target cells. Interaction of the TCR with a target antigen on the target cell may elicit a cytotoxic response from the immune effector cell against the target cell. A bridging factor is administered to the subject before the modified cells, about the same time as the modified cells, or after the modified cells have been administered to the subject. In the presence of the bridging factor, a complex forms between the targeting component that binds a first target antigen, the bridging factor, and a signaling component that comprises a multimerization domain, optionally a linker polypeptide, and Fcγ, CD3ε, CD3δ or CD3γ, or fragment thereof. Upon formation of the complex, the signaling and targeting components transduce an immunostimulatory signal to the immune effector cell that synergizes with the immune receptor signal and in turn, elicits or augments a cytotoxic response from the immune effector cell against the target cell.

In particular embodiments, the modified immune effector cells contemplated herein are used in the treatment of solid tumors or cancers.

In particular embodiments, the modified immune effector cells contemplated herein are used in the treatment of solid tumors or cancers including, but not limited to: adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain/CNS cancer, breast cancer, bronchial tumors, cardiac tumors, cervical cancer, cholangiocarcinoma, chondrosarcoma, chordoma, colon cancer, colorectal cancer, craniopharyngioma, ductal carcinoma in situ (DCIS) endometrial cancer, ependymoma, esophageal cancer, esthesioneuroblastoma, Ewing's sarcoma, extracranial germ cell tumor, extragonadal germ cell tumor, eye cancer, fallopian tube cancer, fibrous histiosarcoma, fibrosarcoma, gallbladder cancer, gastric cancer, gastrointestinal carcinoid tumors, gastrointestinal stromal tumor (GIST), germ cell tumors, glioma, glioblastoma, head and neck cancer, hemangioblastoma, hepatocellular cancer, hypopharyngeal cancer, intraocular melanoma, kaposi sarcoma, kidney cancer, laryngeal cancer, leiomyosarcoma, lip cancer, liposarcoma, liver cancer, lung cancer, non-small cell lung cancer, lung carcinoid tumor, malignant mesothelioma, medullary carcinoma, medulloblastoma, menangioma, melanoma, Merkel cell carcinoma, midline tract carcinoma, mouth cancer, myxosarcoma, myelodysplastic syndrome, myeloproliferative neoplasms, nasal cavity and paranasal sinus cancer, nasopharyngeal cancer, neuroblastoma, oligodendroglioma, oral cancer, oral cavity cancer, oropharyngeal cancer, osteosarcoma, ovarian cancer, pancreatic cancer, pancreatic islet cell tumors, papillary carcinoma, paraganglioma, parathyroid cancer, penile cancer, pharyngeal cancer, pheochromocytoma, pinealoma, pituitary tumor, pleuropulmonary blastoma, primary peritoneal cancer, prostate cancer, rectal cancer, retinoblastoma, renal cell carcinoma, renal pelvis and ureter cancer, rhabdomyosarcoma, salivary gland cancer, sebaceous gland carcinoma, skin cancer, soft tissue sarcoma, squamous cell carcinoma, small cell lung cancer, small intestine cancer, stomach cancer, sweat gland carcinoma, synovioma, testicular cancer, throat cancer, thymus cancer, thyroid cancer, urethral cancer, uterine cancer, uterine sarcoma, vaginal cancer, vascular cancer, vulvar cancer, and Wilms Tumor.

In particular embodiments, the modified immune effector cells contemplated herein are used in the treatment of solid tumors or cancers including, without limitation, non-small cell lung carcinoma, head and neck squamous cell carcinoma, colorectal cancer, pancreatic cancer, breast cancer, thyroid cancer, bladder cancer, cervical cancer, esophageal cancer, ovarian cancer, gastric cancer endometrial cancer, gliomas, glioblastomas, and oligodendroglioma.

In particular embodiments, the modified immune effector cells contemplated herein are used in the treatment of solid tumors or cancers including, without limitation, non-small-cell lung cancer, metastatic colorectal cancer, glioblastoma, head and neck cancer, pancreatic cancer, and breast cancer.

In particular embodiments, the modified immune effector cells contemplated herein are used in the treatment of glioblastoma.

In particular embodiments, the modified immune effector cells contemplated herein are used in the treatment of liquid cancers or hematological cancers.

In particular embodiments, the modified immune effector cells contemplated herein are used in the treatment of B-cell malignancies, including but not limited to: leukemias, lymphomas, and multiple myeloma.

In particular embodiments, the modified immune effector cells contemplated herein are used in the treatment of liquid cancers including, but not limited to leukemias, lymphomas, and multiple myelomas: acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CML), chronic myelomonocytic leukemia (CMML) and polycythemia vera, Hodgkin lymphoma, nodular lymphocyte-predominant Hodgkin lymphoma, Burkitt lymphoma, small lymphocytic lymphoma (SLL), diffuse large B-cell lymphoma, follicular lymphoma, immunoblastic large cell lymphoma, precursor B-lymphoblastic lymphoma, mantle cell lymphoma, marginal zone lymphoma, mycosis fungoides, anaplastic large cell lymphoma, Sézary syndrome, precursor T-lymphoblastic lymphoma, multiple myeloma, overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

In particular embodiments, the modified immune effector cells contemplated herein are used in the treatment of acute myeloid leukemia (AML).

Preferred cells for use in the methods contemplated herein include autologous/autogeneic (“self”) cells, preferably hematopoietic cells, more preferably T cells, and more preferably immune effector cells.

In particular embodiments, a method comprises administering a therapeutically effective amount of modified immune effector cells that express one or more engineered immune receptor components, and optionally an exogenous lymphocyte receptor or engineered antigen receptor or another targeting or signaling component, or a composition comprising the same, to a patient in need thereof, and optionally also administering a bridging factor to the subject. In certain embodiments, the cells are used in the treatment of patients at risk for developing an immune disorder. Thus, particular embodiments comprise the treatment or prevention or amelioration of at least one symptom of an immune-related disease or disorder, e.g., cancer or autoimmune disease, comprising administering to a subject in need thereof, a therapeutically effective amount of the modified immune effector cells contemplated herein and, optionally, a bridging factor.

The quantity and frequency of administration of modified immune effector cells, targeting components, and/or bridging factor will be determined by such factors as the condition of the patient, and the type and severity of the patient's disease, although appropriate dosages and dose schedules may be determined by clinical trials.

In one illustrative embodiment, the effective amount of modified immune effector cells provided to a subject is at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, or at least 10×10⁶ cells/kg, or more cells/kg, including all intervening doses of cells.

In another illustrative embodiment, the effective amount of modified immune effector cells provided to a subject is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, or about 10×10⁶ cells/kg, or more cells/kg, including all intervening doses of cells.

In another illustrative embodiment, the effective amount of modified immune effector cells provided to a subject is from about 2×10⁶ cells/kg to about 10×10⁶ cells/kg, about 3×10⁶ cells/kg to about 10×10⁶ cells/kg, about 4×10⁶ cells/kg to about 10×10⁶ cells/kg, about 5×10⁶ cells/kg to about 10×10⁶ cells/kg, 2×10⁶ cells/kg to about 6×10⁶ cells/kg, 2×10⁶ cells/kg to about 7×10⁶ cells/kg, 2×10⁶ cells/kg to about 8×10⁶ cells/kg, 3×10⁶ cells/kg to about 6×10⁶ cells/kg, 3×10⁶ cells/kg to about 7×10⁶ cells/kg, 3×10⁶ cells/kg to about 8×10⁶ cells/kg, 4×10⁶ cells/kg to about 6×10⁶ cells/kg, 4×10⁶ cells/kg to about 7×10⁶ cells/kg, 4×10⁶ cells/kg to about 8×10⁶ cells/kg, 5×10⁶ cells/kg to about 6×10⁶ cells/kg, 5×10⁶ cells/kg to about 7×10⁶ cells/kg, 5×10⁶ cells/kg to about 8×10⁶ cells/kg, or 6×10⁶ cells/kg to about 8×10⁶ cells/kg, including all intervening doses of cells.

One of ordinary skill in the art would recognize that multiple administrations of the compositions contemplated in particular embodiments may be required to affect the desired therapy. For example, a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more. Modified immune effector cells, engineered immune receptor components, and/or bridging factor may be administered in the same or different compositions; in one or more compositions at the same time; or more than one composition at different times. Modified immune effector cells, engineered immune receptor components (e.g., targeting components), and/or bridging factor may be administered through the same route of administration or different routes.

In certain embodiments, it may be desirable to administer activated T cells to a subject and then subsequently redraw blood (or have an apheresis performed), activate T cells therefrom, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, 100 cc, 150 cc, 200 cc, 250 cc, 300 cc, 350 cc, or400 cc or more. Not to be bound by theory, using this multiple blood draw/multiple reinfusion protocol may serve to select out certain populations of T cells.

In one embodiment, a method of treating a subject diagnosed with an immune-related disease or disorder (e.g., cancer or autoimmune disease), comprises removing immune effector cells from the subject, modifying the immune effector cells by introducing one or more vectors encoding one or more engineered immune receptor components into the cell and producing a population of modified immune effector cells, and administering the population of modified immune effector cells to the same subject. In a preferred embodiment, the immune effector cells comprise T cells.

In one embodiment, a method of treating a subject diagnosed with a cancer or autoimmune disease, comprises administering one or more vectors encoding one or more engineered immune receptor components to the subject. In a preferred embodiment, the immune effector cells comprise T cells.

In one embodiment, a method of treating a subject diagnosed with a cancer, comprises removing immune effector cells from the subject, modifying the immune effector cells by introducing one or more vectors encoding one or more engineered immune receptor components and optionally an exogenous lymphocyte receptor or engineered antigen receptor or another targeting or signaling component into the cell and producing a population of modified immune effector cells, and administering the population of modified immune effector cells to the same subject. In a preferred embodiment, the immune effector cells comprise T cells.

In one embodiment, a method of treating a subject diagnosed with a cancer, comprises administering one or more vectors encoding one or more engineered immune receptor components to the subject. In a preferred embodiment, the immune effector cells comprise T cells.

The methods for administering the cell compositions contemplated in particular embodiments include any method which is effective to result in reintroduction of ex vivo modified immune effector cells or reintroduction of modified progenitors of immune effector cells that upon introduction into a subject differentiate into mature immune effector cells. One method comprises modifying peripheral blood T cells ex vivo by introducing one or more vectors encoding one or more engineered immune receptor components and optionally an exogenous lymphocyte receptor or engineered antigen receptor or another targeting or signaling component into the cell and returning the transduced cells into the subject.

The methods for administering the cell compositions contemplated in particular embodiments include any method which is effective to result in reintroduction of ex vivo modified immune effector cells or reintroduction of modified progenitors of immune effector cells that upon introduction into a subject differentiate into mature immune effector cells. One method comprises modifying peripheral blood T cells ex vivo by introducing one or more vectors encoding one or more engineered immune receptor components and optionally an exogenous lymphocyte receptor or engineered antigen receptor or another targeting or signaling component into the cell and returning the transduced cells into the subject.

The methods for administering the vector compositions contemplated in particular embodiments include any method which is effective to result in in vivo modified immune effector cells.

### J. Sequence Listing

| SEQ ID | | |
|---|---|---|
| NO: | Description | Sequence |
| 1 | FRB* | ILWHEMWHEGLEEASRLYFGERNVKGMFEVLEPLHAMMERGPQTL |
| | | KETSFNQAYGRDLMEAQEWCRKYMKSGNVKDLLQAWDLYYHVERR |
| | | ISK |
| 2 | FRB | ILWHEMWHEGLEEASRLYFGERNVKGMFEVLEPLHAMMERGPQTL |
| | | KETSFNQAYGRDLMEAQEWCRKYMKSGNVKDLTQAWDLYYHVERR |
| | | ISK |
| 3 | FKBP12 | GVQVETISPGDGRTFPKRGQTCVVHYTGMLEDGKKFDSSRDRNKP |
| | | FKFMLGKQEVIRGWEEGVAQMSVGQRAKLTISPDYAYGATGHPGI |
| | | IPPHATLVFDVELLKLE |
| 4 | FKBP12 F36V | GVQVETISPGDGRTFPKRGQTCVVHYTGMLEDGKKVDSSRDRNKP |
| | | FKFMLGKQEVIRGWEEGVAQMSVGQRAKLTISPDYAYGATGHPGI |
| | | IPPHATLVFDVELLKLE |
| 5 | CH3-Ab (S354C T366W) | GQPREPQVYTLPPCRDELTKNQVSLWCLVKGFYPSDIAVEWESNG |
| | | QPENNYKTTPPVLDSDGSFFLYSKLTVDKSRWQQGNVFSCSVMHE |
| | | ALHNHYTQKSLSLSPGK |
| 6 | CH3-IA (Y349C T366S | GQPREPQVCTLPPSRDELTKNQVSLSCAVKGFYPSDIAVEWESNG |
| | L368A Y407V) | QPENNYKTTPPVLDSDGSFFLVSKLTVDKSRWQQGNVFSCSVMHE |
| | | ALHNHYTQKSLSLSPGK |
| 7 | CH3-DE (L351D L368E) | GQPREPQVYTDPPSRDELTKNQVSLTCEVKGFYPSDIAVEWESNG |
| | | QPENNYKTTPPVLDSDGSFFLYSKLTVDKSRWQQGNVFSCSVMHE |
| | | ALHNHYTQKSLSLSPGK |
| 8 | CH3-KK (L351K T366K) | GQPREPQVYTKPPSRDELTKNQVSLKCLVKGFYPSDIAVEWESNG |
| | | QPENNYKTTPPVLDSDGSFFLYSKLTVDKSRWQQGNVFSCSVMHE |
| | | ALHNHYTQKSLSLSPGK |
| 9 | CH3-GA (SEED 1) | GQPREPQVYTLPPPSEELALNELVTLTCLVKGFYPSDIAVEWLQG |
| | | SQELPREKYLTWAPVLDSDGSFFLYSILRVAAEDWKKGDTFSCSV |
| | | MHEALHNHYTQKSLDR |
| 10 | CH3-AG (SEED 2) | GQPFRPEVHLLPPSREEMTKNQWSLTCLARGFYPKDIAVEWESNG |
| | | QPENNYKTTPSRQEPSQGTTTFAVTSKLTVDKSRWQQGNWFSCSV |
| | | MHEALHNHYTQKTISL |
| 11 | 1xG4S linker | GGGGS |
| 12 | 2xG4S linker | GGGGSGGGGS |
| 13 | 3xG4S linker | GGGGSGGGGSGGGGS |
| 14 | 4xG4S linker | GGGGSGGGGSGGGGSGGGGS |
| 15 | 5xG4S linker | GGGGSGGGGSGGGGSGGGGSGGGGS |
| 16 | Linker | DGGGS |
| 17 | Linker | TGEKP |
| 18 | Linker | GGRR |
| 19 | Linker | EGKSSGSGSESKVD |
| 20 | Linker | KESGSVSSEQLAQFRSLD |
| 21 | Linker | GGRRGGGS |
| 22 | Linker | LRQRDGERP |
| 23 | Linker | LRQKDGGGSERP |
| 24 | Linker | LRQKDGGGSGGGSERP |
| 25 | Linker | GSTSGSGKPGSGEGSTKG |
| 26 | linker | GSTSGSGKSSEGSGSTKG |
| 27 | linker | GSTSGSGKSSEGKG |
| 28 | linker | GSTSGSGKPGSGEGS |
| 29 | linker | GGGS |
| 30 | yuTCR linker (uLNK) | LEKT |
| 31 | uLNK + G4S linker | LEKTGGGGS |
| 32 | CD3ϵ | DGNEEMGGITQTPYKVSISGTTVILTCPQYPGSEILWQHNDKNIG |
| | | GDEDDKNIGSDEDHLSLKEFSELEQSGYYVCYPRGSKPEDANFYL |
| | | YLRARVCENCMEMDVMSVATIVIVDICITGGLLLLVYYWSKNRKA |
| | | KAKPVTRGAGAGGRQRGQNKERPPPVPNPDYEPIRKGQRDLYSGL |
| | | NQRRI |
| 33 | CD3γ | QSIKGNHLVKVYDYQEDGSVLLTCDAEAKNITWFKDGKMIGFLTE |
| | | DKKKWNLGSNAKDPRGMYQCKGSQNKSKPLQVYYRMCQNCIELNA |
| | | ATISGFLFAEIVSIFVLAVGVYFIAGQDGVRQSRASDKQTLLPND |
| | | QLYQPLKDREDDQYSHLQGNQLRRN |
| 34 | CD3δ | EHSTFLSGLVLATLLSQVSPFKIPIEELEDRVFVNCNTSITWVEG |
| | | TVGTLLSDITRLDLGKRILDPRGIYRCNGTDIYKDKESTVQVHYR |
| | | MCQSCVELDPATVAGIIVTDVIATLLLALGVFCFAGHETGRLSGA |
| | | ADTQALLRNDQVYQPLRDRDDAQYSHLGGNWARNK |
| 35 | FcϵR1γ - v1 | GEPLCYILDAILFLYGIVLTLLYCRLKIQVRKAAITSYEKSDGVY |
| | | TGLSTRNQETYETLKHEKPPQ |
| 36 | FcϵR1γ - v2 | LGEPQLCYILDAILFLYGIVLTLLYCRLKIQVRKAAITSYEKSDG |
| | | VYTGLSTRNQETYETLKHEKPPQ |
| 37 | Igα/CD79a (BCR) | LWMHKVPASLMVSLGEDAHFQCPHNSSNNANVTWWRVLHGNYTWP |
| | | PEFLGPGEDPNGTLIIQNVNKSHGGIYVCRVQEGNESYQQSCGTY |
| | | LRVRQPPPRPFLDMGEGTKNRIITAEGIILLFCAVVPGTLLLFRK |
| | | RWQNEKLGLDAGDEYEDENLYEGLNLDDCSMYEDISRGLQGTYQD |
| | | VGSLNIGDVQLEKP |
| 38 | Igβ/CD79b (BCR) | MARLALSPVPSHWMVALLLLLSGTEPTTRPVGFALISSCLGPAAL |
| | | PPACSTLLLCLSLFTACLPLMALGPGSVGVSCTW |
| 39 | DAP10 | QTTPGERSSLPAFYPGTSGSCSGCGSLSLPLLAGLVAADAVASLL |
| | | IVGAVFLCARPRRSPAQEDGKVYINMPGRG |
| 40 | DAP12 | LRPVQAQAQSDCSCSTVSPGVLAGIVMGDLVLTVLIALAVYFLGR |
| | | LVPRGRGAAEAATRKQRITETESPYQELQGQRSDVYSDLNTQRPY |
| | | YK |
| 41 | Minimal CD4 hinge | SNIKVLPTWSTPVQP |
| 42 | Minimal CD28 hinge | KGKHLCPSPLFPGPSKP |
| 43 | IgG4 hinge | ESKYGPPCPSCP |
| 44 | CD8 hinge | TTTPAPRPPTPAPTIASQPLSLRPEACRPAAGGAVHTRGLDFACD |
| | Hinge linker | GGR |
| 45 | CD4 transmembrane | MALIVLGGVAGLLLFIGLGIFF |
| 46 | CD28 transmembrane | FWVLVVVGGVLACYSLLVTVAFIIFWV |
| 47 | CD8 transmembrane | AGTGSDIYIWAPLAGTCGVLLLSLVIT |
| 48 | Truncated | CVRC |
| | intracellular CD4 - | |
| | v1 | |
| 49 | Truncated | CVRCRHRRRQ |
| | intracellular CD4 - | |
| | v2 | |
| 50 | anti-BCMA_scFv_1 | DIVLTQSPPSLAMSLGKRATISCRASESVTILGSHLIHWYQQKPG |
| | | QPPTLLIQLASNVQTGVPARFSGSGSRTDFTLTIDPVEEDDVAVY |
| | | YCLQSRTIPRTFGGGTKLEIKGSTSGSGKPGSGEGSTKGQIQLVQ |
| | | SGPELKKPGETVKISCKASGYTFTDYSINWVKRAPGKGLKWMGWI |
| | | NTETREPAYAYDFRGRFAFSLETSASTAYLQINNLKYEDTATYFC |
| | | ALDYSYAMDYWGQGTSVTVSS |
| 51 | anti-BCMA_scFv_2 | DIVLTQSPASLAVSLGERATINCRASESVSVIGAHLIHWYQQKPG |
| | | QPPKLLIYLASNLETGVPARFSGSGSGTDFTLTISSLQAEDAAIY |
| | | YCLQSRIFPRTFGQGTKLEIKGSTSGSGKPGSGEGSTKGQVQLVQ |
| | | SGSELKKPGASVKVSCKASGYTFTDYSINWVRQAPGQGLEWMGWI |
| | | NTETREPAYAYDFRGRFVFSLDTSVSTAYLQISSLKAEDTAVYYC |
| | | ARDYSYAMDYWGQGTLVTVSS |
| 52 | anti-BCMA_scFv_3 | DIQMTQSPSSLSASVGDRITITCRASQDIRNYLGWYQQKPGKAPK |
| | | VLIFAASSLQSGVPSRFSGSGSGTDFTLTISSLQPEDFATYYCLQ |
| | | DYIYPWTFAQGTKVEIKGGGGSGGGGSGGGGSQVQLVESGGGVVQ |
| | | PGRSLRLSCAASGFTFSSYGMHWVRQAPGKGLEWVAVISYDGRNK |
| | | NYADSVKGRFTISRDNSKNTLYLQMNSLRAEDTAVYYCAREGEAT |
| | | YYDILTGPFDYWGQGTLVTVSS |
| 53 | anti-CD19_scFv | DIQMTQTTSSLSASLGDRVTISCRASQDISKYLNWYQQKPDGTVK |
| | | LLIYHTSRLHSGVPSRFSGSGSGTDYSLTISNLEQEDIATYFCQQ |
| | | GNTLPYTFGGGTKLEITGGGGSGGGGSGGGGSEVKLQESGPGLVA |
| | | PSQSLSVTCTVSGVSLPDYGVSWIRQPPRKGLEWLGVIWGSETTY |
| | | YNSALKSRLTIIKDNSKSQVFLKMNSLQTDDTAIYYCAKHYYYGG |
| | | SYAMDYWGQGTSVTVSS |
| 54 | anti-CD20_scFv_1 | EVQLQQSGAELVKPGASVKMSCKASGYTFTSYNMHWVKQTPGQGL |
| | | EWIGAIYPGNGDTSYNQKFKGKATLTADKSSSTAYMQLSSLTSED |
| | | SADYYCARSNYYGSSYWFFDVWGAGTTVTVSSGGGGSGGGGSGGG |
| | | GSDIVLTQSPAILSASPGEKVTMTCRASSSVNYMDWYQKKPGSSP |
| | | KPWIYATSNLASGVPARFSGSGSGTSYSLTISRVEAEDAATYYCQ |
| | | QWSFNPPTFGGGTKLEIK |
| 55 | anti-CD20_scFv_2 | QVQLQQPGAELVKPGASVKMSCKASGYTFTSYNMHWVKQTPGRGL |
| | | EWIGAIYPGNGDTSYNQKFKGKATLTADKSSSTAYMQLSSLTSED |
| | | SAVYYCARSTYYGGDWYFNVWGAGTTVTVSAGGGGSGGGGSGGGG |
| | | SQIVLSQSPAILSASPGEKVTMTCRASSSVSYIHWFQQKPGSSPK |
| | | PWIYATSNLASGVPVRFSGSGSGTSYSLTISRVEAEDAATYYCQQ |
| | | WTSNPPTFGGGTKLEIKR |
| 56 | anti-CD20_scFv_3 | QVQLVQSGAEVKKPGSSVKVSCKASGYAFSYSWINWVRQAPGQGL |
| | | EWMGRIFPGDGDTDYNGKFKGRVTITADKSTSTAYMELSSLRSED |
| | | TAVYYCARNVFDGYWLVYWGQGTLVTVSSGGGGSGGGGSGGGGSD |
| | | IVMTQTPLSLPVTPGEPASISCRSSKSLLHSNGITYLYWYLQKPG |
| | | QSPQLLIYQMSNLVSGVPDRFSGSGSGTDFTLKISRVEAEDVGVY |
| | | YCAQNLELPYTFGGGTKVEIKRTV |
| 57 | anti-CD22_scFv | QVQLQQSGPGLVKPSQTLSLTCAISGDSVSSNSAAWNWIRQSPSR |
| | | GLEWLGRTYYRSKWYNDYAVSVKSRITINPDTSKNQFSLQLNSVT |
| | | PEDTAVYYCAREVTGDLEDAFDIWGQGTMVTVSSGGGGSDIQMTQ |
| | | SPSSLSASVGDRVTITCRASQTIWSYLNWYQQRPGKAPNLLIYAA |
| | | SSLQSGVPSRFSGRGSGTDFTLTISSLQAEDFATYYCQQSYSIPQ |
| | | TFGQGTKLEIK |
| 58 | anti-CD33_scFv | EIVLTQSPGSLAVSPGERVTMSCKSSQSVFFSSSQKNYLAWYQQI |
| | | PGQSPRLLIYWASTRESGVPDRFTGSGSGTDFTLTISSVQPEDLA |
| | | IYYCHQYLSSRTFGQGTKLEIKGGGGSGGGGSGGGGSQVQLQQPG |
| | | AEVVKPGASVKMSCKASGYTFTSYYIHWIKQTPGQGLEWVGVIYP |
| | | GNDDISYNQKFQGKATLTADKSSTTAYMQLSSLTSEDSAVYYCAR |
| | | EVRLRYFDVWGQGTTVTVSS |
| 59 | anti-CD79A_scFv | DVLMTQIPLSLPVSLGDQASISCRSSQSIVHSNGNTYLEWYLQKP |
| | | GQSPKLLIYKVSNRFSGVPDRFSGSGSGTDFTLKISRVEAEDLGV |
| | | YYCFQGSHVPFTFGSGTKLEIKRGGGGSGGGGSGGGGSQVQLQQS |
| | | GPELVKPGASVKISCKASGYTFSTSWMNWVKQRPGQGLEWIGRIY |
| | | PGDGDTNYNGKFKGKATLTADKSSNTAYMQLSSLTSVDSAVYFCE |
| | | RFYYGNTFAMDYWGQGTSVTVSS |
| 60 | anti-CD79B_scFv_1 | QIQLVQSGPELKKPGETVKISCKASGYTFTDYSMHWVKQAPGEGL |
| | | KWMGWINTETGEPTYADDFKGRFAFSLETSASTAYLQINNLKNED |
| | | TATYFCYYGSYWGQGTLVTVSAGGGGSGGGGSGGGGSDIVLTQSP |
| | | ASLAVSLGQRATISCKASQSVDYDGDGYMDWYQQKPGQPPKLLIF |
| | | AASNLKSGIPARFSGSGSGTDFTLNIHPVEEEDAATYYCQQTNEY |
| | | PWTFGGGTKLEIK |
| 61 | Anti-CD79B_scFv_2 | DIQLTQSPSSLSASVGDRVTITCKASQSVDYEGDSFLNWYQQKPG |
| | | KAPKLLIYAASNLESGVPSRFSGSGSGTDFTLTISSLQPEDFATY |
| | | YCQQSNEDPLTFGQGTKVEIKRGGGGSGGGGSGGGGSEVQLVESG |
| | | GGLVQPGGSLRLSCAASGYTFSSYWIEWVRQAPGKGLEWIGEILP |
| | | GGGDTNYNEIFKGRATFSADTSKNTAYLQMNSLRAEDTAVYYCTR |
| | | RVPIRLDYWGQGTLVTVSS |
| 62 | anti-B7H3_scFv | DIVMTQSHKFMSTSIGARVSITCKASQDVRTAVAWYQQKPGQSPK |
| | | LLIYSASYRYTGVPDRFTGSGSGTDFTFTISSVQAEDLAVYYCQQ |
| | | HYGTPPWTFGGGTKLEIKGGGGSGGGGSGGGGSEVQLVESGGGLV |
| | | KPGGSLKLSCEASRFTFSSYAMSWVRQTPEKRLEWVAAISGGGRY |
| | | TYYPDSMKGRFTISRDNAKNFLYLQMSSLRSEDTAMYYCARHYDG |
| | | YLDYWGQGTTLTVSSTR |
| 63 | anti-Muc16_scFv | VKLQESGGGFVKPGGSLKVSCAASGFTFSSYAMSWVRLSPEMRLE |
| | | WVATISSAGGYIFYSDSVQGRFTISRDNAKNTLHLQMGSLRSGDT |
| | | AMYYCARQGFGNYGDYYAMDYWGQGTTVTVSSGGGGSGGGGSGGG |
| | | GSDIELTQSPSSLAVSAGEKVTMSCKSSQSLLNSRTRKNQLAWYQ |
| | | QKPGQSPELLIYWASTRQSGVPDRFTGSGSGTDFTLTISSVQAED |
| | | LAVYYCQQSYNLLTFGPGTKLEVKR |
| 64 | anti-HER2_scFv | DIQMTQSPSSLSASVGDRVTITCRASQDVNTAVAWYQQKPGKAPK |
| | | LLIYSASFLYSGVPSRFSGSRSGTDFTLTISSLQPEDFATYYCQQ |
| | | HYTTPPTFGQGTKVEIKGSTSGSGKPGSGEGSGEVQLVESGGGLV |
| | | QPGGSLRLSCAASGFNIKDTYIHWVRQAPGKGLEWVARIYPTNGY |
| | | TRYADSVKGRFTISADTSKNTAYLQMNSLRAEDTAVYYCSRWGGD |
| | | GFYAMDVWGQGTLVTVSS |
| 65 | anti-EGFR_scFv | DILLTQSPVILSVSPGERVSFSCRASQSIGTNIHWYQQRTNGSPR |
| | | LLIKYASESISGIPSRFSGSGSGTDFTLSINSVESEDIADYYCQQ |
| | | NNNWPTTFGAGTKLELKGGGGSGGGGSGGGGSQVQLKQSGPGLVQ |
| | | PSQSLSITCTVSGFSLTNYGVHWVRQSPGKGLEWLGVIWSGGNTD |
| | | YNTPFTSRLSINKDNSKSQVFFKMNSLQSNDTAIYYCARALTYYD |
| | | YEFAYWGQGTLVTVSS |
| 66 | anti-FN-EDB_scFv | EVQLLESGGGLVQPGGSLRLSCAASGFTFSSFSMSWVRQAPGKGL |
| | | EWVSSISGSSGTTYYADSVKGRFTISRDNSKNTLYLQMNSLRAED |
| | | TAVYYCAKPFPYFDYWGQGTLVTVSSGDGSSGGSGGASEIVLTQS |
| | | PGTLSLSPGERATLSCRASQSVSSSFLAWYQQKPGQAPRLLIYYA |
| | | SSRATGIPDRFSGSGSGTDFTLTISRLEPEDFAVYYCQQTGRIPP |
| | | TFGQGTKVEIK |
| 67 | anti-CLDN18.2_scFv | QIQLVQSGPELKKPGETVKISCKASGYTFTNYGMNWVKQAPGKGL |
| | | KWMGWINTNTGEPTYAEEFKGRFAFSLETSASTAYLQINNLKNED |
| | | TATYFCARLGFGNAMDYWGQGTSVTVSSGGGGSGGGGSGGGGSDI |
| | | VMTQSPSSLTVTAGEKVTMSCKSSQSLLNSGNQKNYLTWYQQKPG |
| | | QPPKLLIYWASTRESGVPDRFTGSGSGTDFTLTISSVQAEDLAVY |
| | | YCQNDYSYPLTFGAGTKLELK |
| 68 | anti-DLL3_scFv | QVQLQESGPGLVKPSETLSLTCTVSGDSISSYYWTWIRQPPGKGL |
| | | EWIGYIYYSGTTNYNPSLKSRVTISVDTSKSQFSLKLSSVTAADT |
| | | AVYYCASIAVRGFFFDYWGQGTLVTVSSGGGGSGGGGSGGGGSEI |
| | | VLTQSPGTLSLSPGERATLSCRASQSVSSSYLAWYQQKPGQAPRL |
| | | LIYGASTRATGIPDRESGSGSGTDFTLTISRLEPEDFAVYYCQQY |
| | | GTSPLTFGGGTKVEIK |
| 69 | anti-FLT3_scFv_1 | QVTLKESGPVLVKPTETLTLTCTVSGFSLINARMGVSWIRQPPGK |
| | | ALEWLAHIFSNAEKSYRTSLKSRLTISKDTSKSQVVLTMTNMDPV |
| | | DTATYYCARIPGYGGNGDYHYYGMDVWGQGTTVTVSSGGGGSGGG |
| | | GSGGGGSDIQMTQSPSSLSASLGDRVTITCRASQGIRNDLGWYQQ |
| | | KPGKAPKRLIYASSTLQSGVPSRFSGSGSGTEFTLTISSLQPEDF |
| | | ATYYCLQHNNFPWTFGQGTKVEIK |
| 70 | anti-FLT3_scFv_2 | EVQLVESGGGLVQPGGSLRLSCAASGFTFSSYGMHWVRQAPGKGL |
| | | EWVAVISYDGSNKYYADSVKGRFTISRDNSKNTLYLQMNSLRAED |
| | | TAVYYCANLAPWAAYWGQGTLVTVSSGGGGSGGGGSGGGGSEIVL |
| | | TQSPLSLPVTPGEPASISCRSSQSLLHSNGYNYLDWYLQKPGQSP |
| | | QLLIYLGSNRASGVPDRFSGSGSGTDFTLKISRVEAEDVGVYYCM |
| | | QALQTPHTFGQGTKLEIK |
| 71 | anti-ROR1 scFv | DIQMTQSPSFLSASVGDRVTINCKASQNIDRYLNWYQQKLGEAPK |
| | | RLLYNTNKLQTGIPSRFSGSGSATDFTLTISSLQPEDFATYFCLQ |
| | | YNSLPLTFGSGTKLEIKGGGGSGGGGSGGGGSEVQLVESGGGLVQ |
| | | PGRSLKLSCAASGFIFSEHNMAWVRQAPKKGLEWVATISDDGRNT |
| | | YYRDSMRGRFTISRENARSTLYLQLDSLRSEDTATYYCASHRYNL |
| | | FDSWGQGVMVTVSS |
| 72 | anti-CD33_VHH_1 | EVQLVESGGGEVQPGGSLRLSCAASRSSGIDVMGWYRQAPGKERE |
| | | LVAEISGVGDTNYAASLADRFTVSRDNAKNTVYLQMSSLRAEDTA |
| | | VYYCNAHSFLDLVGAWGQGTLVTVKP |
| 73 | anti-CD33_VHH_2 | EVQLVESGGGEVQPGGSLRLSCAASGRTFSGYIMGWFRQAPGKER |
| | | ELVARISGNNLSTEYAESVKGRFTISRDNAKNTLYLQMSSLRAED |
| | | TAVYYCAAEYDYSSGDFVYWGQGTLVTVKP |
| 74 | anti-CD33_VHH_3 | EVQLVESGGGEVQPGGSLRLSCAASGSTLNIDHIGWYRQAPGKER |
| | | ELVGVISSGAGPNYAESVKGRFTISRDNAKNTVYLQMSSLRAEDT |
| | | AVYYCNAWIDYGSGLPQNYWGQGTLVTVKP |
| 75 | anti-CLL1_VHH_1 | EVQLVESGGGEVQPGGSLRLSCAASGFLFSIYDMNWYRQAPGKER |
| | | EWVAGITNNGYSTAYAESVKGRFTISRDNAKNTIYLQMSSLRAED |
| | | TAVYYCHADLTKAYDVEYAWGQGTLVTVKP |
| 76 | anti-CLL1_VHH_2 | EVQLVESGGGEVQPGGSLRLSCAASGLLFSIYDMNWYRQAPGKER |
| | | EWVAGITNNGYSTAYAESVKGRFTISRDNAKNTVYLQMSSLRAED |
| | | TAVYYCHTDEWGREYWGQGTLVTVKP |
| 77 | anti-CD123_VHH_1 | EVQLVESGGGEVQPGGSLRLSCTASGRAINMYAMGWFRQAPGKER |
| | | EFVAAINWNGAYTQYAESVKGRFTISRDNAKNTLYLQMSSLRAED |
| | | TAVYYCSADADYNTYVSPNKRVSYWGQGTLVTVKP |
| 78 | anti-CD123_VHH_2 | EVQLVESGGGLVQPGGSLRLSCAASGRAINAYNMGWFRQAPGKGR |
| | | EFVSAINWNAARTYYAESVKGRFTISRDNAKNTLYLQMSSLRAED |
| | | TAVYYCAASGRWSAAVPSGEDQYNFWGQGTLVTVKP |
| 79 | anti-CD20_VHH | QVQLQESGGGLVQAGGSLRLSCAASGRTFSNYNMGWFRQAPGKER |
| | | EFVAAIDWSGGSPYYAASVRGRFTISRDNAENTVYLQMNSLKPED |
| | | TAVYYCAAPLSYGSTWLADYWGQGTQVTVSS |
| 80 | anti-EGFR_VHH | QVKLEESGGGSVQTGGSLRLTCAASGRTSRSYGMGWFRQAPGKER |
| | | EFVSGISWRGDSTGYADSVKGRFTISRDNAKNTVDLQMNSLKPED |
| | | TAIYYCAAAAGSAWYGTLYEYDYWGQGTQVTVSS |
| 81 | anti-BCMA_VHH_1 | QVKLEESGGGLVQAGRSLRLSCAASEHTFSSHVMGWFRQAPGKER |
| | | ESVAVIGWRDISTSYADSVKGRFTISRDNAKKTLYLQMNSLKPED |
| | | TAVYYCAARRIDAADFDSWGQGTQVTVSS |
| 82 | anti-BCMA_VHH_2 | EVQLVESGGGLVQAGGSLRLSCAASGRTFTMGWFRQAPGKEREFV |
| | | AAISLSPTLAYYAESVKGRFTISRDNAKNTVVLQMNSLKPEDTAL |
| | | YYCAADRKSVMSIRPDYWGQGTQVTVSS |
| 83 | anti-CD19_VHH | QVKLEESGGELVQPGGPLRLSCAASGNIFSINRMGWYRQAPGKQR |
| | | AFVASITVRGITNYADSVKGRFTISVDKSKNTIYLQMNALKPEDT |
| | | AVYYCNAVSSNRDPDYWGQGTQVTVSS |
| 84 | PD1 ectodomain | NPPTFSPALLVVTEGDNATFTCSFSNTSESFVLNWYRMSPSNQTD |
| | | KLAAFPEDRSQPGQDSRFRVTQLPNGRDFHMSVVRARRNDSGTYL |
| | | CGAISLAPKAQIKESLRAELRVTE |
| 85 | PD1 high affinity | NPPTFSPALLVVTEGDNATFTCSFSNTSESFVLNWYRMSPSNQTD |
| | ectodomain | KLAAFPEDRSQPGQDSRFRVTQLPNGRDFHMSVVRARRNDSGTYL |
| | | CGAISLAPKVQIKESLRAELRVTE |
| 86 | Human A | SVLHLVPINATSKDDSDVTEVMWQPALRRGRGLQAQGYGVRIQDA |
| | Proliferation- | GVYLLYSQVLFQDVTFTMGQVVSREGQGRQETLFRCIRSMPSHPD |
| | Inducing Ligand | RAYNSCYSAGVFHLHQGDILSVIIPRARAKLNLSPHGTFLGFVKL |
| | (APRIL) | |
| 87 | Trimerized human | SVLHLVPINATSKDDSDVTEVMWQPALRRGRGLQAQGYGVRIQDA |
| | APRIL | GVYLLYSQVLFQDVTFTMGQVVSREGQGRQETLFRCIRSMPSHPD |
| | | RAYNSCYSAGVFHLHQGDILSVIIPRARAKLNLSPHGTFLGFVKL |
| | | GGGGSGGGGSGGGGSSVLHLVPINATSKDDSDVTEVMWQPALRRG |
| | | RGLQAQGYGVRIQDAGVYLLYSQVLFQDVTFTMGQVVSREGQGRQ |
| | | ETLFRCIRSMPSHPDRAYNSCYSAGVFHLHQGDILSVIIPRARAK |
| | | LNLSPHGTFLGFVKLGGGGSGGGGSGGGGSSVLHLVPINATSKDD |
| | | SDVTEVMWQPALRRGRGLQAQGYGVRIQDAGVYLLYSQVLFQDVT |
| | | FTMGQVVSREGQGRQETLFRCIRSMPSHPDRAYNSCYSAGVFHLH |
| | | QGDILSVIIPRARAKLNLSPHGTFLGFVKL |
| 88 | NKG2D membrane | FNQEVQIPLTESYCGPCPKNWICYKNNCYQFFDESKNWYESQASC |
| | protein | MSQNASLLKVYSKEDQDLLKLVKSYHWMGLVHIPINGSWQWEDGS |
| | | ILSPNLLTIIEMQKGDCALYASSFKGYIENCSTPNTYICMQRTV |
| 89 | anti-CD33_VHH_1 CDR1 | RSSGIDVMG |
| 90 | anti-CD33_VHH_1 CDR2 | EISGVGDTN |
| 91 | anti-CD33_VHH_1 CDR3 | HSFLDLVGA |
| 92 | anti-CLL1_VHH_1 CDR1 | GFLFSIYDMN |
| 93 | anti-CLL1_VHH_1 CDR2 | GITNNGYSTA |
| 94 | anti-CLL1_VHH_1 CDR3 | DLTKAYDVEYA |
| 95 | IgK signal sequence | ETPAQLLFLLLLWLPDTTG |
| 96 | CD8 signal sequence | ALPVTALLLPLALLLHAARP |
| 97 | PD1 signal sequence | QIPQAPWPVVWAVLQLGWRPGW |
| 98 | 41BB intracellular | KRGRKKLLYIFKQPFMRPVQTTQEEDGCSCRFPEEEEGGCEL |
| | signaling domain | |
| 99 | CD28 intracellular | RSKRSRLLHSDYMNMTPRRPGPTRKHYQPYAPPRDFAAYRS |
| | signaling domain | |
| 100 | CD4 coreceptor | CVRCRHRRRQAERMSQIKRLLSEKKTCQCPHRFQKTCSPI |
| | domain | |
| 101 | CD8 coreceptor | LYCNHRNRRRVCKCPRPVVKSGDKPSLSARYV |
| | domain | |
| 102 | LAT domain | HCHRLPGSYDSTSSDSLYPRGIQFKRPHTVAPWPPAYPPVTSYPP |
| | | LSQPDLLPIPRSPQPLGGSHRTPSSRRDSDGANSVASYENEGASG |
| | | IRGAQAGWGVWGPSWTRLTPVSLPPEPACEDADEDEDDYHNPGYL |
| | | VVLPDSTPATSTAAPSAPALSTPGIRDSAFSMESIDDYVNVPESG |
| | | ESAEASLDGSREYVNVSQELHPGAAKTEPAALSSQEAEEVEEEGA |
| | | PDYENLQELN |
| 103 | IL7 receptor alpha | KKRIKPIVWPSLPDHKKTLEHLCKKPRKNLNVSFNPESFLDCQIH |
| | signaling domain | RVDDIQARDEVEGFLQDTFPQQLEESEKQRLGGDVQSPNCPSEDV |
| | | VITPESFGRDSSLTCLAGNVSACDAPILSSSRSLDCRESGKNGPH |
| | | VYQDLLLSLGTTNSTLPPPFSLQSGILTLNPVAQGQPILTSLGSN |
| | | QEEAYVTMSSFYQNQ |
| 104 | IL2 receptor beta | NCRNTGPWLKKVLKCNTPDPSKFFSQLSSEHGGDVQKWLSSPFPS |
| | signaling domain, | SSFSPGGLAPEISPLEVLERDKVTQLL |
| | truncated | |
| 105 | IL2 receptor beta | NCRNTGPWLKKVLKCNTPDPSKFFSQLSSEHGGDVQKWLSSPFPS |
| | signaling domain | SSFSPGGLAPEISPLEVLERDKVTQLLLQQDKVPEPASLSSNHSL |
| | | TSCFTNQGYFFFHLPDALEIEACQVYFTYDPYSEEDPDEGVAGAP |
| | | TGSSPQPLQPLSGEDDAYCTFPSRDDLLLFSPSLLGGPSPPSTAP |
| | | GGSGAGEERMPPSLQERVPRDWDPQPLGPPTPGVPDLVDFQPPPE |
| | | LVLREAGEEVPDAGPREGVSFPWSRPPGQGEFRALNARLPLNTDA |
| | | YLSLQELQGQDPTHLV |
| 106 | Common gamma chain | ERTMPRIPTLKNLEDLVTEYHGNFSAWSGVSKGLAESLQPDYSER |
| | signaling domain | LCLVSEIPPKGGALGEGPGASPCNQHSPYWAPPCYTLKPET |
| 107 | FRB.3xG4S.CD3e | WHEMWHEGLEEASRLYFGERNVKGMFEVLEPLHAMMERGPQTLKE |
| | | TSFNQAYGRDLMEAQEWCRKYMKSGNVKDLTQAWDLYYHVFRRIS |
| | | KGGGGSGGGGSGGGGSDGNEEMGGITQTPYKVSISGTTVILTCPQ |
| | | YPGSEILWQHNDKNIGGDEDDKNIGSDEDHLSLKEFSELEQSGYY |
| | | VCYPRGSKPEDANFYLYLRARVCENCMEMDVMSVATIVIVDICIT |
| | | GGLLLLVYYWSKNRKAKAKPVTRGAGAGGRQRGQNKERPPPVPNP |
| | | DYEPIRKGQRDLYSGLNQRRI |
| 108 | FRB.3xG4S.CD3g | WHEMWHEGLEEASRLYFGERNVKGMFEVLEPLHAMMERGPQTLKE |
| | | TSFNQAYGRDLMEAQEWCRKYMKSGNVKDLTQAWDLYYHVFRRIS |
| | | KGGGGSGGGGSGGGGSQSIKGNHLVKVYDYQEDGSVLLTCDAEAK |
| | | NITWFKDGKMIGFLTEDKKKWNLGSNAKDPRGMYQCKGSQNKSKP |
| | | LQVYYRMCQNCIELNAATISGFLFAEIVSIFVLAVGVYFIAGQDG |
| | | VRQSRASDKQTLLPNDQLYQPLKDREDDQYSHLQGNQLRRN |
| 109 | FKBP12.G4S.CD3e | QVETISPGDGRTFPKRGQTCVVHYTGMLEDGKKFDSSRDRNKPFK |
| | | FMLGKQEVIRGWEEGVAQMSVGQRAKLTISPDYAYGATGHPGIIP |
| | | PHATLVFDVELLKLEGGGGSDGNEEMGGITQTPYKVSISGTTVIL |
| | | TCPQYPGSEILWQHNDKNIGGDEDDKNIGSDEDHLSLKEFSELEQ |
| | | SGYYVCYPRGSKPEDANFYLYLRARVCENCMEMDVMSVATIVIVD |
| | | ICITGGLLLLVYYWSKNRKAKAKPVTRGAGAGGRQRGQNKERPPP |
| | | VPNPDYEPIRKGQRDLYSGLNQRRI |
| 110 | FRB.G4S.CD3e | WHEMWHEGLEEASRLYFGERNVKGMFEVLEPLHAMMERGPQTLKE |
| | | TSFNQAYGRDLMEAQEWCRKYMKSGNVKDLTQAWDLYYHVFRRIS |
| | | KGGGGSDGNEEMGGITQTPYKVSISGTTVILTCPQYPGSEILWQH |
| | | NDKNIGGDEDDKNIGSDEDHLSLKEFSELEQSGYYVCYPRGSKPE |
| | | DANFYLYLRARVCENCMEMDVMSVATIVIVDICITGGLLLLVYYW |
| | | SKNRKAKAKPVTRGAGAGGRQRGQNKERPPPVPNPDYEPIRKGQR |
| | | DLYSGLNQRRI |
| 111 | FRBstar.3xG4S.CD3e | WHEMWHEGLEEASRLYFGERNVKGMFEVLEPLHAMMERGPQTLKE |
| | | TSFNQAYGRDLMEAQEWCRKYMKSGNVKDLLQAWDLYYHVFRRIS |
| | | KGGGGSGGGGSGGGGSDGNEEMGGITQTPYKVSISGTTVILTCPQ |
| | | YPGSEILWQHNDKNIGGDEDDKNIGSDEDHLSLKEFSELEQSGYY |
| | | VCYPRGSKPEDANFYLYLRARVCENCMEMDVMSVATIVIVDICIT |
| | | GGLLLLVYYWSKNRKAKAKPVTRGAGAGGRQRGQNKERPPPVPNP |
| | | DYEPIRKGQRDLYSGLNQRRI |
| 112 | IACH3.3xG4S.CD3e | GQPREPQVCTLPPSRDELTKNQVSLSCAVKGFYPSDIAVEWESNG |
| | | QPENNYKTTPPVLDSDGSFFLVSKLTVDKSRWQQGNVFSCSVMHE |
| | | ALHNHYTQKSLSLSPGKGGGGSGGGGSGGGGSDGNEEMGGITQTP |
| | | YKVSISGTTVILTCPQYPGSEILWQHNDKNIGGDEDDKNIGSDED |
| | | HLSLKEFSELEQSGYYVCYPRGSKPEDANFYLYLRARVCENCMEM |
| | | DVMSVATIVIVDICITGGLLLLVYYWSKNRKAKAKPVTRGAGAGG |
| | | RQRGQNKERPPPVPNPDYEPIRKGQRDLYSGLNQRRI |
| 113 | CH3AG.3xG4S.CD3e | GQPFRPEVHLLPPSREEMTKNQWSLTCLARGFYPKDIAVEWESNG |
| | | QPENNYKTTPSRQEPSQGTTTFAVTSKLTVDKSRWQQGNWFSCSV |
| | | MHEALHNHYTQKTISLGGGGSGGGGSGGGGSDGNEEMGGITQTPY |
| | | KVSISGTTVILTCPQYPGSEILWQHNDKNIGGDEDDKNIGSDEDH |
| | | LSLKEFSELEQSGYYVCYPRGSKPEDANFYLYLRARVCENCMEMD |
| | | VMSVATIVIVDICITGGLLLLVYYWSKNRKAKAKPVTRGAGAGGR |
| | | QRGQNKERPPPVPNPDYEPIRKGQRDLYSGLNQRRI |
| 114 | CH3DE.3xG4S.CD3e | GQPREPQVYTDPPSRDELTKNQVSLTCEVKGFYPSDIAVEWESNG |
| | | QPENNYKTTPPVLDSDGSFFLYSKLTVDKSRWQQGNVFSCSVMHE |
| | | ALHNHYTQKSLSLSPGKGGGGSGGGGSGGGGSDGNEEMGGITQTP |
| | | YKVSISGTTVILTCPQYPGSEILWQHNDKNIGGDEDDKNIGSDED |
| | | HLSLKEFSELEQSGYYVCYPRGSKPEDANFYLYLRARVCENCMEM |
| | | DVMSVATIVIVDICITGGLLLLVYYWSKNRKAKAKPVTRGAGAGG |
| | | RQRGQNKERPPPVPNPDYEPIRKGQRDLYSGLNQRRI |
| 115 | FRBstar.2xG4S.FCER1G | WHEMWHEGLEEASRLYFGERNVKGMFEVLEPLHAMMERGPQTLKE |
| | | TSFNQAYGRDLMEAQEWCRKYMKSGNVKDLLQAWDLYYHVFRRIS |
| | | KGGGGSGGGGSGEPLCYILDAILFLYGIVLTLLYCRLKIQVRKAA |
| | | ITSYEKSDGVYTGLSTRNQETYETLKHEKPPQ |
| 116 | antiCD33.G4S.FKBP. | EVQLVESGGGEVQPGGSLRLSCAASRSSGIDVMGWYRQAPGKERE |
| | minCD28hinge.CD28tm. | LVAEISGVGDTNYAASLADRFTVSRDNAKNTVYLQMSSLRAEDTA |
| | CD4ic | VYYCNAHSFLDLVGAWGQGTLVTVKPGGGGSGVQVETISPGDGRT |
| | | FPKRGQTCVVHYTGMLEDGKKFDSSRDRNKPFKFMLGKQEVIRGW |
| | | EEGVAQMSVGQRAKLTISPDYAYGATGHPGIIPPHATLVFDVELL |
| | | KLEKGKHLCPSPLFPGPSKPFWVLVVVGGVLACYSLLVTVAFIIF |
| | | WVCVRCRHRRRQ |
| 117 | antiCD33.G4S.FKBP. | EVQLVESGGGEVQPGGSLRLSCAASRSSGIDVMGWYRQAPGKERE |
| | minCD4hinge.CD4tm. | LVAEISGVGDTNYAASLADRFTVSRDNAKNTVYLQMSSLRAEDTA |
| | CD4ic | VYYCNAHSFLDLVGAWGQGTLVTVKPGGGGSGVQVETISPGDGRT |
| | | FPKRGQTCVVHYTGMLEDGKKFDSSRDRNKPFKFMLGKQEVIRGW |
| | | EEGVAQMSVGQRAKLTISPDYAYGATGHPGIIPPHATLVFDVELL |
| | | KLESNIKVLPTWSTPVQPMALIVLGGVAGLLLFIGLGIFFCVRCR |
| | | HRRRQ |
| 118 | antiCD33.G4S.FKBP. | EVQLVESGGGEVQPGGSLRLSCAASRSSGIDVMGWYRQAPGKERE |
| | GGR.CD4tm.CD4ic | LVAEISGVGDTNYAASLADRFTVSRDNAKNTVYLQMSSLRAEDTA |
| | | VYYCNAHSFLDLVGAWGQGTLVTVKPGGGGSGVQVETISPGDGRT |
| | | FPKRGQTCVVHYTGMLEDGKKFDSSRDRNKPFKFMLGKQEVIRGW |
| | | EEGVAQMSVGQRAKLTISPDYAYGATGHPGIIPPHATLVFDVELL |
| | | KLEGGRMALIVLGGVAGLLLFIGLGIFFCVRCRHRRRQ |
| 119 | FRB.2xG4S.antiCD33. | WHEMWHEGLEEASRLYFGERNVKGMFEVLEPLHAMMERGPQTLKE |
| | minCD4hinge.CD4tm. | TSFNQAYGRDLMEAQEWCRKYMKSGNVKDLTQAWDLYYHVFRRIS |
| | CD4ic | KGGGGSGGGGSEVQLVESGGGEVQPGGSLRLSCAASRSSGIDVMG |
| | | WYRQAPGKERELVAEISGVGDTNYAASLADRFTVSRDNAKNTVYL |
| | | QMSSLRAEDTAVYYCNAHSFLDLVGAWGQGTLVTVKPSNIKVLPT |
| | | WSTPVQPMALIVLGGVAGLLLFIGLGIFFCVRCRHRRRQ |
| 120 | FKBP.2xG4S.antiCD33. | GVQVETISPGDGRTFPKRGQTCVVHYTGMLEDGKKFDSSRDRNKP |
| | minCD4hinge.CD4tm. | FKFMLGKQEVIRGWEEGVAQMSVGQRAKLTISPDYAYGATGHPGI |
| | CD4ic | IPPHATLVFDVELLKLEGGGGSGGGGSEVQLVESGGGEVQPGGSL |
| | | RLSCAASRSSGIDVMGWYRQAPGKERELVAEISGVGDTNYAASLA |
| | | DRFTVSRDNAKNTVYLQMSSLRAEDTAVYYCNAHSFLDLVGAWGQ |
| | | GTLVTVKPSNIKVLPTWSTPVQPMALIVLGGVAGLLLFIGLGIFF |
| | | CVRCRHRRRQ |
| 121 | antiCLL1.G4S.FKBP. | EVQLVESGGGEVQPGGSLRLSCAASGFLFSIYDMNWYRQAPGKER |
| | minCD4hinge.CD4tm. | EWVAGITNNGYSTAYAESVKGRFTISRDNAKNTIYLQMSSLRAED |
| | CD4ic | TAVYYCHADLTKAYDVEYAWGQGTLVTVKPGGGGSGVQVETISPG |
| | | DGRTFPKRGQTCVVHYTGMLEDGKKFDSSRDRNKPFKFMLGKQEV |
| | | IRGWEEGVAQMSVGQRAKLTISPDYAYGATGHPGIIPPHATLVFD |
| | | VELLKLESNIKVLPTWSTPVQPMALIVLGGVAGLLLFIGLGIFFC |
| | | VRCRHRRRQ |
| 122 | antiCLL1.G4S.antiCD33. | EVQLVESGGGEVQPGGSLRLSCAASGFLFSIYDMNWYRQAPGKER |
| | G4S.FKBP.minCD4hinge. | EWVAGITNNGYSTAYAESVKGRFTISRDNAKNTIYLQMSSLRAED |
| | CD4tm.CD4ic | TAVYYCHADLTKAYDVEYAWGQGTLVTVKPGGGGSEVQLVESGGG |
| | | EVQPGGSLRLSCAASRSSGIDVMGWYRQAPGKERELVAEISGVGD |
| | | TNYAASLADRFTVSRDNAKNTVYLQMSSLRAEDTAVYYCNAHSFL |
| | | DLVGAWGQGTLVTVKPGGGGSGVQVETISPGDGRTFPKRGQTCVV |
| | | HYTGMLEDGKKFDSSRDRNKPFKFMLGKQEVIRGWEEGVAQMSVG |
| | | QRAKLTISPDYAYGATGHPGIIPPHATLVFDVELLKLESNIKVLP |
| | | TWSTPVQPMALIVLGGVAGLLLFIGLGIFFCVRCRHRRRQ |
| 123 | FRB.2xG4S.antiCLL1. | WHEMWHEGLEEASRLYFGERNVKGMFEVLEPLHAMMERGPQTLKE |
| | minCD4hinge.CD4tm. | TSFNQAYGRDLMEAQEWCRKYMKSGNVKDLTQAWDLYYHVFRRIS |
| | CD4ic | KGGGGSGGGGSEVQLVESGGGEVQPGGSLRLSCAASGFLFSIYDM |
| | | NWYRQAPGKEREWVAGITNNGYSTAYAESVKGRFTISRDNAKNTI |
| | | YLQMSSLRAEDTAVYYCHADLTKAYDVEYAWGQGTLVTVKPSNIK |
| | | VLPTWSTPVQPMALIVLGGVAGLLLFIGLGIFFCVRCRHRRRQ |
| 124 | antiCLL1.G4S.FRB. | EVQLVESGGGEVQPGGSLRLSCAASGFLFSIYDMNWYRQAPGKER |
| | 2xG4S.antiCD33. | EWVAGITNNGYSTAYAESVKGRFTISRDNAKNTIYLQMSSLRAED |
| | minCD4hinge.CD4tm. | TAVYYCHADLTKAYDVEYAWGQGTLVTVKPGGGGSLWHEMWHEGL |
| | CD4ic | EEASRLYFGERNVKGMFEVLEPLHAMMERGPQTLKETSFNQAYGR |
| | | DLMEAQEWCRKYMKSGNVKDLTQAWDLYYHVFRRISKGGGGSGGG |
| | | GSEVQLVESGGGEVQPGGSLRLSCAASRSSGIDVMGWYRQAPGKE |
| | | RELVAEISGVGDTNYAASLADRFTVSRDNAKNTVYLQMSSLRAED |
| | | TAVYYCNAHSFLDLVGAWGQGTLVTVKPSNIKVLPTWSTPVQPMA |
| | | LIVLGGVAGLLLFIGLGIFFCVRCRHRRRQ |
| 125 | antiCD33.G4S.FKBP. | EVQLVESGGGEVQPGGSLRLSCAASRSSGIDVMGWYRQAPGKERE |
| | minCD4hinge.CD4tm. | LVAEISGVGDTNYAASLADRFTVSRDNAKNTVYLQMSSLRAEDTA |
| | CD28 | VYYCNAHSFLDLVGAWGQGTLVTVKPGGGGSGVQVETISPGDGRT |
| | | FPKRGQTCVVHYTGMLEDGKKFDSSRDRNKPFKFMLGKQEVIRGW |
| | | EEGVAQMSVGQRAKLTISPDYAYGATGHPGIIPPHATLVFDVELL |
| | | KLESNIKVLPTWSTPVQPMALIVLGGVAGLLLFIGLGIFFCVRCR |
| | | SKRSRLLHSDYMNMTPRRPGPTRKHYQPYAPPRDFAAYRS |
| 126 | antiCD33.G4S.FKBP. | EVQLVESGGGEVQPGGSLRLSCAASRSSGIDVMGWYRQAPGKERE |
| | minCD4hinge.CD4tm. | LVAEISGVGDTNYAASLADRFTVSRDNAKNTVYLQMSSLRAEDTA |
| | 41BB | VYYCNAHSFLDLVGAWGQGTLVTVKPGGGGSGVQVETISPGDGRT |
| | | FPKRGQTCVVHYTGMLEDGKKFDSSRDRNKPFKFMLGKQEVIRGW |
| | | EEGVAQMSVGQRAKLTISPDYAYGATGHPGIIPPHATLVFDVELL |
| | | KLESNIKVLPTWSTPVQPMALIVLGGVAGLLLFIGLGIFFCVRCK |
| | | RGRKKLLYIFKQPFMRPVQTTQEEDGCSCRFPEEEEGGCEL |
| 127 | antiCD33.G4S.AbCH3. | EVQLVESGGGEVQPGGSLRLSCAASRSSGIDVMGWYRQAPGKERE |
| | minCD4hinge.CD4tm. | LVAEISGVGDTNYAASLADRFTVSRDNAKNTVYLQMSSLRAEDTA |
| | CD4ic | VYYCNAHSFLDLVGAWGQGTLVTVKPGGGGSGQPREPQVYTLPPC |
| | | RDELTKNQVSLWCLVKGFYPSDIAVEWESNGQPENNYKTTPPVLD |
| | | SDGSFFLYSKLTVDKSRWQQGNVFSCSVMHEALHNHYTQKSLSLS |
| | | PGKSNIKVLPTWSTPVQPMALIVLGGVAGLLLFIGLGIFFCVRCR |
| | | HRRRQ |
| 128 | antiCD33.G4S.CH3GA. | EVQLVESGGGEVQPGGSLRLSCAASRSSGIDVMGWYRQAPGKERE |
| | minCD4hinge.CD4tm. | LVAEISGVGDTNYAASLADRFTVSRDNAKNTVYLQMSSLRAEDTA |
| | CD4ic | VYYCNAHSFLDLVGAWGQGTLVTVKPGGGGSGQPREPQVYTLPPP |
| | | SEELALNELVTLTCLVKGFYPSDIAVEWLQGSQELPREKYLTWAP |
| | | VLDSDGSFFLYSILRVAAEDWKKGDTFSCSVMHEALHNHYTQKSL |
| | | DRSNIKVLPTWSTPVQPMALIVLGGVAGLLLFIGLGIFFCVRCRH |
| | | RRRQ |
| 129 | antiCD33.G4S.CH3KK. | EVQLVESGGGEVQPGGSLRLSCAASRSSGIDVMGWYRQAPGKERE |
| | minCD4hinge.CD4tm. | LVAEISGVGDTNYAASLADRFTVSRDNAKNTVYLQMSSLRAEDTA |
| | CD4ic | VYYCNAHSFLDLVGAWGQGTLVTVKPGGGGSGQPREPQVYTKPPS |
| | | RDELTKNQVSLKCLVKGFYPSDIAVEWESNGQPENNYKTTPPVLD |
| | | SDGSFFLYSKLTVDKSRWQQGNVFSCSVMHEALHNHYTQKSLSLS |
| | | PGKSNIKVLPTWSTPVQPMALIVLGGVAGLLLFIGLGIFFCVRCR |
| | | HRRRQ |
| 130 | antiCD33.G4S.FKBP. | EVQLVESGGGEVQPGGSLRLSCAASRSSGIDVMGWYRQAPGKERE |
| | minCD4hinge.CD4tm. | LVAEISGVGDTNYAASLADRFTVSRDNAKNTVYLQMSSLRAEDTA |
| | CD4ic-full | VYYCNAHSFLDLVGAWGQGTLVTVKPGGGGSGVQVETISPGDGRT |
| | | FPKRGQTCVVHYTGMLEDGKKFDSSRDRNKPFKFMLGKQEVIRGW |
| | | EEGVAQMSVGQRAKLTISPDYAYGATGHPGIIPPHATLVFDVELL |
| | | KLESNIKVLPTWSTPVQPMALIVLGGVAGLLLFIGLGIFFCVRCR |
| | | HRRRQAERMSQIKRLLSEKKTCQCPHRFQKTCSPI |
| 131 | antiCD33.G4S.FKBP. | EVQLVESGGGEVQPGGSLRLSCAASRSSGIDVMGWYRQAPGKERE |
| | minCD4hinge.CD4tm. | LVAEISGVGDTNYAASLADRFTVSRDNAKNTVYLQMSSLRAEDTA |
| | CD8ic-full | VYYCNAHSFLDLVGAWGQGTLVTVKPGGGGSGVQVETISPGDGRT |
| | | FPKRGQTCVVHYTGMLEDGKKFDSSRDRNKPFKFMLGKQEVIRGW |
| | | EEGVAQMSVGQRAKLTISPDYAYGATGHPGIIPPHATLVFDVELL |
| | | KLESNIKVLPTWSTPVQPMALIVLGGVAGLLLFIGLGIFFLYCNH |
| | | RNRRRVCKCPRPVVKSGDKPSLSARYV |
| 132 | CD19scFv.G4S.FKBP. | DIQMTQTTSSLSASLGDRVTISCRASQDISKYLNWYQQKPDGTVK |
| | minCD4hinge.CD4tm. | LLIYHTSRLHSGVPSRFSGSGSGTDYSLTISNLEQEDIATYFCQQ |
| | CD4ic | GNTLPYTFGGGTKLEITGGGGSGGGGSGGGGSEVKLQESGPGLVA |
| | | PSQSLSVTCTVSGVSLPDYGVSWIRQPPRKGLEWLGVIWGSETTY |
| | | YNSALKSRLTIIKDNSKSQVFLKMNSLQTDDTAIYYCAKHYYYGG |
| | | SYAMDYWGQGTSVTVSSGGGGSGVQVETISPGDGRTFPKRGQTCV |
| | | VHYTGMLEDGKKFDSSRDRNKPFKFMLGKQEVIRGWEEGVAQMSV |
| | | GQRAKLTISPDYAYGATGHPGIIPPHATLVFDVELLKLESNIKVL |
| | | PTWSTPVQPMALIVLGGVAGLLLFIGLGIFFCVRCRHRRRQ |
| 133 | PD1(A132V)ex.G4S. | FLDSPDRPWNPPTFSPALLVVTEGDNATFTCSFSNTSESFVLNWY |
| | FKBP.minCD4hinge.CD4tm. | RMSPSNQTDKLAAFPEDRSQPGQDCRFRVTQLPNGRDFHMSVVRA |
| | CD4ic | RRNDSGTYLCGAISLAPKVQIKESLRAELRVTERRAEVPTAHPSP |
| | | SPRPAGQFQTLVGGGGSGVQVETISPGDGRTFPKRGQTCVVHYTG |
| | | MLEDGKKFDSSRDRNKPFKFMLGKQEVIRGWEEGVAQMSVGQRAK |
| | | LTISPDYAYGATGHPGIIPPHATLVFDVELLKLESNIKVLPTWST |
| | | PVQPMALIVLGGVAGLLLFIGLGIFFCVRCRHRRRQ |
| 134 | PD1ex.G4S.FKBP. | FLDSPDRPWNPPTFSPALLVVTEGDNATFTCSFSNTSESFVLNWY |
| | minCD4hinge.CD4tm. | RMSPSNQTDKLAAFPEDRSQPGQDCRFRVTQLPNGRDFHMSVVRA |
| | CD4ic | RRNDSGTYLCGAISLAPKAQIKESLRAELRVTERRAEVPTAHPSP |
| | | SPRPAGQFQTLVGGGGSGVQVETISPGDGRTFPKRGQTCVVHYTG |
| | | MLEDGKKFDSSRDRNKPFKFMLGKQEVIRGWEEGVAQMSVGQRAK |
| | | LTISPDYAYGATGHPGIIPPHATLVFDVELLKLESNIKVLPTWST |
| | | PVQPMALIVLGGVAGLLLFIGLGIFFCVRCRHRRRQ |
| 135 | antiCD33.G4S.FKBP. | EVQLVESGGGEVQPGGSLRLSCAASRSSGIDVMGWYRQAPGKERE |
| | CD4htm.trIL2Rb | LVAEISGVGDTNYAASLADRFTVSRDNAKNTVYLQMSSLRAEDTA |
| | | VYYCNAHSFLDLVGAWGQGTLVTVKPGGGGSGVQVETISPGDGRT |
| | | FPKRGQTCVVHYTGMLEDGKKFDSSRDRNKPFKFMLGKQEVIRGW |
| | | EEGVAQMSVGQRAKLTISPDYAYGATGHPGIIPPHATLVFDVELL |
| | | KLESNIKVLPTWSTPVQPMALIVLGGVAGLLLFIGLGIFFNCRNT |
| | | GPWLKKVLKCNTPDPSKFFSQLSSEHGGDVQKWLSSPFPSSSFSP |
| | | GGLAPEISPLEVLERDKVTQLLPLNTDAYLSLQELQGQDPTHLV |
| 136 | antiROR1.G4S.FKBP. | DIQMTQSPSFLSASVGDRVTINCKASQNIDRYLNWYQQKLGEAPK |
| | minCD4hinge.CD4tm. | RLLYNTNKLQTGIPSRFSGSGSATDFTLTISSLQPEDFATYFCLQ |
| | CD4iC | YNSLPLTFGSGTKLEIKGGGGSGGGGSGGGGSEVQLVESGGGLVQ |
| | | PGRSLKLSCAASGFIFSEHNMAWVRQAPKKGLEWVATISDDGRNT |
| | | YYRDSMRGRFTISRENARSTLYLQLDSLRSEDTATYYCASHRYNL |
| | | FDSWGQGVMVTVSSGGGGSGVQVETISPGDGRTFPKRGQTCVVHY |
| | | TGMLEDGKKFDSSRDRNKPFKFMLGKQEVIRGWEEGVAQMSVGQR |
| | | AKLTISPDYAYGATGHPGIIPPHATLVFDVELLKLESNIKVLPTW |
| | | STPVQPMALIVLGGVAGLLLFIGLGIFFCVRCRHRRRQ |
| 137 | FRBstar.CD4htm.IL7Ra | ILWHEMWHEGLEEASRLYFGERNVKGMFEVLEPLHAMMERGPQTL |
| | | KETSFNQAYGRDLMEAQEWCRKYMKSGNVKDLLQAWDLYYHVFRR |
| | | ISKSNIKVLPTWSTPVQPMALIVLGGVAGLLLFIGLGIFFKKRIK |
| | | PIVWPSLPDHKKTLEHLCKKPRKNLNVSFNPESFLDCQIHRVDDI |
| | | QARDEVEGFLQDTFPQQLEESEKQRLGGDVQSPNCPSEDVVITPE |
| | | SFGRDSSLTCLAGNVSACDAPILSSSRSLDCRESGKNGPHVYQDL |
| | | LLSLGTTNSTLPPPFSLQSGILTLNPVAQGQPILTSLGSNQEEAY |
| | | VTMSSFYQNQ |
| 138 | FRBstar.CD4htm.IL2Rg | ILWHEMWHEGLEEASRLYFGERNVKGMFEVLEPLHAMMERGPQTL |
| | | KETSFNQAYGRDLMEAQEWCRKYMKSGNVKDLLQAWDLYYHVFRR |
| | | ISKSNIKVLPTWSTPVQPMALIVLGGVAGLLLFIGLGIFFERTMP |
| | | RIPTLKNLEDLVTEYHGNFSAWSGVSKGLAESLQPDYSERLCLVS |
| | | EIPPKGGALGEGPGASPCNQHSPYWAPPCYTLKPET |
| 139 | SR020 - | ALPVTALLLPLALLLHAARPGSILWHEMWHEGLEEASRLYFGERN |
| | CD8ss.FRB.CD3e.P2A. | VKGMFEVLEPLHAMMERGPQTLKETSFNQAYGRDLMEAQEWCRKY |
| | IgKss.antiCD33.FKBP. | MKSGNVKDLTQAWDLYYHVFRRISKGGGGSGGGGSGGGGSDGNEE |
| | minCD28hinge.CD28tm. | MGGITQTPYKVSISGTTVILTCPQYPGSEILWQHNDKNIGGDEDD |
| | CD4ic | KNIGSDEDHLSLKEFSELEQSGYYVCYPRGSKPEDANFYLYLRAR |
| | | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGE |
| | | VQLVESGGGEVQPGGSLRLSCAASRSSGIDVMGWYRQAPGKEREL |
| | | VAEISGVGDTNYAASLADRFTVSRDNAKNTVYLQMSSLRAEDTAV |
| | | YYCNAHSFLDLVGAWGQGTLVTVKPGGGGSGVQVETISPGDGRTF |
| | | PKRGQTCVVHYTGMLEDGKKFDSSRDRNKPFKFMLGKQEVIRGWE |
| | | EGVAQMSVGQRAKLTISPDYAYGATGHPGIIPPHATLVFDVELLK |
| | | LEKGKHLCPSPLFPGPSKPFWVLVVVGGVLACYSLLVTVAFIIFW |
| | | VCVRCRHRRRQ |
| 140 | SR021 - | ALPVTALLLPLALLLHAARPGSILWHEMWHEGLEEASRLYFGERN |
| | CD8ss.FRB.CD3g.P2A. | VKGMFEVLEPLHAMMERGPQTLKETSFNQAYGRDLMEAQEWCRKY |
| | IgKss.antiCD33.FKBP. | MKSGNVKDLTQAWDLYYHVFRRISKGGGGSGGGGSGGGGSQSIKG |
| | minCD28hinge.CD28tm. | NHLVKVYDYQEDGSVLLTCDAEAKNITWFKDGKMIGFLTEDKKKW |
| | CD4ic | NLGSNAKDPRGMYQCKGSQNKSKPLQVYYRMCQNCIELNAATISG |
| | | FLFAEIVSIFVLAVGVYFIAGQDGVRQSRASDKQTLLPNDQLYQP |
| | | LKDREDDQYSHLQGNQLRRNSGSGATNFSLLKQAGDVEENPGPSM |
| | | ETPAQLLFLLLLWLPDTTGEVQLVESGGGEVQPGGSLRLSCAASR |
| | | SSGIDVMGWYRQAPGKERELVAEISGVGDTNYAASLADRFTVSRD |
| | | NAKNTVYLQMSSLRAEDTAVYYCNAHSFLDLVGAWGQGTLVTVKP |
| | | GGGGSGVQVETISPGDGRTFPKRGQTCVVHYTGMLEDGKKFDSSR |
| | | DRNKPFKFMLGKQEVIRGWEEGVAQMSVGQRAKLTISPDYAYGAT |
| | | GHPGIIPPHATLVFDVELLKLEKGKHLCPSPLFPGPSKPFWVLVV |
| | | VGGVLACYSLLVTVAFIIFWVCVRCRHRRRQ |
| 141 | SR022 - | ALPVTALLLPLALLLHAARPGSILWHEMWHEGLEEASRLYFGERN |
| | CD8ss.FRB.CD3e.P2A. | VKGMFEVLEPLHAMMERGPQTLKETSFNQAYGRDLMEAQEWCRKY |
| | IgKss.antiCD33.FKBP. | MKSGNVKDLTQAWDLYYHVFRRISKGGGGSGGGGSGGGGSDGNEE |
| | minCD4hinge.CD4tm. | MGGITQTPYKVSISGTTVILTCPQYPGSEILWQHNDKNIGGDEDD |
| | CD4ic | KNIGSDEDHLSLKEFSELEQSGYYVCYPRGSKPEDANFYLYLRAR |
| | | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGE |
| | | VQLVESGGGEVQPGGSLRLSCAASRSSGIDVMGWYRQAPGKEREL |
| | | VAEISGVGDTNYAASLADRFTVSRDNAKNTVYLQMSSLRAEDTAV |
| | | YYCNAHSFLDLVGAWGQGTLVTVKPGGGGSGVQVETISPGDGRTF |
| | | PKRGQTCVVHYTGMLEDGKKFDSSRDRNKPFKFMLGKQEVIRGWE |
| | | EGVAQMSVGQRAKLTISPDYAYGATGHPGIIPPHATLVFDVELLK |
| | | LESNIKVLPTWSTPVQPMALIVLGGVAGLLLFIGLGIFFCVRCRH |
| | | RRRQ |
| 142 | SR023 - | ALPVTALLLPLALLLHAARPGSILWHEMWHEGLEEASRLYFGERN |
| | CD8ss.FRB.CD3g.P2A. | VKGMFEVLEPLHAMMERGPQTLKETSFNQAYGRDLMEAQEWCRKY |
| | IgKss.antiCD33.FKBP. | MKSGNVKDLTQAWDLYYHVFRRISKGGGGSGGGGSGGGGSQSIKG |
| | minCD4hinge.CD4tm. | NHLVKVYDYQEDGSVLLTCDAEAKNITWFKDGKMIGFLTEDKKKW |
| | CD4ic | NLGSNAKDPRGMYQCKGSQNKSKPLQVYYRMCQNCIELNAATISG |
| | | FLFAEIVSIFVLAVGVYFIAGQDGVRQSRASDKQTLLPNDQLYQP |
| | | LKDREDDQYSHLQGNQLRRNSGSGATNFSLLKQAGDVEENPGPSM |
| | | ETPAQLLFLLLLWLPDTTGEVQLVESGGGEVQPGGSLRLSCAASR |
| | | SSGIDVMGWYRQAPGKERELVAEISGVGDTNYAASLADRFTVSRD |
| | | NAKNTVYLQMSSLRAEDTAVYYCNAHSFLDLVGAWGQGTLVTVKP |
| | | GGGGSGVQVETISPGDGRTFPKRGQTCVVHYTGMLEDGKKFDSSR |
| | | DRNKPFKFMLGKQEVIRGWEEGVAQMSVGQRAKLTISPDYAYGAT |
| | | GHPGIIPPHATLVFDVELLKLESNIKVLPTWSTPVQPMALIVLGG |
| | | VAGLLLFIGLGIFFCVRCRHRRRQ |
| 143 | SR024 - | ALPVTALLLPLALLLHAARPGSILWHEMWHEGLEEASRLYFGERN |
| | CD8ss.FRB.CD3e.P2A. | VKGMFEVLEPLHAMMERGPQTLKETSFNQAYGRDLMEAQEWCRKY |
| | IgKss.antiCD33.FKBP. | MKSGNVKDLTQAWDLYYHVFRRISKGGGGSGGGGSGGGGSDGNEE |
| | CD4tm.CD4ic | MGGITQTPYKVSISGTTVILTCPQYPGSEILWQHNDKNIGGDEDD |
| | | KNIGSDEDHLSLKEFSELEQSGYYVCYPRGSKPEDANFYLYLRAR |
| | | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGE |
| | | VQLVESGGGEVQPGGSLRLSCAASRSSGIDVMGWYRQAPGKEREL |
| | | VAEISGVGDTNYAASLADRFTVSRDNAKNTVYLQMSSLRAEDTAV |
| | | YYCNAHSFLDLVGAWGQGTLVTVKPGGGGSGVQVETISPGDGRTF |
| | | PKRGQTCVVHYTGMLEDGKKFDSSRDRNKPFKFMLGKQEVIRGWE |
| | | EGVAQMSVGQRAKLTISPDYAYGATGHPGIIPPHATLVFDVELLK |
| | | LEGGRMALIVLGGVAGLLLFIGLGIFFCVRCRHRRRQ |
| 144 | SR026 - | ALPVTALLLPLALLLHAARPGSGVQVETISPGDGRTFPKRGQTCV |
| | CD8ss.FKBP.CD3e.P2A. | VHYTGMLEDGKKFDSSRDRNKPFKFMLGKQEVIRGWEEGVAQMSV |
| | IgKss.FRB.antiCD33. | GQRAKLTISPDYAYGATGHPGIIPPHATLVFDVELLKLEGGGGSD |
| | minCD4hinge.CD4tm. | GNEEMGGITQTPYKVSISGTTVILTCPQYPGSEILWQHNDKNIGG |
| | CD4ic | DEDDKNIGSDEDHLSLKEFSELEQSGYYVCYPRGSKPEDANFYLY |
| | | LRARVCENCMEMDVMSVATIVIVDICITGGLLLLVYYWSKNRKAK |
| | | AKPVTRGAGAGGRQRGQNKERPPPVPNPDYEPIRKGQRDLYSGLN |
| | | QRRISGSGATNFSLLKQAGDVEENPGPSMETPAQLLFLLLLWLPD |
| | | TTGLWHEMWHEGLEEASRLYFGERNVKGMFEVLEPLHAMMERGPQ |
| | | TLKETSFNQAYGRDLMEAQEWCRKYMKSGNVKDLTQAWDLYYHVF |
| | | RRISKGGGGSGGGGSEVQLVESGGGEVQPGGSLRLSCAASRSSGI |
| | | DVMGWYRQAPGKERELVAEISGVGDTNYAASLADRFTVSRDNAKN |
| | | TVYLQMSSLRAEDTAVYYCNAHSFLDLVGAWGQGTLVTVKPSNIK |
| | | VLPTWSTPVQPMALIVLGGVAGLLLFIGLGIFFCVRCRHRRRQ |
| 145 | SR028 - | ALPVTALLLPLALLLHAARPGSLWHEMWHEGLEEASRLYFGERNV |
| | CD8ss.FRB.CD3e.P2A. | KGMFEVLEPLHAMMERGPQTLKETSFNQAYGRDLMEAQEWCRKYM |
| | IgKss.FKBP.antiCD33. | KSGNVKDLTQAWDLYYHVFRRISKGGGGSDGNEEMGGITQTPYKV |
| | minCD4hinge.CD4tm. | SISGTTVILTCPQYPGSEILWQHNDKNIGGDEDDKNIGSDEDHLS |
| | CD4ic | LKEFSELEQSGYYVCYPRGSKPEDANFYLYLRARVCENCMEMDVM |
| | | SVATIVIVDICITGGLLLLVYYWSKNRKAKAKPVTRGAGAGGRQR |
| | | GQNKERPPPVPNPDYEPIRKGQRDLYSGLNQRRISGSGATNFSLL |
| | | KQAGDVEENPGPSMETPAQLLFLLLLWLPDTTGGVQVETISPGDG |
| | | RTFPKRGQTCVVHYTGMLEDGKKFDSSRDRNKPFKFMLGKQEVIR |
| | | GWEEGVAQMSVGQRAKLTISPDYAYGATGHPGIIPPHATLVFDVE |
| | | LLKLEGGGGSGGGGSEVQLVESGGGEVQPGGSLRLSCAASRSSGI |
| | | DVMGWYRQAPGKERELVAEISGVGDTNYAASLADRFTVSRDNAKN |
| | | TVYLQMSSLRAEDTAVYYCNAHSFLDLVGAWGQGTLVTVKPSNIK |
| | | VLPTWSTPVQPMALIVLGGVAGLLLFIGLGIFFCVRCRHRRRQ |
| 146 | SR001 - | ALPVTALLLPLALLLHAARPGSILWHEMWHEGLEEASRLYFGERN |
| | CD8ss.FRBstar.CD3e. | VKGMFEVLEPLHAMMERGPQTLKETSFNQAYGRDLMEAQEWCRKY |
| | P2A.IgKss.antiCD33. | MKSGNVKDLLQAWDLYYHVFRRISKGGGGSGGGGSGGGGSDGNEE |
| | FKBP.minCD4hinge. | MGGITQTPYKVSISGTTVILTCPQYPGSEILWQHNDKNIGGDEDD |
| | CD4tm.CD4ic | KNIGSDEDHLSLKEFSELEQSGYYVCYPRGSKPEDANFYLYLRAR |
| | | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGE |
| | | VQLVESGGGEVQPGGSLRLSCAASRSSGIDVMGWYRQAPGKEREL |
| | | VAEISGVGDTNYAASLADRFTVSRDNAKNTVYLQMSSLRAEDTAV |
| | | YYCNAHSFLDLVGAWGQGTLVTVKPGGGGSGVQVETISPGDGRTF |
| | | PKRGQTCVVHYTGMLEDGKKFDSSRDRNKPFKFMLGKQEVIRGWE |
| | | EGVAQMSVGQRAKLTISPDYAYGATGHPGIIPPHATLVFDVELLK |
| | | LESNIKVLPTWSTPVQPMALIVLGGVAGLLLFIGLGIFFCVRCRH |
| | | RRRQ |
| 147 | SR003 - | ALPVTALLLPLALLLHAARPGSILWHEMWHEGLEEASRLYFGERN |
| | CD8ss.FRB.CD3e.P2A. | VKGMFEVLEPLHAMMERGPQTLKETSENQAYGRDLMEAQEWCRKY |
| | IgKss.antiCLL1.FKBP. | MKSGNVKDLTQAWDLYYHVFRRISKGGGGSGGGGSGGGGSDGNEE |
| | minCD4hinge.CD4tm. | MGGITQTPYKVSISGTTVILTCPQYPGSEILWQHNDKNIGGDEDD |
| | CD4ic | KNIGSDEDHLSLKEFSELEQSGYYVCYPRGSKPEDANFYLYLRAR |
| | | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGE |
| | | VQLVESGGGEVQPGGSLRLSCAASGFLFSIYDMNWYRQAPGKERE |
| | | WVAGITNNGYSTAYAESVKGRFTISRDNAKNTIYLQMSSLRAEDT |
| | | AVYYCHADLTKAYDVEYAWGQGTLVTVKPGGGGSGVQVETISPGD |
| | | GRTFPKRGQTCVVHYTGMLEDGKKFDSSRDRNKPFKFMLGKQEVI |
| | | RGWEEGVAQMSVGQRAKLTISPDYAYGATGHPGIIPPHATLVFDV |
| | | ELLKLESNIKVLPTWSTPVQPMALIVLGGVAGLLLFIGLGIFFCV |
| | | RCRHRRRQ |
| 148 | SR004 - | ALPVTALLLPLALLLHAARPGSILWHEMWHEGLEEASRLYFGERN |
| | CD8ss.FRB.CD3e.P2A. | VKGMFEVLEPLHAMMERGPQTLKETSFNQAYGRDLMEAQEWCRKY |
| | IgKss.antiCLL1. | MKSGNVKDLTQAWDLYYHVFRRISKGGGGSGGGGSGGGGSDGNEE |
| | antiCD33.FKBP. | MGGITQTPYKVSISGTTVILTCPQYPGSEILWQHNDKNIGGDEDD |
| | minCD4hinge.CD4tm. | KNIGSDEDHLSLKEFSELEQSGYYVCYPRGSKPEDANFYLYLRAR |
| | CD4ic | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGE |
| | | VQLVESGGGEVQPGGSLRLSCAASGFLFSIYDMNWYRQAPGKERE |
| | | WVAGITNNGYSTAYAESVKGRFTISRDNAKNTIYLQMSSLRAEDT |
| | | AVYYCHADLTKAYDVEYAWGQGTLVTVKPGGGGSEVQLVESGGGE |
| | | VQPGGSLRLSCAASRSSGIDVMGWYRQAPGKERELVAEISGVGDT |
| | | NYAASLADRFTVSRDNAKNTVYLQMSSLRAEDTAVYYCNAHSFLD |
| | | LVGAWGQGTLVTVKPGGGGSGVQVETISPGDGRTFPKRGQTCVVH |
| | | YTGMLEDGKKFDSSRDRNKPFKFMLGKQEVIRGWEEGVAQMSVGQ |
| | | RAKLTISPDYAYGATGHPGIIPPHATLVFDVELLKLESNIKVLPT |
| | | WSTPVQPMALIVLGGVAGLLLFIGLGIFFCVRCRHRRRQ |
| 149 | SR005 - | ALPVTALLLPLALLLHAARPGSGVQVETISPGDGRTFPKRGQTCV |
| | CD8ss.FKBP.CD3e.P2A. | VHYTGMLEDGKKFDSSRDRNKPFKFMLGKQEVIRGWEEGVAQMSV |
| | IgKss.FRB.antiCLL1. | GQRAKLTISPDYAYGATGHPGIIPPHATLVFDVELLKLEGGGGSD |
| | minCD4hinge.CD4tm. | GNEEMGGITQTPYKVSISGTTVILTCPQYPGSEILWQHNDKNIGG |
| | CD4ic | DEDDKNIGSDEDHLSLKEFSELEQSGYYVCYPRGSKPEDANFYLY |
| | | LRARVCENCMEMDVMSVATIVIVDICITGGLLLLVYYWSKNRKAK |
| | | AKPVTRGAGAGGRQRGQNKERPPPVPNPDYEPIRKGQRDLYSGLN |
| | | QRRISGSGATNFSLLKQAGDVEENPGPSMETPAQLLFLLLLWLPD |
| | | TTGLWHEMWHEGLEEASRLYFGERNVKGMFEVLEPLHAMMERGPQ |
| | | TLKETSFNQAYGRDLMEAQEWCRKYMKSGNVKDLTQAWDLYYHVF |
| | | RRISKGGGGSGGGGSEVQLVESGGGEVQPGGSLRLSCAASGFLFS |
| | | IYDMNWYRQAPGKEREWVAGITNNGYSTAYAESVKGRFTISRDNA |
| | | KNTIYLQMSSLRAEDTAVYYCHADLTKAYDVEYAWGQGTLVTVKP |
| | | SNIKVLPTWSTPVQPMALIVLGGVAGLLLFIGLGIFFCVRCRHRR |
| | | RQ |
| 150 | SR006 - | ALPVTALLLPLALLLHAARPGSGVQVETISPGDGRTFPKRGQTCV |
| | CD8ss.FKBP.CD3e.P2A. | VHYTGMLEDGKKFDSSRDRNKPFKFMLGKQEVIRGWEEGVAQMSV |
| | IgKss.antiCLL1.FRB. | GQRAKLTISPDYAYGATGHPGIIPPHATLVFDVELLKLEGGGGSD |
| | antiCD33.minCD4hinge. | GNEEMGGITQTPYKVSISGTTVILTCPQYPGSEILWQHNDKNIGG |
| | CD4tm.CD4ic | DEDDKNIGSDEDHLSLKEFSELEQSGYYVCYPRGSKPEDANFYLY |
| | | LRARVCENCMEMDVMSVATIVIVDICITGGLLLLVYYWSKNRKAK |
| | | AKPVTRGAGAGGRQRGQNKERPPPVPNPDYEPIRKGQRDLYSGLN |
| | | QRRISGSGATNFSLLKQAGDVEENPGPSMETPAQLLFLLLLWLPD |
| | | TTGEVQLVESGGGEVQPGGSLRLSCAASGFLFSIYDMNWYRQAPG |
| | | KEREWVAGITNNGYSTAYAESVKGRFTISRDNAKNTIYLQMSSLR |
| | | AEDTAVYYCHADLTKAYDVEYAWGQGTLVTVKPGGGGSLWHEMWH |
| | | EGLEEASRLYFGERNVKGMFEVLEPLHAMMERGPQTLKETSFNQA |
| | | YGRDLMEAQEWCRKYMKSGNVKDLTQAWDLYYHVERRISKGGGGS |
| | | GGGGSEVQLVESGGGEVQPGGSLRLSCAASRSSGIDVMGWYRQAP |
| | | GKERELVAEISGVGDTNYAASLADRFTVSRDNAKNTVYLQMSSLR |
| | | AEDTAVYYCNAHSFLDLVGAWGQGTLVTVKPSNIKVLPTWSTPVQ |
| | | PMALIVLGGVAGLLLFIGLGIFFCVRCRHRRRQ |
| 151 | SR008 | ALPVTALLLPLALLLHAARPGSILWHEMWHEGLEEASRLYFGERN |
| | CD8ss.FRBstar.CD3e. | VKGMFEVLEPLHAMMERGPQTLKETSFNQAYGRDLMEAQEWCRKY |
| | P2A.IgKss.antiCLL1. | MKSGNVKDLLQAWDLYYHVFRRISKGGGGSGGGGSGGGGSDGNEE |
| | antiCD33.FKBP. | MGGITQTPYKVSISGTTVILTCPQYPGSEILWQHNDKNIGGDEDD |
| | minCD4hinge.CD4tm. | KNIGSDEDHLSLKEFSELEQSGYYVCYPRGSKPEDANFYLYLRAR |
| | CD4ic | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGE |
| | | VQLVESGGGEVQPGGSLRLSCAASGFLFSIYDMNWYRQAPGKERE |
| | | WVAGITNNGYSTAYAESVKGRFTISRDNAKNTIYLQMSSLRAEDT |
| | | AVYYCHADLTKAYDVEYAWGQGTLVTVKPGGGGSEVQLVESGGGE |
| | | VQPGGSLRLSCAASRSSGIDVMGWYRQAPGKERELVAEISGVGDT |
| | | NYAASLADRFTVSRDNAKNTVYLQMSSLRAEDTAVYYCNAHSFLD |
| | | LVGAWGQGTLVTVKPGGGGSGVQVETISPGDGRTFPKRGQTCVVH |
| | | YTGMLEDGKKFDSSRDRNKPFKFMLGKQEVIRGWEEGVAQMSVGQ |
| | | RAKLTISPDYAYGATGHPGIIPPHATLVFDVELLKLESNIKVLPT |
| | | WSTPVQPMALIVLGGVAGLLLFIGLGIFFCVRCRHRRRQ |
| 152 | SR030 - | ALPVTALLLPLALLLHAARPGSILWHEMWHEGLEEASRLYFGERN |
| | CD8ss.FRBstar.CD3e. | VKGMFEVLEPLHAMMERGPQTLKETSFNQAYGRDLMEAQEWCRKY |
| | P2A.IgKss.antiCLL1. | MKSGNVKDLLQAWDLYYHVFRRISKGGGGSGGGGSGGGGSDGNEE |
| | antiCD33.FKBP. | MGGITQTPYKVSISGTTVILTCPQYPGSEILWQHNDKNIGGDEDD |
| | minCD4hinge.CD4tm. | KNIGSDEDHLSLKEFSELEQSGYYVCYPRGSKPEDANFYLYLRAR |
| | 41BB | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGE |
| | | VQLVESGGGEVQPGGSLRLSCAASGFLFSIYDMNWYRQAPGKERE |
| | | WVAGITNNGYSTAYAESVKGRFTISRDNAKNTIYLQMSSLRAEDT |
| | | AVYYCHADLTKAYDVEYAWGQGTLVTVKPGGGGSEVQLVESGGGE |
| | | VQPGGSLRLSCAASRSSGIDVMGWYRQAPGKERELVAEISGVGDT |
| | | NYAASLADRFTVSRDNAKNTVYLQMSSLRAEDTAVYYCNAHSFLD |
| | | LVGAWGQGTLVTVKPGGGGSGVQVETISPGDGRTFPKRGQTCVVH |
| | | YTGMLEDGKKFDSSRDRNKPFKFMLGKQEVIRGWEEGVAQMSVGQ |
| | | RAKLTISPDYAYGATGHPGIIPPHATLVFDVELLKLESNIKVLPT |
| | | WSTPVQPMALIVLGGVAGLLLFIGLGIFFCVRCKRGRKKLLYIFK |
| | | QPFMRPVQTTQEEDGCSCRFPEEEEGGCEL |
| 153 | SR001-28 - | ALPVTALLLPLALLLHAARPGSILWHEMWHEGLEEASRLYFGERN |
| | CD8ss.FRBstar.CD3e. | VKGMFEVLEPLHAMMERGPQTLKETSFNQAYGRDLMEAQEWCRKY |
| | P2A.IgKss.antiCD33. | MKSGNVKDLLQAWDLYYHVFRRISKGGGGSGGGGSGGGGSDGNEE |
| | FKBP.minCD4hinge. | MGGITQTPYKVSISGTTVILTCPQYPGSEILWQHNDKNIGGDEDD |
| | CD4tm.CD28 | KNIGSDEDHLSLKEFSELEQSGYYVCYPRGSKPEDANFYLYLRAR |
| | | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGE |
| | | VQLVESGGGEVQPGGSLRLSCAASRSSGIDVMGWYRQAPGKEREL |
| | | VAEISGVGDTNYAASLADRFTVSRDNAKNTVYLQMSSLRAEDTAV |
| | | YYCNAHSFLDLVGAWGQGTLVTVKPGGGGSGVQVETISPGDGRTF |
| | | PKRGQTCVVHYTGMLEDGKKFDSSRDRNKPFKFMLGKQEVIRGWE |
| | | EGVAQMSVGQRAKLTISPDYAYGATGHPGIIPPHATLVFDVELLK |
| | | LESNIKVLPTWSTPVQPMALIVLGGVAGLLLFIGLGIFFCVRCRS |
| | | KRSRLLHSDYMNMTPRRPGPTRKHYQPYAPPRDFAAYRS |
| 154 | SR001-41BB - | ALPVTALLLPLALLLHAARPGSILWHEMWHEGLEEASRLYFGERN |
| | CD8ss.FRBstar.CD3e. | VKGMFEVLEPLHAMMERGPQTLKETSFNQAYGRDLMEAQEWCRKY |
| | P2A.IgKss.antiCD33. | MKSGNVKDLLQAWDLYYHVFRRISKGGGGSGGGGSGGGGSDGNEE |
| | FKBP.minCD4hinge. | MGGITQTPYKVSISGTTVILTCPQYPGSEILWQHNDKNIGGDEDD |
| | CD4tm.41BB | KNIGSDEDHLSLKEFSELEQSGYYVCYPRGSKPEDANFYLYLRAR |
| | | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGE |
| | | VQLVESGGGEVQLVESGGGEVQPGGSLRLSCAASRSSGIDVMGWY |
| | | RQAPGKERELVAEISGVGDTNYAASLADRFTVSRDNAKNTVYLQM |
| | | SSLRAEDTAVYYCNAHSFLDLVGAWGQGTLVTVKPGGGGSGVQVE |
| | | TISPGDGRTFPKRGQTCVVHYTGMLEDGKKFDSSRDRNKPFKFML |
| | | GKQEVIRGWEEGVAQMSVGQRAKLTISPDYAYGATGHPGIIPPHA |
| | | TLVFDVELLKLESNIKVLPTWSTPVQPMALIVLGGVAGLLLFIGL |
| | | GIFFCVRCKRGRKKLLYIFKQPFMRPVQTTQEEDGCSCRFPEEEE |
| | | GGCEL |
| 155 | SR292 - | ALPVTALLLPLALLLHAARPGSGQPREPQVCTLPPSRDELTKNQV |
| | CD8ss.IACH3.CD3e.P2A. | SLSCAVKGFYPSDIAVEWESNGQPENNYKTTPPVLDSDGSFFLVS |
| | IgKss.antiCD33.AbCH3. | KLTVDKSRWQQGNVFSCSVMHEALHNHYTQKSLSLSPGKGGGGSG |
| | minCD4hinge.CD4tm. | GGGSGGGGSDGNEEMGGITQTPYKVSISGTTVILTCPQYPGSEIL |
| | 41BB | WQHNDKNIGGDEDDKNIGSDEDHLSLKEFSELEQSGYYVCYPRGS |
| | | KPEDANFYLYLRARVCENCMEMDVMSVATIVIVDICITGGLLLLV |
| | | YYWSKNRKAKAKPVTRGAGAGGRQRGQNKERPPPVPNPDYEPIRK |
| | | GQRDLYSGLNQRRISGSGATNFSLLKQAGDVEENPGPSMETPAQL |
| | | LFLLLLWLPDTTGEVQLVESGGGEVQPGGSLRLSCAASRSSGIDV |
| | | MGWYRQAPGKERELVAEISGVGDTNYAASLADRFTVSRDNAKNTV |
| | | YLQMSSLRAEDTAVYYCNAHSFLDLVGAWGQGTLVTVKPGGGGSG |
| | | QPREPQVYTLPPCRDELTKNQVSLWCLVKGFYPSDIAVEWESNGQ |
| | | PENNYKTTPPVLDSDGSFFLYSKLTVDKSRWQQGNVFSCSVMHEA |
| | | LHNHYTQKSLSLSPGKSNIKVLPTWSTPVQPMALIVLGGVAGLLL |
| | | FIGLGIFFCVRCRHRRRQ |
| 156 | SR293 - | ALPVTALLLPLALLLHAARPGSGQPFRPEVHLLPPSREEMTKNQW |
| | CD8ss.CH3AG.CD3e.P2A. | SLTCLARGFYPKDIAVEWESNGQPENNYKTTPSRQEPSQGTTTFA |
| | IgKss.antiCD33.CH3GA. | VTSKLTVDKSRWQQGNWFSCSVMHEALHNHYTQKTISLGGGGSGG |
| | minCD4hinge.CD4tm. | GGSGGGGSDGNEEMGGITQTPYKVSISGTTVILTCPQYPGSEILW |
| | CD4ic | QHNDKNIGGDEDDKNIGSDEDHLSLKEFSELEQSGYYVCYPRGSK |
| | | PEDANFYLYLRARVCENCMEMDVMSVATIVIVDICITGGLLLLVY |
| | | YWSKNRKAKAKPVTRGAGAGGRQRGQNKERPPPVPNPDYEPIRKG |
| | | QRDLYSGLNQRRISGSGATNFSLLKQAGDVEENPGPSMETPAQLL |
| | | FLLLLWLPDTTGEVQLVESGGGEVQPGGSLRLSCAASRSSGIDVM |
| | | GWYRQAPGKERELVAEISGVGDTNYAASLADRFTVSRDNAKNTVY |
| | | LQMSSLRAEDTAVYYCNAHSFLDLVGAWGQGTLVTVKPGGGGSGQ |
| | | PREPQVYTLPPPSEELALNELVTLTCLVKGFYPSDIAVEWLQGSQ |
| | | ELPREKYLTWAPVLDSDGSFFLYSILRVAAEDWKKGDTFSCSVMH |
| | | EALHNHYTQKSLDRSNIKVLPTWSTPVQPMALIVLGGVAGLLLFI |
| | | GLGIFFCVRCRHRRRQ |
| 157 | SR296 - | ALPVTALLLPLALLLHAARPGSGQPREPQVYTDPPSRDELTKNQV |
| | CD8ss.CH3DE.CD3e.P2A. | SLTCEVKGFYPSDIAVEWESNGQPENNYKTTPPVLDSDGSFFLYS |
| | IgKss.antiCD33.CH3KK. | KLTVDKSRWQQGNVFSCSVMHEALHNHYTQKSLSLSPGKGGGGSG |
| | minCD4hinge.CD4tm. | GGGSGGGGSDGNEEMGGITQTPYKVSISGTTVILTCPQYPGSEIL |
| | CD4ic | WQHNDKNIGGDEDDKNIGSDEDHLSLKEFSELEQSGYYVCYPRGS |
| | | KPEDANFYLYLRARVCENCMEMDVMSVATIVIVDICITGGLLLLV |
| | | YYWSKNRKAKAKPVTRGAGAGGRQRGQNKERPPPVPNPDYEPIRK |
| | | GQRDLYSGLNQRRISGSGATNFSLLKQAGDVEENPGPSMETPAQL |
| | | LFLLLLWLPDTTGEVQLVESGGGEVQPGGSLRLSCAASRSSGIDV |
| | | MGWYRQAPGKERELVAEISGVGDTNYAASLADRFTVSRDNAKNTV |
| | | YLQMSSLRAEDTAVYYCNAHSFLDLVGAWGQGTLVTVKPGGGGSG |
| | | QPREPQVYTKPPSRDELTKNQVSLKCLVKGFYPSDIAVEWESNGQ |
| | | PENNYKTTPPVLDSDGSFFLYSKLTVDKSRWQQGNVFSCSVMHEA |
| | | LHNHYTQKSLSLSPGKSNIKVLPTWSTPVQPMALIVLGGVAGLLL |
| | | FIGLGIFFCVRCRHRRRQ |
| 158 | SR001-CD4 - | ALPVTALLLPLALLLHAARPGSILWHEMWHEGLEEASRLYFGERN |
| | CD8ss.FRB.CD3e.P2A. | VKGMFEVLEPLHAMMERGPQTLKETSFNQAYGRDLMEAQEWCRKY |
| | huIgKss.CD33.FKBP. | MKSGNVKDLTQAWDLYYHVFRRISKGGGGSGGGGSGGGGSDGNEE |
| | minCD4hinge.CD4tm. | MGGITQTPYKVSISGTTVILTCPQYPGSEILWQHNDKNIGGDEDD |
| | CD4ic-full | KNIGSDEDHLSLKEFSELEQSGYYVCYPRGSKPEDANFYLYLRAR |
| | | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGE |
| | | VQLVESGGGEVQPGGSLRLSCAASRSSGIDVMGWYRQAPGKEREL |
| | | VAEISGVGDTNYAASLADRFTVSRDNAKNTVYLQMSSLRAEDTAV |
| | | YYCNAHSFLDLVGAWGQGTLVTVKPGGGGSGVQVETISPGDGRTF |
| | | PKRGQTCVVHYTGMLEDGKKFDSSRDRNKPFKFMLGKQEVIRGWE |
| | | EGVAQMSVGQRAKLTISPDYAYGATGHPGIIPPHATLVFDVELLK |
| | | LESNIKVLPTWSTPVQPMALIVLGGVAGLLLFIGLGIFFCVRCRH |
| | | RRRQAERMSQIKRLLSEKKTCQCPHRFQKTCSPI |
| 159 | SR001-CD8 - | ALPVTALLLPLALLLHAARPGSILWHEMWHEGLEEASRLYFGERN |
| | CD8ss.FRB.CD3e.P2A. | VKGMFEVLEPLHAMMERGPQTLKETSFNQAYGRDLMEAQEWCRKY |
| | huIgKss.antiCD33.FKBP. | MKSGNVKDLTQAWDLYYHVFRRISKGGGGSGGGGSGGGGSDGNEE |
| | minCD4hinge.CD4tm. | MGGITQTPYKVSISGTTVILTCPQYPGSEILWQHNDKNIGGDEDD |
| | CD8ic-full | KNIGSDEDHLSLKEFSELEQSGYYVCYPRGSKPEDANFYLYLRAR |
| | | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGE |
| | | VQLVESGGGEVQPGGSLRLSCAASRSSGIDVMGWYRQAPGKEREL |
| | | VAEISGVGDTNYAASLADRFTVSRDNAKNTVYLQMSSLRAEDTAV |
| | | YYCNAHSFLDLVGAWGQGTLVTVKPGGGGSGVQVETISPGDGRTF |
| | | PKRGQTCVVHYTGMLEDGKKFDSSRDRNKPFKFMLGKQEVIRGWE |
| | | EGVAQMSVGQRAKLTISPDYAYGATGHPGIIPPHATLVFDVELLK |
| | | LESNIKVLPTWSTPVQPMALIVLGGVAGLLLFIGLGIFFLYCNHR |
| | | NRRRVCKCPRPVVKSGDKPSLSARYV |
| 160 | TCR-SR001 - | GFRLLCCVAFCLLGAGPVDSGVTQTPKHLITATGQRVTLRCSPRS |
| | TCR.T2A.SR001 | GDLSVYWYQQSLDQGLQFLIQYYNGEERAKGNILERFSAQQFPDL |
| | | HSELNLSSLELGDSALYFCASSGGDGDEQFFGPGTRLTVLEDLKN |
| | | VFPPEVAVFEPSKAEIAHTQKATLVCLATGFYPDHVELSWWVNGK |
| | | EVHSGVSTDPQPLKEQPALNDSRYCLSSRLRVSATFWQNPRNHFR |
| | | CQVQFYGLSENDEWTQDRAKPVTQIVSAEAWGRADCGITSASYHQ |
| | | GVLSATILYEILLGKATLYAVLVSALVLMAMVKRKDSRGRAKRGS |
| | | GATNFSLLKQAGDVEENPGPMSLSSLLKVVTASLWLGPGIAQKIT |
| | | QTQPGMFVQEKEAVTLDCTYDTSDPSYGLFWYKQPSSGEMIFLIY |
| | | QGSYDQQNATEGRYSLNFQKARKSANLVISASQLGDSAMYFCAMS |
| | | GGYTGGFKTIFGAGTRLFVKANIQNPDPAVYQLRDSKSSDKSVCL |
| | | FTDFDSQTNVSQSKDSDVYITDKTVLDMRSMDFKSNSAVAWSNKS |
| | | DFACANAFNNSIIPEDTFFPSSDVPCDVKLVEKSFETDINLNFQN |
| | | LLVIVLRILLLKVAGFNLLMTLRLWSSGSGEGRGSLLTCGDVEEN |
| | | PGPMALPVTALLLPLALLLHAARPGSILWHEMWHEGLEEASRLYF |
| | | GERNVKGMFEVLEPLHAMMERGPQTLKETSFNQAYGRDLMEAQEW |
| | | CRKYMKSGNVKDLLQAWDLYYHVFRRISKGGGGSGGGGSGGGGSD |
| | | GNEEMGGITQTPYKVSISGTTVILTCPQYPGSEILWQHNDKNIGG |
| | | DEDDKNIGSDEDHLSLKEFSELEQSGYYVCYPRGSKPEDANFYLY |
| | | LRARVCENCMEMDVMSVATIVIVDICITGGLLLLVYYWSKNRKAK |
| | | AKPVTRGAGAGGRQRGQNKERPPPVPNPDYEPIRKGQRDLYSGLN |
| | | QRRISGSGATNFSLLKQAGDVEENPGPSMETPAQLLFLLLLWLPD |
| | | TTGEVQLVESGGGEVQPGGSLRLSCAASRSSGIDVMGWYRQAPGK |
| | | ERELVAEISGVGDTNYAASLADRFTVSRDNAKNTVYLQMSSLRAE |
| | | DTAVYYCNAHSFLDLVGAWGQGTLVTVKPGGGGSGVQVETISPGD |
| | | GRTFPKRGQTCVVHYTGMLEDGKKFDSSRDRNKPFKFMLGKQEVI |
| | | RGWEEGVAQMSVGQRAKLTISPDYAYGATGHPGIIPPHATLVFDV |
| | | ELLKLESNIKVLPTWSTPVQPMALIVLGGVAGLLLFIGLGIFFCV |
| | | RCRHRRRQ |
| 161 | SR007 - | ALPVTALLLPLALLLHAARPGSILWHEMWHEGLEEASRLYFGERN |
| | CD8ss.FRBstar.CD3e. | VKGMFEVLEPLHAMMERGPQTLKETSFNQAYGRDLMEAQEWCRKY |
| | P2A.huIgKss.antiCLL1. | MKSGNVKDLLQAWDLYYHVFRRISKGGGGSGGGGSGGGGSDGNEE |
| | FKBP.minCD4hinge. | MGGITQTPYKVSISGTTVILTCPQYPGSEILWQHNDKNIGGDEDD |
| | CD4tm.CD4ic | KNIGSDEDHLSLKEFSELEQSGYYVCYPRGSKPEDANFYLYLRAR |
| | | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGE |
| | | VQLVESGGGEVQPGGSLRLSCAASGFLFSIYDMNWYRQAPGKERE |
| | | WVAGITNNGYSTAYAESVKGRFTISRDNAKNTIYLQMSSLRAEDT |
| | | AVYYCHADLTKAYDVEYAWGQGTLVTVKPGGGGSGVQVETISPGD |
| | | GRTFPKRGQTCVVHYTGMLEDGKKFDSSRDRNKPFKFMLGKQEVI |
| | | RGWEEGVAQMSVGQRAKLTISPDYAYGATGHPGIIPPHATLVFDV |
| | | ELLKLESNIKVLPTWSTPVQPMALIVLGGVAGLLLFIGLGIFFCV |
| | | RCRHRRRQ |
| 162 | SR10168 - | ALPVTALLLPLALLLHAARPGSILWHEMWHEGLEEASRLYFGERN |
| | CD8ss.FRBstar- | VKGMFEVLEPLHAMMERGPQTLKETSFNQAYGRDLMEAQEWCRKY |
| | CD3e.P2A.IgKss. | MKSGNVKDLLQAWDLYYHVFRRISKGGGGSGGGGSGGGGSDGNEE |
| | CD19sCFv.FKBP. | MGGITQTPYKVSISGTTVILTCPQYPGSEILWQHNDKNIGGDEDD |
| | minCD4hinge.CD4tm. | KNIGSDEDHLSLKEFSELEQSGYYVCYPRGSKPEDANFYLYLRAR |
| | CD4ic | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGD |
| | | IQMTQTTSSLSASLGDRVTISCRASQDISKYLNWYQQKPDGTVKL |
| | | LIYHTSRLHSGVPSRFSGSGSGTDYSLTISNLEQEDIATYFCQQG |
| | | NTLPYTFGGGTKLEITGGGGSGGGGSGGGGSEVKLQESGPGLVAP |
| | | SQSLSVTCTVSGVSLPDYGVSWIRQPPRKGLEWLGVIWGSETTYY |
| | | NSALKSRLTIIKDNSKSQVFLKMNSLQTDDTAIYYCAKHYYYGGS |
| | | YAMDYWGQGTSVTVSSGGGGSGVQVETISPGDGRTFPKRGQTCVV |
| | | HYTGMLEDGKKFDSSRDRNKPFKFMLGKQEVIRGWEEGVAQMSVG |
| | | QRAKLTISPDYAYGATGHPGIIPPHATLVFDVELLKLESNIKVLP |
| | | TWSTPVQPMALIVLGGVAGLLLFIGLGIFFCVRCRHRRRQ |
| 163 | SR001 + IL7a - | ALPVTALLLPLALLLHAARPGSILWHEMWHEGLEEASRLYFGERN |
| | CD8ss.FRBstar.CD3eP2A. | VKGMFEVLEPLHAMMERGPQTLKETSFNQAYGRDLMEAQEWCRKY |
| | huIgKss.FRBstar. | MKSGNVKDLLQAWDLYYHVFRRISKGGGGSGGGGSGGGGSDGNEE |
| | CD4htm.IL7Ra.P2A. | MGGITQTPYKVSISGTTVILTCPQYPGSEILWQHNDKNIGGDEDD |
| | huIgKss.antiCD33.FKBP. | KNIGSDEDHLSLKEFSELEQSGYYVCYPRGSKPEDANFYLYLRAR |
| | minCD4hinge.CD4tm. | VCENCMEMDVMSVATIVIVDICITGGLLLLVYYWSKNRKAKAKPV |
| | CD4ic | TRGAGAGGRQRGQNKERPPPVPNPDYEPIRKGQRDLYSGLNQRRI |
| | | SGSGATNFSLLKQAGDVEENPGPMETPAQLLFLLLLWLPDTTGSI |
| | | LWHEMWHEGLEEASRLYFGERNVKGMFEVLEPLHAMMERGPQTLK |
| | | ETSFNQAYGRDLMEAQEWCRKYMKSGNVKDLLQAWDLYYHVFRRI |
| | | SKSNIKVLPTWSTPVQPMALIVLGGVAGLLLFIGLGIFFKKRIKP |
| | | IVWPSLPDHKKTLEHLCKKPRKNLNVSFNPESFLDCQIHRVDDIQ |
| | | ARDEVEGFLQDTFPQQLEESEKQRLGGDVQSPNCPSEDVVITPES |
| | | FGRDSSLTCLAGNVSACDAPILSSSRSLDCRESGKNGPHVYQDLL |
| | | LSLGTTNSTLPPPFSLQSGILTLNPVAQGQPILTSLGSNQEEAYV |
| | | TMSSFYQNQSGSGATNFSLLKQAGDVEENPGPSMETPAQLLFLLL |
| | | LWLPDTTGEVQLVESGGGEVQPGGSLRLSCAASRSSGIDVMGWYR |
| | | QAPGKERELVAEISGVGDTNYAASLADRFTVSRDNAKNTVYLQMS |
| | | SLRAEDTAVYYCNAHSFLDLVGAWGQGTLVTVKPGGGGSGVQVET |
| | | ISPGDGRTFPKRGQTCVVHYTGMLEDGKKFDSSRDRNKPFKFMLG |
| | | KQEVIRGWEEGVAQMSVGQRAKLTISPDYAYGATGHPGIIPPHAT |
| | | LVFDVELLKLESNIKVLPTWSTPVQPMALIVLGGVAGLLLFIGLG |
| | | IFFCVRCRHRRRQ |
| 164 | SR001 + gC - | ALPVTALLLPLALLLHAARPGSILWHEMWHEGLEEASRLYFGERN |
| | CD8ss.FRBstar.CD3e. | VKGMFEVLEPLHAMMERGPQTLKETSFNQAYGRDLMEAQEWCRKY |
| | P2A.huIgKss.FRBstar. | MKSGNVKDLLQAWDLYYHVFRRISKGGGGSGGGGSGGGGSDGNEE |
| | CD4htm.IL2Rg.P2A. | MGGITQTPYKVSISGTTVILTCPQYPGSEILWQHNDKNIGGDEDD |
| | huIgKss.antiCD33. | KNIGSDEDHLSLKEFSELEQSGYYVCYPRGSKPEDANFYLYLRAR |
| | FKBP.CD4htm.CD4ic | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPMETPAQLLFLLLLWLPDTTGSI |
| | | LWHEMWHEGLEEASRLYFGERNVKGMFEVLEPLHAMMERGPQTLK |
| | | ETSFNQAYGRDLMEAQEWCRKYMKSGNVKDLLQAWDLYYHVFRRI |
| | | SKSNIKVLPTWSTPVQPMALIVLGGVAGLLLFIGLGIFFERTMPR |
| | | IPTLKNLEDLVTEYHGNFSAWSGVSKGLAESLQPDYSERLCLVSE |
| | | IPPKGGALGEGPGASPCNQHSPYWAPPCYTLKPETSGSGATNFSL |
| | | LKQAGDVEENPGPSMETPAQLLFLLLLWLPDTTGEVQLVESGGGE |
| | | VQPGGSLRLSCAASRSSGIDVMGWYRQAPGKERELVAEISGVGDT |
| | | NYAASLADRFTVSRDNAKNTVYLQMSSLRAEDTAVYYCNAHSFLD |
| | | LVGAWGQGTLVTVKPGGGGSGVQVETISPGDGRTFPKRGQTCVVH |
| | | YTGMLEDGKKFDSSRDRNKPFKFMLGKQEVIRGWEEGVAQMSVGQ |
| | | RAKLTISPDYAYGATGHPGIIPPHATLVFDVELLKLESNIKVLPT |
| | | WSTPVQPMALIVLGGVAGLLLFIGLGIFFCVRCRHRRRQ |
| 165 | SR001 + IL2Rb - | ALPVTALLLPLALLLHAARPGSILWHEMWHEGLEEASRLYFGERN |
| | CD8ss.FRBstar.CD3e. | VKGMFEVLEPLHAMMERGPQTLKETSFNQAYGRDLMEAQEWCRKY |
| | P2A.huIgKss.antiCD33. | MKSGNVKDLLQAWDLYYHVFRRISKGGGGSGGGGSGGGGSDGNEE |
| | FKBP.CD4htm.trIL2Rb | MGGITQTPYKVSISGTTVILTCPQYPGSEILWQHNDKNIGGDEDD |
| | | KNIGSDEDHLSLKEFSELEQSGYYVCYPRGSKPEDANFYLYLRAR |
| | | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGE |
| | | VQLVESGGGEVQPGGSLRLSCAASRSSGIDVMGWYRQAPGKEREL |
| | | VAEISGVGDTNYAASLADRFTVSRDNAKNTVYLQMSSLRAEDTAV |
| | | YYCNAHSFLDLVGAWGQGTLVTVKPGGGGSGVQVETISPGDGRTF |
| | | PKRGQTCVVHYTGMLEDGKKFDSSRDRNKPFKFMLGKQEVIRGWE |
| | | EGVAQMSVGQRAKLTISPDYAYGATGHPGIIPPHATLVFDVELLK |
| | | LESNIKVLPTWSTPVQPMALIVLGGVAGLLLFIGLGIFFNCRNTG |
| | | PWLKKVLKCNTPDPSKFFSQLSSEHGGDVQKWLSSPFPSSSFSPG |
| | | GLAPEISPLEVLERDKVTQLLPLNTDAYLSLQELQGQDPTHLV |
| 166 | SR001 + IL2Rb + gC - | ALPVTALLLPLALLLHAARPGSILWHEMWHEGLEEASRLYFGERN |
| | CD8ss.FRBstar.CD3e. | VKGMFEVLEPLHAMMERGPQTLKETSFNQAYGRDLMEAQEWCRKY |
| | P2A.huIgKss.FRBstar. | MKSGNVKDLLQAWDLYYHVFRRISKGGGGSGGGGSGGGGSDGNEE |
| | CD4htm.IL2Rg.P2A. | MGGITQTPYKVSISGTTVILTCPQYPGSEILWQHNDKNIGGDEDD |
| | huIgKss.antiCD33. | KNIGSDEDHLSLKEFSELEQSGYYVCYPRGSKPEDANFYLYLRAR |
| | FKBP.CD4htm.trIL2Rb | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPMETPAQLLFLLLLWLPDTTGSI |
| | | LWHEMWHEGLEEASRLYFGERNVKGMFEVLEPLHAMMERGPQTLK |
| | | ETSFNQAYGRDLMEAQEWCRKYMKSGNVKDLLQAWDLYYHVFRRI |
| | | SKSNIKVLPTWSTPVQPMALIVLGGVAGLLLFIGLGIFFERTMPR |
| | | IPTLKNLEDLVTEYHGNFSAWSGVSKGLAESLQPDYSERLCLVSE |
| | | IPPKGGALGEGPGASPCNQHSPYWAPPCYTLKPETSGSGATNFSL |
| | | LKQAGDVEENPGPSMETPAQLLFLLLLWLPDTTGEVQLVESGGGE |
| | | VQPGGSLRLSCAASRSSGIDVMGWYRQAPGKERELVAEISGVGDT |
| | | NYAASLADRFTVSRDNAKNTVYLQMSSLRAEDTAVYYCNAHSFLD |
| | | LVGAWGQGTLVTVKPGGGGSGVQVETISPGDGRTFPKRGQTCVVH |
| | | YTGMLEDGKKFDSSRDRNKPFKFMLGKQEVIRGWEEGVAQMSVGQ |
| | | RAKLTISPDYAYGATGHPGIIPPHATLVFDVELLKLESNIKVLPT |
| | | WSTPVQPMALIVLGGVAGLLLFIGLGIFFNCRNTGPWLKKVLKCN |
| | | TPDPSKFFSQLSSEHGGDVQKWLSSPFPSSSFSPGGLAPEISPLE |
| | | VLERDKVTQLLPLNTDAYLSLQELQGQDPTHLV |
| 167 | SR300 - | ALPVTALLLPLALLLHAARPGSILWHEMWHEGLEEASRLYFGERN |
| | CD8ss.FRB.CD3e.P2A. | VKGMFEVLEPLHAMMERGPQTLKETSFNQAYGRDLMEAQEWCRKY |
| | PD1ss.PD1(A132V)ex. | MKSGNVKDLTQAWDLYYHVFRRISKGGGGSGGGGSGGGGSDGNEE |
| | FKBP.minCD4hinge. | MGGITQTPYKVSISGTTVILTCPQYPGSEILWQHNDKNIGGDEDD |
| | CD4tm.CD4ic | KNIGSDEDHLSLKEFSELEQSGYYVCYPRGSKPEDANFYLYLRAR |
| | | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMQIPQAPWPVVWAVLQLGWRP |
| | | GWFLDSPDRPWNPPTFSPALLVVTEGDNATFTCSFSNTSESFVLN |
| | | WYRMSPSNQTDKLAAFPEDRSQPGQDCRFRVTQLPNGRDFHMSVV |
| | | RARRNDSGTYLCGAISLAPKVQIKESLRAELRVTERRAEVPTAHP |
| | | SPSPRPAGQFQTLVGGGGSGVQVETISPGDGRTFPKRGQTCVVHY |
| | | TGMLEDGKKFDSSRDRNKPFKFMLGKQEVIRGWEEGVAQMSVGQR |
| | | AKLTISPDYAYGATGHPGIIPPHATLVFDVELLKLESNIKVLPTW |
| | | STPVQPMALIVLGGVAGLLLFIGLGIFFCVRCRHRRRQ |
| 168 | SR301 - | ALPVTALLLPLALLLHAARPGSILWHEMWHEGLEEASRLYFGERN |
| | CD8ss.FRB.CD3e.P2A. | VKGMFEVLEPLHAMMERGPQTLKETSFNQAYGRDLMEAQEWCRKY |
| | PD1ss.PD1ex.FKBP. | MKSGNVKDLTQAWDLYYHVFRRISKGGGGSGGGGSGGGGSDGNEE |
| | minCD4hinge.CD4tm. | MGGITQTPYKVSISGTTVILTCPQYPGSEILWQHNDKNIGGDEDD |
| | CD4ic | KNIGSDEDHLSLKEFSELEQSGYYVCYPRGSKPEDANFYLYLRAR |
| | | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMQIPQAPWPVVWAVLQLGWRP |
| | | GWFLDSPDRPWNPPTFSPALLVVTEGDNATFTCSFSNTSESFVLN |
| | | WYRMSPSNQTDKLAAFPEDRSQPGQDCRFRVTQLPNGRDFHMSVV |
| | | RARRNDSGTYLCGAISLAPKAQIKESLRAELRVTERRAEVPTAHP |
| | | SPSPRPAGQFQTLVGGGGSGVQVETISPGDGRTFPKRGQTCVVHY |
| | | TGMLEDGKKFDSSRDRNKPFKFMLGKQEVIRGWEEGVAQMSVGQR |
| | | AKLTISPDYAYGATGHPGIIPPHATLVFDVELLKLESNIKVLPTW |
| | | STPVQPMALIVLGGVAGLLLFIGLGIFFCVRCRHRRRQ |
| 169 | SR354 - | ALPVTALLLPLALLLHAARPGSILWHEMWHEGLEEASRLYFGERN |
| | CD8ss.FRBstar.FCER1G. | VKGMFEVLEPLHAMMERGPQTLKETSFNQAYGRDLMEAQEWCRKY |
| | P2A.huIgKss.antiCD33. | MKSGNVKDLLQAWDLYYHVFRRISKGGGGSGGGGSGEPLCYILDA |
| | FKBP.CD4tm.CD4ic | ILFLYGIVLTLLYCRLKIQVRKAAITSYEKSDGVYTGLSTRNQET |
| | | YETLKHEKPPQSGSGATNFSLLKQAGDVEENPGPSMETPAQLLFL |
| | | LLLWLPDTTGEVQLVESGGGEVQPGGSLRLSCAASRSSGIDVMGW |
| | | YRQAPGKERELVAEISGVGDTNYAASLADRFTVSRDNAKNTVYLQ |
| | | MSSLRAEDTAVYYCNAHSFLDLVGAWGQGTLVTVKPGGGGSGVQV |
| | | ETISPGDGRTFPKRGQTCVVHYTGMLEDGKKFDSSRDRNKPFKFM |
| | | LGKQEVIRGWEEGVAQMSVGQRAKLTISPDYAYGATGHPGIIPPH |
| | | ATLVFDVELLKLESNIKVLPTWSTPVQPMALIVLGGVAGLLLFIG |
| | | LGIFFCVRCRHRRRQ |
| 170 | SR303 - | ALPVTALLLPLALLLHAARPGSILWHEMWHEGLEEASRLYFGERN |
| | CD8ss.FRBstar.CD3e. | VKGMFEVLEPLHAMMERGPQTLKETSFNQAYGRDLMEAQEWCRKY |
| | P2A.huIgKss.antiROR1. | MKSGNVKDLLQAWDLYYHVFRRISKGGGGSGGGGSGGGGSDGNEE |
| | FKBP.minCD4hinge. | MGGITQTPYKVSISGTTVILTCPQYPGSEILWQHNDKNIGGDEDD |
| | CD4tm.CD4ic | KNIGSDEDHLSLKEFSELEQSGYYVCYPRGSKPEDANFYLYLRAR |
| | | VCENCMEMDVMSVATIVIVDICITGGLLLLVYYWSKNRKAKAKPV |
| | | TRGAGAGGRQRGQNKERPPPVPNPDYEPIRKGQRDLYSGLNQRRI |
| | | SGSGATNFSLLKQAGDVEENPGPSMETPAQLLFLLLLWLPDTTGD |
| | | IQMTQSPSFLSASVGDRVTINCKASQNIDRYLNWYQQKLGEAPKR |
| | | LLYNTNKLQTGIPSRFSGSGSATDFTLTISSLQPEDFATYFCLQY |
| | | NSLPLTFGSGTKLEIKGGGGSGGGGSGGGGSEVQLVESGGGLVQP |
| | | GRSLKLSCAASGFIFSEHNMAWVRQAPKKGLEWVATISDDGRNTY |
| | | YRDSMRGRFTISRENARSTLYLQLDSLRSEDTATYYCASHRYNLF |
| | | DSWGQGVMVTVSSGGGGSGVQVETISPGDGRTFPKRGQTCVVHYT |
| | | GMLEDGKKFDSSRDRNKPFKFMLGKQEVIRGWEEGVAQMSVGQRA |
| | | KLTISPDYAYGATGHPGIIPPHATLVFDVELLKLESNIKVLPTWS |
| | | TPVQPMALIVLGGVAGLLLFIGLGIFFCVRCRHRRRQ |

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Construction of Rapamycin-Inducible TCRs (T Cell Receptors)

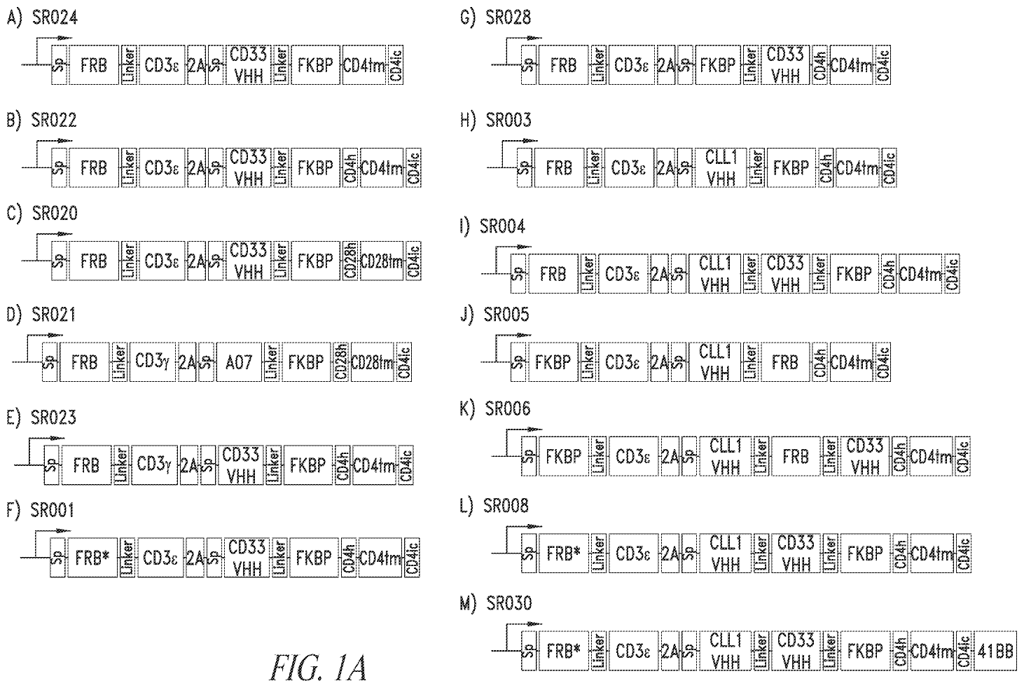

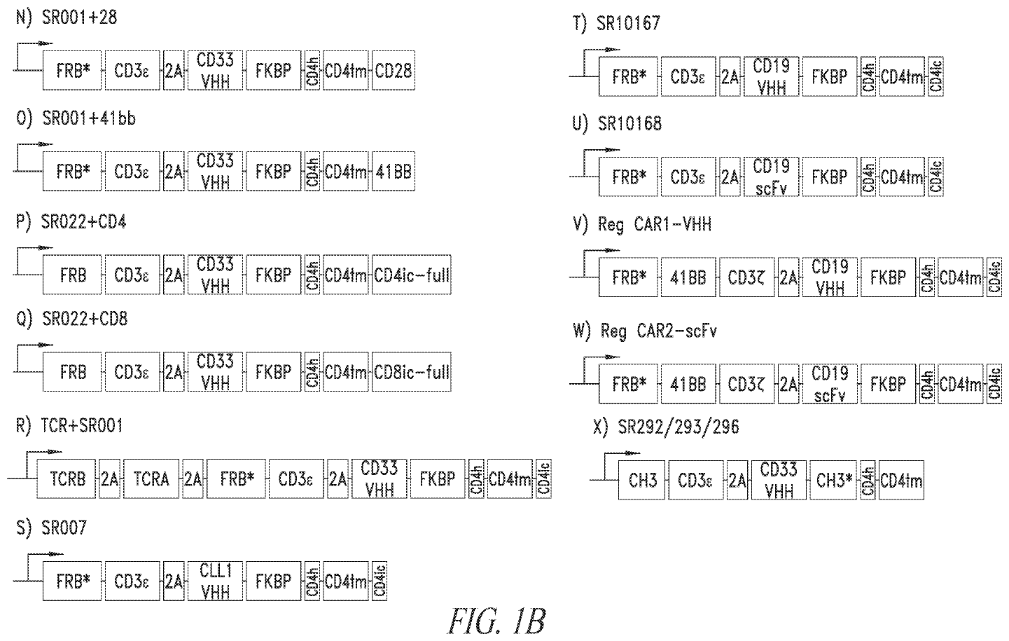

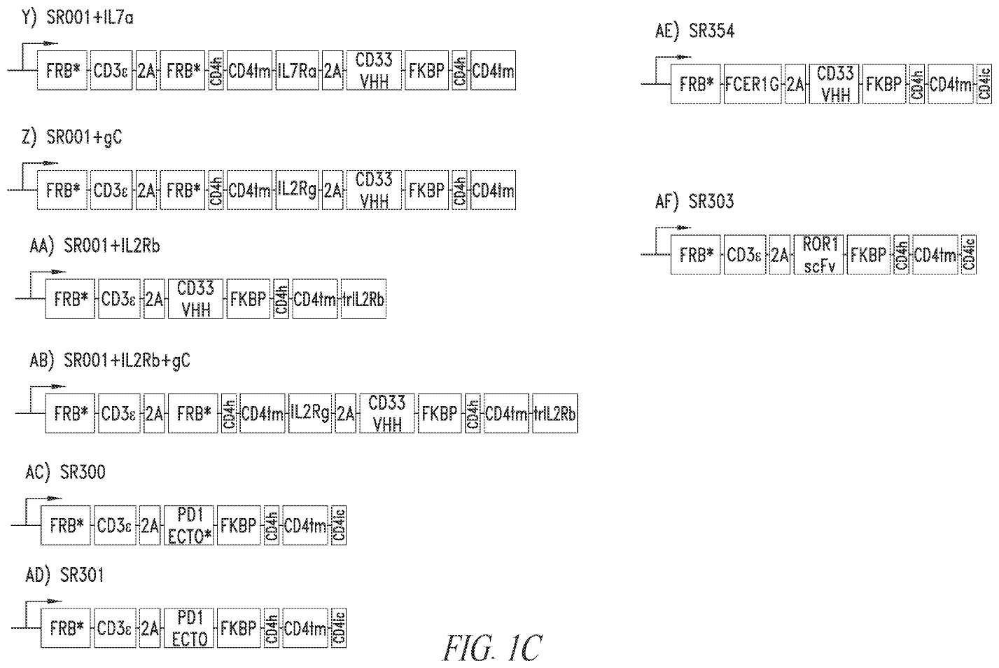

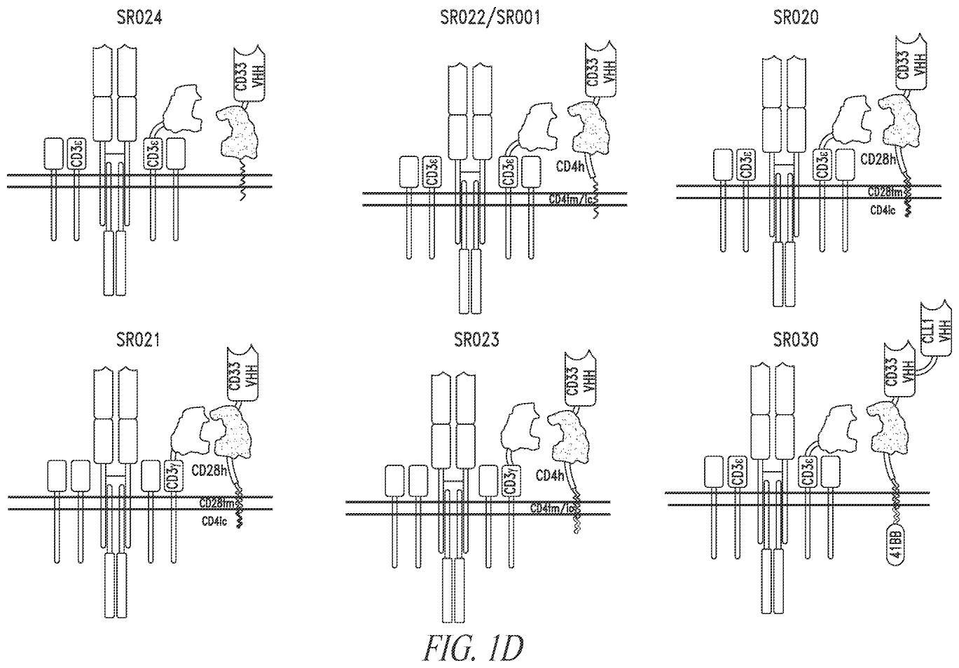

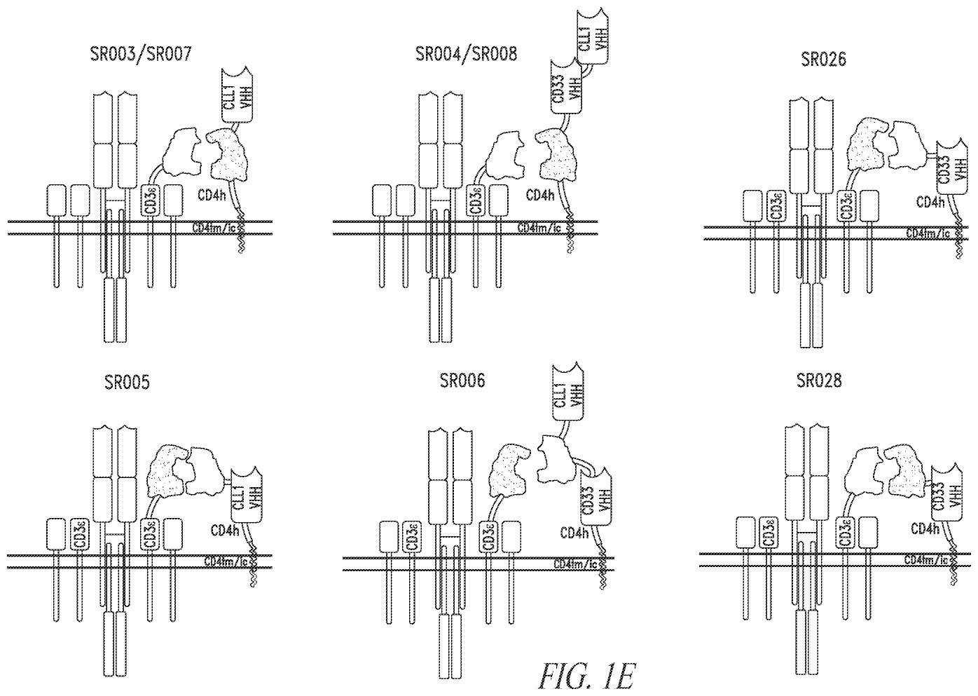

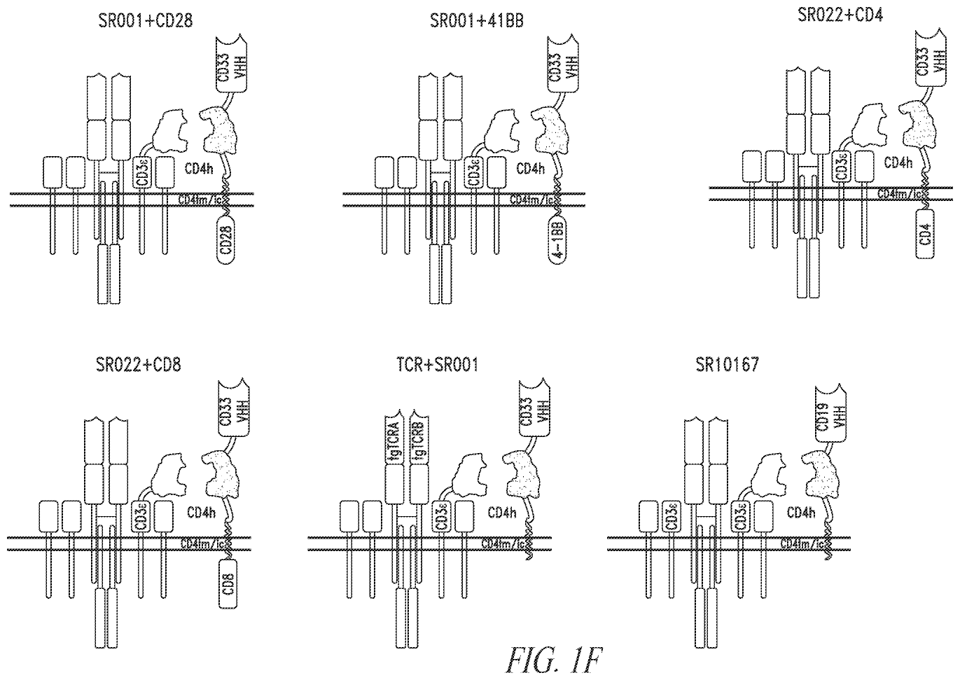

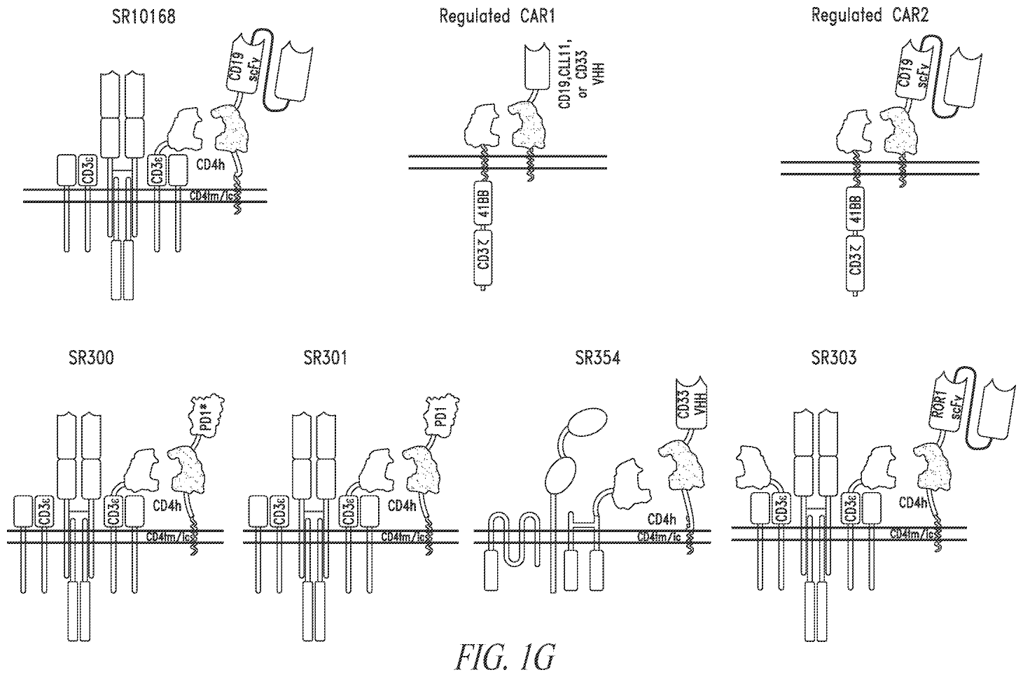

Lentiviral vectors comprising constructs that include at least a multimerization domain (e.g., a rapamycin-inducible dimerization domain), a CD3 subunit, and an extracellular antigen targeting domain were designed, cloned, and sequence verified. The constructs comprise various combinations of the following units: a signal sequence (e.g., a CD8α or IgK derived signal sequence), one or more multimerization domains (e.g., an FK506-binding protein (FKBP12 or FKBP) and an FKBP-rapamycin binding protein (FRB or FRB*)), a CD3 subunit, one or more viral self-cleaving peptides (e.g., P2A or T2A self-cleaving peptides) one or more extracellular antigen targeting domains (e.g., an antibody derived targeting domain or a natural ligand derived targeting domain, various hinges and transmembrane domains (e.g., those derived from CD4 or CD28), one or more intracellular domains (e.g., costimulatory domains), and one or more coreceptor or cytokine receptor domains (see FIGS. 1A-1H). Transgenic TCRs were also expressed alongside the above components in some constructs.

### Example 2

### Evaluation of Rapamycin-Inducible TCR T Cells with No Hinge Domain Anchoring FKBP12

T cells expressing rapamycin-inducible T cell receptors (SR-T cells) were generated using a 10-day transduction and expansion process then evaluated for expression and biological activity against specific target antigens (see Leung et al., JCI Insight. 2019 Apr. 30; 5(11)). Briefly, peripheral blood mononuclear cells (PBMCs) were cultured in an IL-2 containing media and activated with antibodies against human CD3 and human CD28. Lentiviral vectors encoding rapamycin-inducible TCRs with no hinge domain between the FKBP12 multimerization domain and the CD4 transmembrane domain (CD4tm) of the targeting component (SR024) were used to transduce the PBMCs one day after culture initiation, then cells were transferred to a 24-well G-REX®culture system (Wilson Wolf) 48 hours later. After a total of 10 days in culture, SR-T cells were evaluated for rapamycin-dependent, antigen-dependent activity, as well as rapamycin-independent, antigen-independent background activity.

SR-T cells transduced with lentiviral vectors described above (SR024-T cells) were analyzed for extracellular expression of key components of the rapamycin-inducible system by flow cytometry, including targeting domain (e.g. anti-CD33 VHH), FRB multimerization domain and FKBP12 multimerization domain, using a recombinant CD33-Fc fusion, anti-FRB antibody and anti-FKBP12 antibody, respectively. As shown in FIG. 2, FRB and CD33 components were highly expressed compared to control cells that were untransduced.

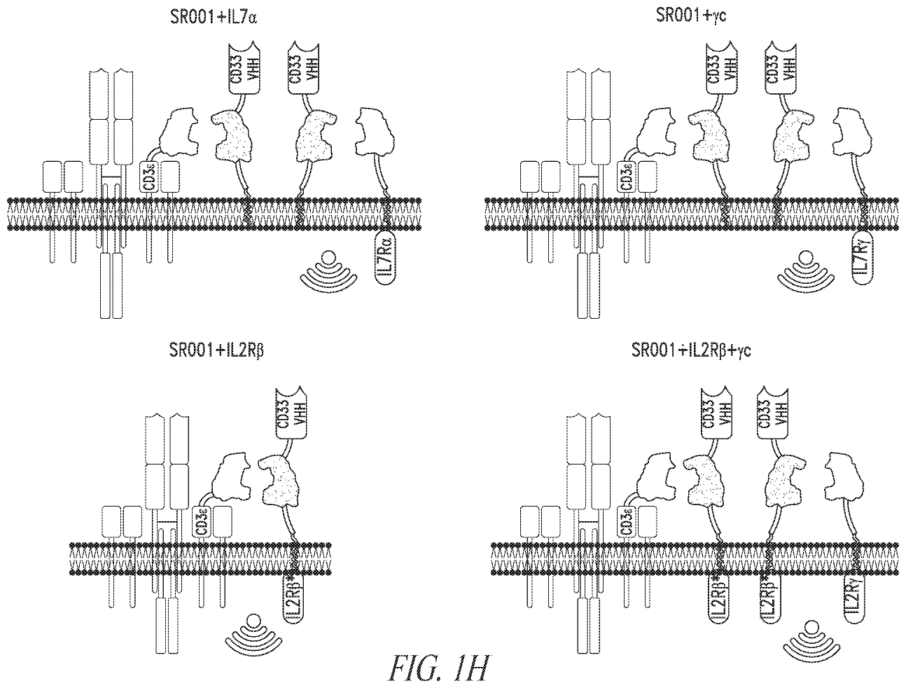

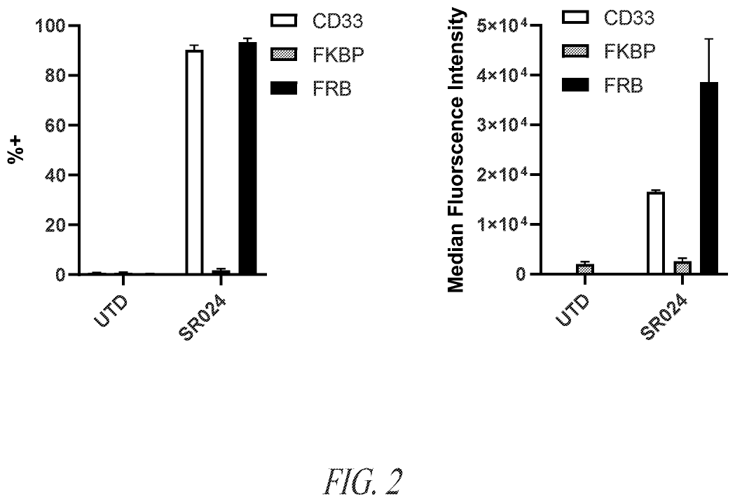

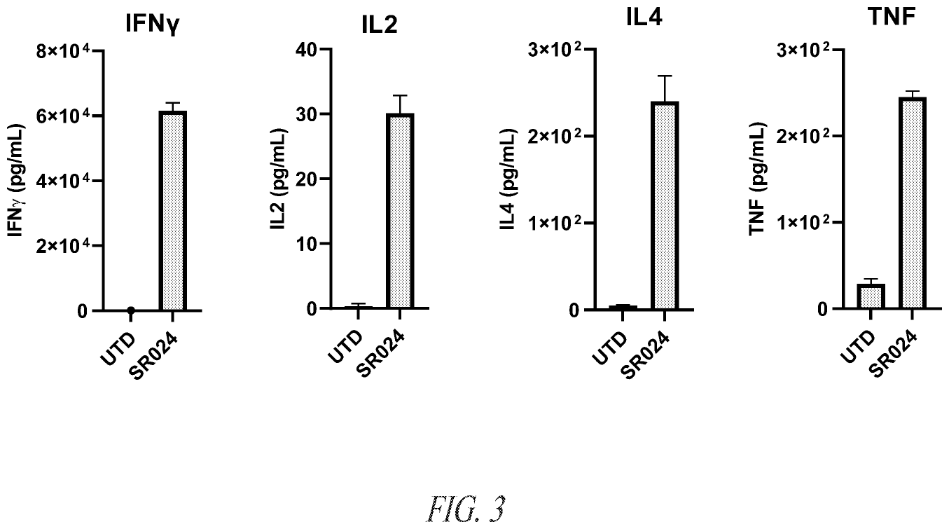

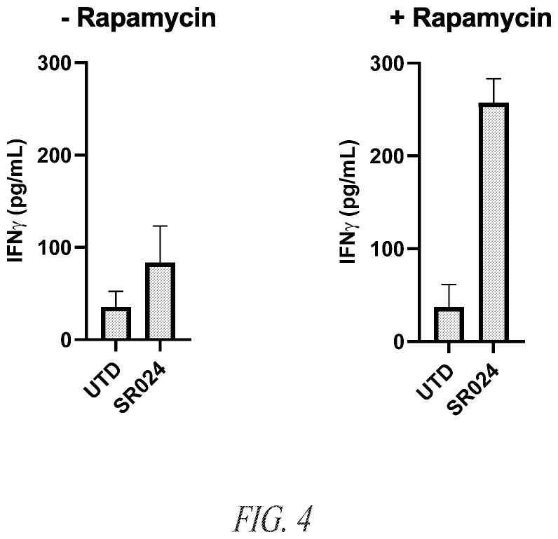

SR024-T cells' rapamycin-dependent, antigen-dependent activity was evaluated in co-cultures with target antigen positive tumor cells in the presence of 1 nM rapamycin. Specifically, IFNγ, IL2, IL4 and TNFα production was quantified using MSD from supernatants of cultures with SR024-T cells and a cell line engineered to express high levels of CD33 (FIG. 3). SR024-T cells released each of these 4 cytokines at levels that were highly distinguishable from baseline (untransduced control; UTD). Additionally, T cells were cultured alone with no antigen present, both in the presence of and without 1 nM of rapamycin (FIG. 4). In both cases, SR024-T cells produced 2 orders of magnitude less IFNγ than if they were cultured in the presence of antigen.

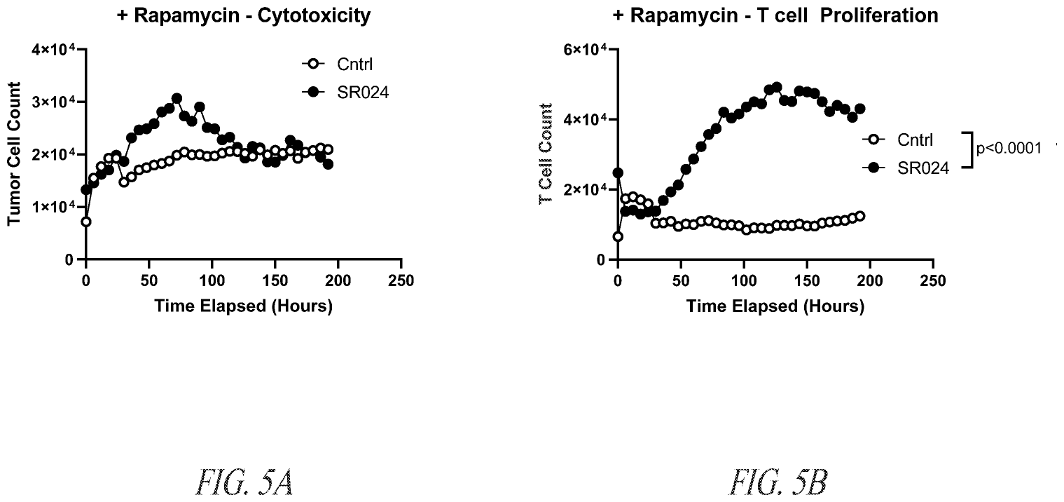

The cytotoxicity of SR024-T cells was assessed by incubating them with cells that express target antigen and a nuclear red fluorescent molecule. Cells expressing nuclear fluorescence (antigen positive target line) and cells not expressing nuclear fluorescence (SR-T cells) were both tracked over approximately 200 hours using an Incucyte® live cell imaging system (Sartorius). While SR024-T cells did not effectively kill CD33+ target cells (FIG. 5A), they significantly expanded in these cultures compared to a T cell control (FIG. 5B).

### Example 3

### Evaluation of Rapamycin-Inducible TCR T Cells with FKBP12 Anchored to CD4 or CD28 Hinge Domains

T cells expressing rapamycin-inducible T cell receptors (SR-T cells) were generated using a 10-day transduction and expansion process, then evaluated for expression and biological activity against specific target antigens. Briefly, PBMCs were cultured in an IL-2 containing media and activated with antibodies against human CD3 and human CD28. Lentiviral vectors encoding rapamycin-inducible TCRs with an FKBP12 containing targeting component anchored to CD4 or CD28 hinge domains (CD4h or CD28h), or no hinge domain (SR022, SR020, and SR024), were used to transduce the PBMCs one day after culture initiation, then cells were transferred to a 24-well G-REX® culture system 48 hours later. After a total of 10 days in culture, SR-T cells were evaluated for rapamycin-dependent, antigen-dependent activity; as well as rapamycin-independent, antigen-independent background activity.

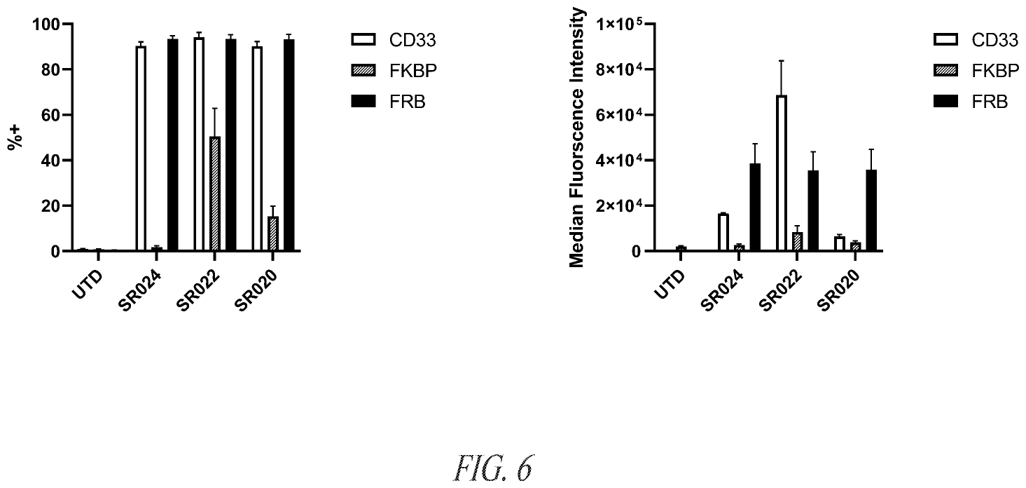

SR-T cells transduced with lentiviral vectors described above (SR022-,SR020-, and SR024-T cells) were analyzed for extracellular expression of key components of the rapamycin-inducible system by flow cytometry, including targeting domain (e.g., anti-CD33 VHH), FRB multimerization domain and FKBP12 multimerization domain, using a recombinant CD33-Fc fusion, anti-FRB antibody and anti-FKBP12 antibody, respectively. FRB and CD33 components were highly expressed compared to control cells that were untransduced (FIG. 6).

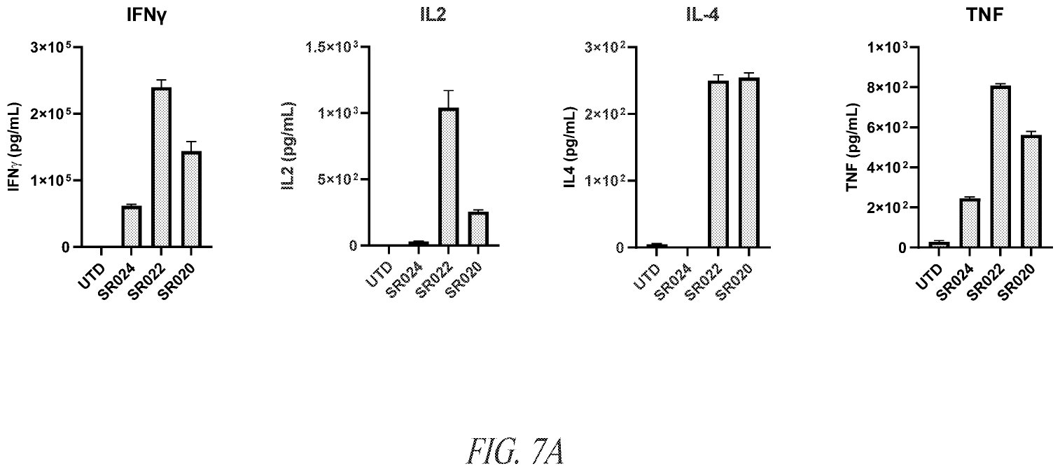

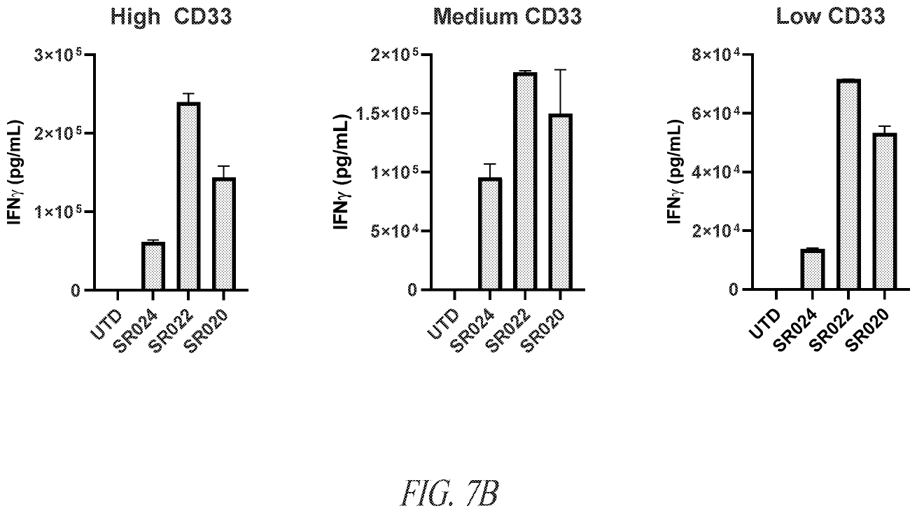

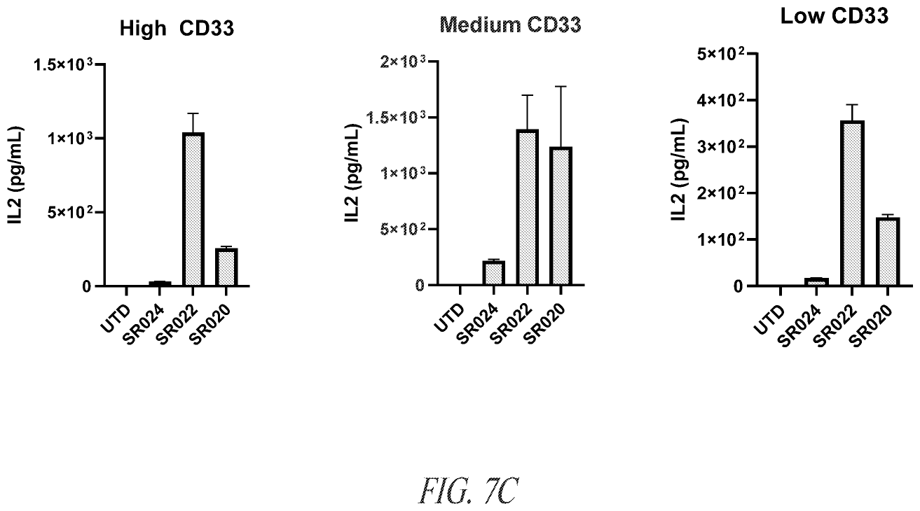

The rapamycin-dependent, antigen-dependent activity of SR022-, SR020-, and SR024-T cells was evaluated in co-cultures with target antigen positive tumor cells in the presence of 1 nM rapamycin. Specifically, IFNγ, IL2, IL4 and TNFα production was quantified using MSD from supernatants of cultures with SR022-, SR020-, or SR024-T cells; and a cell line engineered to express high levels of CD33 (FIG. 7A). Both SR022- and SR020-T cells released each of these 4 cytokines at levels that were highly distinguishable from baseline (untransduced control; UTD). SR020 showed decreased IFNγ, IL2, and TNFα compared with SR022, but comparable IL4. SR024-T cells had diminished cytokine release across all 4 cytokines compared to SR022- and SR020-T cells.

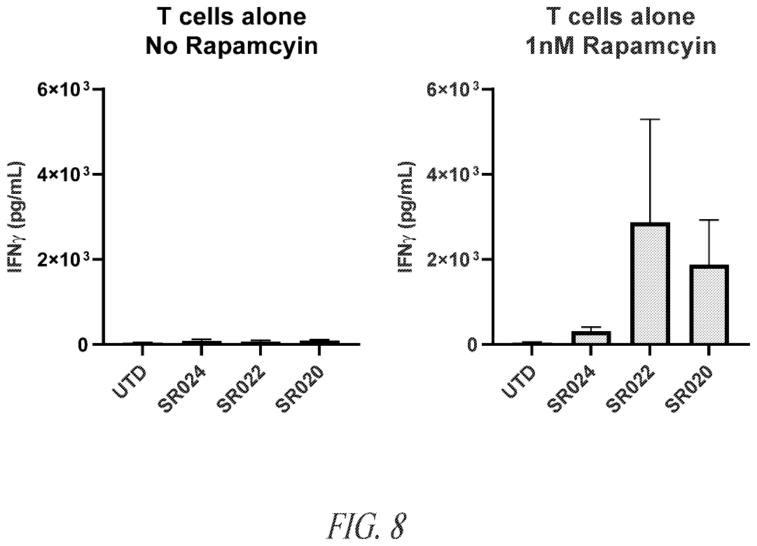

T cells were also co-cultured with CD33+ cell lines with high, medium and low antigen density. SR022- and SR020-T cells produce more IFNγ than SR024-T cells at all antigen densities, particularly at low antigen density (FIG. 7B). Additionally, SR022- and SR020-T cells generate IL2 in cultures at all antigen densities, whereas SR024-T produced little to no IL2 (FIG. 7C). SR022-T cells secrete more IFNγ and IL2 across high, medium and low CD33+ antigen density target lines. T cells were also cultured alone with no antigen present, both in the presence of and without 1 nM of rapamycin (FIG. 8). Without antigen present, SR024, SR022 and SR020-T cells generated 2 orders of magnitude less IFNγ than when antigen was present.

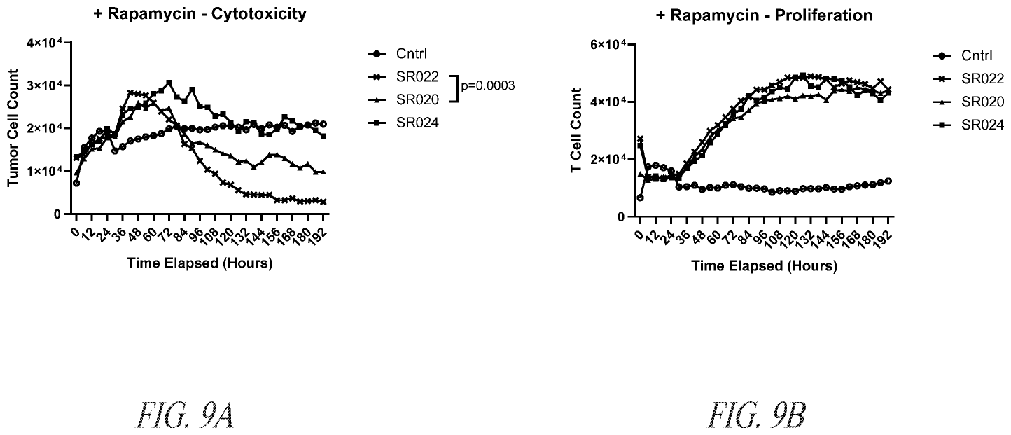

The cytotoxicity of SR024-, SR022- and SR020-T cells was assessed by incubating them with cells that express target antigen and a nuclear red stain. Cells expressing nuclear stain (antigen positive target line) and cells not expressing nuclear stain (SR-T cells) were both tracked over approximately 200 hours using an Incucyte® live cell imaging system (Sartorius). While SR024-T cells displayed minimal killing of CD33+ target cells (FIG. 9A), they significantly expanded in these cultures compared to a T cell control (FIG. 9B). SR022-T cells were able to effectively kill CD33+ target cells (FIG. 9A) and expand compared to a T cell control (FIG. 9B). While SR020-T cells expanded well compared to control (FIG. 9B), they performed intermediately in the cytotoxicity assay (FIG. 9A). Overall, SR022-T cells performed significantly better in a cytotoxicity assay compared to SR020- or SR024-T cells.

FIGS. 7A-7C, 8, and 9A-9B demonstrate that SR020 and SR022, which comprise a hinge domain, exhibit improved cytokine release profiles and increased killing of CD33+ target cells as compared to SR024, which lacks a hinge domain (see FIGS. 1A and 1D). Without wishing to be bound by any particular theory, it is contemplated that the addition of the hinge domain to the signaling components improves the function of the architecture by allowing the first multimerization domain of the signaling component to spatially align better with the multimerization domain of the targeting component, surprisingly increasing the efficacy of the targeting components that comprise a hinge domain compared to targeting components lacking a hinge domain.

### Example 4

### Evaluation of Rapamycin-Inducible TCR T Cells with FRB Appended to CD3γ

T cells expressing rapamycin-inducible T cell receptors (SR-T cells) were generated using a 10-day transduction and expansion process, then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding rapamycin-inducible TCRs with FRB appended to CD3γ instead of CD3ε (SR021) were used to transduce the PBMCs one day after culture initiation, then cells were transferred to a 24-well G-REX® culture system 48 hours later. After a total of 10 days in culture, SR-T cells were evaluated for rapamycin-dependent, antigen-dependent activity, as well as rapamycin-independent, antigen-independent background activity.

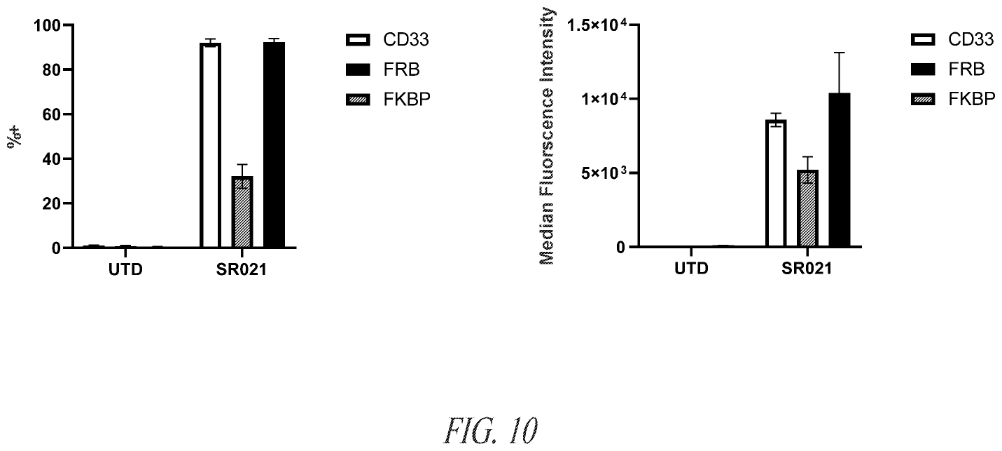

SR-T cells transduced with lentiviral vectors described above (SR021-T cells) were analyzed for extracellular expression of key components of the rapamycin-inducible system by flow cytometry, including targeting domain (e.g., anti-CD33 VHH), FRB dimerization domain and FKBP12 dimerization domain, using recombinant CD33-Fc fusion, anti-FRB antibody and anti-FKBP12 antibody, respectively. FRB and CD33 components were highly expressed compared to control cells that were untransduced (FIG. 10).

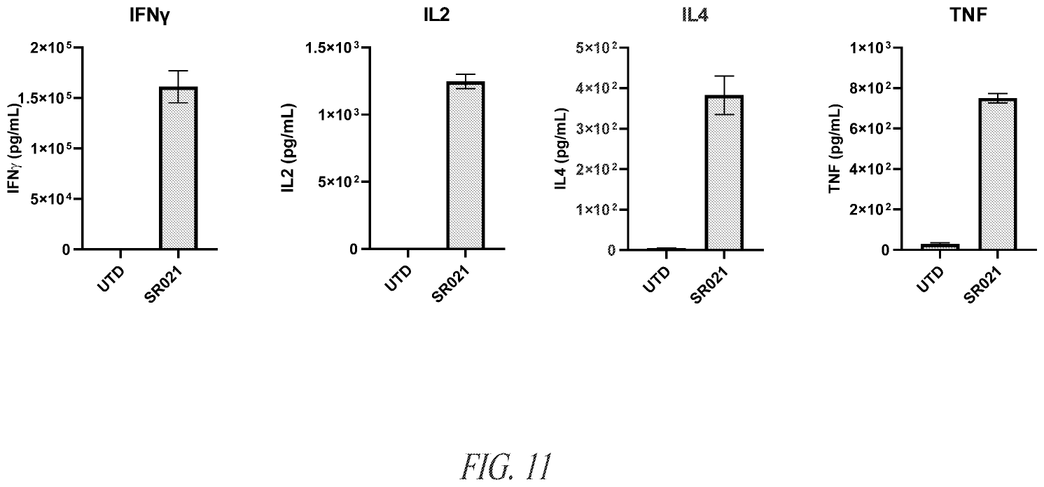

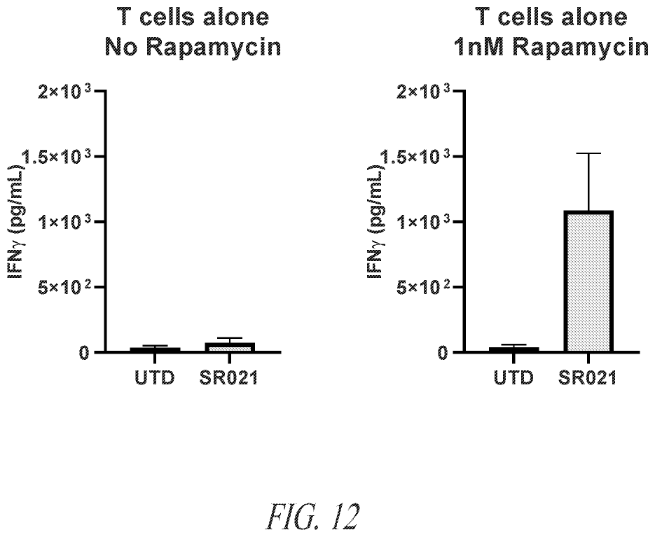

The rapamycin-dependent, antigen-dependent activity of SR021-T cells was evaluated in co-cultures with target antigen positive tumor cells in the presence of 1 nM rapamycin. Specifically, IFNγ, IL2, IL4 and TNFα production was quantified using MSD from supernatants of cultures with SR021-T cells and a cell line engineered to express high levels of CD33 (FIG. 11). SR021-T cells released each of these 4 cytokines at levels that were highly distinguishable from baseline (untransduced control; UTD). Additionally, T cells were cultured alone with no antigen present, both without and in the presence of 1 nM of rapamycin (FIG. 12). Without antigen present, SR021-T cells generated 2 orders of magnitude less IFNγ than when antigen was present.

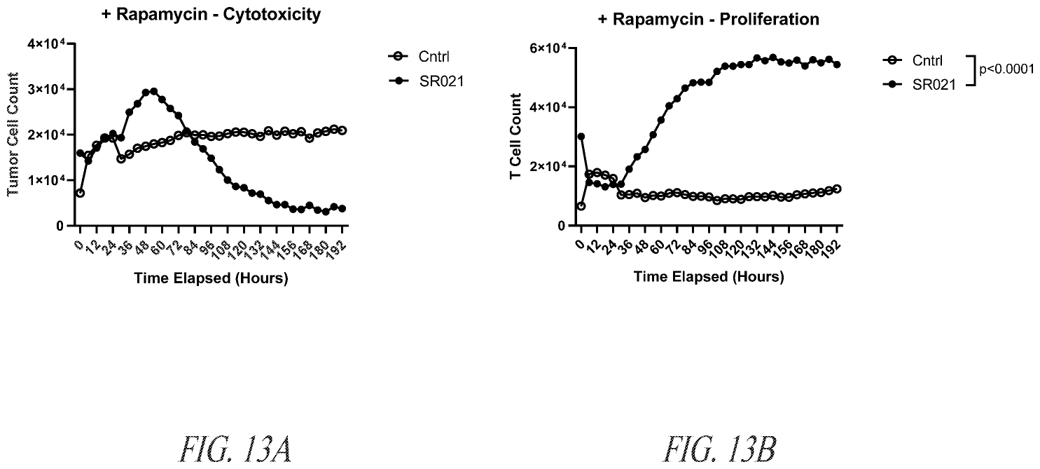

The cytotoxicity of SR021-T cells to cells that express target antigen was assessed using a target line that expressed a nuclear red stain and tracked over time using an Incucyte® live cell imaging system (Sartorius). Cells expressing nuclear stain (antigen positive target line) and cells not expressing nuclear stain (SR-T cells) were both tracked over approximately 200 hours. SR021-T cells displayed a trend toward killing CD33+ cells (FIG. 13A) and significantly expanded compared to a T cell control (FIG. 13B).

### Example 5

### Evaluation of Rapamycin-Inducible TCR T Cells with FRB* (T2098L)

T cells expressing rapamycin-inducible T cell receptors (SR-T cells) were generated using a 10-day transduction and expansion process then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding rapamycin-inducible TCRs with either FRB or a mutant FRB-FRB (T2098L)-that allows for the binding of certain rapamycin analogs (denoted FRB*) (SR022 and SR001, respectively) were used to transduce the PBMCs one day after culture initiation, then cells were transferred to a 24-well G-REX® culture system 48 hours later. After a total of 10 days in culture, SR-T cells were evaluated for rapamycin-dependent, antigen-dependent activity, as well as rapamycin-independent, antigen-independent background activity.

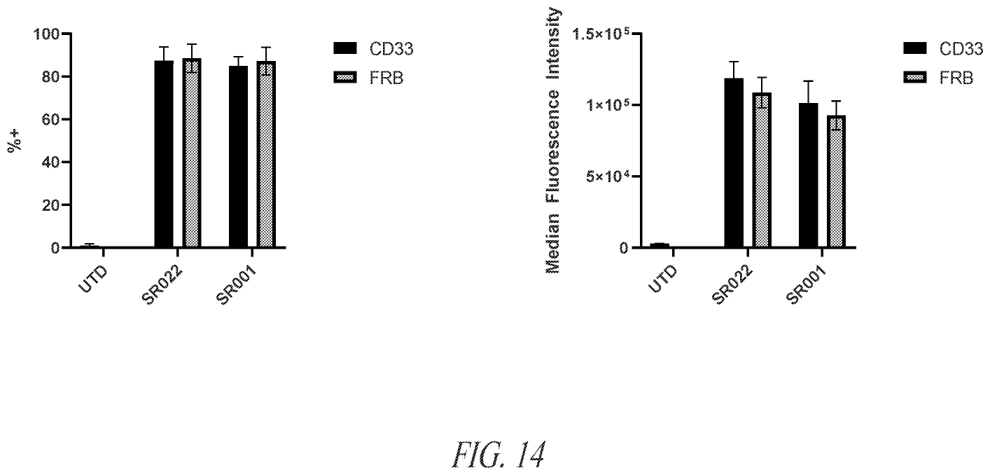

SR-T cells transduced with lentiviral vectors described above (SR022-T cells and SR001-T cells for FRB and FRB*, respectively) were analyzed for extracellular expression of key components of the rapamycin-inducible system by flow cytometry, including targeting domain (e.g., anti-CD33 VHH) and FRB multimerization domain, using recombinant CD33-Fc fusion and anti-FRB antibodies, respectively. FRB and CD33 components were highly expressed compared to control cells that were untransduced (FIG. 14).

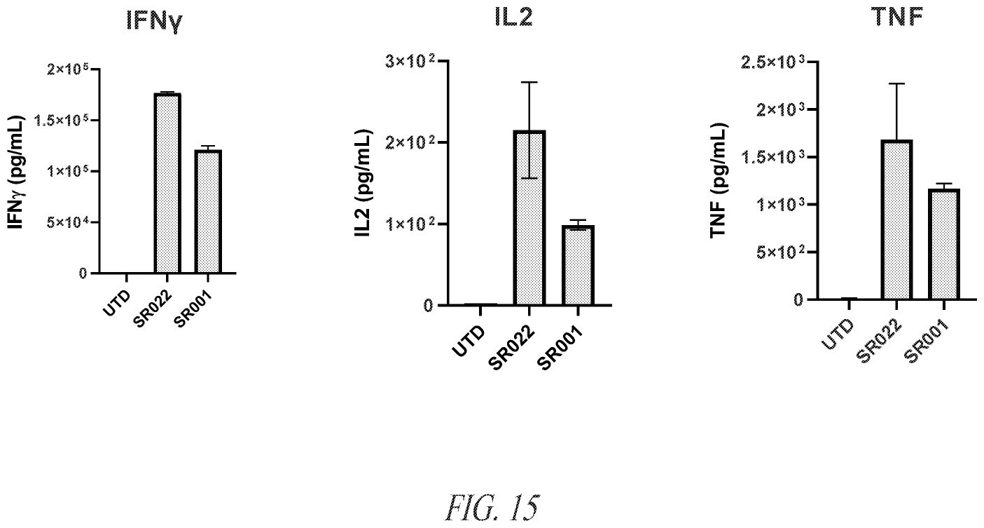

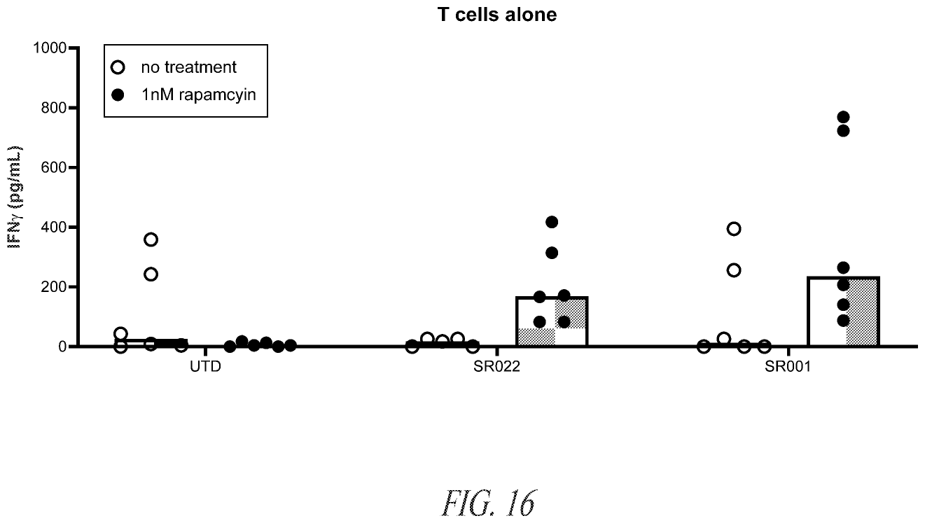

The rapamycin-dependent, antigen-dependent activity of SR022-T cells and SR001 T cells was evaluated in co-cultures with target antigen positive tumor cells in the presence of 1 nM rapamycin. Specifically, IFNγ, IL2, and TNFα production was quantified using MSD from supernatants of cultures with SR022- or SR001-T cells, and a cell line engineered to express high levels of CD33 (FIG. 15). Both SR022- and SR001-T cells released each of these 3 cytokines at levels that were highly distinguishable from baseline (untransduced control; UTD). Additionally, T cells were cultured alone with no antigen present, both without and in the presence of 1 nM of rapamycin (FIG. 16). Without antigen present, SR022- and SR001-T cells generated 3 orders of magnitude less IFNγ than when antigen was present.

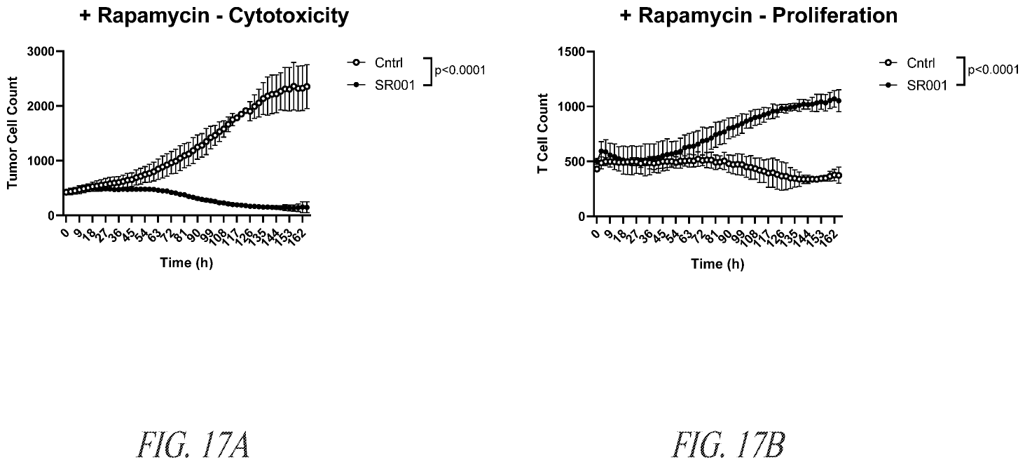

The cytotoxicity of SR001-T cells to cells that express target antigen was assessed using a target line that expressed a nuclear red stain and tracked over time using an Incucyte® live cell imaging system (Sartorius). Cells expressing nuclear stain (antigen positive target line) and cells not expressing nuclear stain (SR-T cells) were both tracked over approximately 160 hours. SR001-T cells effectively killed CD33+ target cells (FIG. 17A) and significantly expanded compared to a T cell control (FIG. 17B).

### Example 6

### Evaluation of Rapamycin-Inducible TCR T Cells with FKBP12 Distal to Antigen Targeting Domains

In another experiment, T cells expressing rapamycin-inducible T cell receptors (SR-T cells) were generated using a 10-day transduction and expansion process then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding rapamycin-inducible TCRs with CD4 hinge (CD4h), transmembrane (CD4tm) and minimal intracellular (CD4ic) domains anchoring an antigen targeting domain followed by an FKBP12 multimerization domain (SR028) were used to transduce the PBMCs one day after culture initiation, then cells were transferred to a 24-well G-REX® culture system 48 hours later. After a total of 10 days in culture, SR-T cells were evaluated for rapamycin-dependent, antigen-dependent activity, as well as rapamycin-independent, antigen-independent background activity.

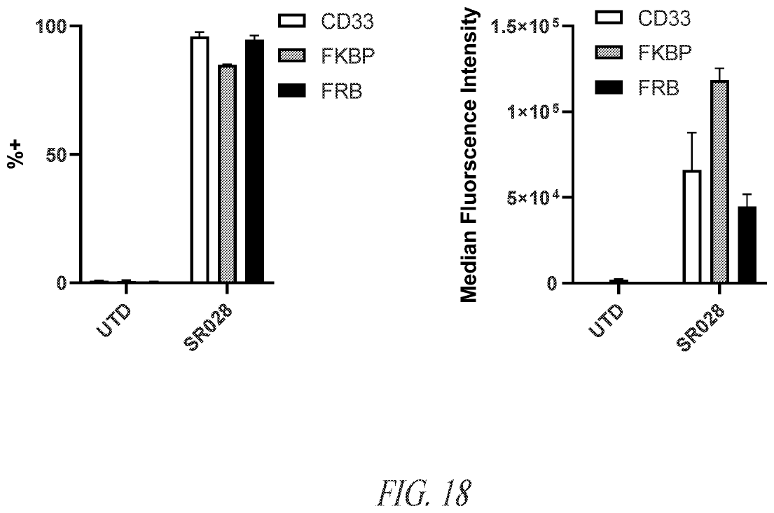

SR-T cells transduced with lentiviral vectors described above (SR028-T cells) were analyzed for extracellular expression of key components of the rapamycin-inducible system by flow cytometry, including targeting domain (e.g., anti-CD33 VHH), FRB multimerization domain and FKBP12 multimerization domain, using a recombinant CD33-Fc fusion, anti-FRB antibody and anti-FKBP12 antibody, respectively. FRB, FKBP12 and CD33 components were highly expressed compared to control cells that were untransduced (FIG. 18).

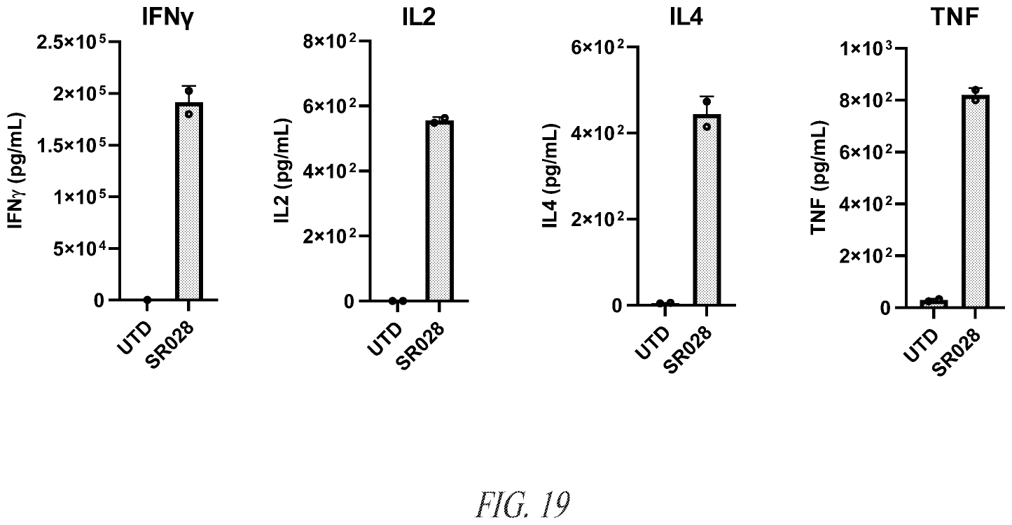

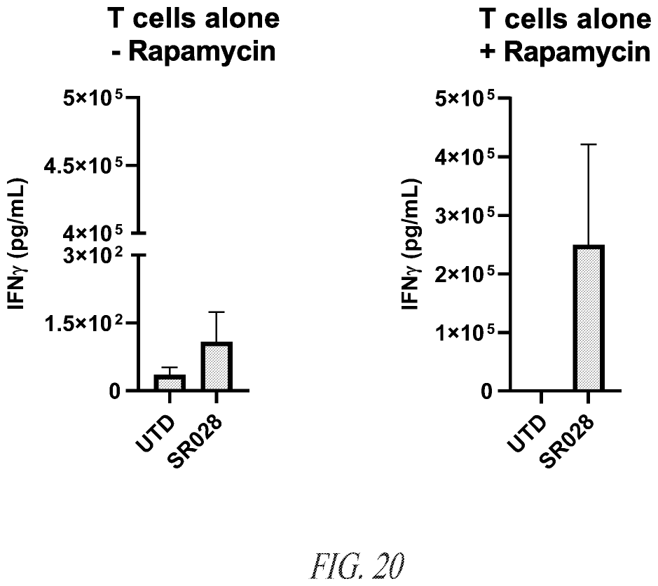

SR028-T cell rapamycin-dependent, antigen-dependent activity was evaluated in co-cultures with target antigen positive tumor cells in the presence of 1 nM rapamycin. Specifically, IFNγ, IL2, IL4 and TNFα production was quantified using MSD from supernatants of cultures with SR028-T cells and a cell line engineered to express high levels of CD33 (FIG. 19). SR028-T cells released each of these 4 cytokines at levels that were highly distinguishable from baseline (untransduced control; UTD). Additionally, T cells were cultured alone with no antigen present, both without and in the presence of 1 nM of rapamycin. While SR028-T cells produced no IFNγ when rapamycin was absent, when 1 nM of rapamycin was added to the SR028-T cells with no antigen present, IFNγ was released at levels comparable to when antigen was present (FIG. 20). SR028-T cells produced a significant amount of IFNγ without antigen present, comparable to what was produced in the presence of antigen.

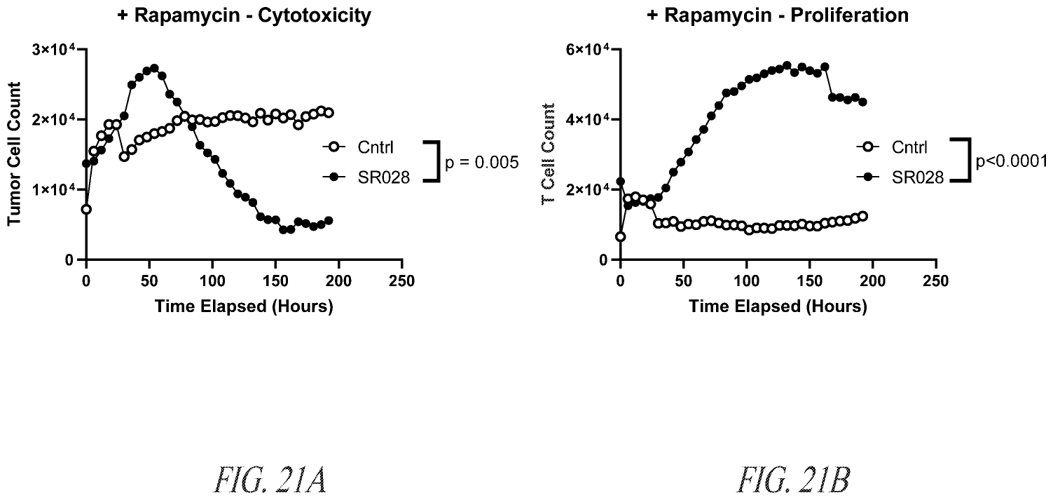

The cytotoxicity of SR028-T cells to cells that express target antigen was assessed using a target line that expressed a nuclear red stain and tracked over time using an Incucyte® live cell imaging system (Sartorius). Cells expressing nuclear stain (antigen positive target line) and cells not expressing nuclear stain (SR-T cells) were both tracked over approximately 200 hours. SR028-T cells effectively killed CD33+ target cells (FIG. 21A) and significantly expanded compared to a T cell control (FIG. 21B).

### Example 7

### Evaluation of Rapamycin-Inducible TCR T Cells with Multi-Antigen Targeting

In another experiment, T cells expressing rapamycin-inducible T cell receptors (SR-T cells) were generated using a 10-day transduction and expansion process, then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding rapamycin-inducible TCRs with CD4 hinge, transmembrane and intracellular domains anchoring a FKBP12 or FRB multimerization domain and two antigen targeting domains (SR004 and SR006) were used to transduce the PBMCs one day after culture initiation, then cells were transferred to a 24-well G-REX® culture system (Wilson Wolf) 48 hours later. After a total of 10 days in culture, SR-T cells were evaluated for rapamycin-dependent, antigen-dependent activity, as well as rapamycin-independent, antigen-independent background activity.

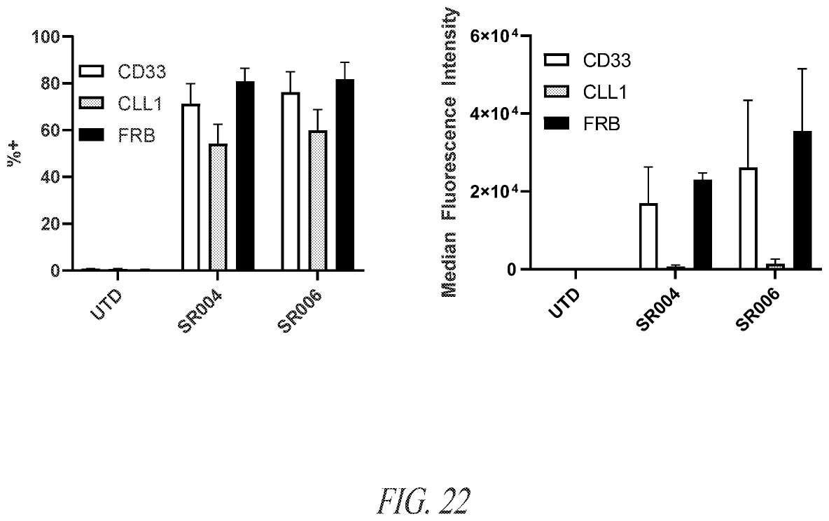

SR-T cells transduced with lentiviral vectors described above (SR004-T cells and SR006-T cells) were analyzed for extracellular expression of key components of the rapamycin-inducible system by flow cytometry, including targeting domains (e.g., anti-CD33 and anti-CLL1 VHH) and an FRB multimerization domain, using a recombinant CD33-Fc fusion, recombinant CLL1-Fc fusion and anti-FRB antibody, respectively. FRB, anti-CD33 and anti-CLL1 components were highly expressed compared to control cells that were untransduced (FIG. 22).

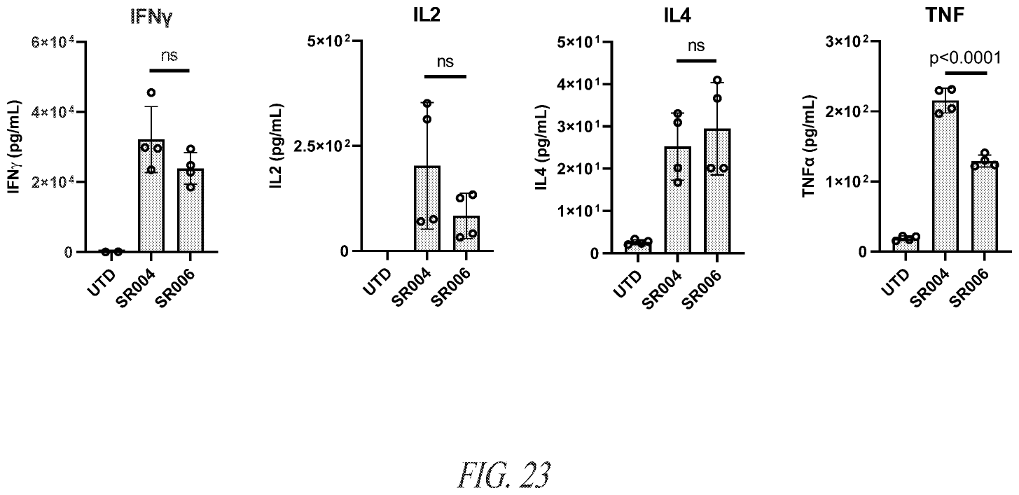

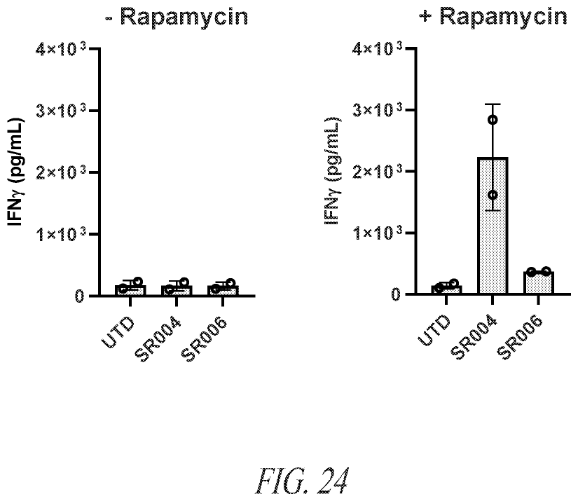

SR0004- and SR0006-T cell rapamycin-dependent, antigen-dependent activity was evaluated in co-cultures with target antigen positive tumor cells in the presence of 1 nM rapamycin. Specifically, IFNγ, IL2, IL4 and TNFα production was quantified using MSD from supernatants of cultures with SR004- and SR006-T cells and a cell line engineered to express high levels of CD33 and CLL1. SR004- and SR006-T cells released each of these 4 cytokines at levels that were distinguishable from baseline (untransduced control; UTD). SR004-T cells trended toward showing increased IFNγ and IL2 release over SR006-T cells, and displayed significantly more TNFα release (FIG. 23). Additionally, T cells were cultured alone with no antigen present, both without and in the presence of 1 nM of rapamycin. While SR004-T cells and SR006-T cells produced no IFNγ when rapamycin was absent, when 1 nM of rapamycin was added to the SR004-T cells with no antigen present, IFNγ was released at levels below that observed with antigen present, but above baseline (FIG. 24).

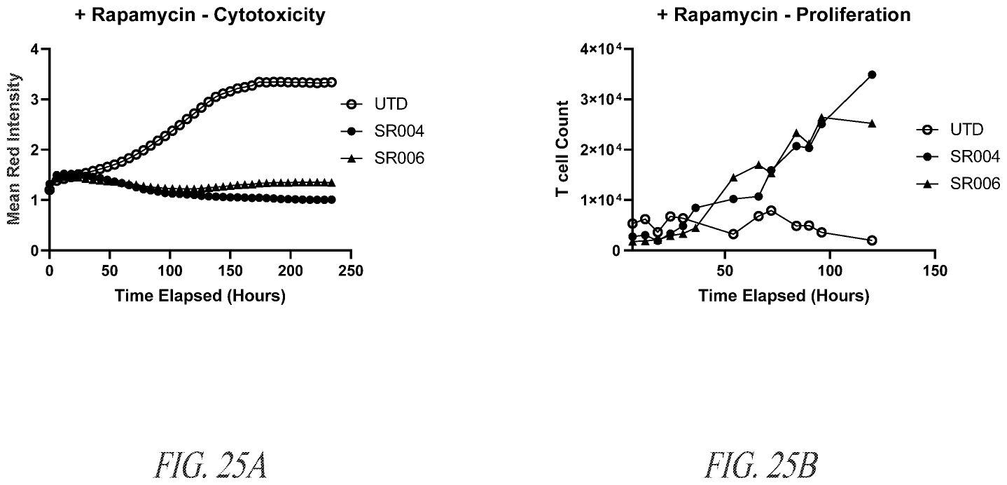

The cytotoxicity of SR004 and SR006-T cells to cells that express both target antigens was assessed using a target line that expressed a nuclear red stain and tracked over time using an Incucyte® live cell imaging system (Sartorius). Cells expressing nuclear stain (antigen positive target line) and cells not expressing nuclear stain (SR-T cells) were both tracked over approximately 250 hours. SR004- and SR006-T cells effectively kill CD33+CLL1+ target cells (FIG. 25A) and significantly expanded in these cultures compared to an untransduced T cell control (FIG. 25B).

### Example 8

### Evaluation of Rapamycin-Inducible TCR T Cells with 4-1BB Costimulatory Domain

In another experiment, T cells expressing rapamycin-inducible T cell receptors (SR-T cells) were generated using a 10-day transduction and expansion process, then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding rapamycin-inducible TCRs with CD4 hinge and transmembrane domains anchoring a FKBP12 multimerization domain and two antigen targeting domains, with and without a 4-1BB costimulatory domain (SR030 and SR008, respectively) were used to transduce the PBMCs one day after culture initiation, then cells were transferred to a 24-well G-REX® culture system 48 hours later. After a total of 10 days in culture, SR-T cells were evaluated for rapamycin-dependent, antigen-dependent activity, as well as rapamycin-independent, antigen-independent background activity.

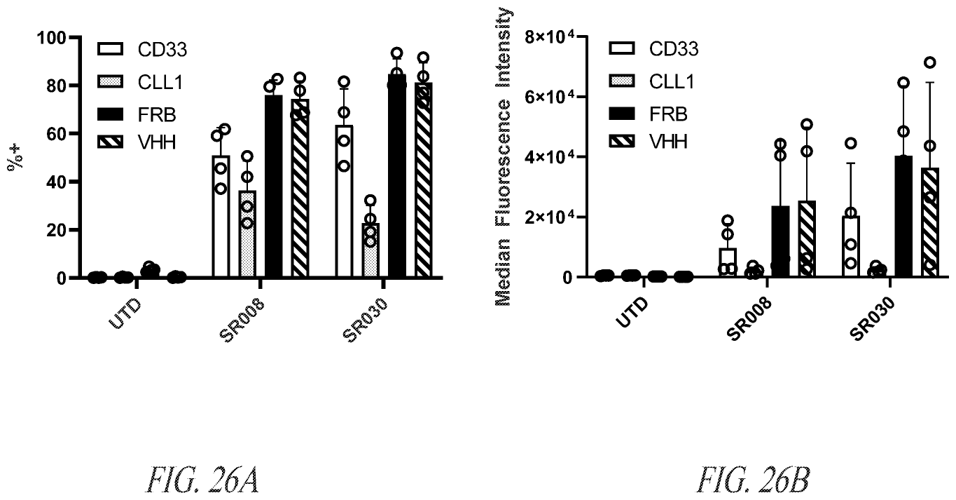

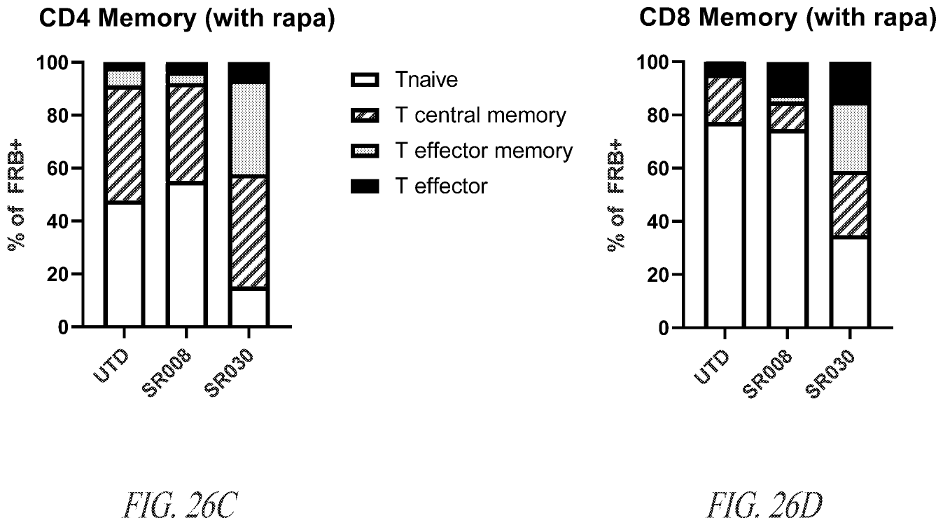

SR-T cells transduced with lentiviral vectors described above (SR008-T cells and SR030-T cells) were analyzed for extracellular expression of key components of the rapamycin-inducible system by flow cytometry, including targeting domains (e.g., anti-CD33 and anti-CLL1 VHH) and an FRB multimerization domain, using a recombinant CD33-Fc fusion, recombinant CLL1-Fc fusion and anti-FRB antibody, respectively. Additionally, an antigen agnostic anti-VHH antibody was used to detect any targeting domain with a VHH format. FRB, anti-CD33 and anti-CLL1 components were highly expressed compared to control cells that were untransduced (FIGS. 26A and 26B). This was validated using anti-VHH.

Additionally, SR008- and SR030-T cells were stained using antibodies against CD62L and CD45RA and expression of each molecule was determined using flow cytometry. Cells were then differentiated into CD62L+CD45RA+ (T naive), CD62L+CD45RA− (T central memory), CD62L-CD45RA+ (T effector memory) and CD62L-CD45RA− (T effector) populations using Flowjo to gate stained cells. While SR008-T cells resembled an untransduced cell in phenotype, the addition of 4-1BB costimulation altered the T cell phenotype to be skewed towards effector memory differentiation compared to untransduced controls in CD4+ (FIG. 26C) and CD8+ (FIG. 26D) T-cell compartments.

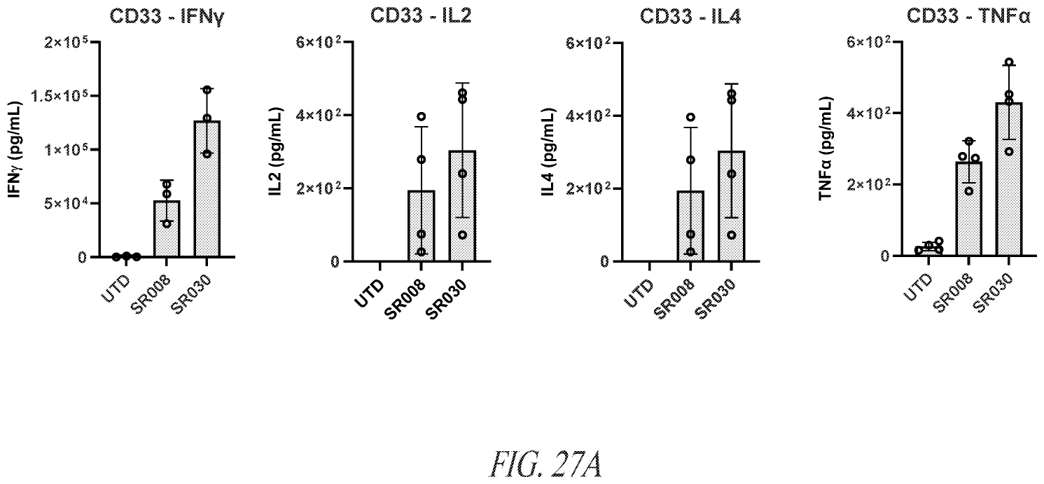

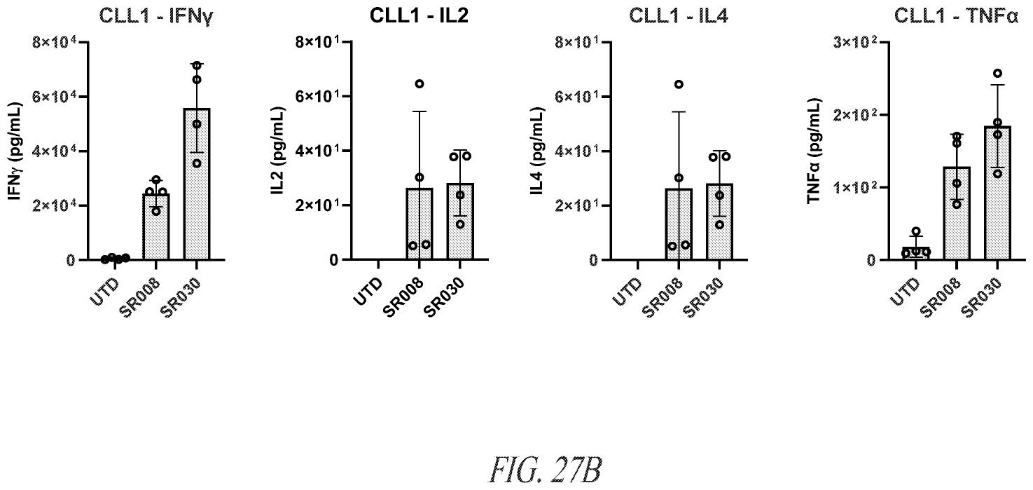

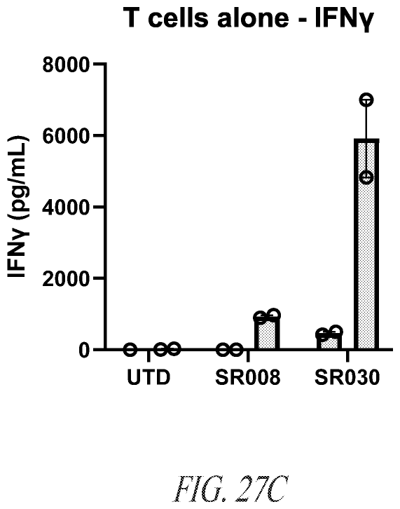

SR0008- and SR0030-T cell rapamycin-dependent, antigen-dependent activity was evaluated in co-cultures with target antigen positive tumor cells in the presence of 1 nM rapamycin. Specifically, IFNγ, IL2, IL4 and TNFα production was quantified using MSD from supernatants of cultures with SR008- and SR030-T cells and a cell line engineered to express high levels of CD33 and CLL1 (FIGS. 27A and 27B, respectively). SR008- and SR030-T cells released each of these 4 cytokines at levels that were highly distinguishable from baseline (untransduced control; UTD). Furthermore, SR030-T cells produced more IFNγ and TNFα compared to SR008-T cells that lacked 4-1BB costimulation.

SR0008- and SR0030-T cell rapamycin-dependent, antigen-independent activity was evaluated in cultures with 1 nM rapamycin alone. Specifically, IFNγ production was quantified using MSD from supernatants of cultures with SR008- and SR030-T cells alone, with and without rapamycin (FIG. 27C). SR030-T cells released significantly more IFNγ than SR008-T cells with and without rapamycin and with no antigen stimulation.

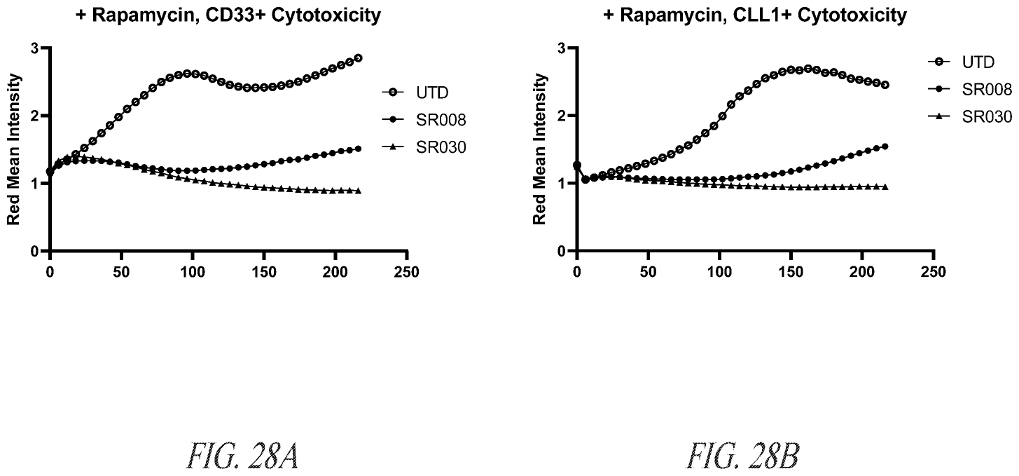

The cytotoxicity of SR008 and SR030-T cells to cells that express both target antigens was assessed using a target line that expressed a nuclear red stain and tracked over time using an Incucyte® live cell imaging system (Sartorius). Cells expressing nuclear stain (antigen positive target line) and cells not expressing nuclear stain (SR-T cells) were both tracked over approximately 250 hours. SR008 and SR030-T cells effectively killed CD33+ target cells, with SR030-T cells controlling tumor growth for a longer duration of time (FIG. 28A). Similarly, in a co-culture with CLL1+ target cells, SR030-T cells controlled tumor cell growth for longer compared to SR008-T cells (FIG. 28B).

### Example 9

### Evaluation of Rapamycin-Inducible TCR T Cells with CD28 Costimulatory Domain

In another experiment, T cells expressing rapamycin-inducible T cell receptors (SR-T cells) were generated using a 10-day transduction and expansion process, then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding rapamycin-inducible TCRs with CD4 hinge and transmembrane domains anchoring an FKBP12 multimerization domain and one antigen targeting domain, with and without a CD28 costimulatory domain (SR001-28 and SR001, respectively) were used to transduce the PBMCs one day after culture initiation, then cells were transferred to a 24-well G-REX® culture system 48 hours later. After a total of 10 days in culture, SR-T cells were evaluated for rapamycin-dependent, antigen-dependent activity, as well as rapamycin-independent, antigen-independent background activity.

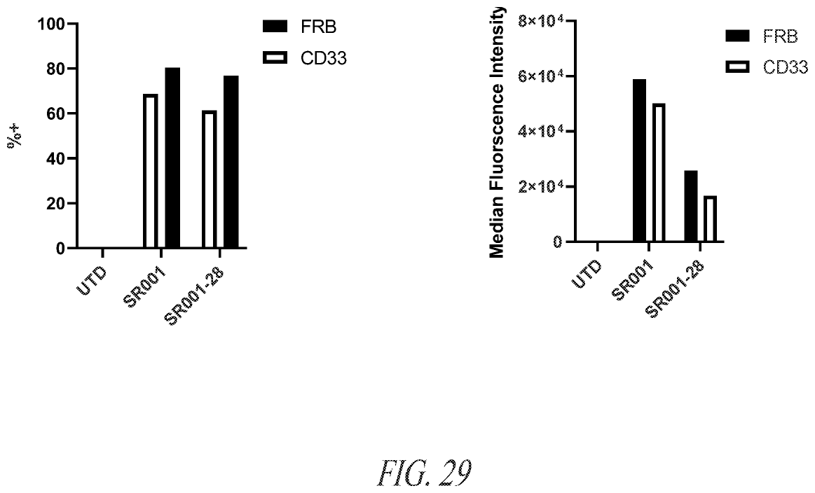

SR-T cells transduced with lentiviral vectors described above (SR001-T cells and SR001-28-T cells) were analyzed for extracellular expression of key components of the rapamycin-inducible system by flow cytometry, including targeting domains (e.g., anti-CD33) and FRB multimerization domains, using a recombinant CD33-Fc fusion and anti-FRB antibody, respectively. FRB and anti-CD33 components were highly expressed compared to control cells that were untransduced (FIG. 29).

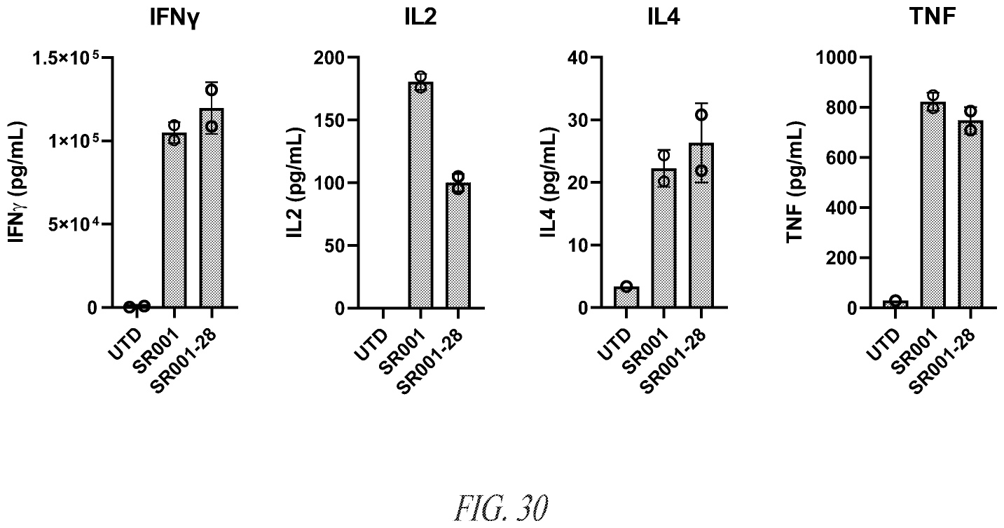

SR001- and SR001-28-T cell rapamycin-dependent, antigen-dependent activity was evaluated in co-cultures with target antigen positive tumor cells in the presence of 1 nM rapamycin. Specifically, IFNγ, IL2, IL4 and TNFα production was quantified using MSD from supernatants of cultures with SR001- and SR001-28-T cells and a cell line endogenously expressing high levels of CD33 (FIG. 30). SR001- and SR001-28-T cells released each of these 4 cytokines at levels that were highly distinguishable from baseline (untransduced control; UTD).

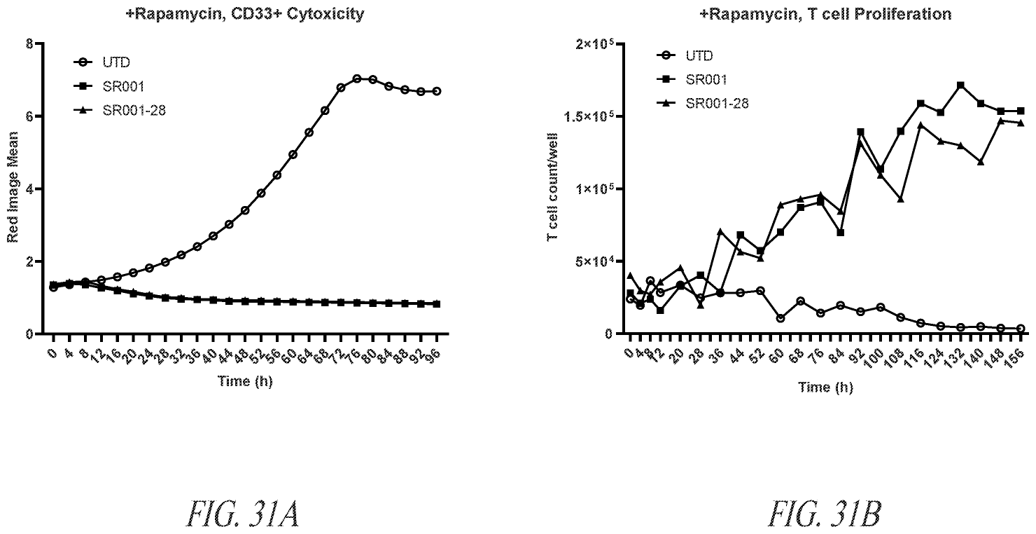

The cytotoxicity of SR001 and SR001-28-T cells to cells that express both target antigens was assessed using a target line that expressed a nuclear red stain and tracked over time using an Incucyte® live cell imaging system (Sartorius). Cells expressing nuclear stain (antigen positive target line) and cells not expressing nuclear stain (SR-T cells) were both tracked over approximately 150 hours. SR001 and SR001-28-T cell effectively killed CD33+ target cells (FIG. 31A) and significantly expanded in these cultures compared to an untransduced T cell control (FIG. 31B).

### Example 10

### Evaluation of Rapamycin-Inducible TCR T Cells with 4-1Bb or CD28 Costimulatory Domain In Vivo

In another experiment, T cells expressing rapamycin-inducible T cell receptors (SR-T cells) were generated using a 10-day transduction and expansion process, then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding rapamycin-inducible TCRs with CD4 hinge and transmembrane domains anchoring a FKBP12 multimerization domain and one antigen targeting domain to a 4-1BB or a CD28 costimulatory domain (SR001, SR001-41BB, SR001-28) were used to transduce the PBMCs one day after culture initiation, then cells were transferred to a 1 L G-REX® culture system 48 hours later. After a total of 10 days in culture, SR-T cells were evaluated for rapamycin-dependent, antigen-dependent activity, as well as rapamycin-independent, antigen-independent background activity.

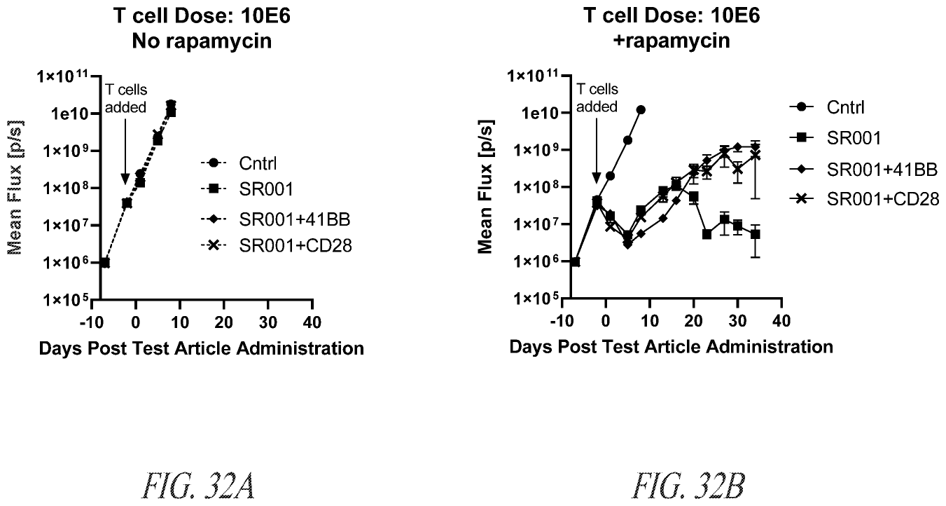

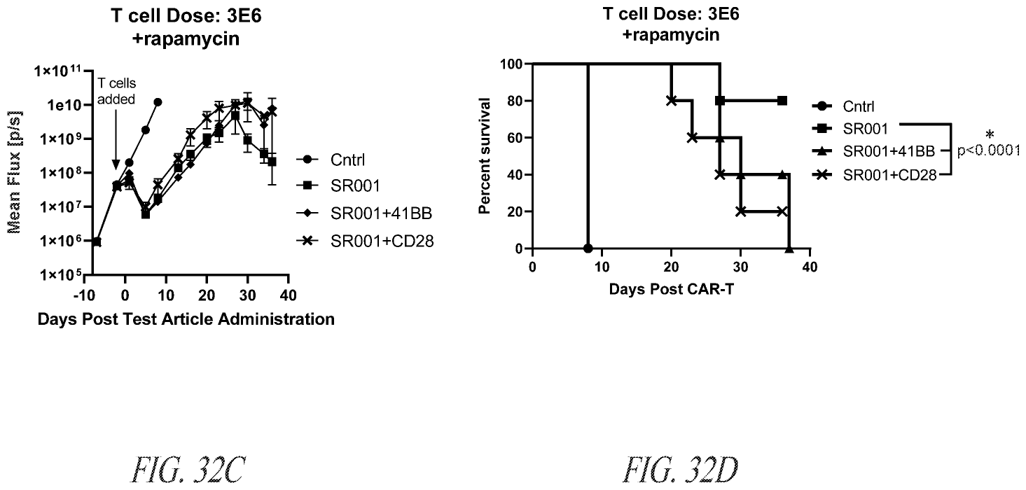

Female NSG mice were dosed intravenously with a CD33+ HL-60 xenograft tumor cells expressing firefly luciferase. After 10 days of tumor growth, 10E6 untransduced, SR001, SR001-41BB, or SR001-28 T cells were administered intravenously without rapamycin (FIG. 32A) or with rapamycin dosed three times per week (FIG. 32B). Alternatively, 3E6 untransduced, SR001, SR001-41BB or SR001-28 T cells were administered intravenously with rapamycin dosed three times per week (FIG. 32C) and survival was monitored for 38 days (FIG. 32D). In all instances, SR001 performed or trended better than SR001-41BB and SR001-28 T cells.

### Example 11

### Evaluation of Rapamycin-Inducible TCR T Cells with a CD4 or CD8 Coreceptor Domain

In another experiment, T cells expressing rapamycin-inducible T cell receptors (SR-T cells) were generated using a 10-day transduction and expansion process, then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding rapamycin-inducible TCRs with CD4 hinge and transmembrane domains anchoring a FKBP12 multimerization domain and one antigen targeting domain to a CD4 or CD8 coreceptor domain (SR022, SR022+CD4 and SR022+CD8) were used to transduce the PBMCs one day after culture initiation, then cells were transferred to a 24-well G-REX® culture system 48 hours later. After a total of 10 days in culture, SR-T cells were evaluated for rapamycin-dependent, antigen-dependent activity, as well as rapamycin-independent, antigen-independent background activity.

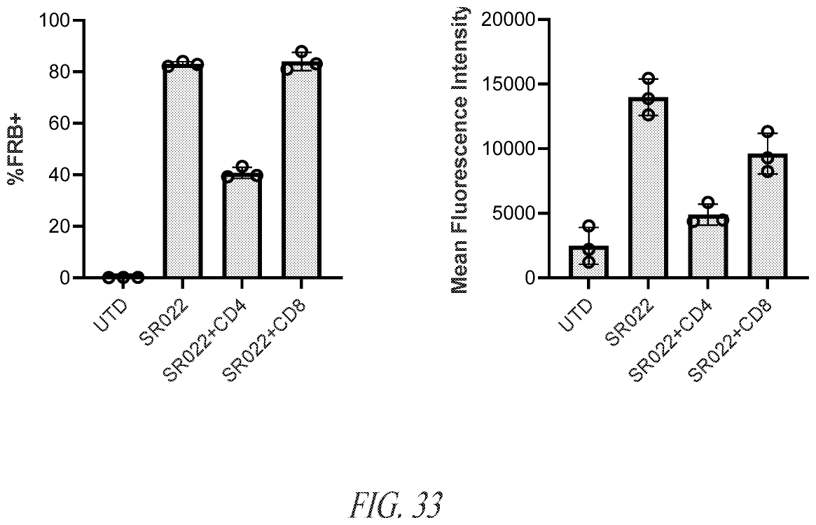

SR-T cells transduced with lentiviral vectors described above (SR022, SR022+CD4 and SR022+CD8) were analyzed for extracellular expression of key components of the rapamycin-inducible system by flow cytometry, specifically the FRB multimerization domain, using an anti-FRB antibody. FRB was highly expressed compared to control cells that were untransduced (FIG. 33).

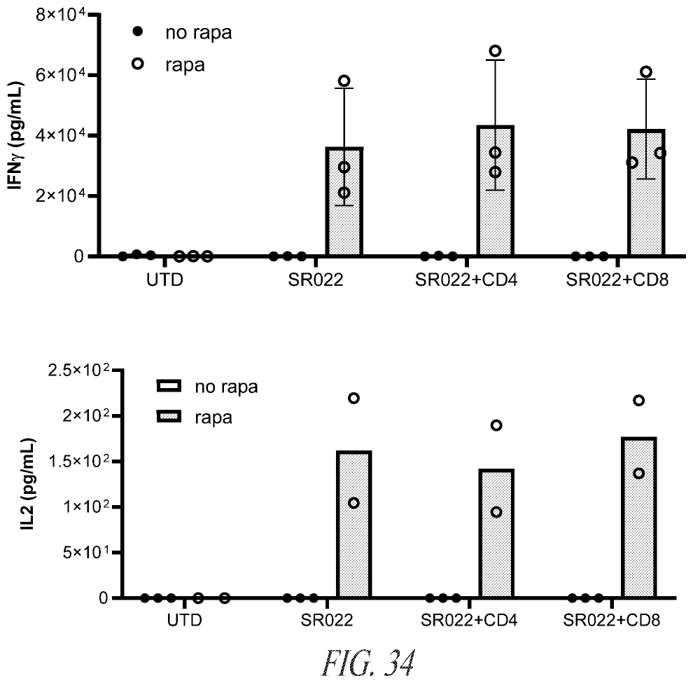

SR022, SR022+CD4 and SR022+CD8-T cell rapamycin-dependent, antigen-dependent activity was evaluated in co-cultures with target antigen positive tumor cells in the presence of 1 nM rapamycin. Specifically, IFNγ and IL2 production was quantified using MSD from supernatants of cultures with SR022, SR022+CD4 and SR022+CD8-T cells and a cell line endogenously expressing high levels of CD33 (FIG. 34). SR022, SR022+CD4 and SR022+CD8-T cells released each of these 2 cytokines at levels that were highly distinguishable from baseline (untransduced control; UTD).

### Example 12

### Evaluation of Rapamycin-Inducible TCR T Cells with a Transgenic TCR

In another experiment, T cells expressing rapamycin-inducible T cell receptors and a transgenic TCR (SR-T cells) were generated using a 10-day transduction and expansion process, then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding rapamycin-inducible TCRs with CD4 hinge and transmembrane domains anchoring a FKBP12 multimerization domain and one antigen targeting domain. Additionally, the same lentiviral vector encodes a transgenic TCR (SR001 and TCR+SR001) This vector was used to transduce the PBMCs one day after culture initiation, then cells were transferred to a 24-well G-REX® culture system 48 hours later. After a total of 10 days in culture, SR-T cells were evaluated for rapamycin-dependent, antigen-dependent activity, as well as rapamycin-independent, antigen-independent background activity.

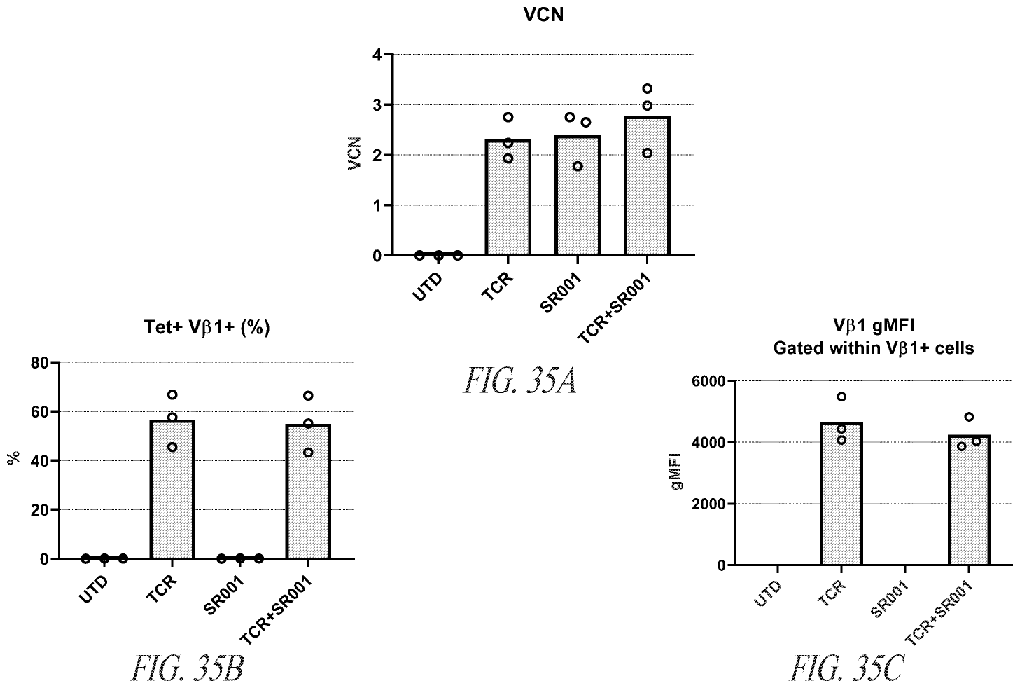

SR-T cells transduced with lentiviral vectors described above (SR001 and TCR+SR001) were analyzed for transgene integration using a qPCR-based assay to determine vector copy number (FIG. 35A) and extracellular expression of key components of the rapamycin-inducible+transgenic TCR system by flow cytometry, including the TCR, using the beta chain-specific antibody and an epitope-specific tetramer (FIGS. 35B and 35C), and the targeting domains using an anti-VHH. All components were highly expressed compared to control cells that were untransduced (FIGS. 35D and 35E).

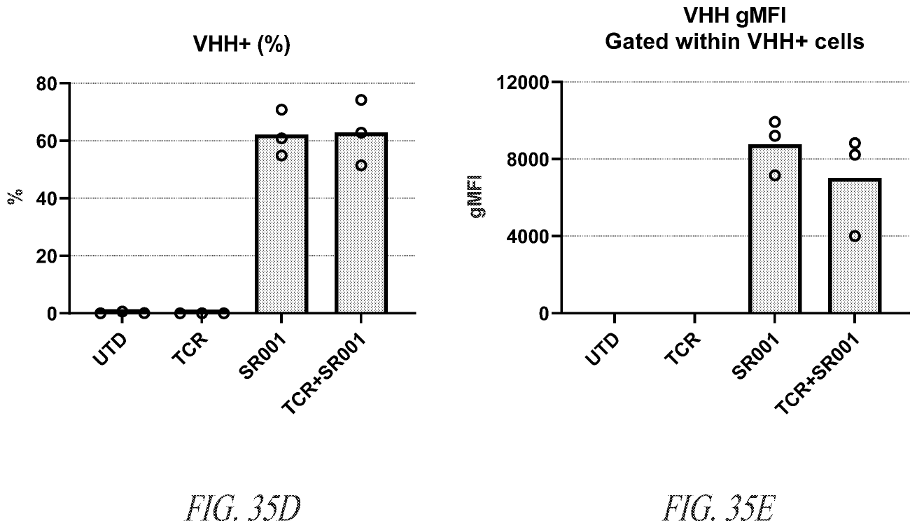

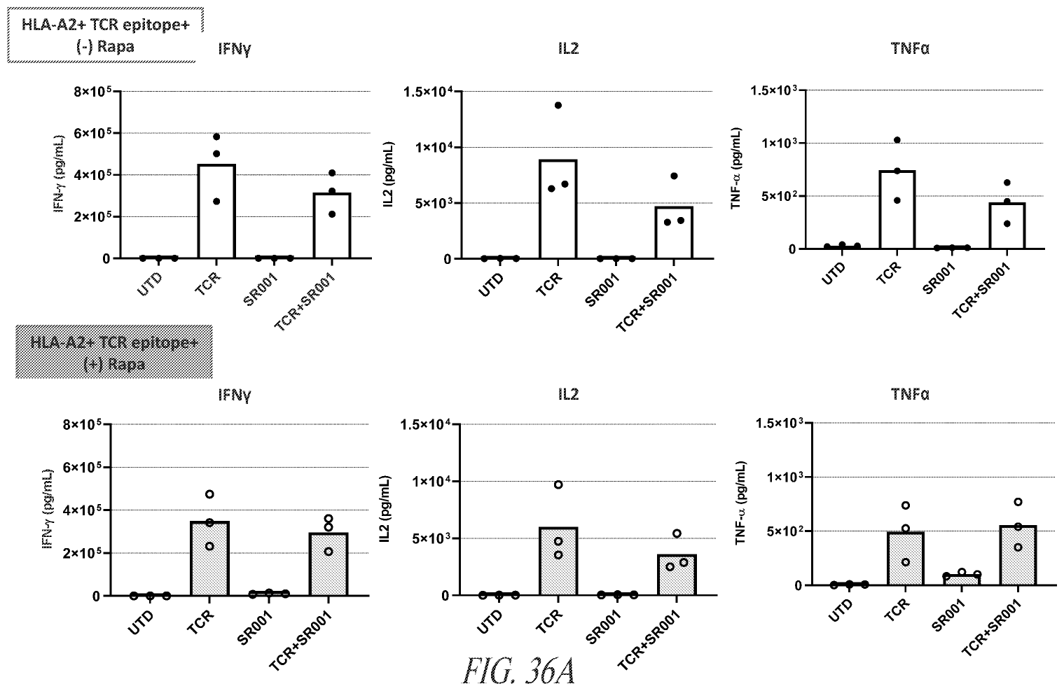

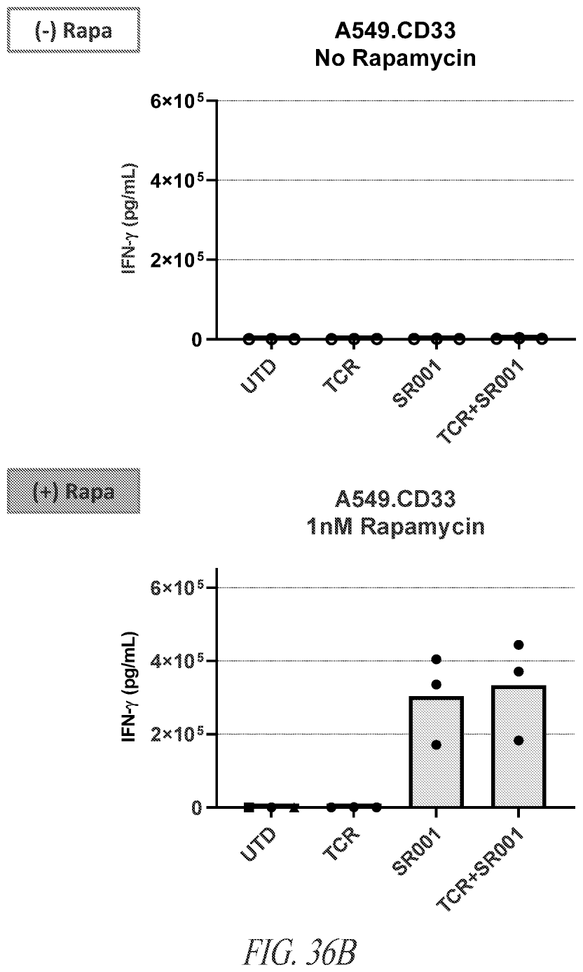

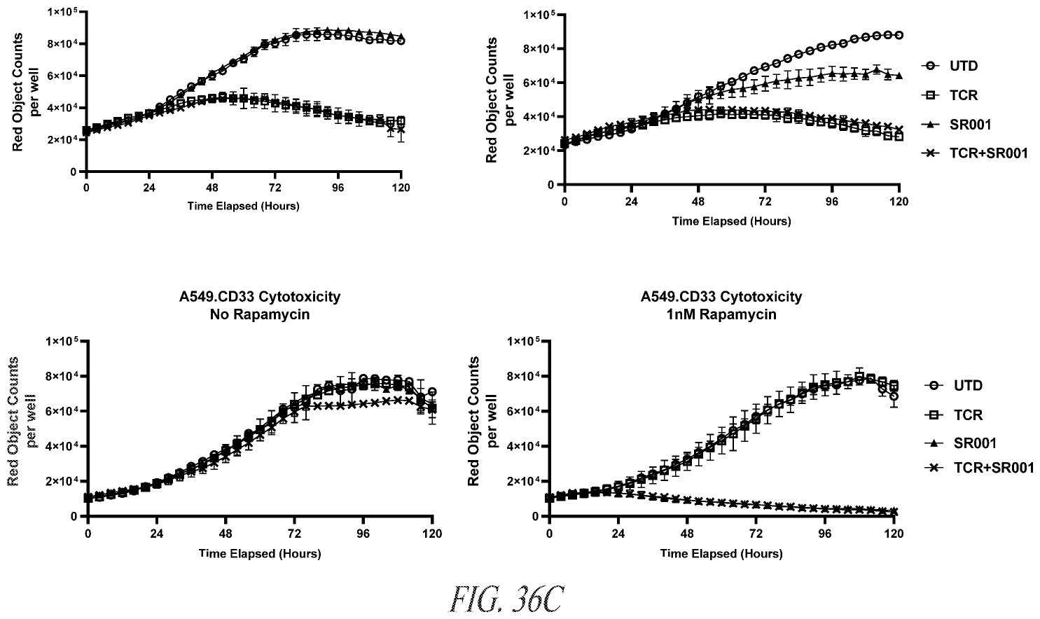

SR001 and TCR+SR001-T cell rapamycin-dependent, antigen-dependent activity was evaluated in co-cultures with target antigen positive tumor cells in the presence of 1 nM rapamycin. Specifically, IFNγ, IL2 and TNFα production was quantified using MSD from supernatants of cultures with SR001 and TCR+SR001-T cells and a cell line endogenously expressing high levels of the HLA-A2+TCR epitope, with and without rapamycin (FIG. 36A). IFNγ production was also quantified using MSD from supernatants of cultures with SR001 and TCR+SR001-T cells and a cell line endogenously expressing high levels of CD33, with and without rapamycin (FIG. 36B). In all cases, SR001 and TCR+SR001-T cells released each cytokine at levels that were highly distinguishable from baseline (untransduced control; UTD). Additionally, the combination of the transgenic TCR with SR001 in T cells did not diminish antigen-specific cytokine release compared to T cells expressing only TCR or SR001.

The cytotoxicity of SR001 and TCR+SR001-T cells to cells that express either target antigens was assessed using two target lines that expressed a nuclear red stain and tracked over time using an Incucyte® live cell imaging system (Sartorius). Cells expressing nuclear stain (antigen positive target line) and cells not expressing nuclear stain (SR-T cells) were both tracked over approximately 120 hours. TCR or TCR+SR001-T cells effectively killed TCR epitope+target cells (FIG. 36C, top panel) while SR001 and TCR+SR001-T cell effectively killed CD33+ target cells (FIG. 36C, bottom panel).

### Example 13

### Evaluation of Rapamycin-Inducible TCR T Cells at Very Low Antigen Density

In another experiment, T cells expressing rapamycin-inducible T cell receptors and a transgenic TCR (SR-T cells) were generated using a 10-day transduction and expansion process, then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding rapamycin-inducible TCRs with CD4 hinge and transmembrane domains anchoring a FKBP12 multimerization domain and one antigen targeting domain (SR001) were used to transduce the PBMCs one day after culture initiation, then cells were transferred to a 24-well G-REX® culture system 48 hours later. After a total of 10 days in culture, SR-T cells were evaluated for rapamycin-dependent, antigen-dependent activity, as well as rapamycin-independent, antigen-independent background activity.

SR001-T cell rapamycin-dependent, antigen-dependent activity was evaluated in co-cultures with target antigen positive tumor cells in the presence of 1 nM rapamycin.

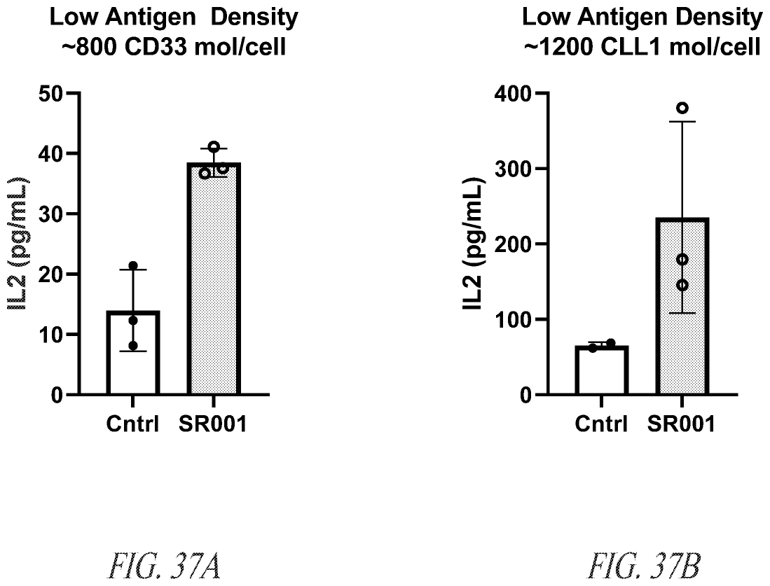

Specifically, target cells were transfected with CD33- or CLL1-encoding mRNA to generate target cells expressing less than 1500 molecules of each per cell. IL2 production was quantified using MSD from supernatants of cultures with SR001 (FIGS. 37A and 37B). SR001-T cells released IL2 at levels that were highly distinguishable from baseline (untransduced control; UTD).

### Example 14

### Evaluation of Rapamycin-Inducible TCR T Cells In Vivo to Control Low Antigen Tumors

In another experiment, T cells expressing rapamycin-inducible T cell receptors (SR-T cells) were generated using a 10-day transduction and expansion process then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding rapamycin-inducible TCRs with CD4 hinge and transmembrane domains anchoring a FKBP12 multimerization domain and one antigen targeting domain (i.e., a CD33 VHH or a CLL1 VHH) (SR001 and SR007, respectively) were used to transduce the PBMCs one day after culture initiation, then cells were transferred to a 1 L G-REX® culture system 48 hours later. After a total of 10 days in culture, SR-T cells were evaluated for rapamycin-dependent, antigen-dependent activity, as well as rapamycin-independent, antigen-independent background activity.

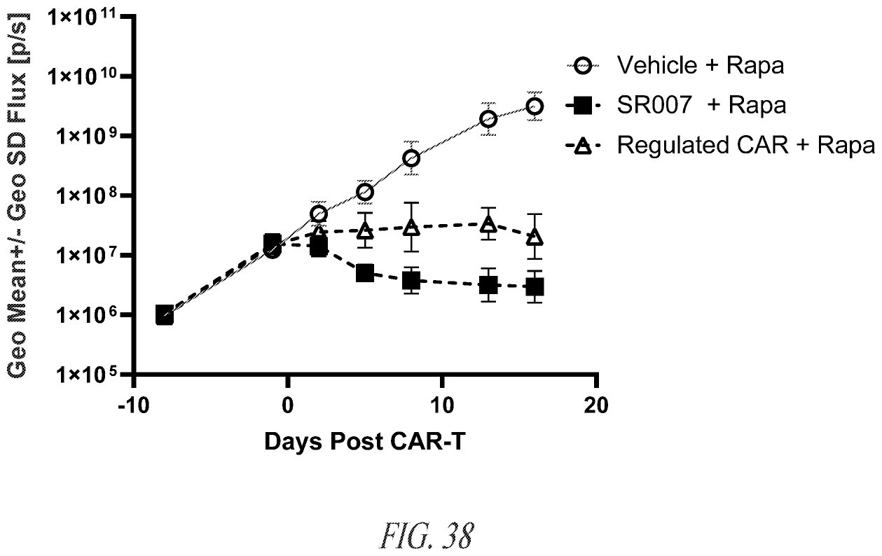

Female NSG mice were dosed intravenously with a CD33+CLL1+ OCI-AML3 xenograft tumor cells expressing firefly luciferase. After 10 days of tumor growth, 10E6 untransduced, SR007-, or comparator regulated CAR-T cells were administered intravenously with rapamycin dosed three times per week (FIG. 38). The comparator regulated CAR (Reg CAR 1) was a 2^{nd}-generation 41BB-CD3ζ split into two, rapamycin regulated domains, using the same CLL1 binder as SR007 (see, e.g., Leung et al., JCI Insight. 2019 Apr. 30; 5(11), for general regulated CAR design). Mice treated with SR007-T cells controlled their tumors faster and more deeply than mice treated with comparator regulated CAR T cells.

### Example 15

### Evaluation of Rapamycin-Inducible TCR T Cells Targeting CD19

In another experiment, T cells expressing rapamycin-inducible T cell receptors (SR-T cells) were generated using a 10-day transduction and expansion process, then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding rapamycin-inducible TCRs with CD4 hinge, transmembrane, and intracellular domains anchoring either a FKBP12 or FRB multimerization domain and two antigen targeting domains (SR10167 and SR10168; each having a CD19 VHH or a CD19 scFv, respectively) were used to transduce the PBMCs one day after culture initiation, then cells were transferred to a 24-well G-REX® culture system (Wilson Wolf) 48 hours later. After a total of 10 days in culture, SR-T cells were evaluated for rapamycin-dependent, antigen-dependent activity, as well as rapamycin-independent, antigen-independent background activity.

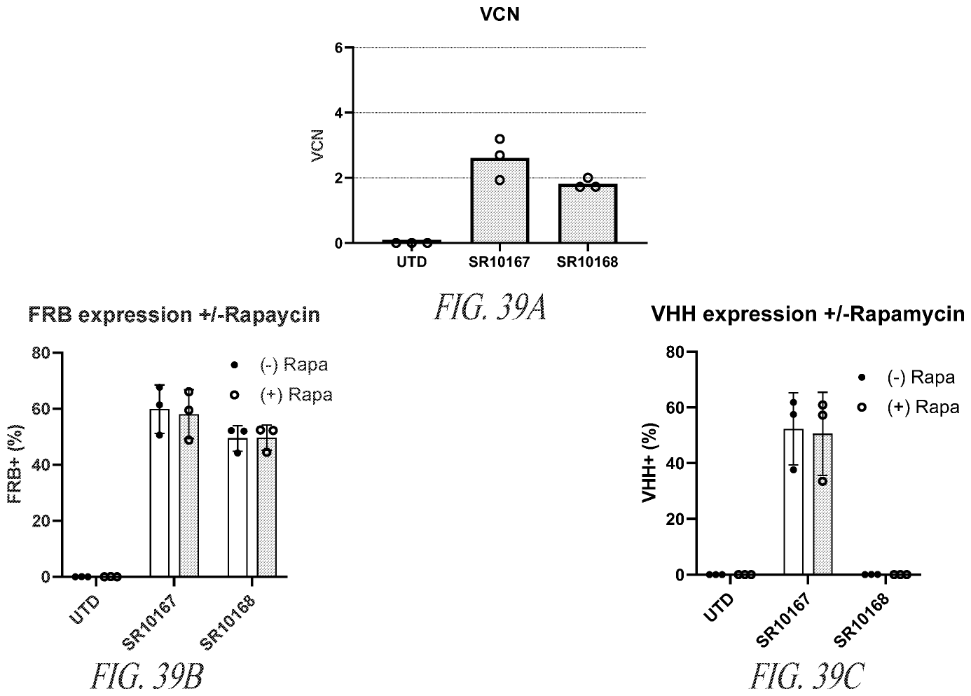

SR-T cells transduced with lentiviral vectors described above (SR10167- and SR10168-T cells) were analyzed for transgene integration using a qPCR-based assay to determine vector copy number (FIG. 39A) and for extracellular expression of key components of the rapamycin-inducible system by flow cytometry, including targeting domains (e.g., CD19 VHH or scFv) and the FRB multimerization domain, using a recombinant CD19-Fc fusion and anti-FRB antibody, respectively. FRB and anti-CD19 components were highly expressed compared to control cells that were untransduced (FIGS. 39B and 39C).

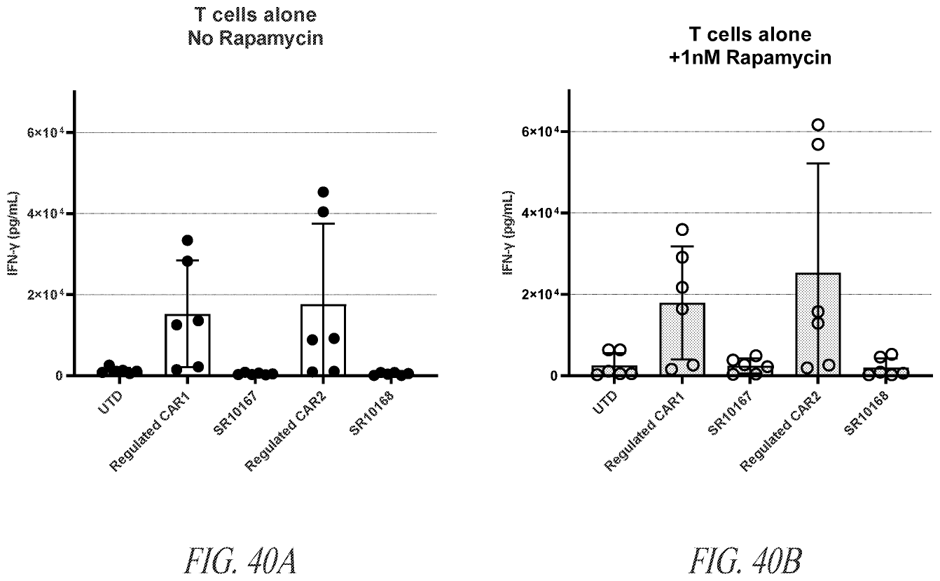

T cells were cultured alone with no antigen present, both without and in the presence of 1 nM of rapamycin. SR10167- and SR10168-T cells produced no IFNγ when rapamycin was absent and only small amounts comparable to untransduced when 1 nM of rapamycin was added to the culture. (FIGS. 40A and 40B). By comparison, comparator regulated CARs that were targeted using either the CD19 VHH (Reg CAR1) or the CD19 scFv (Reg CAR 2) produced IFNγ at levels significantly higher than baseline both with and without 1 nM rapamycin in the cultures.

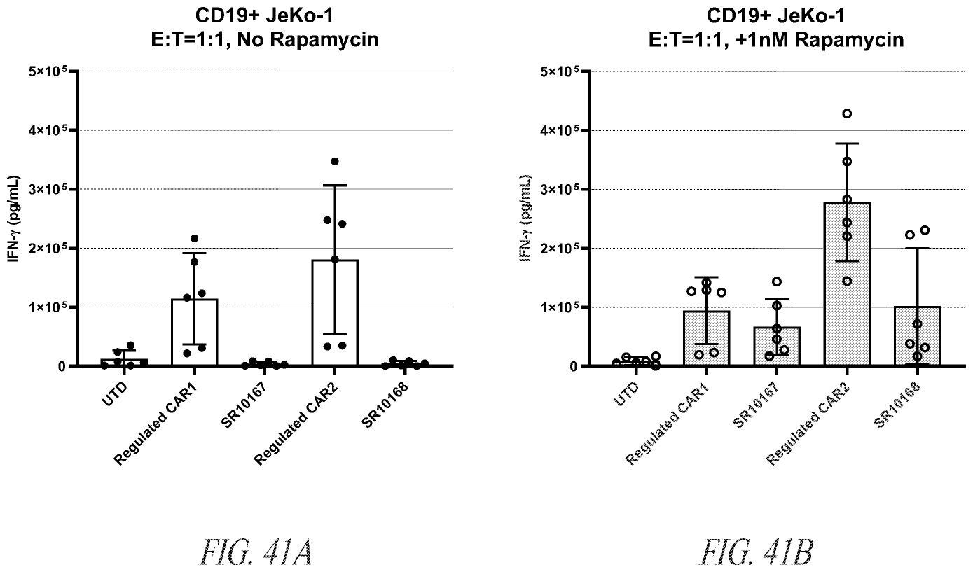

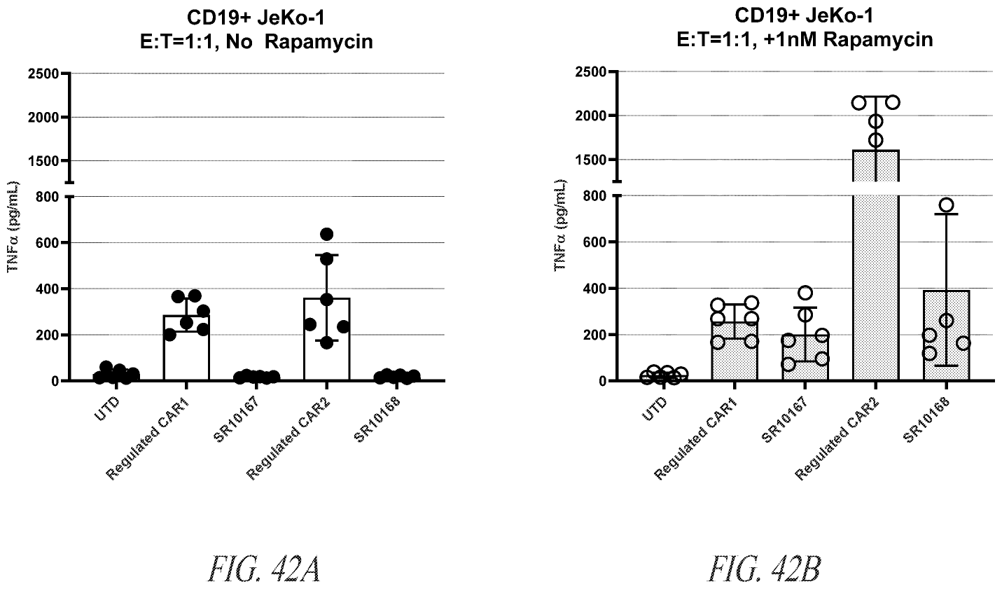

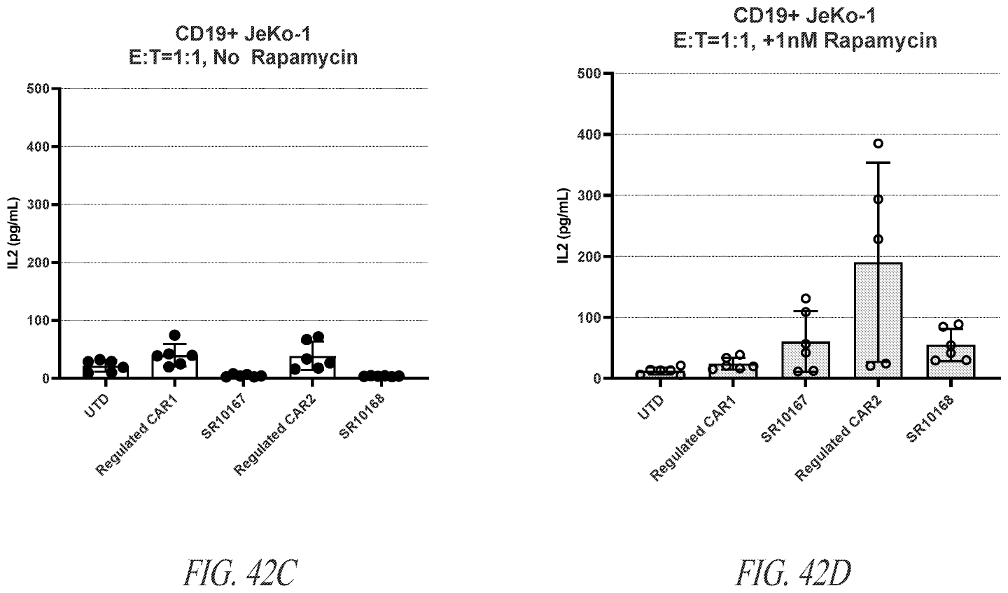

SR10167- and SR10168-T cell rapamycin-dependent, antigen-dependent activity was evaluated in co-cultures with target antigen positive tumor cells in the presence or absence of 1 nM rapamycin. Specifically, IFNγ production was quantified using MSD from supernatants of cultures with SR10167- and SR10168-T cells and cell lines that endogenously express CD19 (FIGS. 41A and 41B). Additionally, TNFα and IL2 were quantified. SR10167- and SR10168-T cells released each of these 3 cytokines at levels that were distinguishable from baseline (untransduced control; UTD) but lower than comparable regulated CAR Ts. (FIGS. 42A-42D).

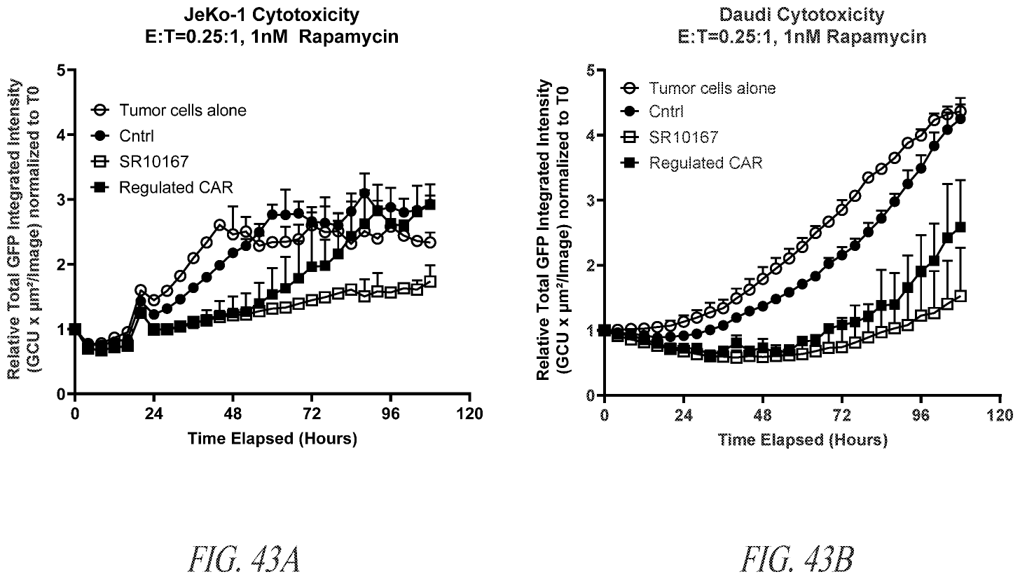

The cytotoxicity of SR10167-T cells to cells that express target antigens was assessed using two target lines that expressed GFP and tracked over time using an Incucyte® live cell imaging system (Sartorius). Cells expressing GFP (antigen positive target line) and cells not expressing GFP (SR-T cells) were both tracked over approximately 120 hours. SR10167-T cells effectively kill CD19+ target cells (FIGS. 43A and 43B) For both CD19+ target cultures, SR10167-T cells trended to having superior cytotoxicity than the comparator regulated CAR (Reg CAR 1).

### Example 16

### Construction and Evaluation of Constitutive T Cell Receptor-Like Architectures

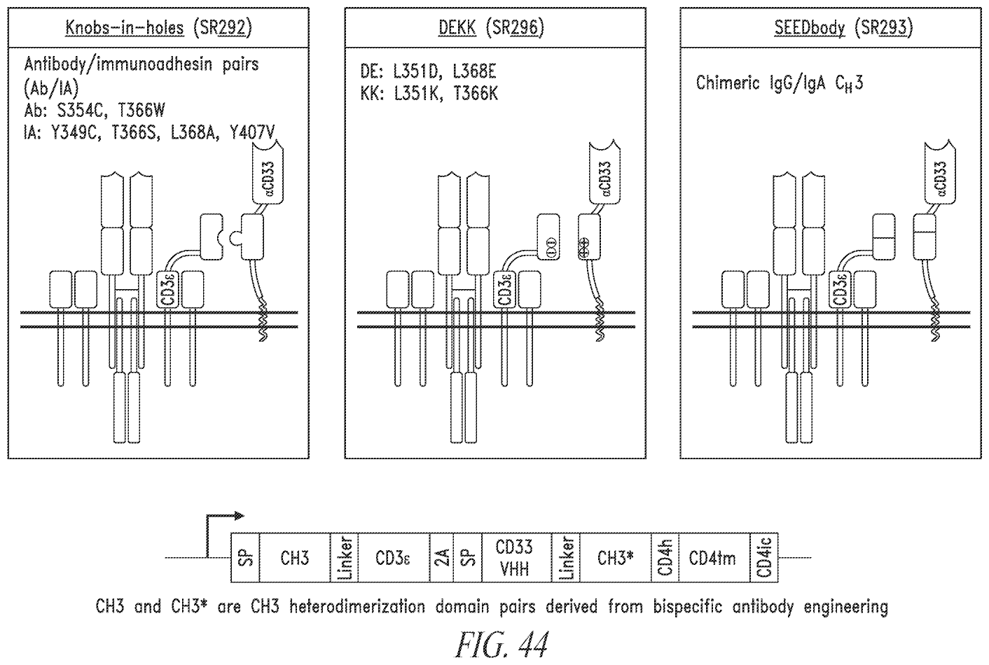

To generate a rapamycin-independent, constitutive TCR-like architecture, the FRB and FKBP12 domains were substituted with heterodimerization domains derived from the CH3 region of Fc-engineered bispecific antibodies. Lentiviral vectors comprising constructs that include constitutive heterodimerization domain pairs, CD3 subunits and an antigen targeting domain were designed, cloned and sequences were verified. Constructs comprise a promoter driving expression of a combination of the following units: a CD8α or IgKa signal sequence, antibody Fc CH3 heterodimerization domain pairs, an antibody derived targeting domain (e.g., CD33 VHH), CD4 hinge, transmembrane and intracellular domains. (FIG. 44; constructs SR292, SR293, and SR296). Specifically, for constructs SR292, SR293, and SR296, the CH3 heterodimerization domain pairs were derived using mutations from previously described knobs-in-holes antibodies, SEEDbody using chimeric IgA/IgG CH3 domains, and DEKK electrostatic pairs, respectively (see, e.g., Merchant, A., Zhu, Z., Yuan, J. et al An efficient route to human bispecific IgG. Nat Biotechnol 16, 677-681 (1998).Nardis, C., Hendriks, L., Poirier, E. et a. A new approach for generating bispecific antibodies based on a common light chain format and the stable architecture of human immunoglobulin G1. J Biol Chem 292, 14706-14717 (2017). Davis, J., Aperlo, C., Li, Y. et at. SEEDbodies: fusion proteins based on strand-exchange engineered domain (SEED)C-13 heterdimers in an Fe analogue platform for asymmetric binders or immunofusions and bispecific antibodies Protein Engineering, Design and Selection, Volume 23, Issue 4, April 2010, Pages 195-202. While the pairs were tested in one orientation within the construct, it is anticipated that the opposite orientation would function similarly.

T cells expressing constitutive T cell receptor-like architectures (SR-T cells) were generated using a 10-day transduction and expansion process, then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding the constructs were used to transduce the PBMCs one day after culture initiation, then cells were transferred to a 24-well G-REX® culture system 48 hours later. After a total of 10 days in culture, SR-T cells were evaluated for antigen-dependent activity as well as antigen-independent background activity. A standard CAR, regulated CAR, architecture comparator and SR001 targeting the same antigen were used as functional controls and comparators.

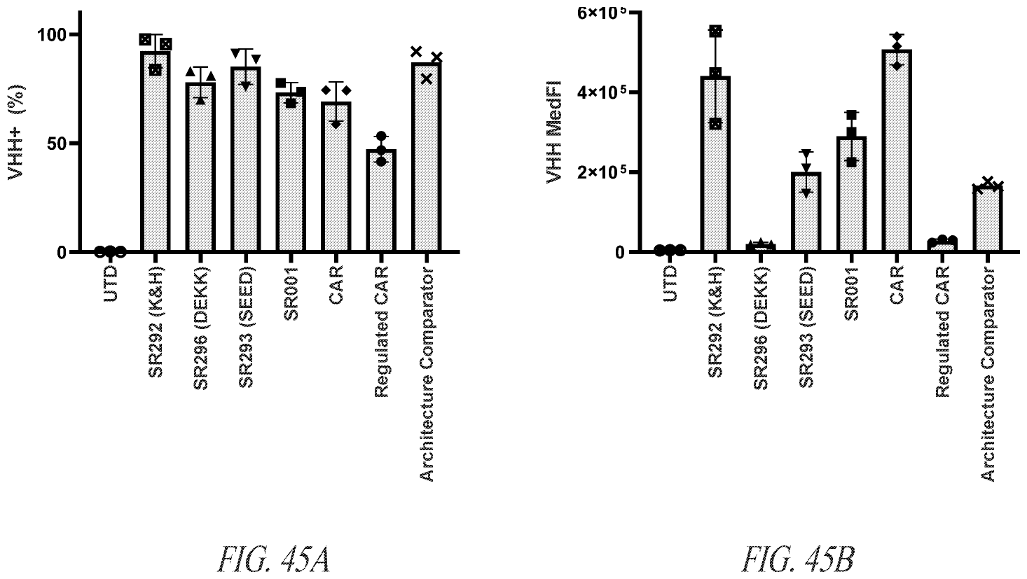

SR-T cells transduced with lentiviral vectors described above (SR292, SR293, and SR296 T cells) were analyzed by flow cytometry for extracellular expression of the targeting domain fused to one half of the CH3 heterodimerization domains linked to a transmembrane domain using a recombinant anti-VHH antibody (FIGS. 45A and 45B). All constructs expressed and were evaluable.

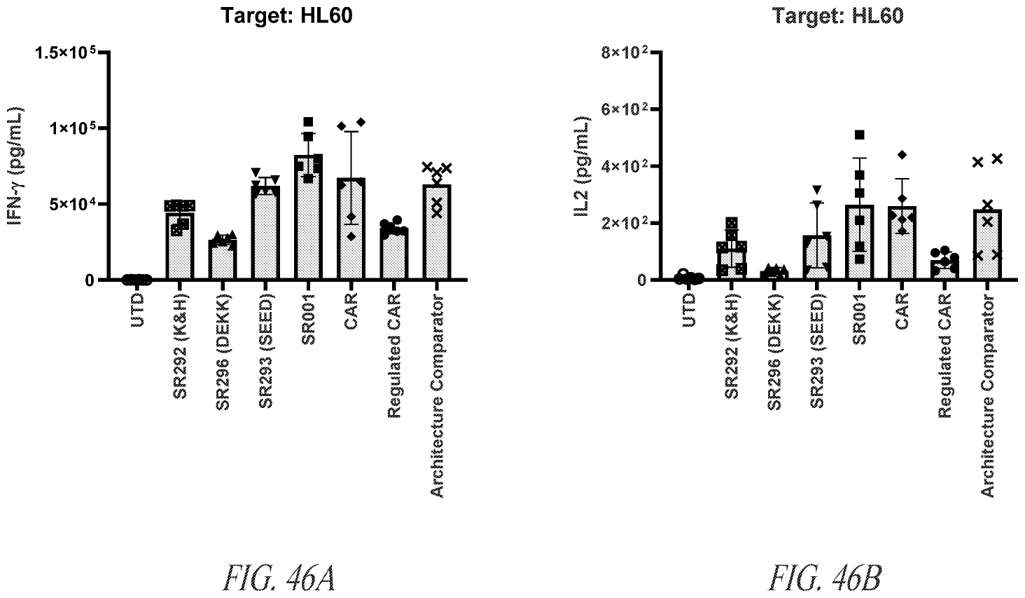

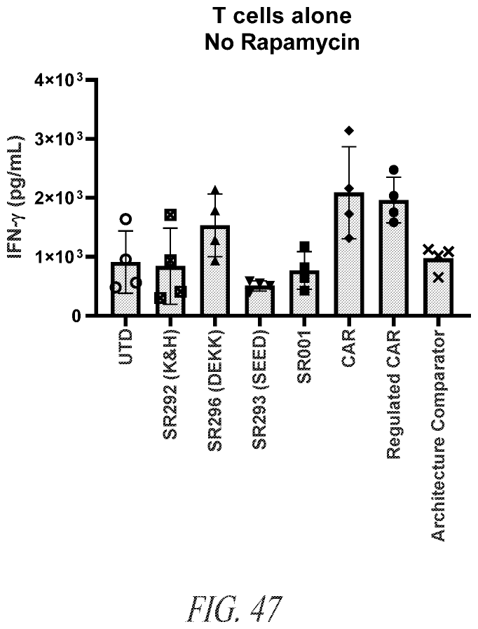

SR292, SR293, and SR296 T cell antigen-dependent activity was evaluated in co-cultures with target antigen positive tumor cells. The induced dimerization control constructs, regulated CAR and SR001 T cells, included the addition of a dimerization agent to promote activity. Specifically, IFNγ and IL2 production was quantified using MSD from supernatants of co-cultures with T cells and a HL60 cell line expressing the target antigen CD33 (FIGS. 46A and 46B). SR292, SR293, and SR296-T cells released cytokines at levels that were amplified from baseline, untransduced control T cells, indicating antigen-dependent activation. SR292, SR293, and SR296 T cells did not exhibit robust cytokine secretion when cultured alone with no antigen present and the levels were comparable to untransduced T cells (FIG. 47).

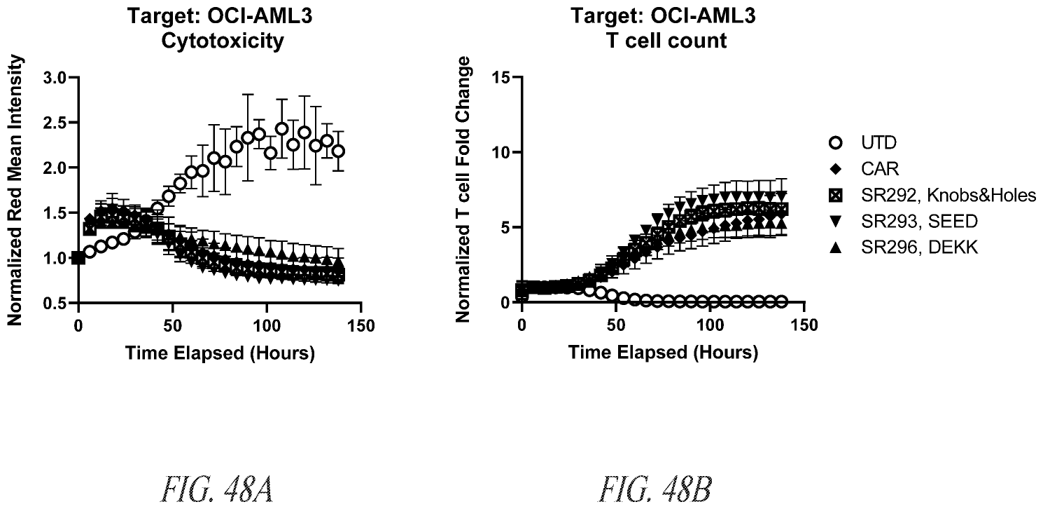

The cytotoxicity of SR292, SR293, and SR296-T cells was assessed by co-culturing transduced SR-T cells with antigen positive tumor cells engineered to express a nuclear red fluorescent protein. Cells displaying nuclear fluorescence (antigen positive target line) and cells not expressing nuclear fluorescence (SR-T cells) were both tracked over approximately 140 hours using an Incucyte® live cell imaging system (Sartorius). SR292, SR293, and SR296 modified T cells effectively eliminated red-labeled antigen positive tumor cells and modified T cells expanded at levels comparable to a control anti-CD33 CAR molecule (FIGS. 48A and 48B).

Altogether, these data are indicative of the generation of a constitutive, antigen-dependent TCR-like architecture capable of re-directing T cell activity.

### Example 17

### Evaluation of Rapamycin-Inducible TCR T Cells with Cytokine Receptor Domains

In another experiment, T cells expressing rapamycin-inducible T cell receptors (SR-T cells) were generated using a 10-day transduction and expansion process, then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding rapamycin-inducible TCRs with CD4 hinge, transmembrane and intracellular domains anchoring an FKBP12 or FRB multimerization domain and antigen targeting domains to cytokine receptor intracellular signaling domains (SR001±IL7Rα, IL2Rβ, common γ chain or both IL2Rβ and common γ chain) were used to transduce the PBMCs one day after culture initiation, then cells were transferred to a 24-well G-REX® culture system (Wilson Wolf) 48 hours later. After a total of 10 days in culture, SR-T cells were evaluated for rapamycin-dependent, antigen-dependent activity, as well as rapamycin-independent, antigen-independent background activity.

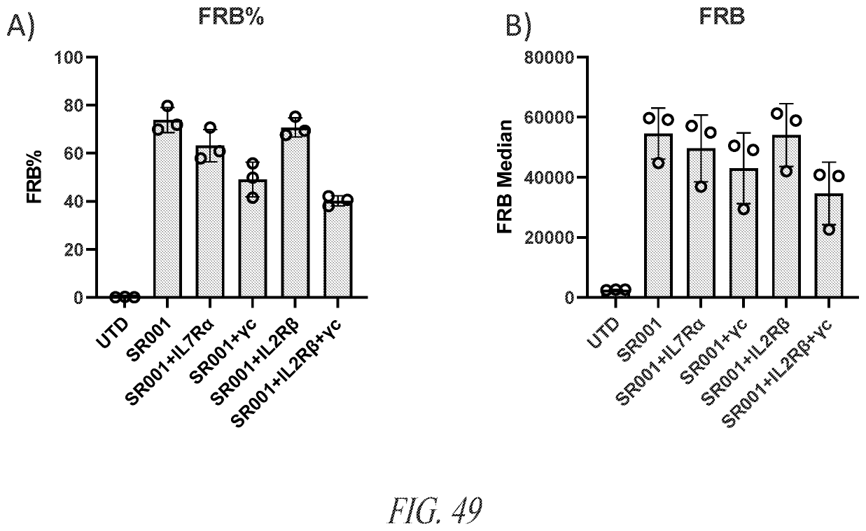

SR-T cells transduced with lentiviral vectors described above (SR001±IL7Rα, IL2Rβ, common γ chain, or both IL2Rβ and common γ chain-T cells) were analyzed for extracellular expression of key components of the rapamycin-inducible system by flow cytometry, specifically FRB dimerization domain using anti-FRB antibody. FRB components were highly expressed compared to control cells that were untransduced (FIG. 49).

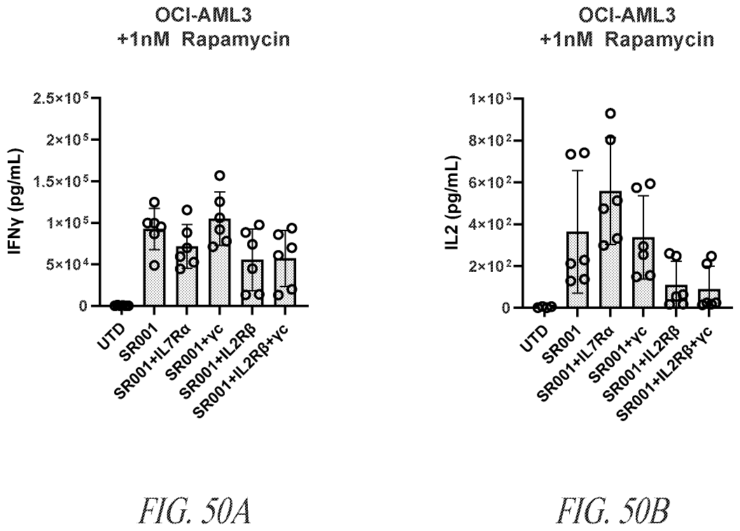

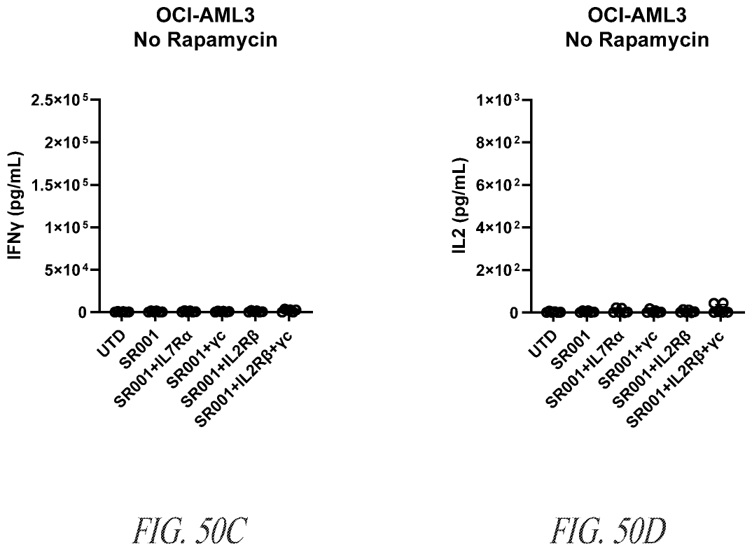

SR001±IL7Rα, IL2Rβ, common γ chain, or both IL2Rβ and common γ chain-T cell rapamycin-dependent, antigen-dependent activity was evaluated in co-cultures with target antigen positive tumor cells in the presence or absence of 1 nM rapamycin. Specifically, IFNγ and IL2 production was quantified using MSD from supernatants of cultures with SR001±IL7Rα, IL2Rβ, common γ chain, or both IL2Rβ and common γ chain-T cells and cell lines that endogenously express CD33. SR001±IL7Rα, IL2Rβ, common γ chain, or both IL2Rβ and common γ chain-T cells released each of these 2 cytokines at levels that were distinguishable from baseline (untransduced control) but lower than comparable regulated CAR Ts. (FIGS. 50A-50D).

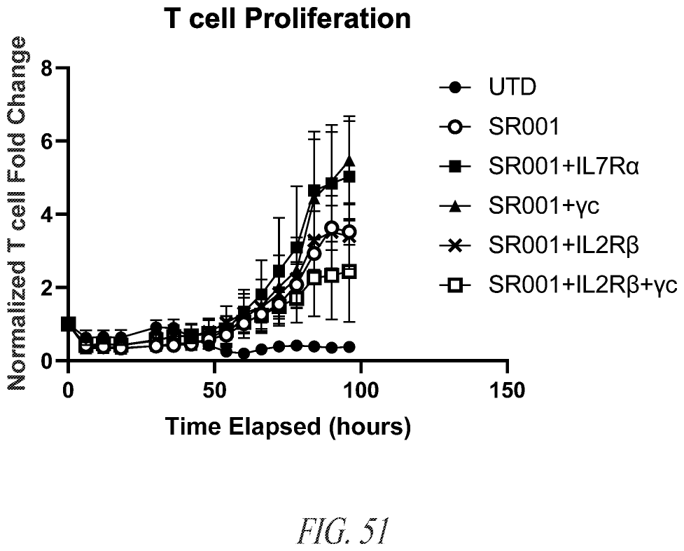

Antigen-specific T cell proliferation of SR001±IL7Rα, IL2Rβ, common γ chain, or both IL2Rβ and common γ chain-T cells was assessed by culturing a CD33+ target line that expresses a red nuclear fluorescent stain and tracking T cell growth over time using an Incucyte® live cell imaging system (Sartorius). Cells expressing nuclear stain (antigen positive target line) and cells not expressing nuclear stain (SR-T cells) were both tracked over approximately 100 hours. SR001±IL7Rα, IL2Rβ, common γ chain or both IL2Rβ and common γ chain-T cells proliferated in the presence of CD33+ target cells (FIG. 51), particularly those that had IL7Ra and common γ chain signaling.

### Example 18

### Evaluation of Rapamycin-Inducible TCR T Cells Targeting with Natural Ligands

In another experiment, T cells expressing rapamycin-inducible T cell receptors (SR-T cells) were generated using a 10-day transduction and expansion process, then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding rapamycin-inducible TCRs with CD4 hinge, transmembrane and intracellular domains anchoring an FKBP12 or FRB multimerization domain and natural ligand antigen targeting domains (e.g., high-affinity PD1 (see, e.g., US20220378873A1) and PD1 ectodomains; SR300 and SR301, respectively) were used to transduce the PBMCs one day after culture initiation, then cells were transferred to a 24-well G-REX® culture system (Wilson Wolf) 48 hours later. After a total of 10 days in culture, SR-T cells were evaluated for rapamycin-dependent, antigen-dependent activity, as well as rapamycin-independent, antigen-independent background activity.

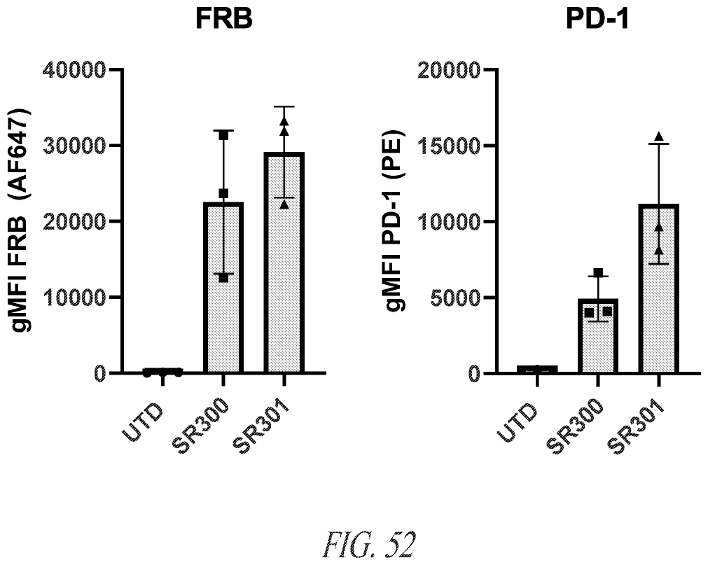

SR-T cells transduced with lentiviral vectors described above (SR300 and SR301) were analyzed for extracellular expression of key components of the rapamycin-inducible system by flow cytometry, including FRB multimerization domain and natural ligand, PD1 using anti-FRB and anti-PD1 antibodies, respectively. FRB and PD1 targeting components were highly expressed compared to control cells that were untransduced (FIG. 52).

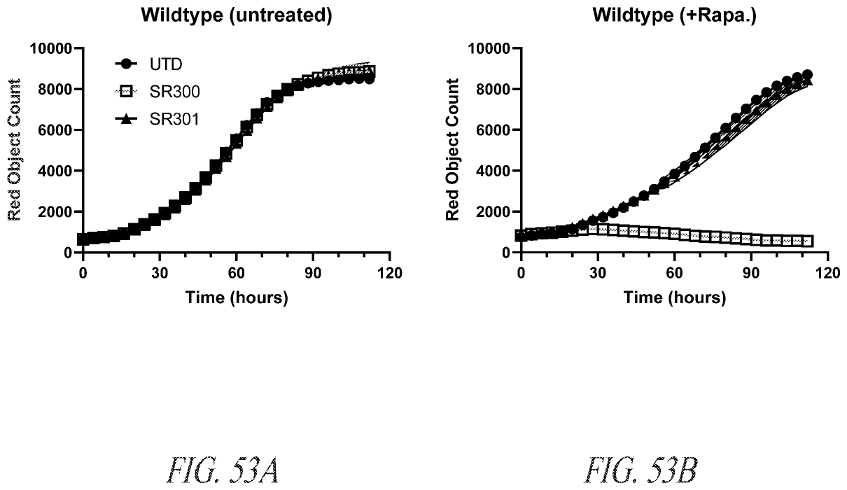

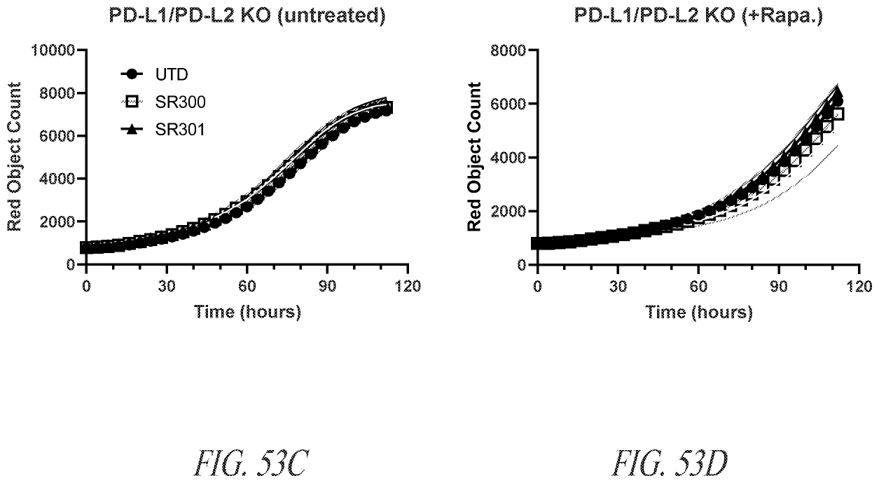

Antigen-specific cytotoxicity of SR300 and SR301-T cells was assessed by culturing a PDL1+ or PDL1/PDL2 knock-out target line that expresses a red nuclear fluorescent stain and tracking T cell growth over time using an Incucyte® live cell imaging system (Sartorius). Cells expressing nuclear stain (antigen positive target line) and cells not expressing nuclear stain (SR-T cells) were both tracked over approximately 115 hours. SR300 and SR301-T cells killed PDL1+ target cells only when rapamycin is present (FIGS. 53A-53D), but not when PDL1/PDL2 is knocked out. The high-affinity PD1-ectodomain (SR300-T cells) more efficiently killed PDL1+ target cells lines compared to natural PD1-ectodomain.

### Example 19

### Evaluation of Rapamycin-Inducible Fc Receptor NK Cells

In another experiment, NK cells expressing rapamycin-inducible Fc receptors (SR-NK cells) were generated using a 10-day transduction and expansion process, then evaluated for expression and biological activity against specific target antigens. Briefly, peripheral blood mononuclear cells (PBMCs) were activated with microbeads coated with anti-CD2 and anti-NKp46, then cultured in growth media containing human serum, HEPES, glutamine and a NK-specific cocktail of cytokines, including IL2, IL15, IL18, IL21 and IL12. Lentiviral vectors encoding rapamycin-inducible Fc epsilon receptor gamma with CD4 hinge, transmembrane and intracellular domains anchoring an FKBP12 or FRB multimerization domain and antigen targeting domains (SR354) were used to transduce the PBMCs two days after culture initiation. Cells were washed then transferred to a 24-well G-REX® culture system (Wilson Wolf) 48 hours later in NK growth medium containing IL2, IL15, IL18, IL21 and IL12. After a total of 10 days in culture, CD3⁺ T cells remaining in the culture were removed using magnetic bead sorting, resulting in a population of only NK cells. SR-NK cells were evaluated for rapamycin-dependent, antigen-dependent activity, as well as rapamycin-independent, antigen-independent background activity.

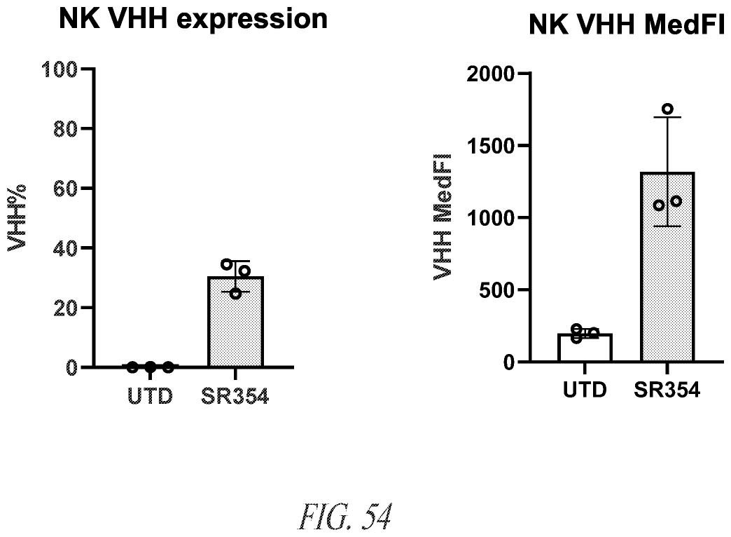

SR-NK cells transduced with lentiviral vectors described above (SR354) were analyzed for extracellular expression of key components of the rapamycin-inducible system by flow cytometry, including VHH targeting domain using an anti-VHH antibody. VHH targeting components were highly expressed compared to control cells that were untransduced (FIG. 54).

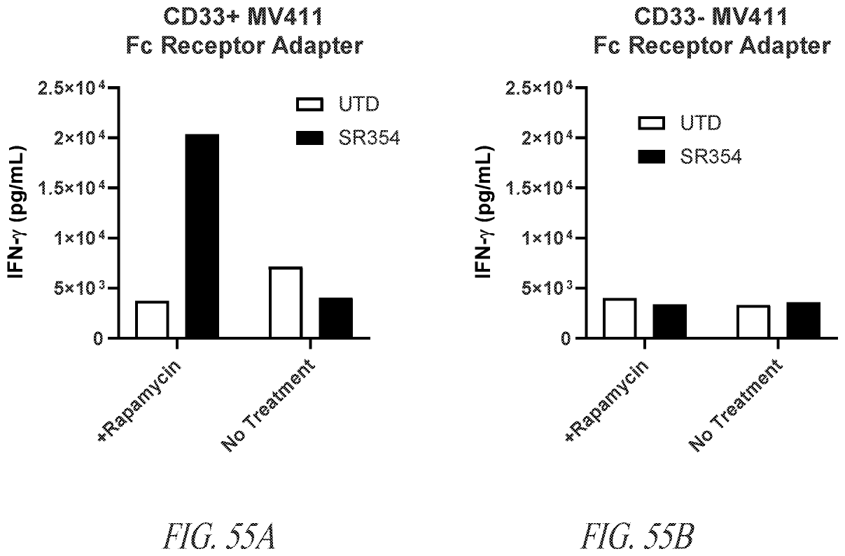

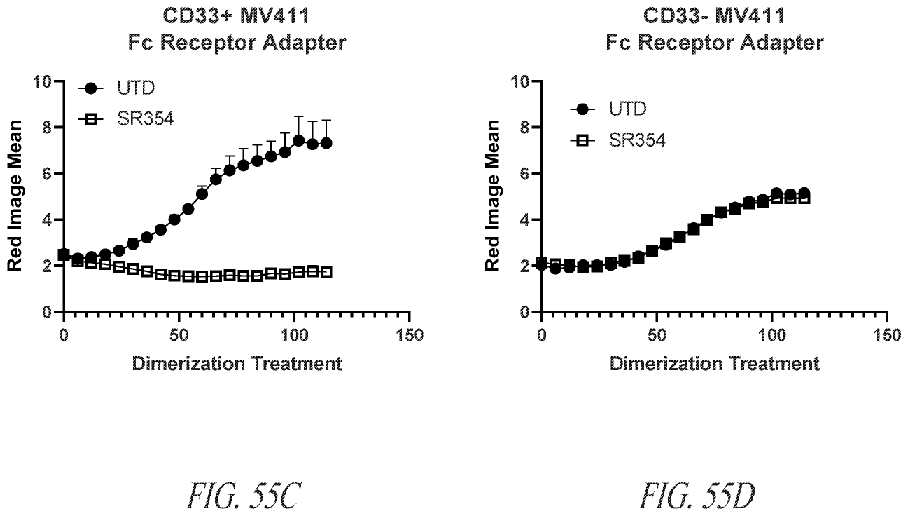

SR354- NK cell rapamycin-dependent, antigen-dependent activity was evaluated in co-cultures with target antigen positive tumor cells in the presence or absence of 1 nM rapamycin. Specifically, IFNγ production was quantified using MSD from supernatants of cultures with SR354-NK cells and cell lines that endogenously express CD33 or had CD33 knocked out. SR354-NK cells released cytokine at levels that were distinguishable from baseline (untransduced control) and were regulated completely by both rapamycin and antigen. (FIGS. 55A and 55B).

Antigen-specific cytotoxicity of SR354-NK cells was assessed by culturing a CD33+ or CD33 knocked-out target line that expresses a red nuclear fluorescent stain and tracking cytotoxicity over time using an Incucyte® live cell imaging system (Sartorius). Cells expressing nuclear stain (antigen positive target line) and cells not expressing nuclear stain (SR-NK cells) were both tracked over approximately 120 hours. SR354-NK cells killed CD33+ target cells only when rapamycin was present (FIGS. 55C and 55D), but not when CD33 is knocked out.

### Example 20

### Evaluation of Rapamycin-Inducible TCR T Cells Targeting ROR1

In another experiment, T cells expressing rapamycin-inducible T cell receptors (SR-T cells) were generated using a 10-day transduction and expansion process, then evaluated for expression and biological activity against specific target antigens. Briefly, peripheral blood mononuclear cells (PBMCs) were cultured in an IL-2 containing media and activated with antibodies against human CD3 and human CD28. Lentiviral vectors encoding rapamycin-inducible TCRs with CD4 hinge and transmembrane domains anchoring a FKBP12 multimerization domain and one antigen targeting domain (SR303) were used to transduce the PBMCs one day after culture initiation, then cells were transferred to a 24-well G-REX® culture system 48 hours later. After a total of 10 days in culture, SR-T cells were evaluated for rapamycin-dependent, antigen-dependent activity, as well as rapamycin-independent, antigen-independent background activity.

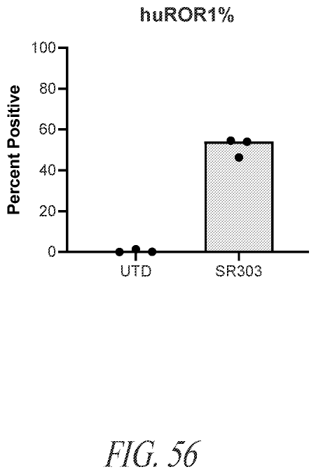

SR-T cells transduced with lentiviral vectors described above (SR303) were analyzed for extracellular expression of key components of the rapamycin-inducible system by flow cytometry, specifically the ROR1-targeted targeting domain, using an anti-ROR1 scFV. ROR1 scFv was highly expressed compared to control cells that were untransduced (FIG. 56).

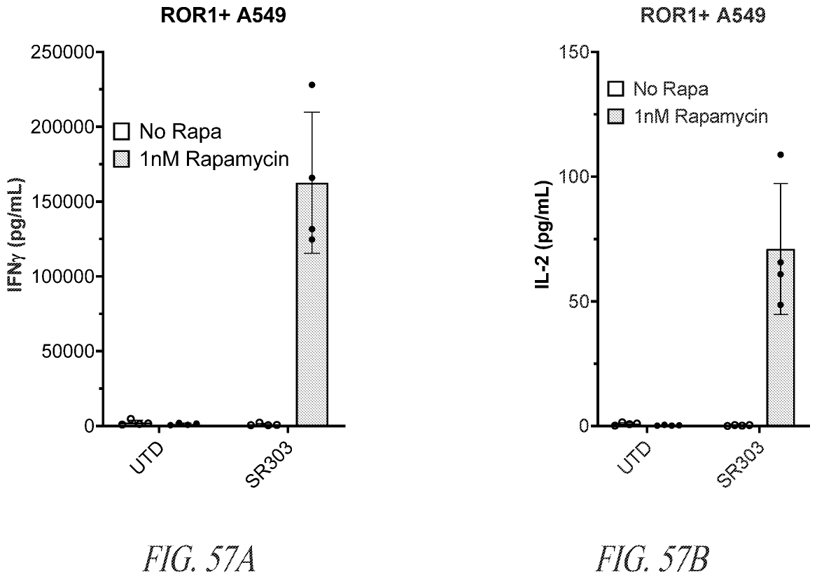

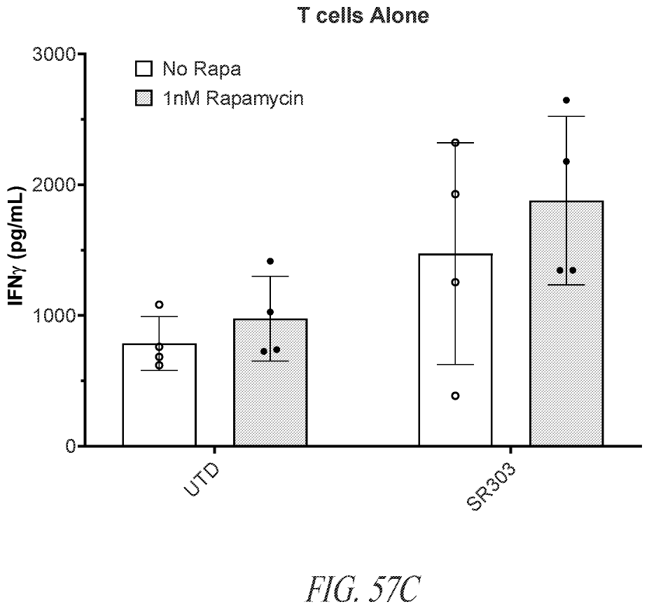

SR303-T cell rapamycin-dependent, antigen-dependent activity was evaluated in co-cultures with target antigen positive tumor cells in the presence of 1 nM rapamycin. Specifically, IFNγ and IL2 production was quantified using MSD from supernatants of cultures with SR303-T cells and a cell line endogenously expressing high levels of human ROR1 (FIGS. 57A and 57B). SR303-T cells released each of these 2 cytokines at levels that were highly distinguishable from baseline (untransduced control; UTD). Additionally, T cells were cultured alone with no antigen present, both without and in the presence of 1 nM of rapamycin. SR303-T cells produced minimal IFNγ when rapamycin was absent or when 1 nM of rapamycin was added with no antigen present (FIG. 57C).

### Example 21

### Illustrative Engineered Immune Receptor Constructs

Contemplated herein are engineered immune receptors comprising signaling and targeting components that can redirect signaling of either a native or engineered/exogenous multimeric immune receptor complex in response to a selected antigen. Signaling components contemplated herein comprise a multimerization domain, an optional linker, and an actuator domain. Targeting components contemplated herein comprise an extracellular domain comprising one or more targeting domains (also referred to herein as “binders” or “antigen binders”), a multimerization domain, an optional hinge domain, a transmembrane domain, and an optional intracellular domain (e.g., truncated intracellular domain, intracellular signaling domain, or costimulatory domain). The engineered immune receptor components and subunits/domains can be surprisingly combined to produce an engineered immune receptor having increased sensitivity over traditional CARs, low tonic or antigen-independent signaling, increased targeting capabilities, and optionally regulatability. In other words, the components can be combined without destroying the functionality of either the native or engineered/exogenous immune receptor complex or the targeting component(s) of the engineered immune receptor(s) described herein. Thus, the engineered immune receptors contemplated herein also surprisingly provide (1) multi-specificity, (2) increased sensitivity to non-MHC presented targets, (3) minimal tonic or antigen-independent signaling, and (4) the ability to simultaneously target both intracellular and extracellular targets.

Engineered immune receptors can be constructed in multiple formats and can be designed and constructed using known subunits/domains/polypeptides (e.g., multimerization domains, polypeptide linkers, actuator domains, targeting domains, hinge domains, transmembrane domains, and intracellular domains) and techniques. For example, a signaling component can be constructed by linking a multimerization domain (e.g., one or more “A” subunits) to an actuator domain (e.g., one or more “C” subunits) with or without one or more polypeptide linkers (e.g., with or without one or more “B” subunits) using standard cloning techniques.

Illustrative general signaling component formulas are provided below:

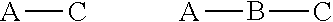

A targeting component can be constructed by linking an extracellular domain (e.g., one or more “D” subunits), to a multimerization domain (e.g., one or more “A” subunits), an optional hinge domain (e.g., one or more “E” subunits), a transmembrane domain (e.g., an “F” subunit), and an optional intracellular domain (e.g., one or more “G” subunits), with or without one or more polypeptide linkers (e.g., with or without one or more “B” subunits) using standard cloning techniques.

Illustrative general targeting component formulas are provided below:

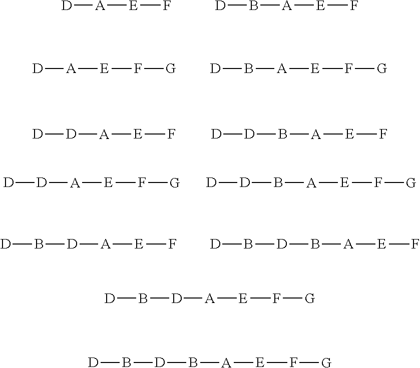

In various embodiments, both components (i.e., signaling and targeting) are expressed by a cell separately (e.g., from different polynucleotides, RNA, or DNA) or together as a fusion protein from a single polynucleotide, RNA or DNA. The engineered immune receptors contemplated herein can be designed and constructed using known subunits/domains/polypeptides (e.g., multimerization domains, polypeptide linkers, actuator domains, targeting domains, hinge domains, transmembrane domains, and intracellular domains) and techniques.

Table 3 provides an illustrative list of known multimerization domains. Table 4 provides an illustrative list of known polypeptide linkers. Table 5 provides an illustrative list of known actuators. Table 6 provides an illustrative list of known targeting domains. Table 7 provides an illustrative list of known hinge domains. Table 8 provides an illustrative list of known transmembrane domains. Table 9 provides an illustrative list of known intracellular domains. However, other known multimerization domains, polypeptide linkers, actuator domains, targeting domains, hinge domains, transmembrane domains, intracellular domains, and TCRs can be found throughout the literature, e.g., including but not limited to US20120082661, WO2016014789, WO2022046730, WO2016033570, U.S. Pat. No. 8,147,832B2, WO2014026054, WO2018145649, WO2014065961, WO2020123947, WO2013049254, WO2019241685, WO2019241688, WO2016049214, WO2018236870, WO2020102240, WO2018183888, U.S. Pat. No. 6,217,866B1, WO2008119566, WO2003055917, WO2018073680, WO2014146672, WO2019200007, WO2016016859, WO2018119279, WO2020227072, WO2020227073, WO2020227071, WO2017153402, WO2007042289, WO2018028647, WO2005113595, US20180273602, WO2019067242, WO2020193767, U.S. Ser. No. 10/538,572B2, U.S. Ser. No. 11/078,252B2, WO2019140100, WO2015009606, WO2021195503, WO2007131092, US20190169260, WO2012045085, WO2016115559, and WO2016187220, each of which are incorporated by reference herein, in their entirety. Since other known multimerization domains, polypeptide linkers, actuator domains, targeting domains, hinge domains, transmembrane domains, intracellular domains, and TCRs are well known in the literature, the invention is not intended to be limited to the illustrative components disclosed in Tables 3-9.

TABLE 3
Illustrative Multimerization Domains (“A” Subunits):

| SUBUNIT | | |
|---|---|---|
| REFERENCE | SUBUNIT NAME | SEQ ID NO: |
| A1 | FRB* | 1 |
| A2 | FRB | 2 |
| A3 | FKBP12 | 3 |
| A4 | FKBP12 F36V | 4 |
| A5 | CH3_AB (S354C T366W) | 5 |
| A6 | CH3_IA (Y349C T366S L368A Y407V) | 6 |
| A7 | CH3_DE (L351D L368E) | 7 |
| A8 | CH3_KK (L351K T366K) | 8 |
| A9 | CH3_GA (SEED 1) | 9 |
| A10 | CH3_AG (SEED 2) | 10 |

TABLE 4
Illustrative Polypeptide Linkers (“B” Subunits):

| SUBUNIT | | SEQ |
|---|---|---|
| REFERENCE | SEQUENCE/NAME | ID NO: |
| B1 | GGGGS (G4S; 1xG4S) | 11 |
| B2 | GGGGSGGGGS (2xG4S) | 12 |
| B3 | GGGGSGGGGSGGGGS (3xG4S) | 13 |
| B4 | GGGGSGGGGSGGGGSGGGGS (4xG4S) | 14 |
| B5 | GGGGSGGGGSGGGGSGGGGSGGGGS | 15 |
| | (5xG4S) | |
| B6 | DGGGS | 16 |
| B7 | TGEKP | 17 |
| B8 | GGRR | 18 |
| B9 | EGKSSGSGSESKVD | 19 |
| B10 | KESGSVSSEQLAQFRSLD | 20 |
| B11 | GGRRGGGS | 21 |
| B12 | LRQRDGERP | 22 |
| B13 | LRQKDGGGSERP | 23 |
| B14 | LRQKDGGGSGGGSERP | 24 |
| B15 | GSTSGSGKPGSGEGSTKG | 25 |
| B16 | GSTSGSGKSSEGSGSTKG | 26 |
| B17 | GSTSGSGKSSEGKG | 27 |
| B18 | GSTSGSGKPGSGEGS | 28 |
| B19 | GGGS | 29 |
| B20 | LEKT (yuTCR linker; uLNK) | 30 |
| B21 | LEKTGGGGS (uLNK + G4S linker) | 31 |
| B22 | none | |

TABLE 5
Illustrative actuators (“C” Subunits):

| SUBUNIT | | |
|---|---|---|
| REFERENCE | SUBUNIT NAME | SEQ ID NO: |
| C1 | CD3ε | 55 |
| C2 | CD3γ | 57 |
| C3 | CD3δ | 59 |
| C4 | FcεR1γ v1 | 61 |
| C5 | FcεR1γ v2 | 63 |
| C6 | Igα/CD79a (BCR) | 65 |
| C7 | Igβ/CD79b (BCR) | 67 |
| C8 | DAP10 | 69 |
| C9 | DAP12 | 71 |

TABLE 6
Illustrative targeting domains (“D” Subunits):

| SUBUNIT | | | |
|---|---|---|---|
| REFERENCE | TARGET/DOMAIN | TYPE | SEQ ID NO: |
| D1 | BCMA | SCFV | 50, 51, OR 52 |
| D2 | CD19 | SCFV | 53 |
| D3 | CD20 | SCFV | 54, 55, OR 56 |
| D4 | CD22 | SCFV | 57 |
| D5 | CD33 | SCFV | 58 |
| D6 | CD79A | SCFV | 59 |
| D7 | CD79B | SCFV | 60 OR 61 |
| D8 | B7H3 | SCFV | 62 |
| D9 | MUC16 | SCFV | 63 |
| D10 | HER2 | SCFV | 64 |
| D11 | EGFR | SCFV | 65 |
| D12 | FN-EDB | SCFV | 66 |
| D13 | CLDN18.2 | SCFV | 67 |
| D14 | DLL3 | SCFV | 68 |
| D15 | FLT3 | SCFV | 69 OR 70 |
| D16 | ROR1 | SCFV | 71 |
| D17 | CD33 | VHH | 72, 73, OR 74 |
| D18 | CLL1 | VHH | 75 OR 76 |
| D19 | CD123 | VHH | 77 OR 78 |
| D20 | CD20 | VHH | 79 |
| D21 | EGFR | VHH | 80 |
| D22 | BCMA | VHH | 81 OR 82 |
| D23 | CD19 | VHH | 83 |
| D24 | PD-L1/PD1 | ECTO | 84 OR 85 |
| D25 | BCMA/APRIL | LIGAND | 86 OR 87 |
| D26 | NKG2DLS/NKG2D | ECTO | 88 |
| D27 | CD33 | CDRS | 89, 90, AND 91 |
| D28 | CLL1 | CDRS | 92, 93, AND 94 |

TABLE 7
Illustrative Hinge Domains (“E” Subunits):

| SUBUNIT | | |
|---|---|---|
| REFERENCE | SUBUNIT NAME | SEQ ID NO: |
| E1 | Minimal CD4 hinge | 41 |
| E2 | Minimal CD28 hinge | 42 |
| E3 | IgG4 hinge | 43 |
| E4 | CD8 hinge | 44 |
| E5 | Hinge linker; GGR | |

TABLE 8
Illustrative Transmembrane Domains (“F” Subunits):

| SUBUNIT | | |
|---|---|---|
| REFERENCE | SUBUNIT NAME | SEQ ID NO: |
| F1 | CD4 transmembrane | 45 |
| F2 | CD28 transmembrane | 46 |
| F3 | CD8 transmembrane | 47 |

TABLE 9
Illustrative Intracellular Domains (“G” Subunits):

| SUBUNIT | | SEQ |
|---|---|---|
| REFERENCE | SUBUNIT NAME | ID NO: |
| G1 | Truncated intracellular CD4 - v1 | 48 |
| G2 | Truncated intracellular CD4 - v2 | 49 |
| G3 | 41BB intracellular signaling domain | 98 |
| G4 | CD28 intracellular signaling domain | 99 |
| G5 | CD4 coreceptor domain | 100 |
| G6 | CD8 coreceptor domain | 101 |
| G7 | LAT domain | 102 |
| G8 | IL7 receptor alpha signaling domain | 103 |
| G9 | IL2 receptor beta signaling domain, truncated | 104 |
| G10 | IL2 receptor beta signaling domain | 105 |
| G11 | Common gamma chain signaling domain | 106 |
| G12 | none | |

As one example of a signaling component, multimerization domain that pairs with a multimerization domain from a corresponding targeting component from Table 3 (e.g., an A1 subunit) can be combined with one or more polypeptide linkers from Table 4 (e.g., a B3 subunit) and an actuator domain from Table 5 (e.g., a C1 subunit), to produce a novel signaling component (e.g., a signaling component as shown in SEQ ID NO: 111). Other illustrative signaling components are shown in SEQ ID NOs: 107-110 and 112-115.

As one example of a targeting component, an extracellular or one or more targeting domains from Table 6 (e.g., a D17 subunit), can be combined with one or more optional polypeptide linkers from Table 4 (e.g., a B1 subunit), a multimerization domain that pairs with a multimerization domain from a corresponding signaling component from Table 3 (e.g., an A3 subunit), a hinge domain from Table 7 (e.g., an E1 subunit), a transmembrane domain from Table 8 (e.g., an F1 subunit), and an intracellular domain from Table 9 (e.g., a G2 subunit), to produce a novel targeting component (e.g., a targeting component as shown in SEQ ID NO: 117). Other illustrative targeting components are shown in SEQ ID NOs: 116 and 118-136.

Additionally, as further shown and contemplated herein, multiple “A” components can be combined to produce multi-specific extracellular domains (e.g., tandem targeting domains), and multiple polypeptide linkers can be combined to produce functional linkers.

Moreover, one of skill in the art would understand that a signal sequence may be linked to the N-terminus of the signaling and/or targeting components to enable efficient trafficking to the cell membrane compartment. Accordingly, in various embodiments, any of the signaling and targeting components disclosed herein comprise a signaling sequence. In some embodiments, the signaling sequence is a IgK, CD8, or PD1 signal sequence. In some embodiments, the signaling sequence is a IgK, CD8, or PD1 signal sequence as set forth in any one of SEQ ID NOs: 95-97.

Tables 10 and 11 provide an illustrative, non-limiting list of signaling and targeting components, respectively, based on the multimerization domains, polypeptide linkers, actuator domains, targeting domains, hinge domains, transmembrane domains, and intracellular domains provided in Tables 3-9.

TABLE 10
Illustrative Signaling Components:

| SIGNALING | “A” | “B” | “C” |
|---|---|---|---|
| COMPONENT # | SUBUNIT | SUBUNIT | SUBUNIT |
| 1 | A1 | B1 | C1 |
| 2 | A1 | B1 | C2 |
| 3 | A1 | B1 | C3 |
| 4 | A1 | B1 | C4 |
| 5 | A1 | B1 | C5 |
| 6 | A1 | B1 | C6 |
| 7 | A1 | B1 | C7 |
| 8 | A1 | B1 | C8 |
| 9 | A1 | B1 | C9 |
| 10 | A1 | B2 | C1 |
| 11 | A1 | B2 | C2 |
| 12 | A1 | B2 | C3 |
| 13 | A1 | B2 | C4 |
| 14 | A1 | B2 | C5 |
| 15 | A1 | B2 | C6 |
| 16 | A1 | B2 | C7 |
| 17 | A1 | B2 | C8 |
| 18 | A1 | B2 | C9 |
| 19 | A1 | B3 | C1 |
| 20 | A1 | B3 | C2 |
| 21 | A1 | B3 | C3 |
| 22 | A1 | B3 | C4 |
| 23 | A1 | B3 | C5 |
| 24 | A1 | B3 | C6 |
| 25 | A1 | B3 | C7 |
| 26 | A1 | B3 | C8 |
| 27 | A1 | B3 | C9 |
| 28 | A1 | B4 | C1 |
| 29 | A1 | B4 | C2 |
| 30 | A1 | B4 | C3 |
| 31 | A1 | B4 | C4 |
| 32 | A1 | B4 | C5 |
| 33 | A1 | B4 | C6 |
| 34 | A1 | B4 | C7 |
| 35 | A1 | B4 | C8 |
| 36 | A1 | B4 | C9 |
| 37 | A1 | ANY ONE OF B4-B22 | C1 |
| 38 | A1 | ANY ONE OF B4-B22 | C2 |
| 39 | A1 | ANY ONE OF B4-B22 | C3 |
| 40 | A1 | ANY ONE OF B4-B22 | C4 |
| 41 | A1 | ANY ONE OF B4-B22 | C5 |
| 42 | A1 | ANY ONE OF B4-B22 | C6 |
| 43 | A1 | ANY ONE OF B4-B22 | C7 |
| 44 | A1 | ANY ONE OF B4-B22 | C8 |
| 45 | A1 | ANY ONE OF B4-B22 | C9 |
| 46 | A2 | B1 | C1 |
| 47 | A2 | B1 | C2 |
| 48 | A2 | B1 | C3 |
| 49 | A2 | B1 | C4 |
| 50 | A2 | B1 | C5 |
| 51 | A2 | B1 | C6 |
| 52 | A2 | B1 | C7 |
| 53 | A2 | B1 | C8 |
| 54 | A2 | B1 | C9 |
| 55 | A2 | B2 | C1 |
| 56 | A2 | B2 | C2 |
| 57 | A2 | B2 | C3 |
| 58 | A2 | B2 | C4 |
| 59 | A2 | B2 | C5 |
| 60 | A2 | B2 | C6 |
| 61 | A2 | B2 | C7 |
| 62 | A2 | B2 | C8 |
| 63 | A2 | B2 | C9 |
| 64 | A2 | B3 | C1 |
| 65 | A2 | B3 | C2 |
| 66 | A2 | B3 | C3 |
| 67 | A2 | B3 | C4 |
| 68 | A2 | B3 | C5 |
| 69 | A2 | B3 | C6 |
| 70 | A2 | B3 | C7 |
| 71 | A2 | B3 | C8 |
| 72 | A2 | B3 | C9 |
| 73 | A2 | ANY ONE OF B4-B22 | C1 |
| 74 | A2 | ANY ONE OF B4-B22 | C2 |
| 75 | A2 | ANY ONE OF B4-B22 | C3 |
| 76 | A2 | ANY ONE OF B4-B22 | C4 |
| 77 | A2 | ANY ONE OF B4-B22 | C5 |
| 78 | A2 | ANY ONE OF B4-B22 | C6 |
| 79 | A2 | ANY ONE OF B4-B22 | C7 |
| 80 | A2 | ANY ONE OF B4-B22 | C8 |
| 81 | A2 | ANY ONE OF B4-B22 | C9 |
| 82 | A3 | B1 | C1 |
| 83 | A3 | B1 | C2 |
| 84 | A3 | B1 | C3 |
| 85 | A3 | B1 | C4 |
| 86 | A3 | B1 | C5 |
| 87 | A3 | B1 | C6 |
| 88 | A3 | B1 | C7 |
| 89 | A3 | B1 | C8 |
| 90 | A3 | B1 | C9 |
| 91 | A3 | B2 | C1 |
| 92 | A3 | B2 | C2 |
| 93 | A3 | B2 | C3 |
| 94 | A3 | B2 | C4 |
| 95 | A3 | B2 | C5 |
| 96 | A3 | B2 | C6 |
| 97 | A3 | B2 | C7 |
| 98 | A3 | B2 | C8 |
| 99 | A3 | B2 | C9 |
| 100 | A3 | B3 | C1 |
| 101 | A3 | B3 | C2 |
| 102 | A3 | B3 | C3 |
| 103 | A3 | B3 | C4 |
| 104 | A3 | B3 | C5 |
| 105 | A3 | B3 | C6 |
| 106 | A3 | B3 | C7 |
| 107 | A3 | B3 | C8 |
| 108 | A3 | B3 | C9 |
| 109 | A3 | ANY ONE OF B4-B22 | C1 |
| 110 | A3 | ANY ONE OF B4-B22 | C2 |
| 111 | A3 | ANY ONE OF B4-B22 | C3 |
| 112 | A3 | ANY ONE OF B4-B22 | C4 |
| 113 | A3 | ANY ONE OF B4-B22 | C5 |
| 114 | A3 | ANY ONE OF B4-B22 | C6 |
| 115 | A3 | ANY ONE OF B4-B22 | C7 |
| 116 | A3 | ANY ONE OF B4-B22 | C8 |
| 117 | A3 | ANY ONE OF B4-B22 | C9 |
| 118 | A4 | B1 | C1 |
| 119 | A4 | B1 | C2 |
| 120 | A4 | B1 | C3 |
| 121 | A4 | B1 | C4 |
| 122 | A4 | B1 | C5 |
| 123 | A4 | B1 | C6 |
| 124 | A4 | B1 | C7 |
| 125 | A4 | B1 | C8 |
| 126 | A4 | B1 | C9 |
| 127 | A4 | B2 | C1 |
| 128 | A4 | B2 | C2 |
| 129 | A4 | B2 | C3 |
| 130 | A4 | B2 | C4 |
| 131 | A4 | B2 | C5 |
| 132 | A4 | B2 | C6 |
| 133 | A4 | B2 | C7 |
| 134 | A4 | B2 | C8 |
| 135 | A4 | B2 | C9 |
| 136 | A4 | B3 | C1 |
| 137 | A4 | B3 | C2 |
| 138 | A4 | B3 | C3 |
| 139 | A4 | B3 | C4 |
| 140 | A4 | B3 | C5 |
| 141 | A4 | B3 | C6 |
| 142 | A4 | B3 | C7 |
| 143 | A4 | B3 | C8 |
| 144 | A4 | B3 | C9 |
| 145 | A4 | ANY ONE OF B4-B22 | C1 |
| 146 | A4 | ANY ONE OF B4-B22 | C2 |
| 147 | A4 | ANY ONE OF B4-B22 | C3 |
| 148 | A4 | ANY ONE OF B4-B22 | C4 |
| 149 | A4 | ANY ONE OF B4-B22 | C5 |
| 150 | A4 | ANY ONE OF B4-B22 | C6 |
| 151 | A4 | ANY ONE OF B4-B22 | C7 |
| 152 | A4 | ANY ONE OF B4-B22 | C8 |
| 153 | A4 | ANY ONE OF B4-B22 | C9 |
| 154 | A5 | B1 | C1 |
| 155 | A5 | B1 | C2 |
| 156 | A5 | B1 | C3 |
| 157 | A5 | B1 | C4 |
| 158 | A5 | B1 | C5 |
| 159 | A5 | B1 | C6 |
| 160 | A5 | B1 | C7 |
| 161 | A5 | B1 | C8 |
| 162 | A5 | B1 | C9 |
| 163 | A5 | B2 | C1 |
| 164 | A5 | B2 | C2 |
| 165 | A5 | B2 | C3 |
| 166 | A5 | B2 | C4 |
| 167 | A5 | B2 | C5 |
| 168 | A5 | B2 | C6 |
| 169 | A5 | B2 | C7 |
| 170 | A5 | B2 | C8 |
| 171 | A5 | B2 | C9 |
| 172 | A5 | B3 | C1 |
| 173 | A5 | B3 | C2 |
| 174 | A5 | B3 | C3 |
| 175 | A5 | B3 | C4 |
| 176 | A5 | B3 | C5 |
| 177 | A5 | B3 | C6 |
| 178 | A5 | B3 | C7 |
| 179 | A5 | B3 | C8 |
| 180 | A5 | B3 | C9 |
| 181 | A5 | ANY ONE OF B4-B22 | C1 |
| 182 | A5 | ANY ONE OF B4-B22 | C2 |
| 183 | A5 | ANY ONE OF B4-B22 | C3 |
| 184 | A5 | ANY ONE OF B4-B22 | C4 |
| 185 | A5 | ANY ONE OF B4-B22 | C5 |
| 186 | A5 | ANY ONE OF B4-B22 | C6 |
| 187 | A5 | ANY ONE OF B4-B22 | C7 |
| 188 | A5 | ANY ONE OF B4-B22 | C8 |
| 189 | A5 | ANY ONE OF B4-B22 | C9 |
| 190 | A6 | B1 | C1 |
| 191 | A6 | B1 | C2 |
| 192 | A6 | B1 | C3 |
| 193 | A6 | B1 | C4 |
| 194 | A6 | B1 | C5 |
| 195 | A6 | B1 | C6 |
| 196 | A6 | B1 | C7 |
| 197 | A6 | B1 | C8 |
| 198 | A6 | B1 | C9 |
| 199 | A6 | B2 | C1 |
| 200 | A6 | B2 | C2 |
| 201 | A6 | B2 | C3 |
| 202 | A6 | B2 | C4 |
| 203 | A6 | B2 | C5 |
| 204 | A6 | B2 | C6 |
| 205 | A6 | B2 | C7 |
| 206 | A6 | B2 | C8 |
| 207 | A6 | B2 | C9 |
| 208 | A6 | B3 | C1 |
| 209 | A6 | B3 | C2 |
| 210 | A6 | B3 | C3 |
| 211 | A6 | B3 | C4 |
| 212 | A6 | B3 | C5 |
| 213 | A6 | B3 | C6 |
| 214 | A6 | B3 | C7 |
| 215 | A6 | B3 | C8 |
| 216 | A6 | B3 | C9 |
| 217 | A6 | ANY ONE OF B4-B22 | C1 |
| 218 | A6 | ANY ONE OF B4-B22 | C2 |
| 219 | A6 | ANY ONE OF B4-B22 | C3 |
| 220 | A6 | ANY ONE OF B4-B22 | C4 |
| 221 | A6 | ANY ONE OF B4-B22 | C5 |
| 222 | A6 | ANY ONE OF B4-B22 | C6 |
| 223 | A6 | ANY ONE OF B4-B22 | C7 |
| 224 | A6 | ANY ONE OF B4-B22 | C8 |
| 225 | A6 | ANY ONE OF B4-B22 | C9 |
| 226 | A7 | B1 | C1 |
| 227 | A7 | B1 | C2 |
| 228 | A7 | B1 | C3 |
| 229 | A7 | B1 | C4 |
| 230 | A7 | B1 | C5 |
| 231 | A7 | B1 | C6 |
| 232 | A7 | B1 | C7 |
| 233 | A7 | B1 | C8 |
| 234 | A7 | B1 | C9 |
| 235 | A7 | B2 | C1 |
| 236 | A7 | B2 | C2 |
| 237 | A7 | B2 | C3 |
| 238 | A7 | B2 | C4 |
| 239 | A7 | B2 | C5 |
| 240 | A7 | B2 | C6 |
| 241 | A7 | B2 | C7 |
| 242 | A7 | B2 | C8 |
| 243 | A7 | B2 | C9 |
| 244 | A7 | B3 | C1 |
| 245 | A7 | B3 | C2 |
| 246 | A7 | B3 | C3 |
| 247 | A7 | B3 | C4 |
| 248 | A7 | B3 | C5 |
| 249 | A7 | B3 | C6 |
| 250 | A7 | B3 | C7 |
| 251 | A7 | B3 | C8 |
| 252 | A7 | B3 | C9 |
| 253 | A7 | ANY ONE OF B4-B22 | C1 |
| 254 | A7 | ANY ONE OF B4-B22 | C2 |
| 255 | A7 | ANY ONE OF B4-B22 | C3 |
| 256 | A7 | ANY ONE OF B4-B22 | C4 |
| 257 | A7 | ANY ONE OF B4-B22 | C5 |
| 258 | A7 | ANY ONE OF B4-B22 | C6 |
| 259 | A7 | ANY ONE OF B4-B22 | C7 |
| 260 | A7 | ANY ONE OF B4-B22 | C8 |
| 261 | A7 | ANY ONE OF B4-B22 | C9 |
| 262 | A8 | B1 | C1 |
| 263 | A8 | B1 | C2 |
| 264 | A8 | B1 | C3 |
| 265 | A8 | B1 | C4 |
| 266 | A8 | B1 | C5 |
| 267 | A8 | B1 | C6 |
| 268 | A8 | B1 | C7 |
| 269 | A8 | B1 | C8 |
| 270 | A8 | B1 | C9 |
| 271 | A8 | B2 | C1 |
| 272 | A8 | B2 | C2 |
| 273 | A8 | B2 | C3 |
| 274 | A8 | B2 | C4 |
| 275 | A8 | B2 | C5 |
| 276 | A8 | B2 | C6 |
| 277 | A8 | B2 | C7 |
| 278 | A8 | B2 | C8 |
| 279 | A8 | B2 | C9 |
| 280 | A8 | B3 | C1 |
| 281 | A8 | B3 | C2 |
| 282 | A8 | B3 | C3 |
| 283 | A8 | B3 | C4 |
| 284 | A8 | B3 | C5 |
| 285 | A8 | B3 | C6 |
| 286 | A8 | B3 | C7 |
| 287 | A8 | B3 | C8 |
| 288 | A8 | B3 | C9 |
| 289 | A8 | ANY ONE OF B4-B22 | C1 |
| 290 | A8 | ANY ONE OF B4-B22 | C2 |
| 291 | A8 | ANY ONE OF B4-B22 | C3 |
| 292 | A8 | ANY ONE OF B4-B22 | C4 |
| 293 | A8 | ANY ONE OF B4-B22 | C5 |
| 294 | A8 | ANY ONE OF B4-B22 | C6 |
| 295 | A8 | ANY ONE OF B4-B22 | C7 |
| 296 | A8 | ANY ONE OF B4-B22 | C8 |
| 297 | A8 | ANY ONE OF B4-B22 | C9 |
| 298 | A9 | B1 | C1 |
| 299 | A9 | B1 | C2 |
| 300 | A9 | B1 | C3 |
| 301 | A9 | B1 | C4 |
| 302 | A9 | B1 | C5 |
| 303 | A9 | B1 | C6 |
| 304 | A9 | B1 | C7 |
| 305 | A9 | B1 | C8 |
| 306 | A9 | B1 | C9 |
| 307 | A9 | B2 | C1 |
| 308 | A9 | B2 | C2 |
| 309 | A9 | B2 | C3 |
| 310 | A9 | B2 | C4 |
| 311 | A9 | B2 | C5 |
| 312 | A9 | B2 | C6 |
| 313 | A9 | B2 | C7 |
| 314 | A9 | B2 | C8 |
| 315 | A9 | B2 | C9 |
| 316 | A9 | B3 | C1 |
| 317 | A9 | B3 | C2 |
| 318 | A9 | B3 | C3 |
| 319 | A9 | B3 | C4 |
| 320 | A9 | B3 | C5 |
| 321 | A9 | B3 | C6 |
| 322 | A9 | B3 | C7 |
| 323 | A9 | B3 | C8 |
| 324 | A9 | B3 | C9 |
| 325 | A9 | ANY ONE OF B4-B22 | C1 |
| 326 | A9 | ANY ONE OF B4-B22 | C2 |
| 327 | A9 | ANY ONE OF B4-B22 | C3 |
| 328 | A9 | ANY ONE OF B4-B22 | C4 |
| 329 | A9 | ANY ONE OF B4-B22 | C5 |
| 330 | A9 | ANY ONE OF B4-B22 | C6 |
| 331 | A9 | ANY ONE OF B4-B22 | C7 |
| 332 | A9 | ANY ONE OF B4-B22 | C8 |
| 333 | A9 | ANY ONE OF B4-B22 | C9 |
| 334 | A10 | B1 | C1 |
| 335 | A10 | B1 | C2 |
| 336 | A10 | B1 | C3 |
| 337 | A10 | B1 | C4 |
| 338 | A10 | B1 | C5 |
| 339 | A10 | B1 | C6 |
| 340 | A10 | B1 | C7 |
| 341 | A10 | B1 | C8 |
| 342 | A10 | B1 | C9 |
| 343 | A10 | B2 | C1 |
| 344 | A10 | B2 | C2 |
| 345 | A10 | B2 | C3 |
| 346 | A10 | B2 | C4 |
| 347 | A10 | B2 | C5 |
| 348 | A10 | B2 | C6 |
| 349 | A10 | B2 | C7 |
| 350 | A10 | B2 | C8 |
| 351 | A10 | B2 | C9 |
| 352 | A10 | B3 | C1 |
| 353 | A10 | B3 | C2 |
| 354 | A10 | B3 | C3 |
| 355 | A10 | B3 | C4 |
| 356 | A10 | B3 | C5 |
| 357 | A10 | B3 | C6 |
| 358 | A10 | B3 | C7 |
| 359 | A10 | B3 | C8 |
| 360 | A10 | B3 | C9 |
| 361 | A10 | ANY ONE OF B4-B22 | C1 |
| 362 | A10 | ANY ONE OF B4-B22 | C2 |
| 363 | A10 | ANY ONE OF B4-B22 | C3 |
| 364 | A10 | ANY ONE OF B4-B22 | C4 |
| 365 | A10 | ANY ONE OF B4-B22 | C5 |
| 366 | A10 | ANY ONE OF B4-B22 | C6 |
| 367 | A10 | ANY ONE OF B4-B22 | C7 |
| 368 | A10 | ANY ONE OF B4-B22 | C8 |
| 369 | A10 | ANY ONE OF B4-B22 | C9 |

TABLE 11
Illustrative Targeting Components:

| TARGETING | | “B” | “A” | “E” | “F” | “G” |
|---|---|---|---|---|---|---|
| COMPONENT # | “D” SUBUNIT | SUBUNIT | SUBUNIT | SUBUNIT | SUBUNIT | SUBUNIT |
| 1 | ANY ONE OR | B1 | A1 | E1 | F1 | G1 |
| | MORE OF | | | | | |
| | D1-D28 | | | | | |
| 2 | ANY ONE OR | B1 | A1 | E1 | F1 | G2 |
| | MORE OF | | | | | |
| | D1-D28 | | | | | |
| 3 | ANY ONE OR | B1 | A1 | E1 | F1 | ANY ONE |
| | MORE OF | | | | | OF G3- |
| | D1-D28 | | | | | G12 |
| 4 | ANY ONE OR | B1 | A1 | E1 | F2 | G1 |
| | MORE OF | | | | | |
| | D1-D28 | | | | | |
| 5 | ANY ONE OR | B1 | A1 | E1 | F2 | G2 |
| | MORE OF D1- | | | | | |
| | D28 | | | | | |
| 6 | ANY ONE OR | B1 | A1 | E1 | F2 | ANY ONE |
| | MORE OF D1- | | | | | OF G3- |
| | D28 | | | | | G12 |
| 7 | ANY ONE OR | B1 | A1 | E1 | F3 | G1 |
| | MORE OF D1- | | | | | |
| | D28 | | | | | |
| 8 | ANY ONE OR | B1 | A1 | E1 | F3 | G2 |
| | MORE OF D1- | | | | | |
| | D28 | | | | | |
| 9 | ANY ONE OR | B1 | A1 | E1 | F3 | ANY ONE |
| | MORE OF D1- | | | | | OF G3- |
| | D28 | | | | | G12 |
| 10 | ANY ONE OR | B1 | A1 | ANY ONE OF | F1 | G1 |
| | MORE OF D1- | | | E2-E4 | | |
| | D28 | | | | | |
| 11 | ANY ONE OR | B1 | A1 | ANY ONE OF | F1 | G2 |
| | MORE OF D1- | | | E2-E4 | | |
| | D28 | | | | | |
| 12 | ANY ONE OR | B1 | A1 | ANY ONE OF | F1 | ANY ONE |
| | MORE OF D1- | | | E2-E4 | | OF G3- |
| | D28 | | | | | G12 |
| 13 | ANY ONE OR | B1 | A1 | ANY ONE OF E2-E4 | F2 | G1 |
| | MORE OF D1- | | | | | |
| | D28 | | | | | |
| 14 | ANY ONE OR | B1 | A1 | ANY ONE OF E2-E4 | F2 | G2 |
| | MORE OF D1- | | | | | |
| | D28 | | | | | |
| 15 | ANY ONE OR | B1 | A1 | ANY ONE OF | F2 | ANY ONE |
| | MORE OF D1- | | | E2-E4 | | OF G3- |
| | D28 | | | | | G12 |
| 16 | ANY ONE OR | B1 | A1 | ANY ONE OF | F3 | G1 |
| | MORE OF D1- | | | E2-E4 | | |
| | D28 | | | | | |
| 17 | ANY ONE OR | B1 | A1 | ANY ONE OF E2-E4 | F3 | G2 |
| | MORE OF D1- | | | | | |
| | D28 | | | | | |
| 18 | ANY ONE OR | B1 | A1 | ANY ONE OF | F3 | ANY ONE |
| | MORE OF D1- | | | E2-E4 | | OF G3- |
| | D28 | | | | | G12 |
| 19 | ANY ONE OR | B1 | A2 | E1 | F1 | G1 |
| | MORE OF D1- | | | | | |
| | D28 | | | | | |
| 20 | ANY ONE OR | B1 | A2 | E1 | F1 | G2 |
| | MORE OF D1- | | | | | |
| | D28 | | | | | |
| 21 | ANY ONE OR | B1 | A2 | E1 | F1 | ANY ONE |
| | MORE OF D1- | | | | | OF G3- |
| | D28 | | | | | G12 |
| 22 | ANY ONE OR | B1 | A2 | E1 | F2 | G1 |
| | MORE OF D1- | | | | | |
| | D28 | | | | | |
| 23 | ANY ONE OR | B1 | A2 | E1 | F2 | G2 |
| | MORE OF D1- | | | | | |
| | D28 | | | | | |
| 24 | ANY ONE OR | B1 | A2 | E1 | F2 | ANY ONE |
| | MORE OF D1- | | | | | OF G3- |
| | D28 | | | | | G12 |
| 25 | ANY ONE OR | B1 | A2 | E1 | F3 | G1 |
| | MORE OF D1- | | | | | |
| | D28 | | | | | |
| 26 | ANY ONE OR | B1 | A2 | E1 | F3 | G2 |
| | MORE OF D1- | | | | | |
| | D28 | | | | | |
| 27 | ANY ONE OR | B1 | A2 | E1 | F3 | ANY ONE |
| | MORE OF D1- | | | | | OF G3- |
| | D28 | | | | | G12 |
| 28 | ANY ONE OR | B1 | A2 | ANY ONE OF | F1 | G1 |
| | MORE OF D1- | | | E2-E4 | | |
| | D28 | | | | | |
| 29 | ANY ONE OR | B1 | A2 | ANY ONE OF | F1 | G2 |
| | MORE OF D1- | | | E2-E4 | | |
| | D28 | | | | | |
| 30 | ANY ONE OR | B1 | A2 | ANY ONE OF | F1 | ANY ONE |
| | MORE OF D1- | | | E2-E4 | | OF G3- |
| | D28 | | | | | G12 |
| 31 | ANY ONE OR | B1 | A2 | ANY ONE OF | F2 | G1 |
| | MORE OF D1- | | | E2-E4 | | |
| | D28 | | | | | |
| 32 | ANY ONE OR | B1 | A2 | ANY ONE OF | F2 | G2 |
| | MORE OF D1- | | | E2-E4 | | |
| | D28 | | | | | |
| 33 | ANY ONE OR | B1 | A2 | ANY ONE OF | F2 | ANY ONE |
| | MORE OF D1- | | | E2-E4 | | OF G3- |
| | D28 | | | | | G12 |
| 34 | ANY ONE OR | B1 | A2 | ANY ONE OF | F3 | G1 |
| | MORE OF D1- | | | E2-E4 | | |
| | D28 | | | | | |
| 35 | ANY ONE OR | B1 | A2 | ANY ONE OF | F3 | G2 |
| | MORE OF D1- | | | E2-E4 | | |
| | D28 | | | | | |
| 36 | ANY ONE OR | B1 | A2 | ANY ONE OF | F3 | ANY ONE |
| | MORE OF D1- | | | E2-E4 | | OF G3- |
| | D28 | | | | | G12 |
| 37 | ANY ONE OR | B1 | A3 | E1 | F1 | G1 |
| | MORE OF D1- | | | | | |
| | D28 | | | | | |
| 38 | ANY ONE OR | B1 | A3 | E1 | F1 | G2 |
| | MORE OF D1- | | | | | |
| | D28 | | | | | |
| 39 | ANY ONE OR | B1 | A3 | E1 | F1 | ANY ONE |
| | MORE OF D1- | | | | | OF G3- |
| | D28 | | | | | G12 |
| 40 | ANY ONE OR | B1 | A3 | E1 | F2 | G1 |
| | MORE OF D1- | | | | | |
| | D28 | | | | | |
| 41 | ANY ONE OR | B1 | A3 | E1 | F2 | G2 |
| | MORE OF D1- | | | | | |
| | D28 | | | | | |
| 42 | ANY ONE OR | B1 | A3 | E1 | F2 | ANY ONE |
| | MORE OF D1- | | | | | OF G3- |
| | D28 | | | | | G12 |
| 43 | ANY ONE OR | B1 | A3 | E1 | F3 | G1 |
| | MORE OF D1- | | | | | |
| | D28 | | | | | |
| 44 | ANY ONE OR | B1 | A3 | E1 | F3 | G2 |
| | MORE OF D1- | | | | | |
| | D28 | | | | | |
| 45 | ANY ONE OR | B1 | A3 | E1 | F3 | ANY ONE |
| | MORE OF D1- | | | | | OF G3- |
| | D28 | | | | | G12 |
| 46 | ANY ONE OR | B1 | A3 | ANY ONE OF | F1 | G1 |
| | MORE OF D1- | | | E2-E4 | | |
| | D28 | | | | | |
| 47 | ANY ONE OR | B1 | A3 | ANY ONE OF | F1 | G2 |
| | MORE OF D1- | | | E2-E4 | | |
| | D28 | | | | | |
| 48 | ANY ONE OR | B1 | A3 | ANY ONE OF | F1 | ANY ONE |
| | MORE OF D1- | | | E2-E4 | | OF G3- |
| | D28 | | | | | G12 |
| 49 | ANY ONE OR | B1 | A3 | ANY ONE OF | F2 | G1 |
| | MORE OF D1- | | | E2-E4 | | |
| | D28 | | | | | |
| 50 | ANY ONE OR | B1 | A3 | ANY ONE OF | F2 | G2 |
| | MORE OF D1- | | | E2-E4 | | |
| | D28 | | | | | |
| 51 | ANY ONE OR | B1 | A3 | ANY ONE OF | F2 | ANY ONE |
| | MORE OF D1- | | | E2-E4 | | OF G3- |
| | D28 | | | | | G12 |
| 52 | ANY ONE OR | B1 | A3 | ANY ONE OF | F3 | G1 |
| | MORE OF D1- | | | E2-E4 | | |
| | D28 | | | | | |
| 53 | ANY ONE OR | B1 | A3 | ANY ONE OF | F3 | G2 |
| | MORE OF D1- | | | E2-E4 | | |
| | D28 | | | | | |
| 54 | ANY ONE OR | B1 | A3 | ANY ONE OF | F3 | ANY ONE |
| | MORE OF D1- | | | E2-E4 | | OF G3- |
| | D28 | | | | | G12 |
| 55 | ANY ONE OR | B1 | A4 | E1 | F1 | G1 |
| | MORE OF D1- | | | | | |
| | D28 | | | | | |
| 56 | ANY ONE OR MORE | B1 | A4 | E1 | F1 | G2 |
| | OF D1-D28 | | | | | |
| 57 | ANY ONE OR | B1 | A4 | E1 | F1 | ANY ONE |
| | MORE OF D1- | | | | | OF G3- |
| | D28 | | | | | G12 |
| 58 | ANY ONE OR | B1 | A4 | E1 | F2 | G1 |
| | MORE OF D1- | | | | | |
| | D28 | | | | | |
| 59 | ANY ONE OR | B1 | A4 | E1 | F2 | G2 |
| | MORE OF D1- | | | | | |
| | D28 | | | | | |
| 60 | ANY ONE OR | B1 | A4 | E1 | F2 | ANY ONE |
| | MORE OF D1- | | | | | OF G3- |
| | D28 | | | | | G12 |
| 61 | ANY ONE OR | B1 | A4 | E1 | F3 | G1 |
| | MORE OF D1- | | | | | |
| | D28 | | | | | |
| 62 | ANY ONE OR | B1 | A4 | E1 | F3 | G2 |
| | MORE OF D1- | | | | | |
| | D28 | | | | | |
| 63 | ANY ONE OR | B1 | A4 | E1 | F3 | ANY ONE |
| | MORE OF D1- | | | | | OF G3- |
| | D28 | | | | | G12 |
| 64 | ANY ONE OR | B1 | A4 | ANY ONE OF | F1 | G1 |
| | MORE OF D1- | | | E2-E4 | | |
| | D28 | | | | | |
| 65 | ANY ONE OR MORE | B1 | A4 | ANY ONE OF | F1 | G2 |
| | OF D1-D28 | | | E2-E4 | | |
| 66 | ANY ONE OR | B1 | A4 | ANY ONE OF | F1 | ANY ONE |
| | MORE OF D1- | | | E2-E4 | | OF G3- |
| | D28 | | | | | G12 |
| 67 | ANY ONE OR MORE | B1 | A4 | ANY ONE OF | F2 | G1 |
| | OF D1-D28 | | | E2-E4 | | |
| 68 | ANY ONE OR | B1 | A4 | ANY ONE OF | F2 | G2 |
| | MORE OF D1- | | | E2-E4 | | |
| | D28 | | | | | |
| 69 | ANY ONE OR | B1 | A4 | ANY ONE OF | F2 | ANY ONE |
| | MORE OF D1- | | | E2-E4 | | OF G3- |
| | D28 | | | | | G12 |
| 70 | ANY ONE OR | B1 | A4 | ANY ONE OF | F3 | G1 |
| | MORE OF D1- | | | E2-E4 | | |
| | D28 | | | | | |
| 71 | ANY ONE OR | B1 | A4 | ANY ONE OF | F3 | G2 |
| | MORE OF D1- | | | E2-E4 | | |
| | D28 | | | | | |
| 72 | ANY ONE OR | B1 | A4 | ANY ONE OF | F3 | ANY ONE |
| | MORE OF D1- | | | E2-E4 | | OF G3- |
| | D28 | | | | | G12 |
| 73 | ANY ONE OR | B1 | A5 | E1 | F1 | G1 |
| | MORE OF D1- | | | | | |
| | D28 | | | | | |
| 74 | ANY ONE OR | B1 | A5 | E1 | F1 | G2 |
| | MORE OF D1- | | | | | |
| | D28 | | | | | |
| 75 | ANY ONE OR | B1 | A5 | E1 | F1 | ANY ONE |
| | MORE OF D1- | | | | | OF G3- |
| | D28 | | | | | G12 |
| 76 | ANY ONE OR | B1 | A5 | E1 | F2 | G1 |
| | MORE OF D1- | | | | | |
| | D28 | | | | | |
| 77 | ANY ONE OR | B1 | A5 | E1 | F2 | G2 |
| | MORE OF D1- | | | | | |
| | D28 | | | | | |
| 78 | ANY ONE OR | B1 | A5 | E1 | F2 | ANY ONE |
| | MORE OF D1- | | | | | OF G3- |
| | D28 | | | | | G12 |
| 79 | ANY ONE OR | B1 | A5 | E1 | F3 | G1 |
| | MORE OF D1- | | | | | |
| | D28 | | | | | |
| 80 | ANY ONE OR | B1 | A5 | E1 | F3 | G2 |
| | MORE OF D1- | | | | | |
| | D28 | | | | | |
| 81 | ANY ONE OR | B1 | A5 | E1 | F3 | ANY ONE |
| | MORE OF D1- | | | | | OF G3- |
| | D28 | | | | | G12 |
| 82 | ANY ONE OR | B1 | A5 | ANY ONE OF | F1 | G1 |
| | MORE OF D1- | | | E2-E4 | | |
| | D28 | | | | | |
| 83 | ANY ONE OR | B1 | A5 | ANY ONE OF | F1 | G2 |
| | MORE OF D1- | | | E2-E4 | | |
| | D28 | | | | | |
| 84 | ANY ONE OR | B1 | A5 | ANY ONE OF | F1 | ANY ONE |
| | MORE OF D1- | | | E2-E4 | | OF G3- |
| | D28 | | | | | G12 |
| 85 | ANY ONE OR | B1 | A5 | ANY ONE OF | F2 | G1 |
| | MORE OF D1- | | | E2-E4 | | |
| | D28 | | | | | |
| 86 | ANY ONE OR | B1 | A5 | ANY ONE OF | F2 | G2 |
| | MORE OF D1- | | | E2-E4 | | |
| | D28 | | | | | |
| 87 | ANY ONE OR | B1 | A5 | ANY ONE OF | F2 | ANY ONE |
| | MORE OF D1- | | | E2-E4 | | OF G3- |
| | D28 | | | | | G12 |
| 88 | ANY ONE OR | B1 | A5 | ANY ONE OF | F3 | G1 |
| | MORE OF D1- | | | E2-E4 | | |
| | D28 | | | | | |
| 89 | ANY ONE OR | B1 | A5 | ANY ONE OF | F3 | G2 |
| | MORE OF D1- | | | E2-E4 | | |
| | D28 | | | | | |
| 90 | ANY ONE OR | B1 | A5 | ANY ONE OF | F3 | ANY ONE |
| | MORE OF D1- | | | E2-E4 | | OF G3- |
| | D28 | | | | | G12 |
| 91 | ANY ONE OR | B1 | A6 | E1 | F1 | G1 |
| | MORE OF D1- | | | | | |
| | D28 | | | | | |
| 92 | ANY ONE OR | B1 | A6 | E1 | F1 | G2 |
| | MORE OF D1- | | | | | |
| | D28 | | | | | |
| 93 | ANY ONE OR | B1 | A6 | E1 | F1 | ANY ONE |
| | MORE OF D1- | | | | | OF G3- |
| | D28 | | | | | G12 |
| 94 | ANY ONE OR | B1 | A6 | E1 | F2 | G1 |
| | MORE OF D1- | | | | | |
| | D28 | | | | | |
| 95 | ANY ONE OR | B1 | A6 | E1 | F2 | G2 |
| | MORE OF D1- | | | | | |
| | D28 | | | | | |
| 96 | ANY ONE OR | B1 | A6 | E1 | F2 | ANY ONE |
| | MORE OF D1- | | | | | OF G3- |
| | D28 | | | | | G12 |
| 97 | ANY ONE OR | B1 | A6 | E1 | F3 | G1 |
| | MORE OF D1- | | | | | |
| | D28 | | | | | |
| 98 | ANY ONE OR | B1 | A6 | E1 | F3 | G2 |
| | MORE OF D1- | | | | | |
| | D28 | | | | | |
| 99 | ANY ONE OR | B1 | A6 | E1 | F3 | ANY ONE |
| | MORE OF D1- | | | | | OF G3- |
| | D28 | | | | | G12 |
| 100 | ANY ONE OR | B1 | A6 | ANY ONE OF | F1 | G1 |
| | MORE OF D1- | | | E2-E4 | | |
| | D28 | | | | | |
| 101 | ANY ONE OR | B1 | A6 | ANY ONE OF | F1 | G2 |
| | MORE OF D1- | | | E2-E4 | | |
| | D28 | | | | | |
| 102 | ANY ONE OR | B1 | A6 | ANY ONE OF | F1 | ANY ONE |
| | MORE OF D1- | | | E2-E4 | | OF G3- |
| | D28 | | | | | G12 |
| 103 | ANY ONE OR | B1 | A6 | ANY ONE OF | F2 | G1 |
| | MORE OF D1- | | | E2-E4 | | |
| | D28 | | | | | |
| 104 | ANY ONE OR | B1 | A6 | ANY ONE OF | F2 | G2 |
| | MORE OF D1- | | | E2-E4 | | |
| | D28 | | | | | |
| 105 | ANY ONE OR | B1 | A6 | ANY ONE OF | F2 | ANY ONE |
| | MORE OF D1- | | | E2-E4 | | OF G3- |
| | D28 | | | | | G12 |
| 106 | ANY ONE OR | B1 | A6 | ANY ONE OF | F3 | G1 |
| | MORE OF D1- | | | E2-E4 | | |
| | D28 | | | | | |
| 107 | ANY ONE OR | B1 | A6 | ANY ONE OF | F3 | G2 |
| | MORE OF D1- | | | E2-E4 | | |
| | D28 | | | | | |
| 108 | ANY ONE OR | B1 | A6 | ANY ONE OF | F3 | ANY ONE |
| | MORE OF D1- | | | E2-E4 | | OF G3- |
| | D28 | | | | | G12 |
| 109 | ANY ONE OR | B1 | A7 | E1 | F1 | G1 |
| | MORE OF D1- | | | | | |
| | D28 | | | | | |
| 110 | ANY ONE OR | B1 | A7 | E1 | F1 | G2 |
| | MORE OF D1- | | | | | |
| | D28 | | | | | |
| 111 | ANY ONE OR | B1 | A7 | E1 | F1 | ANY ONE |
| | MORE OF D1- | | | | | OF G3- |
| | D28 | | | | | G12 |
| 112 | ANY ONE OR | B1 | A7 | E1 | F2 | G1 |
| | MORE OF D1- | | | | | |
| | D28 | | | | | |
| 113 | ANY ONE OR | B1 | A7 | E1 | F2 | G2 |
| | MORE OF D1- | | | | | |
| | D28 | | | | | |
| 114 | ANY ONE OR | B1 | A7 | E1 | F2 | ANY ONE |
| | MORE OF D1- | | | | | OF G3- |
| | D28 | | | | | G12 |
| 115 | ANY ONE OR | B1 | A7 | E1 | F3 | G1 |
| | MORE OF D1- | | | | | |
| | D28 | | | | | |
| 116 | ANY ONE OR | B1 | A7 | E1 | F3 | G2 |
| | MORE OF D1- | | | | | |
| | D28 | | | | | |
| 117 | ANY ONE OR | B1 | A7 | E1 | F3 | ANY ONE |
| | MORE OF D1- | | | | | OF G3- |
| | D28 | | | | | G12 |
| 118 | ANY ONE OR | B1 | A7 | ANY ONE OF | F1 | G1 |
| | MORE OF D1- | | | E2-E4 | | |
| | D28 | | | | | |
| 119 | ANY ONE OR | B1 | A7 | ANY ONE OF | F1 | G2 |
| | MORE OF D1- | | | E2-E4 | | |
| | D28 | | | | | |
| 120 | ANY ONE OR | B1 | A7 | ANY ONE OF | F1 | ANY ONE |
| | MORE OF D1- | | | E2-E4 | | OF G3- |
| | D28 | | | | | G12 |
| 121 | ANY ONE OR | B1 | A7 | ANY ONE OF | F2 | G1 |
| | MORE OF D1- | | | E2-E4 | | |
| | D28 | | | | | |
| 122 | ANY ONE OR | B1 | A7 | ANY ONE OF | F2 | G2 |
| | MORE OF D1- | | | E2-E4 | | |
| | D28 | | | | | |
| 123 | ANY ONE OR | B1 | A7 | ANY ONE OF | F2 | ANY ONE |
| | MORE OF D1- | | | E2-E4 | | OF G3- |
| | D28 | | | | | G12 |
| 124 | ANY ONE OR | B1 | A7 | ANY ONE OF | F3 | G1 |
| | MORE OF D1- | | | E2-E4 | | |
| | D28 | | | | | |
| 125 | ANY ONE OR | B1 | A7 | ANY ONE OF | F3 | G2 |
| | MORE OF D1- | | | E2-E4 | | |
| | D28 | | | | | |
| 126 | ANY ONE OR | B1 | A7 | ANY ONE OF | F3 | ANY ONE |
| | MORE OF D1- | | | E2-E4 | | OF G3- |
| | D28 | | | | | G12 |
| 127 | ANY ONE OR | B1 | A8 | E1 | F1 | G1 |
| | MORE OF D1- | | | | | |
| | D28 | | | | | |
| 128 | ANY ONE OR | B1 | A8 | E1 | F1 | G2 |
| | MORE OF D1- | | | | | |
| | D28 | | | | | |
| 129 | ANY ONE OR | B1 | A8 | E1 | F1 | ANY ONE |
| | MORE OF D1- | | | | | OF G3- |
| | D28 | | | | | G12 |
| 130 | ANY ONE OR | B1 | A8 | E1 | F2 | G1 |
| | MORE OF D1- | | | | | |
| | D28 | | | | | |
| 131 | ANY ONE OR | B1 | A8 | E1 | F2 | G2 |
| | MORE OF D1- | | | | | |
| | D28 | | | | | |
| 132 | ANY ONE OR | B1 | A8 | E1 | F2 | ANY ONE |
| | MORE OF D1- | | | | | OF G3- |
| | D28 | | | | | G12 |
| 133 | ANY ONE OR | B1 | A8 | E1 | F3 | G1 |
| | MORE OF D1- | | | | | |
| | D28 | | | | | |
| 134 | ANY ONE OR | B1 | A8 | E1 | F3 | G2 |
| | MORE OF D1- | | | | | |
| | D28 | | | | | |
| 135 | ANY ONE OR | B1 | A8 | E1 | F3 | ANY ONE |
| | MORE OF D1- | | | | | OF G3- |
| | D28 | | | | | G12 |
| 136 | ANY ONE OR | B1 | A8 | ANY ONE OF | F1 | G1 |
| | MORE OF D1- | | | E2-E4 | | |
| | D28 | | | | | |
| 137 | ANY ONE OR | B1 | A8 | ANY ONE OF | F1 | G2 |
| | MORE OF D1- | | | E2-E4 | | |
| | D28 | | | | | |
| 138 | ANY ONE OR | B1 | A8 | ANY ONE OF | F1 | ANY ONE |
| | MORE OF D1- | | | E2-E4 | | OF G3- |
| | D28 | | | | | G12 |
| 139 | ANY ONE OR | B1 | A8 | ANY ONE OF | F2 | G1 |
| | MORE OF D1- | | | E2-E4 | | |
| | D28 | | | | | |
| 140 | ANY ONE OR | B1 | A8 | ANY ONE OF | F2 | G2 |
| | MORE OF D1- | | | E2-E4 | | |
| | D28 | | | | | |
| 141 | ANY ONE OR | B1 | A8 | ANY ONE OF | F2 | ANY ONE |
| | MORE OF D1- | | | E2-E4 | | OF G3- |
| | D28 | | | | | G12 |
| 142 | ANY ONE OR | B1 | A8 | ANY ONE OF | F3 | G1 |
| | MORE OF D1- | | | E2-E4 | | |
| | D28 | | | | | |
| 143 | ANY ONE OR | B1 | A8 | ANY ONE OF | F3 | G2 |
| | MORE OF D1- | | | E2-E4 | | |
| | D28 | | | | | |
| 144 | ANY ONE OR | B1 | A8 | ANY ONE OF | F3 | ANY ONE |
| | MORE OF D1- | | | E2-E4 | | OF G3- |
| | D28 | | | | | G12 |
| 145 | ANY ONE OR | B1 | A9 | E1 | F1 | G1 |
| | MORE OF D1- | | | | | |
| | D28 | | | | | |
| 146 | ANY ONE OR | B1 | A9 | E1 | F1 | G2 |
| | MORE OF D1- | | | | | |
| | D28 | | | | | |
| 147 | ANY ONE OR | B1 | A9 | E1 | F1 | ANY ONE |
| | MORE OF D1- | | | | | OF G3- |
| | D28 | | | | | G12 |
| 148 | ANY ONE OR | B1 | A9 | E1 | F2 | G1 |
| | MORE OF D1- | | | | | |
| | D28 | | | | | |
| 149 | ANY ONE OR | B1 | A9 | E1 | F2 | G2 |
| | MORE OF D1- | | | | | |
| | D28 | | | | | |
| 150 | ANY ONE OR | B1 | A9 | E1 | F2 | ANY ONE |
| | MORE OF D1- | | | | | OF G3- |
| | D28 | | | | | G12 |
| 151 | ANY ONE OR | B1 | A9 | E1 | F3 | G1 |
| | MORE OF D1- | | | | | |
| | D28 | | | | | |
| 152 | ANY ONE OR | B1 | A9 | E1 | F3 | G2 |
| | MORE OF D1- | | | | | |
| | D28 | | | | | |
| 153 | ANY ONE OR | B1 | A9 | E1 | F3 | ANY ONE |
| | MORE OF D1- | | | | | OF G3- |
| | D28 | | | | | G12 |
| 154 | ANY ONE OR | B1 | A9 | ANY ONE OF | F1 | G1 |
| | MORE OF D1- | | | E2-E4 | | |
| | D28 | | | | | |
| 155 | ANY ONE OR | B1 | A9 | ANY ONE OF | F1 | G2 |
| | MORE OF D1- | | | E2-E4 | | |
| | D28 | | | | | |
| 156 | ANY ONE OR | B1 | A9 | ANY ONE OF | F1 | ANY ONE |
| | MORE OF D1- | | | E2-E4 | | OF G3- |
| | D28 | | | | | G12 |
| 157 | ANY ONE OR | B1 | A9 | ANY ONE OF | F2 | G1 |
| | MORE OF D1- | | | E2-E4 | | |
| | D28 | | | | | |
| 158 | ANY ONE OR | B1 | A9 | ANY ONE OF | F2 | G2 |
| | MORE OF D1- | | | E2-E4 | | |
| | D28 | | | | | |
| 159 | ANY ONE OR | B1 | A9 | ANY ONE OF | F2 | ANY ONE |
| | MORE OF D1- | | | E2-E4 | | OF G3- |
| | D28 | | | | | G12 |
| 160 | ANY ONE OR | B1 | A9 | ANY ONE OF | F3 | G1 |
| | MORE OF D1- | | | E2-E4 | | |
| | D28 | | | | | |
| 161 | ANY ONE OR | B1 | A9 | ANY ONE OF | F3 | G2 |
| | MORE OF D1- | | | E2-E4 | | |
| | D28 | | | | | |
| 162 | ANY ONE OR | B1 | A9 | ANY ONE OF | F3 | ANY ONE |
| | MORE OF D1- | | | E2-E4 | | OF G3- |
| | D28 | | | | | G12 |
| 163 | ANY ONE OR | B1 | A10 | E1 | F1 | G1 |
| | MORE OF D1- | | | | | |
| | D28 | | | | | |
| 164 | ANY ONE OR | B1 | A10 | E1 | F1 | G2 |
| | MORE OF D1- | | | | | |
| | D28 | | | | | |
| 165 | ANY ONE OR | B1 | A10 | E1 | F1 | ANY ONE |
| | MORE OF D1- | | | | | OF G3- |
| | D28 | | | | | G12 |
| 166 | ANY ONE OR | B1 | A10 | E1 | F2 | G1 |
| | MORE OF D1- | | | | | |
| | D28 | | | | | |
| 167 | ANY ONE OR | B1 | A10 | E1 | F2 | G2 |
| | MORE OF D1- | | | | | |
| | D28 | | | | | |
| 168 | ANY ONE OR | B1 | A10 | E1 | F2 | ANY ONE |
| | MORE OF D1- | | | | | OF G3- |
| | D28 | | | | | G12 |
| 169 | ANY ONE OR | B1 | A10 | E1 | F3 | G1 |
| | MORE OF D1- | | | | | |
| | D28 | | | | | |
| 170 | ANY ONE OR | B1 | A10 | E1 | F3 | G2 |
| | MORE OF D1- | | | | | |
| | D28 | | | | | |
| 171 | ANY ONE OR | B1 | A10 | E1 | F3 | ANY ONE |
| | MORE OF D1- | | | | | OF G3- |
| | D28 | | | | | G12 |
| 172 | ANY ONE OR | B1 | A10 | ANY ONE OF | F1 | G1 |
| | MORE OF D1- | | | E2-E4 | | |
| | D28 | | | | | |
| 173 | ANY ONE OR | B1 | A10 | ANY ONE OF | F1 | G2 |
| | MORE OF D1- | | | E2-E4 | | |
| | D28 | | | | | |
| 174 | ANY ONE OR | B1 | A10 | ANY ONE OF | F1 | ANY ONE |
| | MORE OF D1- | | | E2-E4 | | OF G3- |
| | D28 | | | | | G12 |
| 175 | ANY ONE OR | B1 | A10 | ANY ONE OF | F2 | G1 |
| | MORE OF D1- | | | E2-E4 | | |
| | D28 | | | | | |
| 176 | ANY ONE OR | B1 | A10 | ANY ONE OF | F2 | G2 |
| | MORE OF D1- | | | E2-E4 | | |
| | D28 | | | | | |
| 177 | ANY ONE OR | B1 | A10 | ANY ONE OF | F2 | ANY ONE |
| | MORE OF D1- | | | E2-E4 | | OF G3- |
| | D28 | | | | | G12 |
| 178 | ANY ONE OR | B1 | A10 | ANY ONE OF | F3 | G1 |
| | MORE OF D1- | | | E2-E4 | | |
| | D28 | | | | | |
| 179 | ANY ONE OR | B1 | A10 | ANY ONE OF | F3 | G2 |
| | MORE OF D1- | | | E2-E4 | | |
| | D28 | | | | | |
| 180 | ANY ONE OR | B1 | A10 | ANY ONE OF | F3 | ANY ONE |
| | MORE OF D1- | | | E2-E4 | | OF G3- |
| | D28 | | | | | G12 |
| 181 | ANY ONE OR MORE | B2 | A1 | E1 | F1 | G1 |
| | OF D1-D28 | | | | | |
| 182 | ANY ONE OR | B2 | A1 | E1 | F1 | G2 |
| | MORE OF D1- | | | | | |
| | D28 | | | | | |
| 183 | ANY ONE OR | B2 | A1 | E1 | F1 | ANY ONE |
| | MORE OF | | | | | OF G3- |
| | D1-D28 | | | | | G12 |
| 184 | ANY ONE OR | B2 | A1 | E1 | F2 | G1 |
| | MORE OF D1- | | | | | |
| | D28 | | | | | |
| 185 | ANY ONE OR | B2 | A1 | E1 | F2 | G2 |
| | MORE OF D1- | | | | | |
| | D28 | | | | | |
| 186 | ANY ONE OR | B2 | A1 | E1 | F2 | ANY ONE |
| | MORE OF D1- | | | | | OF G3- |
| | D28 | | | | | G12 |
| 187 | ANY ONE OR MORE | B2 | A1 | E1 | F3 | G1 |
| | OF D1-D28 | | | | | |
| 188 | ANY ONE OR | B2 | A1 | E1 | F3 | G2 |
| | MORE OF D1- | | | | | |
| | D28 | | | | | |
| 189 | ANY ONE OR | B2 | A1 | E1 | F3 | ANY ONE |
| | MORE OF D1- | | | | | OF G3- |
| | D28 | | | | | G12 |
| 190 | ANY ONE OR MORE | B2 | A1 | ANY ONE OF | F1 | G1 |
| | OF D1-D28 | | | E2-E4 | | |
| 191 | ANY ONE OR | B2 | A1 | ANY ONE OF | F1 | G2 |
| | MORE OF D1- | | | E2-E4 | | |
| | D28 | | | | | |
| 192 | ANY ONE OR | B2 | A1 | ANY ONE OF | F1 | ANY ONE |
| | MORE OF D1- | | | E2-E4 | | OF G3- |
| | D28 | | | | | G12 |
| 193 | ANY ONE OR | B2 | A1 | ANY ONE OF | F2 | G1 |
| | MORE OF D1- | | | E2-E4 | | |
| | D28 | | | | | |
| 194 | ANY ONE OR | B2 | A1 | ANY ONE OF | F2 | G2 |
| | MORE OF D1- | | | E2-E4 | | |
| | D28 | | | | | |
| 195 | ANY ONE OR | B2 | A1 | ANY ONE OF | F2 | ANY ONE |
| | MORE OF D1- | | | E2-E4 | | OF G3- |
| | D28 | | | | | G12 |
| 196 | ANY ONE OR | B2 | A1 | ANY ONE OF | F3 | G1 |
| | MORE OF D1- | | | E2-E4 | | |
| | D28 | | | | | |
| 197 | ANY ONE OR | B2 | A1 | ANY ONE OF | F3 | G2 |
| | MORE OF D1- | | | E2-E4 | | |
| | D28 | | | | | |
| 198 | ANY ONE OR | B2 | A1 | ANY ONE OF | F3 | ANY ONE |
| | MORE OF D1- | | | E2-E4 | | OF G3- |
| | D28 | | | | | G12 |
| 199 | ANY ONE OR | B2 | A2 | E1 | F1 | G1 |
| | MORE OF D1- | | | | | |
| | D28 | | | | | |
| 200 | ANY ONE OR | B2 | A2 | E1 | F1 | G2 |
| | MORE OF D1- | | | | | |
| | D28 | | | | | |
| 201 | ANY ONE OR | B2 | A2 | E1 | F1 | ANY ONE |
| | MORE OF D1- | | | | | OF G3- |
| | D28 | | | | | G12 |
| 202 | ANY ONE OR | B2 | A2 | E1 | F2 | G1 |
| | MORE OF D1- | | | | | |
| | D28 | | | | | |
| 203 | ANY ONE OR | B2 | A2 | E1 | F2 | G2 |
| | MORE OF D1- | | | | | |
| | D28 | | | | | |
| 204 | ANY ONE OR | B2 | A2 | E1 | F2 | ANY ONE |
| | MORE OF D1- | | | | | OF G3- |
| | D28 | | | | | G12 |
| 205 | ANY ONE OR | B2 | A2 | E1 | F3 | G1 |
| | MORE OF D1- | | | | | |
| | D28 | | | | | |
| 206 | ANY ONE OR | B2 | A2 | E1 | F3 | G2 |
| | MORE OF D1- | | | | | |
| | D28 | | | | | |
| 207 | ANY ONE OR | B2 | A2 | E1 | F3 | ANY ONE |
| | MORE OF D1- | | | | | OF G3- |
| | D28 | | | | | G12 |
| 208 | ANY ONE OR | B2 | A2 | ANY ONE OF | F1 | G1 |
| | MORE OF D1- | | | E2-E4 | | |
| | D28 | | | | | |
| 209 | ANY ONE OR | B2 | A2 | ANY ONE OF | F1 | G2 |
| | MORE OF D1- | | | E2-E4 | | |
| | D28 | | | | | |
| 210 | ANY ONE OR | B2 | A2 | ANY ONE OF | F1 | ANY ONE |
| | MORE OF D1- | | | E2-E4 | | OF G3- |
| | D28 | | | | | G12 |
| 211 | ANY ONE OR | B2 | A2 | ANY ONE OF | F2 | G1 |
| | MORE OF D1- | | | E2-E4 | | |
| | D28 | | | | | |
| 212 | ANY ONE OR | B2 | A2 | ANY ONE OF | F2 | G2 |
| | MORE OF D1- | | | E2-E4 | | |
| | D28 | | | | | |
| 213 | ANY ONE OR | B2 | A2 | ANY ONE OF | F2 | ANY ONE |
| | MORE OF D1- | | | E2-E4 | | OF G3- |
| | D28 | | | | | G12 |
| 214 | ANY ONE OR | B2 | A2 | ANY ONE OF | F3 | G1 |
| | MORE OF D1- | | | E2-E4 | | |
| | D28 | | | | | |
| 215 | ANY ONE OR | B2 | A2 | ANY ONE OF | F3 | G2 |
| | MORE OF D1- | | | E2-E4 | | |
| | D28 | | | | | |
| 216 | ANY ONE OR | B2 | A2 | ANY ONE OF | F3 | ANY ONE |
| | MORE OF D1- | | | E2-E4 | | OF G3- |
| | D28 | | | | | G12 |
| 217 | ANY ONE OR | B2 | A3 | E1 | F1 | G1 |
| | MORE OF D1- | | | | | |
| | D28 | | | | | |
| 218 | ANY ONE OR | B2 | A3 | E1 | F1 | G2 |
| | MORE OF D1- | | | | | |
| | D28 | | | | | |
| 219 | ANY ONE OR | B2 | A3 | E1 | F1 | ANY ONE |
| | MORE OF D1- | | | | | OF G3- |
| | D28 | | | | | G12 |
| 220 | ANY ONE OR | B2 | A3 | E1 | F2 | G1 |
| | MORE OF D1- | | | | | |
| | D28 | | | | | |
| 221 | ANY ONE OR | B2 | A3 | E1 | F2 | G2 |
| | MORE OF D1- | | | | | |
| | D28 | | | | | |
| 222 | ANY ONE OR | B2 | A3 | E1 | F2 | ANY ONE |
| | MORE OF D1- | | | | | OF G3- |
| | D28 | | | | | G12 |
| 223 | ANY ONE OR | B2 | A3 | E1 | F3 | G1 |
| | MORE OF D1- | | | | | |
| | D28 | | | | | |
| 224 | ANY ONE OR | B2 | A3 | E1 | F3 | G2 |
| | MORE OF D1- | | | | | |
| | D28 | | | | | |
| 225 | ANY ONE OR | B2 | A3 | E1 | F3 | ANY ONE |
| | MORE OF D1- | | | | | OF G3- |
| | D28 | | | | | G12 |
| 226 | ANY ONE OR | B2 | A3 | ANY ONE OF | F1 | G1 |
| | MORE OF D1- | | | E2-E4 | | |
| | D28 | | | | | |
| 227 | ANY ONE OR | B2 | A3 | ANY ONE OF | F1 | G2 |
| | MORE OF D1- | | | E2-E4 | | |
| | D28 | | | | | |
| 228 | ANY ONE OR | B2 | A3 | ANY ONE OF | F1 | ANY ONE |
| | MORE OF D1- | | | E2-E4 | | OF G3- |
| | D28 | | | | | G12 |
| 229 | ANY ONE OR | B2 | A3 | ANY ONE OF | F2 | G1 |
| | MORE OF D1- | | | E2-E4 | | |
| | D28 | | | | | |
| 230 | ANY ONE OR | B2 | A3 | ANY ONE OF | F2 | G2 |
| | MORE OF D1- | | | E2-E4 | | |
| | D28 | | | | | |
| 231 | ANY ONE OR | B2 | A3 | ANY ONE OF | F2 | ANY ONE |
| | MORE OF D1- | | | E2-E4 | | OF G3- |
| | D28 | | | | | G12 |
| 232 | ANY ONE OR | B2 | A3 | ANY ONE OF | F3 | G1 |
| | MORE OF D1- | | | E2-E4 | | |
| | D28 | | | | | |
| 233 | ANY ONE OR | B2 | A3 | ANY ONE OF | F3 | G2 |
| | MORE OF D1- | | | E2-E4 | | |
| | D28 | | | | | |
| 234 | ANY ONE OR | B2 | A3 | ANY ONE OF | F3 | ANY ONE |
| | MORE OF D1- | | | E2-E4 | | OF G3- |
| | D28 | | | | | G12 |
| 235 | ANY ONE OR | B2 | A4 | E1 | F1 | G1 |
| | MORE OF D1- | | | | | |
| | D28 | | | | | |
| 236 | ANY ONE OR | B2 | A4 | E1 | F1 | G2 |
| | MORE OF D1- | | | | | |
| | D28 | | | | | |
| 237 | ANY ONE OR | B2 | A4 | E1 | F1 | ANY ONE |
| | MORE OF D1- | | | | | OF G3- |
| | D28 | | | | | G12 |
| 238 | ANY ONE OR | B2 | A4 | E1 | F2 | G1 |
| | MORE OF D1- | | | | | |
| | D28 | | | | | |
| 239 | ANY ONE OR | B2 | A4 | E1 | F2 | G2 |
| | MORE OF D1- | | | | | |
| | D28 | | | | | |
| 240 | ANY ONE OR | B2 | A4 | E1 | F2 | ANY ONE |
| | MORE OF D1- | | | | | OF G3- |
| | D28 | | | | | G12 |
| 241 | ANY ONE OR | B2 | A4 | E1 | F3 | G1 |
| | MORE OF D1- | | | | | |
| | D28 | | | | | |
| 242 | ANY ONE OR | B2 | A4 | E1 | F3 | G2 |
| | MORE OF D1- | | | | | |
| | D28 | | | | | |
| 243 | ANY ONE OR | B2 | A4 | E1 | F3 | ANY ONE |
| | MORE OF D1- | | | | | OF G3- |
| | D28 | | | | | G12 |
| 244 | ANY ONE OR | B2 | A4 | ANY ONE OF | F1 | G1 |
| | MORE OF D1- | | | E2-E4 | | |
| | D28 | | | | | |
| 245 | ANY ONE OR | B2 | A4 | ANY ONE OF | F1 | G2 |
| | MORE OF D1- | | | E2-E4 | | |
| | D28 | | | | | |
| 246 | ANY ONE OR | B2 | A4 | ANY ONE OF | F1 | ANY ONE |
| | MORE OF D1- | | | E2-E4 | | OF G3- |
| | D28 | | | | | G12 |
| 247 | ANY ONE OR | B2 | A4 | ANY ONE OF | F2 | G1 |
| | MORE OF D1- | | | E2-E4 | | |
| | D28 | | | | | |
| 248 | ANY ONE OR | B2 | A4 | ANY ONE OF | F2 | G2 |
| | MORE OF D1- | | | E2-E4 | | |
| | D28 | | | | | |
| 249 | ANY ONE OR | B2 | A4 | ANY ONE OF | F2 | ANY ONE |
| | MORE OF D1- | | | E2-E4 | | OF G3- |
| | D28 | | | | | G12 |
| 250 | ANY ONE OR | B2 | A4 | ANY ONE OF | F3 | G1 |
| | MORE OF D1- | | | E2-E4 | | |
| | D28 | | | | | |
| 251 | ANY ONE OR | B2 | A4 | ANY ONE OF | F3 | G2 |
| | MORE OF D1- | | | E2-E4 | | |
| | D28 | | | | | |
| 252 | ANY ONE OR | B2 | A4 | ANY ONE OF | F3 | ANY ONE |
| | MORE OF D1- | | | E2-E4 | | OF G3- |
| | D28 | | | | | G12 |
| 253 | ANY ONE OR | B2 | A5 | E1 | F1 | G1 |
| | MORE OF D1- | | | | | |
| | D28 | | | | | |
| 254 | ANY ONE OR | B2 | A5 | E1 | F1 | G2 |
| | MORE OF D1- | | | | | |
| | D28 | | | | | |
| 255 | ANY ONE OR | B2 | A5 | E1 | F1 | ANY ONE |
| | MORE OF D1- | | | | | OF G3- |
| | D28 | | | | | G12 |
| 256 | ANY ONE OR | B2 | A5 | E1 | F2 | G1 |
| | MORE OF D1- | | | | | |
| | D28 | | | | | |
| 257 | ANY ONE OR | B2 | A5 | E1 | F2 | G2 |
| | MORE OF D1- | | | | | |
| | D28 | | | | | |
| 258 | ANY ONE OR | B2 | A5 | E1 | F2 | ANY ONE |
| | MORE OF D1- | | | | | OF G3- |
| | D28 | | | | | G12 |
| 259 | ANY ONE OR | B2 | A5 | E1 | F3 | G1 |
| | MORE OF D1- | | | | | |
| | D28 | | | | | |
| 260 | ANY ONE OR | B2 | A5 | E1 | F3 | G2 |
| | MORE OF D1- | | | | | |
| | D28 | | | | | |
| 261 | ANY ONE OR | B2 | A5 | E1 | F3 | ANY ONE |
| | MORE OF D1- | | | | | OF G3- |
| | D28 | | | | | G12 |
| 262 | ANY ONE OR | B2 | A5 | ANY ONE OF | F1 | G1 |
| | MORE OF D1- | | | E2-E4 | | |
| | D28 | | | | | |
| 263 | ANY ONE OR | B2 | A5 | ANY ONE OF | F1 | G2 |
| | MORE OF D1- | | | E2-E4 | | |
| | D28 | | | | | |
| 264 | ANY ONE OR | B2 | A5 | ANY ONE OF | F1 | ANY ONE |
| | MORE OF D1- | | | E2-E4 | | OF G3- |
| | D28 | | | | | G12 |
| 265 | ANY ONE OR | B2 | A5 | ANY ONE OF | F2 | G1 |
| | MORE OF D1- | | | E2-E4 | | |
| | D28 | | | | | |
| 266 | ANY ONE OR | B2 | A5 | ANY ONE OF | F2 | G2 |
| | MORE OF D1- | | | E2-E4 | | |
| | D28 | | | | | |
| 267 | ANY ONE OR | B2 | A5 | ANY ONE OF | F2 | ANY ONE |
| | MORE OF D1- | | | E2-E4 | | OF G3- |
| | D28 | | | | | G12 |
| 268 | ANY ONE OR | B2 | A5 | ANY ONE OF | F3 | G1 |
| | MORE OF D1- | | | E2-E4 | | |
| | D28 | | | | | |
| 269 | ANY ONE OR | B2 | A5 | ANY ONE OF | F3 | G2 |
| | MORE OF D1- | | | E2-E4 | | |
| | D28 | | | | | |
| 270 | ANY ONE OR | B2 | A5 | ANY ONE OF | F3 | ANY ONE |
| | MORE OF D1- | | | E2-E4 | | OF G3- |
| | D28 | | | | | G12 |
| 271 | ANY ONE OR | B2 | A6 | E1 | F1 | G1 |
| | MORE OF D1- | | | | | |
| | D28 | | | | | |
| 272 | ANY ONE OR | B2 | A6 | E1 | F1 | G2 |
| | MORE OF D1- | | | | | |
| | D28 | | | | | |
| 273 | ANY ONE OR | B2 | A6 | E1 | F1 | ANY ONE |
| | MORE OF D1- | | | | | OF G3- |
| | D28 | | | | | G12 |
| 274 | ANY ONE OR | B2 | A6 | E1 | F2 | G1 |
| | MORE OF D1- | | | | | |
| | D28 | | | | | |
| 275 | ANY ONE OR | B2 | A6 | E1 | F2 | G2 |
| | MORE OF D1- | | | | | |
| | D28 | | | | | |
| 276 | ANY ONE OR | B2 | A6 | E1 | F2 | ANY ONE |
| | MORE OF D1- | | | | | OF G3- |
| | D28 | | | | | G12 |
| 277 | ANY ONE OR | B2 | A6 | E1 | F3 | G1 |
| | MORE OF D1- | | | | | |
| | D28 | | | | | |
| 278 | ANY ONE OR | B2 | A6 | E1 | F3 | G2 |
| | MORE OF D1- | | | | | |
| | D28 | | | | | |
| 279 | ANY ONE OR | B2 | A6 | E1 | F3 | ANY ONE |
| | MORE OF D1- | | | | | OF G3- |
| | D28 | | | | | G12 |
| 280 | ANY ONE OR | B2 | A6 | ANY ONE OF | F1 | G1 |
| | MORE OF D1- | | | E2-E4 | | |
| | D28 | | | | | |
| 281 | ANY ONE OR | B2 | A6 | ANY ONE OF | F1 | G2 |
| | MORE OF D1- | | | E2-E4 | | |
| | D28 | | | | | |
| 282 | ANY ONE OR | B2 | A6 | ANY ONE OF | F1 | ANY ONE |
| | MORE OF D1- | | | E2-E4 | | OF G3- |
| | D28 | | | | | G12 |
| 283 | ANY ONE OR | B2 | A6 | ANY ONE OF | F2 | G1 |
| | MORE OF D1- | | | E2-E4 | | |
| | D28 | | | | | |
| 284 | ANY ONE OR | B2 | A6 | ANY ONE OF | F2 | G2 |
| | MORE OF D1- | | | E2-E4 | | |
| | D28 | | | | | |
| 285 | ANY ONE OR | B2 | A6 | ANY ONE OF | F2 | ANY ONE |
| | MORE OF D1- | | | E2-E4 | | OF G3- |
| | D28 | | | | | G12 |
| 286 | ANY ONE OR | B2 | A6 | ANY ONE OF | F3 | G1 |
| | MORE OF D1- | | | E2-E4 | | |
| | D28 | | | | | |
| 287 | ANY ONE OR | B2 | A6 | ANY ONE OF | F3 | G2 |
| | MORE OF D1- | | | E2-E4 | | |
| | D28 | | | | | |
| 288 | ANY ONE OR | B2 | A6 | ANY ONE OF | F3 | ANY ONE |
| | MORE OF D1- | | | E2-E4 | | OF G3- |
| | D28 | | | | | G12 |
| 289 | ANY ONE OR | B2 | A7 | E1 | F1 | G1 |
| | MORE OF D1- | | | | | |
| | D28 | | | | | |
| 290 | ANY ONE OR | B2 | A7 | E1 | F1 | G2 |
| | MORE OF D1- | | | | | |
| | D28 | | | | | |
| 291 | ANY ONE OR | B2 | A7 | E1 | F1 | ANY ONE |
| | MORE OF D1- | | | | | OF G3- |
| | D28 | | | | | G12 |
| 292 | ANY ONE OR | B2 | A7 | E1 | F2 | G1 |
| | MORE OF D1- | | | | | |
| | D28 | | | | | |
| 293 | ANY ONE OR | B2 | A7 | E1 | F2 | G2 |
| | MORE OF D1- | | | | | |
| | D28 | | | | | |
| 294 | ANY ONE OR | B2 | A7 | E1 | F2 | ANY ONE |
| | MORE OF D1- | | | | | OF G3- |
| | D28 | | | | | G12 |
| 295 | ANY ONE OR | B2 | A7 | E1 | F3 | G1 |
| | MORE OF D1- | | | | | |
| | D28 | | | | | |
| 296 | ANY ONE OR | B2 | A7 | E1 | F3 | G2 |
| | MORE OF D1- | | | | | |
| | D28 | | | | | |
| 297 | ANY ONE OR | B2 | A7 | E1 | F3 | ANY ONE |
| | MORE OF D1- | | | | | OF G3- |
| | D28 | | | | | G12 |
| 298 | ANY ONE OR | B2 | A7 | ANY ONE OF | F1 | G1 |
| | MORE OF D1- | | | E2-E4 | | |
| | D28 | | | | | |
| 299 | ANY ONE OR | B2 | A7 | ANY ONE OF | F1 | G2 |
| | MORE OF D1- | | | E2-E4 | | |
| | D28 | | | | | |
| 300 | ANY ONE OR | B2 | A7 | ANY ONE OF | F1 | ANY ONE |
| | MORE OF D1- | | | E2-E4 | | OF G3- |
| | D28 | | | | | G12 |
| 301 | ANY ONE OR | B2 | A7 | ANY ONE OF | F2 | G1 |
| | MORE OF D1- | | | E2-E4 | | |
| | D28 | | | | | |
| 302 | ANY ONE OR | B2 | A7 | ANY ONE OF | F2 | G2 |
| | MORE OF D1- | | | E2-E4 | | |
| | D28 | | | | | |
| 303 | ANY ONE OR | B2 | A7 | ANY ONE OF | F2 | ANY ONE |
| | MORE OF D1- | | | E2-E4 | | OF G3- |
| | D28 | | | | | G12 |
| 304 | ANY ONE OR | B2 | A7 | ANY ONE OF | F3 | G1 |
| | MORE OF D1- | | | E2-E4 | | |
| | D28 | | | | | |
| 305 | ANY ONE OR | B2 | A7 | ANY ONE OF | F3 | G2 |
| | MORE OF D1- | | | E2-E4 | | |
| | D28 | | | | | |
| 306 | ANY ONE OR | B2 | A7 | ANY ONE OF | F3 | ANY ONE |
| | MORE OF D1- | | | E2-E4 | | OF G3- |
| | D28 | | | | | G12 |
| 307 | ANY ONE OR | B2 | A8 | E1 | F1 | G1 |
| | MORE OF D1- | | | | | |
| | D28 | | | | | |
| 308 | ANY ONE OR | B2 | A8 | E1 | F1 | G2 |
| | MORE OF D1- | | | | | |
| | D28 | | | | | |
| 309 | ANY ONE OR | B2 | A8 | E1 | F1 | ANY ONE |
| | MORE OF D1- | | | | | OF G3- |
| | D28 | | | | | G12 |
| 310 | ANY ONE OR | B2 | A8 | E1 | F2 | G1 |
| | MORE OF D1- | | | | | |
| | D28 | | | | | |
| 311 | ANY ONE OR | B2 | A8 | E1 | F2 | G2 |
| | MORE OF D1- | | | | | |
| | D28 | | | | | |
| 312 | ANY ONE OR | B2 | A8 | E1 | F2 | ANY ONE |
| | MORE OF D1- | | | | | OF G3- |
| | D28 | | | | | G12 |
| 313 | ANY ONE OR | B2 | A8 | E1 | F3 | G1 |
| | MORE OF D1- | | | | | |
| | D28 | | | | | |
| 314 | ANY ONE OR | B2 | A8 | E1 | F3 | G2 |
| | MORE OF D1- | | | | | |
| | D28 | | | | | |
| 315 | ANY ONE OR | B2 | A8 | E1 | F3 | ANY ONE |
| | MORE OF D1- | | | | | OF G3- |
| | D28 | | | | | G12 |
| 316 | ANY ONE OR | B2 | A8 | ANY ONE OF | F1 | G1 |
| | MORE OF D1- | | | E2-E4 | | |
| | D28 | | | | | |
| 317 | ANY ONE OR | B2 | A8 | ANY ONE OF | F1 | G2 |
| | MORE OF D1- | | | E2-E4 | | |
| | D28 | | | | | |
| 318 | ANY ONE OR | B2 | A8 | ANY ONE OF | F1 | ANY ONE |
| | MORE OF D1- | | | E2-E4 | | OF G3- |
| | D28 | | | | | G12 |
| 319 | ANY ONE OR | B2 | A8 | ANY ONE OF | F2 | G1 |
| | MORE OF D1- | | | E2-E4 | | |
| | D28 | | | | | |
| 320 | ANY ONE OR | B2 | A8 | ANY ONE OF | F2 | G2 |
| | MORE OF D1- | | | E2-E4 | | |
| | D28 | | | | | |
| 321 | ANY ONE OR | B2 | A8 | ANY ONE OF | F2 | ANY ONE |
| | MORE OF D1- | | | E2-E4 | | OF G3- |
| | D28 | | | | | G12 |
| 322 | ANY ONE OR | B2 | A8 | ANY ONE OF | F3 | G1 |
| | MORE OF D1- | | | E2-E4 | | |
| | D28 | | | | | |
| 323 | ANY ONE OR | B2 | A8 | ANY ONE OF | F3 | G2 |
| | MORE OF D1- | | | E2-E4 | | |
| | D28 | | | | | |
| 324 | ANY ONE OR | B2 | A8 | ANY ONE OF | F3 | ANY ONE |
| | MORE OF D1- | | | E2-E4 | | OF G3- |
| | D28 | | | | | G12 |
| 325 | ANY ONE OR | B2 | A9 | E1 | F1 | G1 |
| | MORE OF D1- | | | | | |
| | D28 | | | | | |
| 326 | ANY ONE OR | B2 | A9 | E1 | F1 | G2 |
| | MORE OF D1- | | | | | |
| | D28 | | | | | |
| 327 | ANY ONE OR | B2 | A9 | E1 | F1 | ANY ONE |
| | MORE OF D1- | | | | | OF G3- |
| | D28 | | | | | G12 |
| 328 | ANY ONE OR | B2 | A9 | E1 | F2 | G1 |
| | MORE OF D1- | | | | | |
| | D28 | | | | | |
| 329 | ANY ONE OR | B2 | A9 | E1 | F2 | G2 |
| | MORE OF D1- | | | | | |
| | D28 | | | | | |
| 330 | ANY ONE OR | B2 | A9 | E1 | F2 | ANY ONE |
| | MORE OF D1- | | | | | OF G3- |
| | D28 | | | | | G12 |
| 331 | ANY ONE OR | B2 | A9 | E1 | F3 | G1 |
| | MORE OF D1- | | | | | |
| | D28 | | | | | |
| 332 | ANY ONE OR | B2 | A9 | E1 | F3 | G2 |
| | MORE OF D1- | | | | | |
| | D28 | | | | | |
| 333 | ANY ONE OR | B2 | A9 | E1 | F3 | ANY ONE |
| | MORE OF D1- | | | | | OF G3- |
| | D28 | | | | | G12 |
| 334 | ANY ONE OR | B2 | A9 | ANY ONE OF | F1 | G1 |
| | MORE OF D1- | | | E2-E4 | | |
| | D28 | | | | | |
| 335 | ANY ONE OR | B2 | A9 | ANY ONE OF | F1 | G2 |
| | MORE OF D1- | | | E2-E4 | | |
| | D28 | | | | | |
| 336 | ANY ONE OR | B2 | A9 | ANY ONE OF | F1 | ANY ONE |
| | MORE OF D1- | | | E2-E4 | | OF G3- |
| | D28 | | | | | G12 |
| 337 | ANY ONE OR | B2 | A9 | ANY ONE OF | F2 | G1 |
| | MORE OF D1- | | | E2-E4 | | |
| | D28 | | | | | |
| 338 | ANY ONE OR | B2 | A9 | ANY ONE OF | F2 | G2 |
| | MORE OF D1- | | | E2-E4 | | |
| | D28 | | | | | |
| 339 | ANY ONE OR | B2 | A9 | ANY ONE OF | F2 | ANY ONE |
| | MORE OF D1- | | | E2-E4 | | OF G3- |
| | D28 | | | | | G12 |
| 340 | ANY ONE OR | B2 | A9 | ANY ONE OF | F3 | G1 |
| | MORE OF D1- | | | E2-E4 | | |
| | D28 | | | | | |
| 341 | ANY ONE OR | B2 | A9 | ANY ONE OF | F3 | G2 |
| | MORE OF D1- | | | E2-E4 | | |
| | D28 | | | | | |
| 342 | ANY ONE OR | B2 | A9 | ANY ONE OF | F3 | ANY ONE |
| | MORE OF D1- | | | E2-E4 | | OF G3- |
| | D28 | | | | | G12 |
| 343 | ANY ONE OR | B2 | A10 | E1 | F1 | G1 |
| | MORE OF D1- | | | | | |
| | D28 | | | | | |
| 344 | ANY ONE OR | B2 | A10 | E1 | F1 | G2 |
| | MORE OF D1- | | | | | |
| | D28 | | | | | |
| 345 | ANY ONE OR | B2 | A10 | E1 | F1 | ANY ONE |
| | MORE OF D1- | | | | | OF G3- |
| | D28 | | | | | G12 |
| 346 | ANY ONE OR | B2 | A10 | E1 | F2 | G1 |
| | MORE OF D1- | | | | | |
| | D28 | | | | | |
| 347 | ANY ONE OR | B2 | A10 | E1 | F2 | G2 |
| | MORE OF D1- | | | | | |
| | D28 | | | | | |
| 348 | ANY ONE OR | B2 | A10 | E1 | F2 | ANY ONE |
| | MORE OF D1- | | | | | OF G3- |
| | D28 | | | | | G12 |
| 349 | ANY ONE OR | B2 | A10 | E1 | F3 | G1 |
| | MORE OF D1- | | | | | |
| | D28 | | | | | |
| 350 | ANY ONE OR | B2 | A10 | E1 | F3 | G2 |
| | MORE OF D1- | | | | | |
| | D28 | | | | | |
| 351 | ANY ONE OR | B2 | A10 | E1 | F3 | ANY ONE |
| | MORE OF D1- | | | | | OF G3- |
| | D28 | | | | | G12 |
| 352 | ANY ONE OR | B2 | A10 | ANY ONE OF | F1 | G1 |
| | MORE OF D1- | | | E2-E4 | | |
| | D28 | | | | | |
| 353 | ANY ONE OR | B2 | A10 | ANY ONE OF | F1 | G2 |
| | MORE OF D1- | | | E2-E4 | | |
| | D28 | | | | | |
| 354 | ANY ONE OR | B2 | A10 | ANY ONE OF | F1 | ANY ONE |
| | MORE OF D1- | | | E2-E4 | | OF G3- |
| | D28 | | | | | G12 |
| 355 | ANY ONE OR | B2 | A10 | ANY ONE OF | F2 | G1 |
| | MORE OF D1- | | | E2-E4 | | |
| | D28 | | | | | |
| 356 | ANY ONE OR | B2 | A10 | ANY ONE OF | F2 | G2 |
| | MORE OF D1- | | | E2-E4 | | |
| | D28 | | | | | |
| 357 | ANY ONE OR | B2 | A10 | ANY ONE OF | F2 | ANY ONE |
| | MORE OF D1- | | | E2-E4 | | OF G3- |
| | D28 | | | | | G12 |
| 358 | ANY ONE OR | B2 | A10 | ANY ONE OF | F3 | G1 |
| | MORE OF D1- | | | E2-E4 | | |
| | D28 | | | | | |
| 359 | ANY ONE OR | B2 | A10 | ANY ONE OF | F3 | G2 |
| | MORE OF D1- | | | E2-E4 | | |
| | D28 | | | | | |
| 360 | ANY ONE OR | B2 | A10 | ANY ONE OF | F3 | ANY ONE |
| | MORE OF D1- | | | E2-E4 | | OF G3- |
| | D28 | | | | | G12 |
| 361 | ANY ONE OR | B3 | A1 | E1 | F1 | G1 |
| | MORE OF D1- | | | | | |
| | D28 | | | | | |
| 362 | ANY ONE OR | B3 | A1 | E1 | F1 | G2 |
| | MORE OF D1- | | | | | |
| | D28 | | | | | |
| 363 | ANY ONE OR | B3 | A1 | E1 | F1 | ANY ONE |
| | MORE OF D1- | | | | | OF G3- |
| | D28 | | | | | G12 |
| 364 | ANY ONE OR | B3 | A1 | E1 | F2 | G1 |
| | MORE OF D1- | | | | | |
| | D28 | | | | | |
| 365 | ANY ONE OR | B3 | A1 | E1 | F2 | G2 |
| | MORE OF D1- | | | | | |
| | D28 | | | | | |
| 366 | ANY ONE OR | B3 | A1 | E1 | F2 | ANY ONE |
| | MORE OF D1- | | | | | OF G3- |
| | D28 | | | | | G12 |
| 367 | ANY ONE OR | B3 | A1 | E1 | F3 | G1 |
| | MORE OF D1- | | | | | |
| | D28 | | | | | |
| 368 | ANY ONE OR | B3 | A1 | E1 | F3 | G2 |
| | MORE OF D1- | | | | | |
| | D28 | | | | | |
| 369 | ANY ONE OR | B3 | A1 | E1 | F3 | ANY ONE |
| | MORE OF D1- | | | | | OF G3- |
| | D28 | | | | | G12 |
| 370 | ANY ONE OR | B3 | A1 | ANY ONE OF | F1 | G1 |
| | MORE OF D1- | | | E2-E4 | | |
| | D28 | | | | | |
| 371 | ANY ONE OR | B3 | A1 | ANY ONE OF | F1 | G2 |
| | MORE OF D1- | | | E2-E4 | | |
| | D28 | | | | | |
| 372 | ANY ONE OR | B3 | A1 | ANY ONE OF | F1 | ANY ONE |
| | MORE OF D1- | | | E2-E4 | | OF G3- |
| | D28 | | | | | G12 |
| 373 | ANY ONE OR | B3 | A1 | ANY ONE OF | F2 | G1 |
| | MORE OF D1- | | | E2-E4 | | |
| | D28 | | | | | |
| 374 | ANY ONE OR MORE | B3 | A1 | ANY ONE OF | F2 | G2 |
| | OF D1-D28 | | | E2-E4 | | |
| 375 | ANY ONE OR | B3 | A1 | ANY ONE OF | F2 | ANY ONE |
| | MORE OF D1- | | | E2-E4 | | OF G3- |
| | D28 | | | | | G12 |
| 376 | ANY ONE OR | B3 | A1 | ANY ONE OF | F3 | G1 |
| | MORE OF D1- | | | E2-E4 | | |
| | D28 | | | | | |
| 377 | ANY ONE OR | B3 | A1 | ANY ONE OF | F3 | G2 |
| | MORE OF D1- | | | E2-E4 | | |
| | D28 | | | | | |
| 378 | ANY ONE OR | B3 | A1 | ANY ONE OF | F3 | ANY ONE |
| | MORE OF D1- | | | E2-E4 | | OF G3- |
| | D28 | | | | | G12 |
| 379 | ANY ONE OR | B3 | A2 | E1 | F1 | G1 |
| | MORE OF D1- | | | | | |
| | D28 | | | | | |
| 380 | ANY ONE OR | B3 | A2 | E1 | F1 | G2 |
| | MORE OF D1- | | | | | |
| | D28 | | | | | |
| 381 | ANY ONE OR | B3 | A2 | E1 | F1 | ANY ONE |
| | MORE OF D1- | | | | | OF G3- |
| | D28 | | | | | G12 |
| 382 | ANY ONE OR | B3 | A2 | E1 | F2 | G1 |
| | MORE OF D1- | | | | | |
| | D28 | | | | | |
| 383 | ANY ONE OR | B3 | A2 | E1 | F2 | G2 |
| | MORE OF D1- | | | | | |
| | D28 | | | | | |
| 384 | ANY ONE OR | B3 | A2 | E1 | F2 | ANY ONE |
| | MORE OF D1- | | | | | OF G3- |
| | D28 | | | | | G12 |
| 385 | ANY ONE OR | B3 | A2 | E1 | F3 | G1 |
| | MORE OF D1- | | | | | |
| | D28 | | | | | |
| 386 | ANY ONE OR | B3 | A2 | E1 | F3 | G2 |
| | MORE OF D1- | | | | | |
| | D28 | | | | | |
| 387 | ANY ONE OR | B3 | A2 | E1 | F3 | ANY ONE |
| | MORE OF D1- | | | | | OF G3- |
| | D28 | | | | | G12 |
| 388 | ANY ONE OR | B3 | A2 | ANY ONE OF | F1 | G1 |
| | MORE OF D1- | | | E2-E4 | | |
| | D28 | | | | | |
| 389 | ANY ONE OR | B3 | A2 | ANY ONE OF | F1 | G2 |
| | MORE OF D1- | | | E2-E4 | | |
| | D28 | | | | | |
| 390 | ANY ONE OR | B3 | A2 | ANY ONE OF | F1 | ANY ONE |
| | MORE OF D1- | | | E2-E4 | | OF G3- |
| | D28 | | | | | G12 |
| 391 | ANY ONE OR | B3 | A2 | ANY ONE OF | F2 | G1 |
| | MORE OF D1- | | | E2-E4 | | |
| | D28 | | | | | |
| 392 | ANY ONE OR | B3 | A2 | ANY ONE OF | F2 | G2 |
| | MORE OF D1- | | | E2-E4 | | |
| | D28 | | | | | |
| 393 | ANY ONE OR | B3 | A2 | ANY ONE OF | F2 | ANY ONE |
| | MORE OF D1- | | | E2-E4 | | OF G3- |
| | D28 | | | | | G12 |
| 394 | ANY ONE OR | B3 | A2 | ANY ONE OF | F3 | G1 |
| | MORE OF D1- | | | E2-E4 | | |
| | D28 | | | | | |
| 395 | ANY ONE OR | B3 | A2 | ANY ONE OF | F3 | G2 |
| | MORE OF D1- | | | E2-E4 | | |
| | D28 | | | | | |
| 396 | ANY ONE OR | B3 | A2 | ANY ONE OF | F3 | ANY ONE |
| | MORE OF D1- | | | E2-E4 | | OF G3- |
| | D28 | | | | | G12 |
| 397 | ANY ONE OR | B3 | A3 | E1 | F1 | G1 |
| | MORE OF D1- | | | | | |
| | D28 | | | | | |
| 398 | ANY ONE OR | B3 | A3 | E1 | F1 | G2 |
| | MORE OF D1- | | | | | |
| | D28 | | | | | |
| 399 | ANY ONE OR | B3 | A3 | E1 | F1 | ANY ONE |
| | MORE OF D1- | | | | | OF G3- |
| | D28 | | | | | G12 |
| 400 | ANY ONE OR | B3 | A3 | E1 | F2 | G1 |
| | MORE OF D1- | | | | | |
| | D28 | | | | | |
| 401 | ANY ONE OR | B3 | A3 | E1 | F2 | G2 |
| | MORE OF D1- | | | | | |
| | D28 | | | | | |
| 402 | ANY ONE OR | B3 | A3 | E1 | F2 | ANY ONE |
| | MORE OF D1- | | | | | OF G3- |
| | D28 | | | | | G12 |
| 403 | ANY ONE OR | B3 | A3 | E1 | F3 | G1 |
| | MORE OF D1- | | | | | |
| | D28 | | | | | |
| 404 | ANY ONE OR | B3 | A3 | E1 | F3 | G2 |
| | MORE OF D1- | | | | | |
| | D28 | | | | | |
| 405 | ANY ONE OR | B3 | A3 | E1 | F3 | ANY ONE |
| | MORE OF D1- | | | | | OF G3- |
| | D28 | | | | | G12 |
| 406 | ANY ONE OR | B3 | A3 | ANY ONE OF | F1 | G1 |
| | MORE OF D1- | | | E2-E4 | | |
| | D28 | | | | | |
| 407 | ANY ONE OR | B3 | A3 | ANY ONE OF | F1 | G2 |
| | MORE OF D1- | | | E2-E4 | | |
| | D28 | | | | | |
| 408 | ANY ONE OR | B3 | A3 | ANY ONE OF | F1 | ANY ONE |
| | MORE OF D1- | | | E2-E4 | | OF G3- |
| | D28 | | | | | G12 |
| 409 | ANY ONE OR | B3 | A3 | ANY ONE OF | F2 | G1 |
| | MORE OF D1- | | | E2-E4 | | |
| | D28 | | | | | |
| 410 | ANY ONE OR | B3 | A3 | ANY ONE OF | F2 | G2 |
| | MORE OF D1- | | | E2-E4 | | |
| | D28 | | | | | |
| 411 | ANY ONE OR | B3 | A3 | ANY ONE OF | F2 | ANY ONE |
| | MORE OF D1- | | | E2-E4 | | OF G3- |
| | D28 | | | | | G12 |
| 412 | ANY ONE OR | B3 | A3 | ANY ONE OF | F3 | G1 |
| | MORE OF D1- | | | E2-E4 | | |
| | D28 | | | | | |
| 413 | ANY ONE OR | B3 | A3 | ANY ONE OF | F3 | G2 |
| | MORE OF D1- | | | E2-E4 | | |
| | D28 | | | | | |
| 414 | ANY ONE OR | B3 | A3 | ANY ONE OF | F3 | ANY ONE |
| | MORE OF D1- | | | E2-E4 | | OF G3- |
| | D28 | | | | | G12 |
| 415 | ANY ONE OR | B3 | A4 | E1 | F1 | G1 |
| | MORE OF D1- | | | | | |
| | D28 | | | | | |
| 416 | ANY ONE OR | B3 | A4 | E1 | F1 | G2 |
| | MORE OF D1- | | | | | |
| | D28 | | | | | |
| 417 | ANY ONE OR | B3 | A4 | E1 | F1 | ANY ONE |
| | MORE OF D1- | | | | | OF G3- |
| | D28 | | | | | G12 |
| 418 | ANY ONE OR | B3 | A4 | E1 | F2 | G1 |
| | MORE OF D1- | | | | | |
| | D28 | | | | | |
| 419 | ANY ONE OR | B3 | A4 | E1 | F2 | G2 |
| | MORE OF D1- | | | | | |
| | D28 | | | | | |
| 420 | ANY ONE OR | B3 | A4 | E1 | F2 | ANY ONE |
| | MORE OF D1- | | | | | OF G3- |
| | D28 | | | | | G12 |
| 421 | ANY ONE OR | B3 | A4 | E1 | F3 | G1 |
| | MORE OF D1- | | | | | |
| | D28 | | | | | |
| 422 | ANY ONE OR | B3 | A4 | E1 | F3 | G2 |
| | MORE OF D1- | | | | | |
| | D28 | | | | | |
| 423 | ANY ONE OR | B3 | A4 | E1 | F3 | ANY ONE |
| | MORE OF D1- | | | | | OF G3- |
| | D28 | | | | | G12 |
| 424 | ANY ONE OR | B3 | A4 | ANY ONE OF | F1 | G1 |
| | MORE OF D1- | | | E2-E4 | | |
| | D28 | | | | | |
| 425 | ANY ONE OR | B3 | A4 | ANY ONE OF | F1 | G2 |
| | MORE OF D1- | | | E2-E4 | | |
| | D28 | | | | | |
| 426 | ANY ONE OR | B3 | A4 | ANY ONE OF | F1 | ANY ONE |
| | MORE OF D1- | | | E2-E4 | | OF G3- |
| | D28 | | | | | G12 |
| 427 | ANY ONE OR | B3 | A4 | ANY ONE OF | F2 | G1 |
| | MORE OF D1- | | | E2-E4 | | |
| | D28 | | | | | |
| 428 | ANY ONE OR | B3 | A4 | ANY ONE OF | F2 | G2 |
| | MORE OF D1- | | | E2-E4 | | |
| | D28 | | | | | |
| 429 | ANY ONE OR | B3 | A4 | ANY ONE OF | F2 | ANY ONE |
| | MORE OF D1- | | | E2-E4 | | OF G3- |
| | D28 | | | | | G12 |
| 430 | ANY ONE OR | B3 | A4 | ANY ONE OF | F3 | G1 |
| | MORE OF D1- | | | E2-E4 | | |
| | D28 | | | | | |
| 431 | ANY ONE OR | B3 | A4 | ANY ONE OF | F3 | G2 |
| | MORE OF D1- | | | E2-E4 | | |
| | D28 | | | | | |
| 432 | ANY ONE OR | B3 | A4 | ANY ONE OF | F3 | ANY ONE |
| | MORE OF D1- | | | E2-E4 | | OF G3- |
| | D28 | | | | | G12 |
| 433 | ANY ONE OR | B3 | A5 | E1 | F1 | G1 |
| | MORE OF D1- | | | | | |
| | D28 | | | | | |
| 434 | ANY ONE OR | B3 | A5 | E1 | F1 | G2 |
| | MORE OF D1- | | | | | |
| | D28 | | | | | |
| 435 | ANY ONE OR | B3 | A5 | E1 | F1 | ANY ONE |
| | MORE OF D1- | | | | | OF G3- |
| | D28 | | | | | G12 |
| 436 | ANY ONE OR | B3 | A5 | E1 | F2 | G1 |
| | MORE OF D1- | | | | | |
| | D28 | | | | | |
| 437 | ANY ONE OR | B3 | A5 | E1 | F2 | G2 |
| | MORE OF D1- | | | | | |
| | D28 | | | | | |
| 438 | ANY ONE OR | B3 | A5 | E1 | F2 | ANY ONE |
| | MORE OF D1- | | | | | OF G3- |
| | D28 | | | | | G12 |
| 439 | ANY ONE OR | B3 | A5 | E1 | F3 | G1 |
| | MORE OF D1- | | | | | |
| | D28 | | | | | |
| 440 | ANY ONE OR | B3 | A5 | E1 | F3 | G2 |
| | MORE OF D1- | | | | | |
| | D28 | | | | | |
| 441 | ANY ONE OR | B3 | A5 | E1 | F3 | ANY ONE |
| | MORE OF D1- | | | | | OF G3- |
| | D28 | | | | | G12 |
| 442 | ANY ONE OR | B3 | A5 | ANY ONE OF | F1 | G1 |
| | MORE OF D1- | | | E2-E4 | | |
| | D28 | | | | | |
| 443 | ANY ONE OR | B3 | A5 | ANY ONE OF | F1 | G2 |
| | MORE OF D1- | | | E2-E4 | | |
| | D28 | | | | | |
| 444 | ANY ONE OR | B3 | A5 | ANY ONE OF | F1 | ANY ONE |
| | MORE OF D1- | | | E2-E4 | | OF G3- |
| | D28 | | | | | G12 |
| 445 | ANY ONE OR | B3 | A5 | ANY ONE OF | F2 | G1 |
| | MORE OF D1- | | | E2-E4 | | |
| | D28 | | | | | |
| 446 | ANY ONE OR | B3 | A5 | ANY ONE OF | F2 | G2 |
| | MORE OF D1- | | | E2-E4 | | |
| | D28 | | | | | |
| 447 | ANY ONE OR | B3 | A5 | ANY ONE OF | F2 | ANY ONE |
| | MORE OF D1- | | | E2-E4 | | OF G3- |
| | D28 | | | | | G12 |
| 448 | ANY ONE OR | B3 | A5 | ANY ONE OF | F3 | G1 |
| | MORE OF D1- | | | E2-E4 | | |
| | D28 | | | | | |
| 449 | ANY ONE OR | B3 | A5 | ANY ONE OF | F3 | G2 |
| | MORE OF D1- | | | E2-E4 | | |
| | D28 | | | | | |
| 450 | ANY ONE OR | B3 | A5 | ANY ONE OF | F3 | ANY ONE |
| | MORE OF D1- | | | E2-E4 | | OF G3- |
| | D28 | | | | | G12 |
| 451 | ANY ONE OR | B3 | A6 | E1 | F1 | G1 |
| | MORE OF D1- | | | | | |
| | D28 | | | | | |
| 452 | ANY ONE OR | B3 | A6 | E1 | F1 | G2 |
| | MORE OF D1- | | | | | |
| | D28 | | | | | |
| 453 | ANY ONE OR | B3 | A6 | E1 | F1 | ANY ONE |
| | MORE OF D1- | | | | | OF G3- |
| | D28 | | | | | G12 |
| 454 | ANY ONE OR | B3 | A6 | E1 | F2 | G1 |
| | MORE OF D1- | | | | | |
| | D28 | | | | | |
| 455 | ANY ONE OR | B3 | A6 | E1 | F2 | G2 |
| | MORE OF D1- | | | | | |
| | D28 | | | | | |
| 456 | ANY ONE OR | B3 | A6 | E1 | F2 | ANY ONE |
| | MORE OF D1- | | | | | OF G3- |
| | D28 | | | | | G12 |
| 457 | ANY ONE OR | B3 | A6 | E1 | F3 | G1 |
| | MORE OF D1- | | | | | |
| | D28 | | | | | |
| 458 | ANY ONE OR | B3 | A6 | E1 | F3 | G2 |
| | MORE OF D1- | | | | | |
| | D28 | | | | | |
| 459 | ANY ONE OR | B3 | A6 | E1 | F3 | ANY ONE |
| | MORE OF D1- | | | | | OF G3- |
| | D28 | | | | | G12 |
| 460 | ANY ONE OR | B3 | A6 | ANY ONE OF | F1 | G1 |
| | MORE OF D1- | | | E2-E4 | | |
| | D28 | | | | | |
| 461 | ANY ONE OR | B3 | A6 | ANY ONE OF | F1 | G2 |
| | MORE OF D1- | | | E2-E4 | | |
| | D28 | | | | | |
| 462 | ANY ONE OR | B3 | A6 | ANY ONE OF | F1 | ANY ONE |
| | MORE OF D1- | | | E2-E4 | | OF G3- |
| | D28 | | | | | G12 |
| 463 | ANY ONE OR | B3 | A6 | ANY ONE OF | F2 | G1 |
| | MORE OF D1- | | | E2-E4 | | |
| | D28 | | | | | |
| 464 | ANY ONE OR | B3 | A6 | ANY ONE OF | F2 | G2 |
| | MORE OF D1- | | | E2-E4 | | |
| | D28 | | | | | |
| 465 | ANY ONE OR | B3 | A6 | ANY ONE OF | F2 | ANY ONE |
| | MORE OF D1- | | | E2-E4 | | OF G3- |
| | D28 | | | | | G12 |
| 466 | ANY ONE OR | B3 | A6 | ANY ONE OF | F3 | G1 |
| | MORE OF D1- | | | E2-E4 | | |
| | D28 | | | | | |
| 467 | ANY ONE OR | B3 | A6 | ANY ONE OF | F3 | G2 |
| | MORE OF D1- | | | E2-E4 | | |
| | D28 | | | | | |
| 468 | ANY ONE OR | B3 | A6 | ANY ONE OF | F3 | ANY ONE |
| | MORE OF D1- | | | E2-E4 | | OF G3- |
| | D28 | | | | | G12 |
| 469 | ANY ONE OR | B3 | A7 | E1 | F1 | G1 |
| | MORE OF D1- | | | | | |
| | D28 | | | | | |
| 470 | ANY ONE OR | B3 | A7 | E1 | F1 | G2 |
| | MORE OF D1- | | | | | |
| | D28 | | | | | |
| 471 | ANY ONE OR | B3 | A7 | E1 | F1 | ANY ONE |
| | MORE OF D1- | | | | | OF G3- |
| | D28 | | | | | G12 |
| 472 | ANY ONE OR | B3 | A7 | E1 | F2 | G1 |
| | MORE OF D1- | | | | | |
| | D28 | | | | | |
| 473 | ANY ONE OR | B3 | A7 | E1 | F2 | G2 |
| | MORE OF D1- | | | | | |
| | D28 | | | | | |
| 474 | ANY ONE OR | B3 | A7 | E1 | F2 | ANY ONE |
| | MORE OF D1- | | | | | OF G3- |
| | D28 | | | | | G12 |
| 475 | ANY ONE OR | B3 | A7 | E1 | F3 | G1 |
| | MORE OF D1- | | | | | |
| | D28 | | | | | |
| 476 | ANY ONE OR | B3 | A7 | E1 | F 3 | G2 |
| | MORE OF D1- | | | | | |
| | D28 | | | | | |
| 477 | ANY ONE OR | B3 | A7 | E1 | F3 | ANY ONE |
| | MORE OF D1- | | | | | OF G3- |
| | D28 | | | | | G12 |
| 478 | ANY ONE OR | B3 | A7 | ANY ONE OF | F1 | G1 |
| | MORE OF D1- | | | E2-E4 | | |
| | D28 | | | | | |
| 479 | ANY ONE OR | B3 | A7 | ANY ONE OF | F1 | G2 |
| | MORE OF D1- | | | E2-E4 | | |
| | D28 | | | | | |
| 480 | ANY ONE OR | B3 | A7 | ANY ONE OF | F1 | ANY ONE |
| | MORE OF D1- | | | E2-E4 | | OF G3- |
| | D28 | | | | | G12 |
| 481 | ANY ONE OR | B3 | A7 | ANY ONE OF | F2 | G1 |
| | MORE OF D1- | | | E2-E4 | | |
| | D28 | | | | | |
| 482 | ANY ONE OR | B3 | A7 | ANY ONE OF | F2 | G2 |
| | MORE OF D1- | | | E2-E4 | | |
| | D28 | | | | | |
| 483 | ANY ONE OR | B3 | A7 | ANY ONE OF | F2 | ANY ONE |
| | MORE OF D1- | | | E2-E4 | | OF G3- |
| | D28 | | | | | G12 |
| 484 | ANY ONE OR | B3 | A7 | ANY ONE OF | F3 | G1 |
| | MORE OF D1- | | | E2-E4 | | |
| | D28 | | | | | |
| 485 | ANY ONE OR | B3 | A7 | ANY ONE OF | F3 | G2 |
| | MORE OF D1- | | | E2-E4 | | |
| | D28 | | | | | |
| 486 | ANY ONE OR | B3 | A7 | ANY ONE OF | F3 | ANY ONE |
| | MORE OF D1- | | | E2-E4 | | OF G3- |
| | D28 | | | | | G12 |
| 487 | ANY ONE OR | B3 | A8 | E1 | F1 | G1 |
| | MORE OF D1- | | | | | |
| | D28 | | | | | |
| 488 | ANY ONE OR | B3 | A8 | E1 | F1 | G2 |
| | MORE OF D1- | | | | | |
| | D28 | | | | | |
| 489 | ANY ONE OR | B3 | A8 | E1 | F1 | ANY ONE |
| | MORE OF D1- | | | | | OF G3- |
| | D28 | | | | | G12 |
| 490 | ANY ONE OR | B3 | A8 | E1 | F2 | G1 |
| | MORE OF D1- | | | | | |
| | D28 | | | | | |
| 491 | ANY ONE OR | B3 | A8 | E1 | F2 | G2 |
| | MORE OF D1- | | | | | |
| | D28 | | | | | |
| 492 | ANY ONE OR | B3 | A8 | E1 | F2 | ANY ONE |
| | MORE OF D1- | | | | | OF G3- |
| | D28 | | | | | G12 |
| 493 | ANY ONE OR | B3 | A8 | E1 | F3 | G1 |
| | MORE OF D1- | | | | | |
| | D28 | | | | | |
| 494 | ANY ONE OR | B3 | A8 | E1 | F3 | G2 |
| | MORE OF D1- | | | | | |
| | D28 | | | | | |
| 495 | ANY ONE OR | B3 | A8 | E1 | F3 | ANY ONE |
| | MORE OF D1- | | | | | OF G3- |
| | D28 | | | | | G12 |
| 496 | ANY ONE OR | B3 | A8 | ANY ONE OF | F1 | G1 |
| | MORE OF D1- | | | E2-E4 | | |
| | D28 | | | | | |
| 497 | ANY ONE OR | B3 | A8 | ANY ONE OF | F1 | G2 |
| | MORE OF D1- | | | E2-E4 | | |
| | D28 | | | | | |
| 498 | ANY ONE OR | B3 | A8 | ANY ONE OF | F1 | ANY ONE |
| | MORE OF D1- | | | E2-E4 | | OF G3- |
| | D28 | | | | | G12 |
| 499 | ANY ONE OR | B3 | A8 | ANY ONE OF | F2 | G1 |
| | MORE OF D1- | | | E2-E4 | | |
| | D28 | | | | | |
| 500 | ANY ONE OR | B3 | A8 | ANY ONE OF | F2 | G2 |
| | MORE OF D1- | | | E2-E4 | | |
| | D28 | | | | | |
| 501 | ANY ONE OR | B3 | A8 | ANY ONE OF | F2 | ANY ONE |
| | MORE OF D1- | | | E2-E4 | | OF G3- |
| | D28 | | | | | G12 |
| 502 | ANY ONE OR | B3 | A8 | ANY ONE OF | F3 | G1 |
| | MORE OF D1- | | | E2-E4 | | |
| | D28 | | | | | |
| 503 | ANY ONE OR | B3 | A8 | ANY ONE OF | F3 | G2 |
| | MORE OF D1- | | | E2-E4 | | |
| | D28 | | | | | |
| 504 | ANY ONE OR | B3 | A8 | ANY ONE OF | F3 | ANY ONE |
| | MORE OF D1- | | | E2-E4 | | OF G3- |
| | D28 | | | | | G12 |
| 505 | ANY ONE OR | B3 | A9 | E1 | F1 | G1 |
| | MORE OF D1- | | | | | |
| | D28 | | | | | |
| 506 | ANY ONE OR | B3 | A9 | E1 | F1 | G2 |
| | MORE OF D1- | | | | | |
| | D28 | | | | | |
| 507 | ANY ONE OR | B3 | A9 | E1 | F1 | ANY ONE |
| | MORE OF D1- | | | | | OF G3- |
| | D28 | | | | | G12 |
| 508 | ANY ONE OR | B3 | A9 | E1 | F2 | G1 |
| | MORE OF D1- | | | | | |
| | D28 | | | | | |
| 509 | ANY ONE OR | B3 | A9 | E1 | F2 | G2 |
| | MORE OF D1- | | | | | |
| | D28 | | | | | |
| 510 | ANY ONE OR | B3 | A9 | E1 | F2 | ANY ONE |
| | MORE OF D1- | | | | | OF G3- |
| | D28 | | | | | G12 |
| 511 | ANY ONE OR | B3 | A9 | E1 | F3 | G1 |
| | MORE OF D1- | | | | | |
| | D28 | | | | | |
| 512 | ANY ONE OR | B3 | A9 | E1 | F3 | G2 |
| | MORE OF D1- | | | | | |
| | D28 | | | | | |
| 513 | ANY ONE OR | B3 | A9 | E1 | F3 | ANY ONE |
| | MORE OF D1- | | | | | OF G3- |
| | D28 | | | | | G12 |
| 514 | ANY ONE OR | B3 | A9 | ANY ONE OF | F1 | G1 |
| | MORE OF D1- | | | E2-E4 | | |
| | D28 | | | | | |
| 515 | ANY ONE OR | B3 | A9 | ANY ONE OF | F1 | G2 |
| | MORE OF D1- | | | E2-E4 | | |
| | D28 | | | | | |
| 516 | ANY ONE OR | B3 | A9 | ANY ONE OF | F1 | ANY ONE |
| | MORE OF D1- | | | E2-E4 | | OF G3- |
| | D28 | | | | | G12 |
| 517 | ANY ONE OR | B3 | A9 | ANY ONE OF | F2 | G1 |
| | MORE OF D1- | | | E2-E4 | | |
| | D28 | | | | | |
| 518 | ANY ONE OR | B3 | A9 | ANY ONE OF | F2 | G2 |
| | MORE OF D1- | | | E2-E4 | | |
| | D28 | | | | | |
| 519 | ANY ONE OR | B3 | A9 | ANY ONE OF | F2 | ANY ONE |
| | MORE OF D1- | | | E2-E4 | | OF G3- |
| | D28 | | | | | G12 |
| 520 | ANY ONE OR | B3 | A9 | ANY ONE OF | F3 | G1 |
| | MORE OF D1- | | | E2-E4 | | |
| | D28 | | | | | |
| 521 | ANY ONE OR | B3 | A9 | ANY ONE OF | F3 | G2 |
| | MORE OF D1- | | | E2-E4 | | |
| | D28 | | | | | |
| 522 | ANY ONE OR | B3 | A9 | ANY ONE OF | F3 | ANY ONE |
| | MORE OF D1- | | | E2-E4 | | OF G3- |
| | D28 | | | | | G12 |
| 523 | ANY ONE OR | B3 | A10 | E1 | F1 | G1 |
| | MORE OF D1- | | | | | |
| | D28 | | | | | |
| 524 | ANY ONE OR | B3 | A10 | E1 | F1 | G2 |
| | MORE OF D1- | | | | | |
| | D28 | | | | | |
| 525 | ANY ONE OR | B3 | A10 | E1 | F1 | ANY ONE |
| | MORE OF D1- | | | | | OF G3- |
| | D28 | | | | | G12 |
| 526 | ANY ONE OR | B3 | A10 | E1 | F2 | G1 |
| | MORE OF D1- | | | | | |
| | D28 | | | | | |
| 527 | ANY ONE OR | B3 | A10 | E1 | F2 | G2 |
| | MORE OF D1- | | | | | |
| | D28 | | | | | |
| 528 | ANY ONE OR MORE | B3 | A10 | E1 | F2 | ANY ONE |
| | OF D1-D28 | | | | | OF G3- |
| | | | | | | G12 |
| 529 | ANY ONE OR | B3 | A10 | E1 | F3 | G1 |
| | MORE OF D1- | | | | | |
| | D28 | | | | | |
| 530 | ANY ONE OR MORE | B3 | A10 | E1 | F3 | G2 |
| | OF D1-D28 | | | | | |
| 531 | ANY ONE OR | B3 | A10 | E1 | F3 | ANY ONE |
| | MORE OF D1- | | | | | OF G3- |
| | D28 | | | | | G12 |
| 532 | ANY ONE OR | B3 | A10 | ANY ONE OF | F1 | G1 |
| | MORE OF D1- | | | E2-E4 | | |
| | D28 | | | | | |
| 533 | ANY ONE OR | B3 | A10 | ANY ONE OF | F1 | G2 |
| | MORE OF D1- | | | E2-E4 | | |
| | D28 | | | | | |
| 534 | ANY ONE OR | B3 | A10 | ANY ONE OF | F1 | ANY ONE |
| | MORE OF D1- | | | E2-E4 | | OF G3- |
| | D28 | | | | | G12 |
| 535 | ANY ONE OR | B3 | A10 | ANY ONE OF | F2 | G1 |
| | MORE OF D1- | | | E2-E4 | | |
| | D28 | | | | | |
| 536 | ANY ONE OR | B3 | A10 | ANY ONE OF | F2 | G2 |
| | MORE OF D1- | | | E2-E4 | | |
| | D28 | | | | | |
| 537 | ANY ONE OR | B3 | A10 | ANY ONE OF | F2 | ANY ONE |
| | MORE OF D1- | | | E2-E4 | | OF G3- |
| | D28 | | | | | G12 |
| 538 | ANY ONE OR | B3 | A10 | ANY ONE OF | F3 | G1 |
| | MORE OF D1- | | | E2-E4 | | |
| | D28 | | | | | |
| 539 | ANY ONE OR | B3 | A10 | ANY ONE OF | F3 | G2 |
| | MORE OF D1- | | | E2-E4 | | |
| | D28 | | | | | |
| 540 | ANY ONE OR | B3 | A10 | ANY ONE OF | F3 | ANY ONE |
| | MORE OF D1- | | | E2-E4 | | OF G3- |
| | D28 | | | | | G12 |
| 541 | ANY ONE OR | ANY ONE OF | A1 | E1 | F1 | G1 |
| | MORE OF D1- | B4-B22 | | | | |
| | D28 | | | | | |
| 542 | ANY ONE OR | ANY ONE OF | A1 | E1 | F1 | G2 |
| | MORE OF D1- | B4-B22 | | | | |
| | D28 | | | | | |
| 543 | ANY ONE OR | ANY ONE OF | A1 | E1 | F1 | ANY ONE |
| | MORE OF D1- | B4-B22 | | | | OF G3- |
| | D28 | | | | | G12 |
| 544 | ANY ONE OR | ANY ONE OF | A1 | E1 | F2 | G1 |
| | MORE OF D1- | B4-B22 | | | | |
| | D28 | | | | | |
| 545 | ANY ONE OR | ANY ONE OF | A1 | E1 | F2 | G2 |
| | MORE OF D1- | B4-B22 | | | | |
| | D28 | | | | | |
| 546 | ANY ONE OR | ANY ONE OF | A1 | E1 | F2 | ANY ONE |
| | MORE OF D1- | B4-B22 | | | | OF G3- |
| | D28 | | | | | G12 |
| 547 | ANY ONE OR | ANY ONE OF | A1 | E1 | F3 | G1 |
| | MORE OF D1- | B4-B22 | | | | |
| | D28 | | | | | |
| 548 | ANY ONE OR | ANY ONE OF | A1 | E1 | F3 | G2 |
| | MORE OF D1- | B4-B22 | | | | |
| | D28 | | | | | |
| 549 | ANY ONE OR | ANY ONE OF | A1 | E1 | F3 | ANY ONE |
| | MORE OF D1- | B4-B22 | | | | OF G3- |
| | D28 | | | | | G12 |
| 550 | ANY ONE OR | ANY ONE OF | A1 | ANY ONE OF | F1 | G1 |
| | MORE OF D1- | B4-B22 | | E2-E4 | | |
| | D28 | | | | | |
| 551 | ANY ONE OR | ANY ONE OF | A1 | ANY ONE OF | F1 | G2 |
| | MORE OF D1- | B4-B22 | | E2-E4 | | |
| | D28 | | | | | |
| 552 | ANY ONE OR | ANY ONE OF | A1 | ANY ONE OF | F1 | ANY ONE |
| | MORE OF D1- | B4-B22 | | E2-E4 | | OF G3- |
| | D28 | | | | | G12 |
| 553 | ANY ONE OR | ANY ONE OF | A1 | ANY ONE OF | F2 | G1 |
| | MORE OF D1- | B4-B22 | | E2-E4 | | |
| | D28 | | | | | |
| 554 | ANY ONE OR | ANY ONE OF | A1 | ANY ONE OF | F2 | G2 |
| | MORE OF D1- | B4-B22 | | E2-E4 | | |
| | D28 | | | | | |
| 555 | ANY ONE OR | ANY ONE OF | A1 | ANY ONE OF | F2 | ANY ONE |
| | MORE OF D1- | B4-B22 | | E2-E4 | | OF G3- |
| | D28 | | | | | G12 |
| 556 | ANY ONE OR | ANY ONE OF | A1 | ANY ONE OF | F3 | G1 |
| | MORE OF D1- | B4-B22 | | E2-E4 | | |
| | D28 | | | | | |
| 557 | ANY ONE OR | ANY ONE OF | A1 | ANY ONE OF | F3 | G2 |
| | MORE OF D1- | B4-B22 | | E2-E4 | | |
| | D28 | | | | | |
| 558 | ANY ONE OR | ANY ONE OF | A1 | ANY ONE OF | F3 | ANY ONE |
| | MORE OF D1- | B4-B22 | | E2-E4 | | OF G3- |
| | D28 | | | | | G12 |
| 559 | ANY ONE OR | ANY ONE OF | A2 | E1 | F1 | G1 |
| | MORE OF D1- | B4-B22 | | | | |
| | D28 | | | | | |
| 560 | ANY ONE OR | ANY ONE OF | A2 | E1 | F1 | G2 |
| | MORE OF D1- | B4-B22 | | | | |
| | D28 | | | | | |
| 561 | ANY ONE OR | ANY ONE OF | A2 | E1 | F1 | ANY ONE |
| | MORE OF D1- | B4-B22 | | | | OF G3- |
| | D28 | | | | | G12 |
| 562 | ANY ONE OR | ANY ONE OF | A2 | E1 | F2 | G1 |
| | MORE OF D1- | B4-B22 | | | | |
| | D28 | | | | | |
| 563 | ANY ONE OR | ANY ONE OF | A2 | E1 | F2 | G2 |
| | MORE OF D1- | B4-B22 | | | | |
| | D28 | | | | | |
| 564 | ANY ONE OR | ANY ONE OF | A2 | E1 | F2 | ANY ONE |
| | MORE OF D1- | B4-B22 | | | | OF G3- |
| | D28 | | | | | G12 |
| 565 | ANY ONE OR | ANY ONE OF | A2 | E1 | F3 | G1 |
| | MORE OF D1- | B4-B22 | | | | |
| | D28 | | | | | |
| 566 | ANY ONE OR | ANY ONE OF | A2 | E1 | F3 | G2 |
| | MORE OF D1- | B4-B22 | | | | |
| | D28 | | | | | |
| 567 | ANY ONE OR | ANY ONE OF | A2 | E1 | F3 | ANY ONE |
| | MORE OF D1- | B4-B22 | | | | OF G3 |
| | D28 | | | | | G12 |
| 568 | ANY ONE OR | ANY ONE OF | A2 | ANY ONE OF | F1 | G1 |
| | MORE OF D1- | B4-B22 | | E2-E4 | | |
| | D28 | | | | | |
| 569 | ANY ONE OR | ANY ONE OF | A2 | ANY ONE OF | F1 | G2 |
| | MORE OF D1- | B4-B22 | | E2-E4 | | |
| | D28 | | | | | |
| 570 | ANY ONE OR | ANY ONE OF | A2 | ANY ONE OF | F1 | ANY ONE |
| | MORE OF D1- | B4-B22 | | E2-E4 | | OF G3- |
| | D28 | | | | | G12 |
| 571 | ANY ONE OR | ANY ONE OF | A2 | ANY ONE OF | F2 | G1 |
| | MORE OF D1- | B4-B22 | | E2-E4 | | |
| | D28 | | | | | |
| 572 | ANY ONE OR | ANY ONE OF | A2 | ANY ONE OF | F2 | G2 |
| | MORE OF D1- | B4-B22 | | E2-E4 | | |
| | D28 | | | | | |
| 573 | ANY ONE OR | ANY ONE OF | A2 | ANY ONE OF | F2 | ANY ONE |
| | MORE OF D1- | B4-B22 | | E2-E4 | | OF G3- |
| | D28 | | | | | G12 |
| 574 | ANY ONE OR | ANY ONE OF | A2 | ANY ONE OF | F3 | G1 |
| | MORE OF D1- | B4-B22 | | E2-E4 | | |
| | D28 | | | | | |
| 575 | ANY ONE OR | ANY ONE OF | A2 | ANY ONE OF | F3 | G2 |
| | MORE OF D1- | B4-B22 | | E2-E4 | | |
| | D28 | | | | | |
| 576 | ANY ONE OR | ANY ONE OF | A2 | ANY ONE OF | F3 | ANY ONE |
| | MORE OF D1- | B4-B22 | | E2-E4 | | OF G3- |
| | D28 | | | | | G12 |
| 577 | ANY ONE OR | ANY ONE OF | A3 | E1 | F1 | G1 |
| | MORE OF D1- | B4-B22 | | | | |
| | D28 | | | | | |
| 578 | ANY ONE OR | ANY ONE OF | A3 | E1 | F1 | G2 |
| | MORE OF D1- | B4-B22 | | | | |
| | D28 | | | | | |
| 579 | ANY ONE OR | ANY ONE OF | A3 | E1 | F1 | ANY ONE |
| | MORE OF D1- | B4-B22 | | | | OF G3- |
| | D28 | | | | | G12 |
| 580 | ANY ONE OR | ANY ONE OF | A3 | E1 | F2 | G1 |
| | MORE OF D1- | B4-B22 | | | | |
| | D28 | | | | | |
| 581 | ANY ONE OR | ANY ONE OF | A3 | E1 | F2 | G2 |
| | MORE OF D1- | B4-B22 | | | | |
| | D28 | | | | | |
| 582 | ANY ONE OR | ANY ONE OF | A3 | E1 | F2 | ANY ONE |
| | MORE OF D1- | B4-B22 | | | | OF G3- |
| | D28 | | | | | G12 |
| 583 | ANY ONE OR | ANY ONE OF | A3 | E1 | F3 | G1 |
| | MORE OF D1- | B4-B22 | | | | |
| | D28 | | | | | |
| 584 | ANY ONE OR | ANY ONE OF | A3 | E1 | F3 | G2 |
| | MORE OF D1- | B4-B22 | | | | |
| | D28 | | | | | |
| 585 | ANY ONE OR | ANY ONE OF | A3 | E1 | F3 | ANY ONE |
| | MORE OF D1- | B4-B22 | | | | OF G3- |
| | D28 | | | | | G12 |
| 586 | ANY ONE OR | ANY ONE OF | A3 | ANY ONE OF | F1 | G1 |
| | MORE OF D1- | B4-B22 | | E2-E4 | | |
| | D28 | | | | | |
| 587 | ANY ONE OR | ANY ONE OF | A3 | ANY ONE OF | F1 | G2 |
| | MORE OF D1- | B4-B22 | | E2-E4 | | |
| | D28 | | | | | |
| 588 | ANY ONE OR | ANY ONE OF | A3 | ANY ONE OF | F1 | ANY ONE |
| | MORE OF D1- | B4-B22 | | E2-E4 | | OF G3- |
| | D28 | | | | | G12 |
| 589 | ANY ONE OR | ANY ONE OF | A3 | ANY ONE OF | F2 | G1 |
| | MORE OF D1- | B4-B22 | | E2-E4 | | |
| | D28 | | | | | |
| 590 | ANY ONE OR | ANY ONE OF | A3 | ANY ONE OF | F2 | G2 |
| | MORE OF D1- | B4-B22 | | E2-E4 | | |
| | D28 | | | | | |
| 591 | ANY ONE OR | ANY ONE OF | A3 | ANY ONE OF | F2 | ANY ONE |
| | MORE OF D1- | B4-B22 | | E2-E4 | | OF G3- |
| | D28 | | | | | G12 |
| 592 | ANY ONE OR | ANY ONE OF | A3 | ANY ONE OF | F3 | G1 |
| | MORE OF D1- | B4-B22 | | E2-E4 | | |
| | D28 | | | | | |
| 593 | ANY ONE OR | ANY ONE OF | A3 | ANY ONE OF | F3 | G2 |
| | MORE OF D1- | B4-B22 | | E2-E4 | | |
| | D28 | | | | | |
| 594 | ANY ONE OR | ANY ONE OF | A3 | ANY ONE OF | F3 | ANY ONE |
| | MORE OF D1- | B4-B22 | | E2-E4 | | OF G3- |
| | D28 | | | | | G12 |
| 595 | ANY ONE OR | ANY ONE OF | A4 | E1 | F1 | G1 |
| | MORE OF D1- | B4-B22 | | | | |
| | D28 | | | | | |
| 596 | ANY ONE OR | ANY ONE OF | A4 | E1 | F1 | G2 |
| | MORE OF D1- | B4-B22 | | | | |
| | D28 | | | | | |
| 597 | ANY ONE OR | ANY ONE OF | A4 | E1 | F1 | ANY ONE |
| | MORE OF D1- | B4-B22 | | | | OF G3- |
| | D28 | | | | | G12 |
| 598 | ANY ONE OR | ANY ONE OF | A4 | E1 | F2 | G1 |
| | MORE OF D1- | B4-B22 | | | | |
| | D28 | | | | | |
| 599 | ANY ONE OR | ANY ONE OF | A4 | E1 | F2 | G2 |
| | MORE OF D1- | B4-B22 | | | | |
| | D28 | | | | | |
| 600 | ANY ONE OR | ANY ONE OF | A4 | E1 | F2 | ANY ONE |
| | MORE OF D1- | B4-B22 | | | | OF G3- |
| | D28 | | | | | G12 |
| 601 | ANY ONE OR | ANY ONE OF | A4 | E1 | F3 | G1 |
| | MORE OF D1- | B4-B22 | | | | |
| | D28 | | | | | |
| 602 | ANY ONE OR | ANY ONE OF | A4 | E1 | F3 | G2 |
| | MORE OF D1- | B4-B22 | | | | |
| | D28 | | | | | |
| 603 | ANY ONE OR | ANY ONE OF | A4 | E1 | F3 | ANY ONE |
| | MORE OF D1- | B4-B22 | | | | OF G3- |
| | D28 | | | | | G12 |
| 604 | ANY ONE OR | ANY ONE OF | A4 | ANY ONE OF | F1 | G1 |
| | MORE OF D1- | B4-B22 | | E2-E4 | | |
| | D28 | | | | | |
| 605 | ANY ONE OR | ANY ONE OF | A4 | ANY ONE OF | F1 | G2 |
| | MORE OF D1- | B4-B22 | | E2-E4 | | |
| | D28 | | | | | |
| 606 | ANY ONE OR | ANY ONE OF | A4 | ANY ONE OF | F1 | ANY ONE |
| | MORE OF D1- | B4-B22 | | E2-E4 | | OF G3- |
| | D28 | | | | | G12 |
| 607 | ANY ONE OR | ANY ONE OF | A4 | ANY ONE OF | F2 | G1 |
| | MORE OF D1- | B4-B22 | | E2-E4 | | |
| | D28 | | | | | |
| 608 | ANY ONE OR | ANY ONE OF | A4 | ANY ONE OF | F2 | G2 |
| | MORE OF D1- | B4-B22 | | E2-E4 | | |
| | D28 | | | | | |
| 609 | ANY ONE OR | ANY ONE OF | A4 | ANY ONE OF | F2 | ANY ONE |
| | MORE OF D1- | B4-B22 | | E2-E4 | | OF G3- |
| | D28 | | | | | G12 |
| 610 | ANY ONE OR | ANY ONE OF | A4 | ANY ONE OF | F3 | G1 |
| | MORE OF D1- | B4-B22 | | E2-E4 | | |
| | D28 | | | | | |
| 611 | ANY ONE OR | ANY ONE OF | A4 | ANY ONE OF | F3 | G2 |
| | MORE OF D1- | B4-B22 | | E2-E4 | | |
| | D28 | | | | | |
| 612 | ANY ONE OR | ANY ONE OF | A4 | ANY ONE OF | F3 | ANY ONE |
| | MORE OF D1- | B4-B22 | | E2-E4 | | OF G3- |
| | D28 | | | | | G12 |
| 613 | ANY ONE OR | ANY ONE OF | A5 | E1 | F1 | G1 |
| | MORE OF D1- | B4-B22 | | | | |
| | D28 | | | | | |
| 614 | ANY ONE OR | ANY ONE OF | A5 | E1 | F1 | G2 |
| | MORE OF D1- | B4-B22 | | | | |
| | D28 | | | | | |
| 615 | ANY ONE OR | ANY ONE OF | A5 | E1 | F1 | ANY ONE |
| | MORE OF D1- | B4-B22 | | | | OF G3- |
| | D28 | | | | | G12 |
| 616 | ANY ONE OR | ANY ONE OF | A5 | E1 | F2 | G1 |
| | MORE OF D1- | B4-B22 | | | | |
| | D28 | | | | | |
| 617 | ANY ONE OR | ANY ONE OF | A5 | E1 | F2 | G2 |
| | MORE OF D1- | B4-B22 | | | | |
| | D28 | | | | | |
| 618 | ANY ONE OR | ANY ONE OF | A5 | E1 | F2 | ANY ONE |
| | MORE OF D1- | B4-B22 | | | | OF G3- |
| | D28 | | | | | G12 |
| 619 | ANY ONE OR | ANY ONE OF | A5 | E1 | F3 | G1 |
| | MORE OF D1- | B4-B22 | | | | |
| | D28 | | | | | |
| 620 | ANY ONE OR | ANY ONE OF | A5 | E1 | F3 | G2 |
| | MORE OF D1- | B4-B22 | | | | |
| | D28 | | | | | |
| 621 | ANY ONE OR | ANY ONE OF | A5 | E1 | F3 | ANY ONE |
| | MORE OF D1- | B4-B22 | | | | OF G3- |
| | D28 | | | | | G12 |
| 622 | ANY ONE OR | ANY ONE OF | A5 | ANY ONE OF | F1 | G1 |
| | MORE OF D1- | B4-B22 | | E2-E4 | | |
| | D28 | | | | | |
| 623 | ANY ONE OR | ANY ONE OF | A5 | ANY ONE OF | F1 | G2 |
| | MORE OF D1- | B4-B22 | | E2-E4 | | |
| | D28 | | | | | |
| 624 | ANY ONE OR | ANY ONE OF | A5 | ANY ONE OF | F1 | ANY ONE |
| | MORE OF D1- | B4-B22 | | E2-E4 | | OF G3- |
| | D28 | | | | | G12 |
| 625 | ANY ONE OR | ANY ONE OF | A5 | ANY ONE OF | F2 | G1 |
| | MORE OF D1- | B4-B22 | | E2-E4 | | |
| | D28 | | | | | |
| 626 | ANY ONE OR | ANY ONE OF | A5 | ANY ONE OF | F2 | G2 |
| | MORE OF D1- | B4-B22 | | E2-E4 | | |
| | D28 | | | | | |
| 627 | ANY ONE OR | ANY ONE OF | A5 | ANY ONE OF | F2 | ANY ONE |
| | MORE OF D1- | B4-B22 | | E2-E4 | | OF G3- |
| | D28 | | | | | G12 |
| 628 | ANY ONE OR | ANY ONE OF | A5 | ANY ONE OF | F3 | G1 |
| | MORE OF D1- | B4-B22 | | E2-E4 | | |
| | D28 | | | | | |
| 629 | ANY ONE OR | ANY ONE OF | A5 | ANY ONE OF | F3 | G2 |
| | MORE OF D1- | B4-B22 | | E2-E4 | | |
| | D28 | | | | | |
| 630 | ANY ONE OR | ANY ONE OF | A5 | ANY ONE OF | F3 | ANY ONE |
| | MORE OF D1- | B4-B22 | | E2-E4 | | OF G3- |
| | D28 | | | | | G12 |
| 631 | ANY ONE OR | ANY ONE OF | A6 | E1 | F1 | G1 |
| | MORE OF D1- | B4-B22 | | | | |
| | D28 | | | | | |
| 632 | ANY ONE OR | ANY ONE OF | A6 | E1 | F1 | G2 |
| | MORE OF D1- | B4-B22 | | | | |
| | D28 | | | | | |
| 633 | ANY ONE OR | ANY ONE OF | A6 | E1 | F1 | ANY ONE |
| | MORE OF D1- | B4-B22 | | | | OF G3- |
| | D28 | | | | | G12 |
| 634 | ANY ONE OR | ANY ONE OF | A6 | E1 | F2 | G1 |
| | MORE OF D1- | B4-B22 | | | | |
| | D28 | | | | | |
| 635 | ANY ONE OR | ANY ONE OF | A6 | E1 | F2 | G2 |
| | MORE OF D1- | B4-B22 | | | | |
| | D28 | | | | | |
| 636 | ANY ONE OR | ANY ONE OF | A6 | E1 | F2 | ANY ONE |
| | MORE OF D1- | B4-B22 | | | | OF G3- |
| | D28 | | | | | G12 |
| 637 | ANY ONE OR | ANY ONE OF | A6 | E1 | F3 | G1 |
| | MORE OF D1- | B4-B22 | | | | |
| | D28 | | | | | |
| 638 | ANY ONE OR | ANY ONE OF | A6 | E1 | F3 | G2 |
| | MORE OF D1- | B4-B22 | | | | |
| | D28 | | | | | |
| 639 | ANY ONE OR | ANY ONE OF | A6 | E1 | F3 | ANY ONE |
| | MORE OF D1- | B4-B22 | | | | OF G3- |
| | D28 | | | | | G12 |
| 640 | ANY ONE OR | ANY ONE OF | A6 | ANY ONE OF | F1 | G1 |
| | MORE OF D1- | B4-B22 | | E2-E4 | | |
| | D28 | | | | | |
| 641 | ANY ONE OR | ANY ONE OF | A6 | ANY ONE OF | F1 | G2 |
| | MORE OF D1- | B4-B22 | | E2-E4 | | |
| | D28 | | | | | |
| 642 | ANY ONE OR | ANY ONE OF | A6 | ANY ONE OF | F1 | ANY ONE |
| | MORE OF D1- | B4-B22 | | E2-E4 | | OF G3- |
| | D28 | | | | | G12 |
| 643 | ANY ONE OR | ANY ONE OF | A6 | ANY ONE OF | F2 | G1 |
| | MORE OF D1- | B4-B22 | | E2-E4 | | |
| | D28 | | | | | |
| 644 | ANY ONE OR | ANY ONE OF | A6 | ANY ONE OF | F2 | G2 |
| | MORE OF D1- | B4-B22 | | E2-E4 | | |
| | D28 | | | | | |
| 645 | ANY ONE OR | ANY ONE OF | A6 | ANY ONE OF | F2 | ANY ONE |
| | MORE OF D1- | B4-B22 | | E2-E4 | | OF G3- |
| | D28 | | | | | G12 |
| 646 | ANY ONE OR | ANY ONE OF | A6 | ANY ONE OF | F3 | G1 |
| | MORE OF D1- | B4-B22 | | E2-E4 | | |
| | D28 | | | | | |
| 647 | ANY ONE OR | ANY ONE OF | A6 | ANY ONE OF | F3 | G2 |
| | MORE OF D1- | B4-B22 | | E2-E4 | | |
| | D28 | | | | | |
| 648 | ANY ONE OR | ANY ONE OF | A6 | ANY ONE OF | F3 | ANY ONE |
| | MORE OF D1- | B4-B22 | | E2-E4 | | OF G3- |
| | D28 | | | | | G12 |
| 649 | ANY ONE OR | ANY ONE OF | A7 | E1 | F1 | G1 |
| | MORE OF D1- | B4-B22 | | | | |
| | D28 | | | | | |
| 650 | ANY ONE OR | ANY ONE OF | A7 | E1 | F1 | G2 |
| | MORE OF D1- | B4-B22 | | | | |
| | D28 | | | | | |
| 651 | ANY ONE OR | ANY ONE OF | A7 | E1 | F1 | ANY ONE |
| | MORE OF D1- | B4-B22 | | | | OF G3- |
| | D28 | | | | | G12 |
| 652 | ANY ONE OR | ANY ONE OF | A7 | E1 | F2 | G1 |
| | MORE OF D1- | B4-B22 | | | | |
| | D28 | | | | | |
| 653 | ANY ONE OR | ANY ONE OF | A7 | E1 | F2 | G2 |
| | MORE OF D1- | B4-B22 | | | | |
| | D28 | | | | | |
| 654 | ANY ONE OR | ANY ONE OF | A7 | E1 | F2 | ANY ONE |
| | MORE OF D1- | B4-B22 | | | | OF G3- |
| | D28 | | | | | G12 |
| 655 | ANY ONE OR | ANY ONE OF | A7 | E1 | F3 | G1 |
| | MORE OF D1- | B4-B22 | | | | |
| | D28 | | | | | |
| 656 | ANY ONE OR | ANY ONE OF | A7 | E1 | F3 | G2 |
| | MORE OF D1- | B4-B22 | | | | |
| | D28 | | | | | |
| 657 | ANY ONE OR | ANY ONE OF | A7 | E1 | F3 | ANY ONE |
| | MORE OF D1- | B4-B22 | | | | OF G3- |
| | D28 | | | | | G12 |
| 658 | ANY ONE OR | ANY ONE OF | A7 | ANY ONE OF | F1 | G1 |
| | MORE OF D1- | B4-B22 | | E2-E4 | | |
| | D28 | | | | | |
| 659 | ANY ONE OR | ANY ONE OF | A7 | ANY ONE OF | F1 | G2 |
| | MORE OF D1- | B4-B22 | | E2-E4 | | |
| | D28 | | | | | |
| 660 | ANY ONE OR | ANY ONE OF | A7 | ANY ONE OF | F1 | ANY ONE |
| | MORE OF D1- | B4-B22 | | E2-E4 | | OF G3- |
| | D28 | | | | | G12 |
| 661 | ANY ONE OR | ANY ONE OF | A7 | ANY ONE OF | F2 | G1 |
| | MORE OF D1- | B4-B22 | | E2-E4 | | |
| | D28 | | | | | |
| 662 | ANY ONE OR | ANY ONE OF | A7 | ANY ONE OF | F2 | G2 |
| | MORE OF D1- | B4-B22 | | E2-E4 | | |
| | D28 | | | | | |
| 663 | ANY ONE OR MORE | ANY ONE OF | A7 | ANY ONE OF | F2 | ANY ONE |
| | OF D1-D28 | B4-B22 | | E2-E4 | | OF G3- |
| | | | | | | G12 |
| 664 | ANY ONE OR | ANY ONE OF | A7 | ANY ONE OF | F3 | G1 |
| | MORE OF D1- | B4-B22 | | E2-E4 | | |
| | D28 | | | | | |
| 665 | ANY ONE OR | ANY ONE OF | A7 | ANY ONE OF | F3 | G2 |
| | MORE OF D1- | B4-B22 | | E2-E4 | | |
| | D28 | | | | | |
| 666 | ANY ONE OR | ANY ONE OF | A7 | ANY ONE OF | F 3 | ANY ONE |
| | MORE OF D1- | B4-B22 | | E2-E4 | | OF G3- |
| | D28 | | | | | G12 |
| 667 | ANY ONE OR | ANY ONE OF | A8 | E1 | F1 | G1 |
| | MORE OF D1- | B4-B22 | | | | |
| | D28 | | | | | |
| 668 | ANY ONE OR | ANY ONE OF | A8 | E1 | F1 | G2 |
| | MORE OF D1- | B4-B22 | | | | |
| | D28 | | | | | |
| 669 | ANY ONE OR MORE OF D1- | ANY ONE OF | A8 | E1 | F1 | ANY ONE |
| | D28 | B4-B22 | | | | OF G3- |
| | | | | | | G12 |
| 670 | ANY ONE OR | ANY ONE OF | A8 | E1 | F2 | G1 |
| | MORE OF D1- | B4-B22 | | | | |
| | D28 | | | | | |
| 671 | ANY ONE OR | ANY ONE OF | A8 | E1 | F2 | G2 |
| | MORE OF D1- | B4-B22 | | | | |
| | D28 | | | | | |
| 672 | ANY ONE OR MORE | ANY ONE OF | A8 | E1 | F2 | ANY ONE |
| | OF D1-D28 | B4-B22 | | | | OF G3- |
| | | | | | | G12 |
| 673 | ANY ONE OR | ANY ONE OF | A8 | E1 | F3 | G1 |
| | MORE OF D1- | B4-B22 | | | | |
| | D28 | | | | | |
| 674 | ANY ONE OR | ANY ONE OF | A8 | E1 | F3 | G2 |
| | MORE OF D1- | B4-B22 | | | | |
| | D28 | | | | | |
| 675 | ANY ONE OR | ANY ONE OF | A8 | E1 | F3 | ANY ONE |
| | MORE OF D1- | B4-B22 | | | | OF G3- |
| | D28 | | | | | G12 |
| 676 | ANY ONE OR | ANY ONE OF | A8 | ANY ONE OF | F1 | G1 |
| | MORE OF D1- | B4-B22 | | E2-E4 | | |
| | D28 | | | | | |
| 677 | ANY ONE OR | ANY ONE OF | A8 | ANY ONE OF | F1 | G2 |
| | MORE OF D1- | B4-B22 | | E2-E4 | | |
| | D28 | | | | | |
| 678 | ANY ONE OR | ANY ONE OF | A8 | ANY ONE OF | F1 | ANY ONE |
| | MORE OF D1- | B4-B22 | | E2-E4 | | OF G3- |
| | D28 | | | | | G12 |
| 679 | ANY ONE OR | ANY ONE OF | A8 | ANY ONE OF | F2 | G1 |
| | MORE OF D1- | B4-B22 | | E2-E4 | | |
| | D28 | | | | | |
| 680 | ANY ONE OR | ANY ONE OF | A8 | ANY ONE OF | F2 | G2 |
| | MORE OF D1- | B4-B22 | | E2-E4 | | |
| | D28 | | | | | |
| 681 | ANY ONE OR | ANY ONE OF | A8 | ANY ONE OF | F2 | ANY ONE |
| | MORE OF D1- | B4-B22 | | E2-E4 | | OF G3- |
| | D28 | | | | | G12 |
| 682 | ANY ONE OR | ANY ONE OF | A8 | ANY ONE OF | F3 | G1 |
| | MORE OF D1- | B4-B22 | | E2-E4 | | |
| | D28 | | | | | |
| 683 | ANY ONE OR | ANY ONE OF | A8 | ANY ONE OF | F3 | G2 |
| | MORE OF D1- | B4-B22 | | E2-E4 | | |
| | D28 | | | | | |
| 684 | ANY ONE OR | ANY ONE OF | A8 | ANY ONE OF | F3 | ANY ONE |
| | MORE OF D1- | B4-B22 | | E2-E4 | | OF G3- |
| | D28 | | | | | G12 |
| 685 | ANY ONE OR | ANY ONE OF | A9 | E1 | F1 | G1 |
| | MORE OF D1- | B4-B22 | | | | |
| | D28 | | | | | |
| 686 | ANY ONE OR | ANY ONE OF | A9 | E1 | F1 | G2 |
| | MORE OF D1- | B4-B22 | | | | |
| | D28 | | | | | |
| 687 | ANY ONE OR | ANY ONE OF | A9 | E1 | F1 | ANY ONE |
| | MORE OF D1- | B4-B22 | | | | OF G3- |
| | D28 | | | | | G12 |
| 688 | ANY ONE OR | ANY ONE OF | A9 | E1 | F2 | G1 |
| | MORE OF D1- | B4-B22 | | | | |
| | D28 | | | | | |
| 689 | ANY ONE OR | ANY ONE OF | A9 | E1 | F2 | G2 |
| | MORE OF D1- | B4-B22 | | | | |
| | D28 | | | | | |
| 690 | ANY ONE OR | ANY ONE OF | A9 | E1 | F2 | ANY ONE |
| | MORE OF D1- | B4-B22 | | | | OF G3- |
| | D28 | | | | | G12 |
| 691 | ANY ONE OR MORE | ANY ONE OF | A9 | E1 | F3 | G1 |
| | OF D1-D28 | B4-B22 | | | | |
| 692 | ANY ONE OR | ANY ONE OF | A9 | E1 | F3 | G2 |
| | MORE OF D1- | B4-B22 | | | | |
| | D28 | | | | | |
| 693 | ANY ONE OR | ANY ONE OF | A9 | E1 | F3 | ANY ONE |
| | MORE OF D1- | B4-B22 | | | | OF G3- |
| | D28 | | | | | G12 |
| 694 | ANY ONE OR | ANY ONE OF | A9 | ANY ONE OF | F1 | G1 |
| | MORE OF D1- | B4-B22 | | E2-E4 | | |
| | D28 | | | | | |
| 695 | ANY ONE OR | ANY ONE OF | A9 | ANY ONE OF | F1 | G2 |
| | MORE OF D1- | B4-B22 | | E2-E4 | | |
| | D28 | | | | | |
| 696 | ANY ONE OR | ANY ONE OF | A9 | ANY ONE OF | F1 | ANY ONE |
| | MORE OF D1- | B4-B22 | | E2-E4 | | OF G3- |
| | D28 | | | | | G12 |
| 697 | ANY ONE OR | ANY ONE OF | A9 | ANY ONE OF | F2 | G1 |
| | MORE OF D1- | B4-B22 | | E2-E4 | | |
| | D28 | | | | | |
| 698 | ANY ONE OR | ANY ONE OF | A9 | ANY ONE OF | F2 | G2 |
| | MORE OF D1- | B4-B22 | | E2-E4 | | |
| | D28 | | | | | |
| 699 | ANY ONE OR | ANY ONE OF | A9 | ANY ONE OF | F2 | ANY ONE |
| | MORE OF D1- | B4-B22 | | E2-E4 | | OF G3- |
| | D28 | | | | | G12 |
| 700 | ANY ONE OR | ANY ONE OF | A9 | ANY ONE OF | F3 | G1 |
| | MORE OF D1- | B4-B22 | | E2-E4 | | |
| | D28 | | | | | |
| 701 | ANY ONE OR | ANY ONE OF | A9 | ANY ONE OF | F3 | G2 |
| | MORE OF D1- | B4-B22 | | E2-E4 | | |
| | D28 | | | | | |
| 702 | ANY ONE OR | ANY ONE OF | A9 | ANY ONE OF | F3 | ANY ONE |
| | MORE OF D1- | B4-B22 | | E2-E4 | | OF G3- |
| | D28 | | | | | G12 |
| 703 | ANY ONE OR | ANY ONE OF | A10 | E1 | F1 | G1 |
| | MORE OF D1- | B4-B22 | | | | |
| | D28 | | | | | |
| 704 | ANY ONE OR | ANY ONE OF | A10 | E1 | F1 | G2 |
| | MORE OF D1- | B4-B22 | | | | |
| | D28 | | | | | |
| 705 | ANY ONE OR | ANY ONE OF | A10 | E1 | F1 | ANY ONE |
| | MORE OF D1- | B4-B22 | | | | OF G3- |
| | D28 | | | | | G12 |
| 706 | ANY ONE OR | ANY ONE OF | A10 | E1 | F2 | G1 |
| | MORE OF D1- | B4-B22 | | | | |
| | D28 | | | | | |
| 707 | ANY ONE OR | ANY ONE OF | A10 | E1 | F2 | G2 |
| | MORE OF D1- | B4-B22 | | | | |
| | D28 | | | | | |
| 708 | ANY ONE OR | ANY ONE OF | A10 | E1 | F2 | ANY ONE |
| | MORE OF D1- | B4-B22 | | | | OF G3- |
| | D28 | | | | | G12 |
| 709 | ANY ONE OR | ANY ONE OF | A10 | E1 | F3 | G1 |
| | MORE OF D1- | B4-B22 | | | | |
| | D28 | | | | | |
| 710 | ANY ONE OR | ANY ONE OF | A10 | E1 | F3 | G2 |
| | MORE OF D1- | B4-B22 | | | | |
| | D28 | | | | | |
| 711 | ANY ONE OR | ANY ONE OF | A10 | E1 | F3 | ANY ONE |
| | MORE OF D1- | B4-B22 | | | | OF G3- |
| | D28 | | | | | G12 |
| 712 | ANY ONE OR | ANY ONE OF | A10 | ANY ONE OF | F1 | G1 |
| | MORE OF D1- | B4-B22 | | E2-E4 | | |
| | D28 | | | | | |
| 713 | ANY ONE OR | ANY ONE OF | A10 | ANY ONE OF | F1 | G2 |
| | MORE OF D1- | B4-B22 | | E2-E4 | | |
| | D28 | | | | | |
| 714 | ANY ONE OR | ANY ONE OF | A10 | ANY ONE OF | F1 | ANY ONE |
| | MORE OF D1- | B4-B22 | | E2-E4 | | OF G3- |
| | D28 | | | | | G12 |
| 715 | ANY ONE OR | ANY ONE OF | A10 | ANY ONE OF | F2 | G1 |
| | MORE OF D1- | B4-B22 | | E2-E4 | | |
| | D28 | | | | | |
| 716 | ANY ONE OR | ANY ONE OF | A10 | ANY ONE OF | F2 | G2 |
| | MORE OF D1- | B4-B22 | | E2-E4 | | |
| | D28 | | | | | |
| 717 | ANY ONE OR | ANY ONE OF | A10 | ANY ONE OF | F2 | ANY ONE |
| | MORE OF D1- | B4-B22 | | E2-E4 | | OF G3- |
| | D28 | | | | | G12 |
| 718 | ANY ONE OR | ANY ONE OF | A10 | ANY ONE OF | F3 | G1 |
| | MORE OF D1- | B4-B22 | | E2-E4 | | |
| | D28 | | | | | |
| 719 | ANY ONE OR | ANY ONE OF | A10 | ANY ONE OF | F3 | G2 |
| | MORE OF D1- | B4-B22 | | E2-E4 | | |
| | D28 | | | | | |
| 720 | ANY ONE OR | ANY ONE OF | A10 | ANY ONE OF | F3 | ANY ONE |
| | MORE OF D1- | B4-B22 | | E2-E4 | | OF G3- |
| | D28 | | | | | G12 |

As disclosed herein, signaling and targeting components having paired multimerization domains (i.e., a multimerization domain pairs that dimerize/multimerize in the presence or absence of a bridging factor) can be combined to yield a functional engineered immune receptor. For example, a signaling component having an FRB (e.g., FRB or FRB*) multimerization domain can be combined/paired with a targeting component having an FKBP (e.g., FKBP or FKBP F36V) multimerization domain. As one example, any one of signaling constructs 1-81 are combined with any one of targeting components 37-72, 217-252, 397-432, and 577-612, to produce a novel engineered immune receptor. Other multimerization pairs include, but are not limited to, CH3_AB (S354C T366W) and CH3_IA (Y349C T366S L368A Y407V); CH3_DE (L351D L368E) and CH3_KK (L351K T366K); and CH3_GA (SEED 1) and CH3_AG (SEED 2).

In various embodiments, paired signaling and targeting components may be combined as a fusion polypeptide. Illustrative fusion polypeptides are shown in SEQ ID NOs: 139-170.

Additionally, any one of the engineered immune receptors disclosed herein can be combined with an engineered/exogenous antigen or lymphocyte receptor (e.g., TCR, CAR, CCR, or flip receptor).

One of skill in the art would understand that other combinations are possible, including combinations using other multimerization domains, polypeptide linkers, actuator domains, targeting domains, hinge domains, transmembrane domains, intracellular domains, and exogenous antigen receptors either known to or newly developed by the skilled artisan.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure

1.-271. (canceled)

272. A polypeptide complex, comprising:
(a) a signaling component comprising
(i) a first multimerization domain comprising an FRB polypeptide or a FKBP polypeptide,
(ii) a first polypeptide linker, and
(iii) a CD3ε polypeptide; and
(b) a targeting component comprising
(i) an anti-CLL1 scFv or single domain antibody (sdAb),
(ii) an anti-CD33 scFv or single domain antibody (sdAb),
(iii) a second polypeptide linker,
(iv) a second multimerization domain comprising an FRB polypeptide or a FKBP polypeptide,
(v) a CD4 hinge polypeptide,
(vi) a CD4 transmembrane polypeptide, and
(vii) a truncated CD4 intracellular polypeptide.

273. The polypeptide complex of claim 272, wherein the targeting component does not comprise a functional intracellular domain or costimulatory domain having signaling capabilities.

274. The polypeptide complex of claim 272, wherein the CD4 hinge polypeptide comprises an amino acid sequence having at least 90% identity to SEQ ID NO: 41, or wherein the CD4 hinge polypeptide comprises an amino acid sequence as set forth in SEQ ID NO: 41.

275. The polypeptide complex of claim 272,
wherein the CD3ε polypeptide comprises an amino acid sequence having at least 90% identity to SEQ ID NO: 32,
wherein the CD3ε polypeptide comprises an amino acid sequence as set forth in SEQ ID NO: 32, or
wherein the CD3ε polypeptide comprises both extracellular and intracellular portions.

276. The polypeptide complex of claim 272, wherein the FRB polypeptide and the FKBP polypeptide each localize extracellularly when the signaling and targeting components are expressed.

277. The polypeptide complex of claim 272, wherein:
(a) the first and second multimerization domains are different;
(b) the first multimerization domain comprises an FRB polypeptide, and the second multimerization domain comprises an FKBP12 polypeptide;
(c) the first multimerization domain comprises an FKBP12 polypeptide, and the second multimerization domain comprises an FRB polypeptide;
(d) the FRB polypeptide is an FRB T2098L variant;
(e) the FRB polypeptide comprises an amino acid sequence having at least 90% identity to, or comprising a sequence set forth as SEQ ID NO: 1;
(f) the FRB polypeptide comprises an amino acid sequence having at least 90% identity to, or comprising a sequence set forth as SEQ ID NO: 2;
(g) the FKBP12 polypeptide comprises an amino acid sequence having at least 90% identity to, or comprising a sequence set forth as SEQ ID NO: 3; and/or
(h) the FKBP12 polypeptide comprises an amino acid sequence having at least 90% identity to, or comprising a sequence set forth as SEQ ID NO: 4.

278. The polypeptide complex of claim 272, wherein the first, second, or third polypeptide linker is selected from the group consisting of: GG, GS, SG, SS, GSS, SSG, GSG, SGS, SGG, G4S, 2×G4S, 3×G4S, 4×G4S, 5×G4S, and any combination thereof.

279. The polypeptide complex of claim 272, wherein the first polypeptide linker is a 3×G4S linker, the second polypeptide linker is a G4S linker, and/or the third polypeptide linker is a G4S linker.

280. The polypeptide complex of claim 272, wherein the CD4 transmembrane polypeptide comprises an amino acid sequence having at least 90%, 95%, 96%, 97%, 98%, or 99% identity to SEQ ID NO: 45, or comprises a sequence set forth as SEQ ID NO: 45.

281. The polypeptide complex of claim 272, wherein the truncated intracellular CD4 polypeptide comprises an amino acid sequence as set forth in SEQ ID NO: 48 or SEQ ID NO: 49.

282. The polypeptide complex of claim 272, wherein the anti-CLL1 and anti-CD33 antibodies are each a sdAb, wherein each sdAb is a camelid VHH.

283. The polypeptide complex of claim 272, wherein the scFv or sdAb is human or humanized.

284. The polypeptide complex of claim 272, wherein the anti-CLL1 scFv or sdAb comprises CDR1, CDR2, and CDR3 amino acid sequences as set forth in SEQ ID NOs: 92, 93, and 94, respectively.

285. The polypeptide complex of claim 272, wherein the anti-CLL1 scFv or sdAb comprises a sequence having at least 90% identity to SEQ ID NO: 75, or comprises a sequence set forth as SEQ ID NO: 75.

286. The polypeptide complex of claim 272, wherein the anti-CD33 scFv or sdAb comprises CDR1, CDR2, and CDR3 amino acid sequences as set forth in SEQ ID NOs: 89, 90, and 91, respectively.

287. The polypeptide complex of claim 272, wherein the anti-CD33 scFv or sdAb comprises a sequence having at least 90% identity to SEQ ID NO: 72, or comprises a sequence set forth as SEQ ID NO: 72.

288. The polypeptide complex of claim 272, wherein the signaling component further comprises a signal sequence, wherein the signal sequence is a CD8 signal sequence comprising an amino acid sequence having at least 90% identity to SEQ ID NO: 96, or comprising a sequence set forth as SEQ ID NO: 96, and/or
wherein the targeting component further comprises a signal sequence, wherein the signal sequence is an IgK signal sequence comprising an amino acid sequence having at least 90% identity to SEQ ID NO: 95, or comprising a sequence set forth as SEQ ID NO: 95.

289. The polypeptide complex of claim 272, wherein the signaling component comprises a sequence having at least 90% identity to SEQ ID NO: 111, or comprises a sequence set forth as SEQ ID NO: 111.

290. The polypeptide complex of claim 272, wherein the targeting component comprises a sequence having at least 90% identity to SEQ ID NO: 122, or comprises a sequence set forth as SEQ ID NO: 122.

291. A fusion polypeptide comprising the polypeptide complex signaling and targeting components of claim 272.

292. The fusion polypeptide of claim 291, wherein the fusion polypeptide comprises a sequence having at least 90% identity to SEQ ID NO: 151, or comprises a sequence set forth as SEQ ID NO: 151.

293. A nucleic acid molecule that encodes both the signaling component and the targeting component of the polypeptide complex of claim 272.

294. A vector comprising the nucleic acid molecule of claim 293.

295. A cell expressing the polypeptide complex of claim 272.

296. The cell of claim 295, wherein the cell further expresses an exogenous TCR, CAR, CCR, flip receptor, zetakine, immune cell engager, or BiTE.

297. The cell of claim 295, wherein:
(a) the cell is a hematopoietic cell;
(b) the cell is a T cell, an αβ-T cell, or a γδ-T cell;
(c) the cell is a CD3+, CD4+, and/or CD8+ cell;
(d) the cell is an immune effector cell;
(e) the cell is a cytotoxic T lymphocyte (CTL), a tumor infiltrating lymphocyte (TIL), or a helper T cell;
(f) the cell is a natural killer (NK) cell or natural killer T (NKT) cell;
(g) the source of the cell is peripheral blood mononuclear cells, bone marrow, lymph nodes tissue, cord blood, thymus issue, tissue from a site of infection, ascites, pleural effusion, spleen tissue, or tumors;
(h) the cell is an isolated cell;
(i) the cell is obtained from a subject; and/or
(j) the cell is a human cell.

298. A composition comprising a physiologically acceptable carrier and a cell according to claim 295.

299. A method of treating a hematological malignancy comprising administering to the subject an effective amount of the composition of claim 298.

300. The method of claim 299, wherein the hematological malignancy is selected from the group consisting of: a leukemia, lymphoma, or multiple myeloma; or wherein the hematological malignancy is acute myelogenous leukemia (AML).

301. A polypeptide complex, comprising:
(a) a signaling component comprising
(i) a first multimerization domain comprising an FRB polypeptide or a FKBP polypeptide,
(ii) a first polypeptide linker, and
(iii) a CD3ε polypeptide; and
(b) a targeting component comprising
(i) a means for binding human CLL1 protein,
(ii) a means for binding a human CD33 protein,
(iii) a second polypeptide linker,
(iv) a second multimerization domain comprising an FRB polypeptide or a FKBP polypeptide,
(v) a CD4 hinge polypeptide,
(vi) a CD4 transmembrane polypeptide, and
(vii) a truncated CD4 intracellular polypeptide.

302. A fusion polypeptide comprising the polypeptide complex signaling and targeting components of claim 301.

303. A nucleic acid molecule that encodes both the signaling component and the targeting component of the polypeptide complex of claim 301.

304. A vector comprising the nucleic acid molecule of claim 303.

305. A cell comprising the polypeptide complex of claim 301.

306. A composition comprising a physiologically acceptable carrier and a cell according to claim 305.

307. A method of treating, preventing, or ameliorating at least one symptom of a cancer, infectious disease, autoimmune disease, inflammatory disease, immunodeficiency, or condition associated therewith in a subject, the method comprising administering to the subject an effective amount of the composition of claim 306.
